(19) United States
(12) Patent Application Publication
(10) Pub. No.: US 2026/0115185 A1
(43) Pub. Date: Apr. 30, 2026

(54) QUINOLINE DERIVATIVES WHICH ACT AS KAPPA-OPIOID RECEPTOR ANTAGONISTS

(71) Applicant: The Scripps Research Institute, La Jolla, CA (US)

(72) Inventors: Edward ROBERTS, Fallbrook, CA (US); Miguel Angel GUERRERO MORALES, San Diego, CA (US); Hugh ROSEN, Rancho Santa Fe, CA (US)

(21) Appl. No.: 19/157,124

(22) PCT Filed: Feb. 16, 2024
(86) PCT No.: PCT/US2024/016230

§ 371 Date: Aug. 15, 2025

Related U.S. Application Data
(60) Provisional application No. 63/485,712, filed on Feb. 17, 2023.
(60) Provisional application No. 63/520,478, filed on Aug. 18, 2023.

(51) Int. Cl. A61K 31/496; A61K 31/4709; A61K 31/5377; C07D 401/14; C07D 405/14; C07D 413/14; C07D 471/04

(52) U.S. Cl. CPC A61K 31/496; A61K 31/4709; A61K 31/5377; C07D 401/14; C07D 405/14; C07D 413/14; C07D 471/04

(57) ABSTRACT

Disclosed herein are kappa-opioid receptor (KOR) antagonist compounds of Formula (I), Formula (II), and their pharmaceutically acceptable salts, and pharmaceutical compositions thereof. The compounds are useful in methods of treatment of various diseases and disorders for which KOR antagonism is indicated, including substance abuse disorders, depression, anxiety, and other psychiatric conditions.

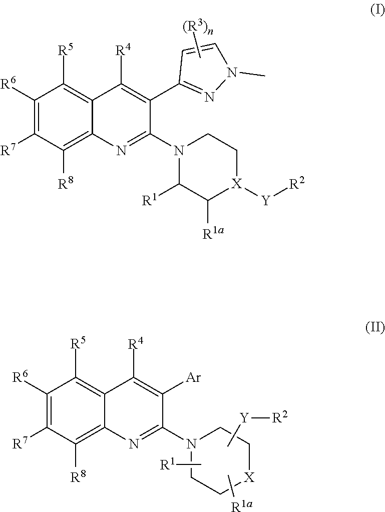

This application claims the benefit of priority to U.S. Provisional Patent Application No. 63/485,712, filed Feb. 17, 2023 and U.S. Provisional Patent Application No. 63/520,478, filed Aug. 18, 2023, which applications are incorporated in their entirety as if fully set forth herein.

### BACKGROUND

The kappa-opioid receptor (KOR) is a member of the opioid receptor family that binds the opioid peptide dynorphin as the primary endogenous ligand. The KOR has a wide, yet distinct, distribution in the brain, spinal cord, and in peripheral tissues, and particularly in brain areas implicated in reward, cognitive function, and stress responsiveness. Evidence indicates that dynorphins are elevated under painful and stressful conditions, and that KOR disruption produces anti-stress effects. Such findings have led to development of KOR antagonists for treatment of depressive, anxiety, addictive disorders, as well as other psychiatric conditions associated with stress [M. Urbano et al., Bioorganic & Medicinal Chemistry Letters, 24:2021-2032, 2014 (“Urbano 2014”); Jacobson et al., Annu. Rev. Pharmacol. Toxicol., 60:615-636 (2020); see also: Handbook in Experimental Pharmacology, 271: Eds. Lee-Yuan Liu-Chen Saadet Inan (2022)].

Pharmacological studies with prototypical KOR antagonists (i.e., the morphinan-derived ligands nor-BNI and GNTI, and the non-morphinan JDTic) have confirmed the therapeutic potential of the KOR/dynorphin system [Urbano 2014]. However, such prototypical KOR antagonists display delayed onset of action in the range of hours to days, followed by antagonism effects lasting weeks at minimally effective doses. Furthermore, such compounds showed poor blood-brain barrier penetration. For these reasons, more recent research has focused on the development of short(er)-acting KOR antagonists with improved pharmacokinetics.

The mechanisms by which KOR antagonists provide therapeutic action are generally understood. Both direct modulation of KOR and the modulation of down-stream signaling pathways modulated by dynorphin-KOR signaling can contribute to therapeutic efficacy. Indeed, KOR antagonists have been extensively studied precisely because they are known to block a prominent stress-induced neuroadaptation; namely, elevated expression of dynorphin in the nucleus accumbens (NAc). The NAc is an element of the mesolimbic system which plays a role in motivation and the pathology of psychiatric illness. Stress, in addition to repeated exposure to drugs of abuse, triggers a complex sequence of intracellular events involving the transcription factor CREB, a cAMP response element binding protein, in the NAc. KOR antagonists mitigate depressive-like signs that are produced though CREB-mediated increases in the expression of dynorphin [W. A. Carlezon et al., Depression and Anxiety, 33:895-906, 2016]. According to the model set forth by Carlezon et al., stress activates CREB in the NAc, which leads to an increase in dynorphin expression. Increased dynorphin, in turn, promotes activation of KORs. KORs are expressed on the cell bodies and terminals of mesocorticolimbic dopamine (DA) neurons, and activation of KORs inhibit DA release. Thus, treatment with a KOR antagonist blocks the action of dynorphin, restoring DA function, and thereby providing antidepressant- and anxiolytic-like effects.

The neuropeptides oxytocin and vasopressin also function in pathways that are operative in neuropsychiatric disorders including depression, anxiety, autism, schizophrenia, PTSD, addiction, ADHD, etc. [Cid-Jofre et al., Int. J. Mol. Sci., 22:12077, (2021)]. These are amplifying neuropeptide pathways, down-stream of dynorphin responses, and they can be modulated by KOR antagonists allowing multi-step modulation of the indicated psychiatric pathologies. Indeed, the multistep interdiction in amplifying pathways is a well-established principle in disease-modifying therapeutics.

Mu and kappa opioid binding sites are found in the pituitary, which is significant for oxytocin and vasopressin release [Jordan et a., J. Neuroendocrinol., 8: 883-887, (1996); Shuster et al., Neuroscience, 96(2), 373-383, (2000): Morris et al., J Clin. Pharmacol., 50:1112-1117, (2010)]. Secretion of oxytocin is inhibited by both mu and kappa agonists centrally and by kappa agonists directly by activation of kappa receptors [Lutz-Bucher & Koch, Euro J Pharmacol., 66: 375-378. (1980)]. Accordingly, kappa and mu opioid antagonists both caused increases in oxytocin levels; however, only kappa antagonists enhanced both oxytocin and vasopressin levels when given by intracerebroventricular (icv) administration [Van de Heijning et al., Eur J Pharmacol., 197:175-180, (1991), ibid, idem, 209:199-206. (1991)]. Additionally, the endogenous kappa opioid receptor agonist dynorphin modulates serotonin (5-HT) release and, for example, it exerts effects on social deficits in rodents during substance withdrawal: as manifested in humans, these effects can lead to relapse [Pomrenze et al., Neuron 110:4125-4143, (2022)].

Physiologically, dynorphin/KOR signaling promotes REM sleep. In addition, while KOR antagonists do not promote somnolence in the absence of pain, they do normalize disrupted sleep in chronic pain, revealing a pathophysiological role of KOR signaling that is selectively recruited to promote vigilance and increased survival. Notably, while this mechanism is likely beneficial in the short-term, disruption of the homeostatic need for sleep over longer periods may become maladaptive resulting in sustained pain chronicity. A novel approach for treatment of chronic pain may thus result from normalization of chronic pain-related sleep disruption by KOR antagonism [Ito, et al., Brain: 00; 1-14 (2022)]. Furthermore, sleep disturbances not only can result from chronic pain, but they are also common symptoms of Major Depressive Disorder (MDD) and they are significant adverse effects of most existing anti-depressant drug classes, most especially selective serotonin reuptake inhibitors (SSRIs) and serotonin-norepinephrine reuptake inhibitors (SNRIs) [E. C. Settle, J. Clin. Psychiatry 59: 25-30 (1998)]. Accordingly, that KOR antagonism can normalize sleep disruption without somnolence is a broad advantage in therapeutic intervention for MDD and in clinical settings that are characterized by sleep disturbance as an adverse effect of drug therapies.

The mechanisms of action, as well as considerable development and testing of KOR antagonists to date, including recent clinical study results (e.g., Aticaprant and ALKS-5461), provide strong evidence that KOR antagonists offer therapeutic effects in humans suffering from a wide range of disorders, including mood disorders, anxiety disorders and substance abuse disorders as defined, for example, in the Diagnostic and Statistical Manual of Mental Disorder (DSM). The Research Domain Criteria (RD C) project provides an additional framework for classifying psychopathology disorders: RD C aims to classify such disorders based on dimensions of observable behavior and neurobiological dimensions. In this context, KOR antagonists have therapeutic effect on at least two types of RD C-defined domains; namely, those related to reward and those related to adverse effects of stress. Within these domains, the use of KOR antagonists is recognized for the treatment of anhedonia (“positive valence system”) and for blocking the adverse effects of stress (“negative valence system”).

Offering benefits from advances made in this field, KOR antagonists are acknowledged for their utility in treating major depression and substance abuse related disorders, particularly in the context of rapidly acting treatments that avoid the drawbacks associated with the prototypical KOR antagonists discussed above. Additional advances have shown that KOR antagonists can be particularly useful for the treatment of stress-mediated symptoms, as well as for treating social anxiety disorder and phobias. Prophylactic therapy has also been suggested to prevent adverse conditions arising from stress, and in this regard KOR antagonism has been proposed as a preventative treatment of PTSD in individuals at risk of the same. Other therapeutic applications of KOR antagonism include the treatment of impairment in a reward-related function because it frequently occurs in patients with mood and anxiety spectrum disorders, and that also may present with other types of conditions such as schizophrenia or a schizoaffective disorder.

KOR antagonism is an established therapeutic pathway for the treatment of a wide variety of disorders and conditions. Despite the advances made in the art, there remains a need for new and improved KOR antagonists to treat a variety of conditions, including substance abuse disorders, major depression, anhedonia, and stress-related symptoms.

### SUMMARY

The present disclosure addresses this need and others by providing, in various embodiments, a compound of Formula (I) or a pharmaceutically acceptable salt thereof:

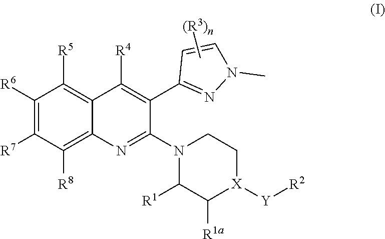

In Formula (I), according to an embodiment, X is CH and Y is NH. In another embodiment, X is N and Y is —C(═N—CN)NR⁹. In still another embodiment, X is NH and —Y—R² is null, i.e., absent.

In an embodiment, R¹ and R^{1a} are independently selected from the group consisting of H, C₁-C₆-alkyl, and halo. In another embodiment, R¹ and R^{1a}, together with the carbon atoms to which they are bound, form a fused C₃-C₈-cycloalkyl or a 3- to 6-membered heterocycloalkyl (wherein 1-4 ring members are independently selected from N, O, and S).

R² is selected from the group consisting of H, C₁-C₆-alkyl, C₃-C₈-cycloalkyl, —(C₁-C₆-alkyl)C₃-C₈-cycloalkyl, and 3- to 6-membered heterocycloalkyl (wherein 1-4 ring members are independently selected from N, O, and S).

R⁹ is selected from the group consisting of H, C₁-C₆-alkyl, C₁-C₆-haloalkyl, C₃-C₈-cycloalkyl, and —(C₁-C₆-alkyl)(C₃-C₈-cycloalkyl).

R³ in each instance is C₁-C₆-alkyl.

Subscript n is 0, 1, or 2.

The substituents R⁴, R⁵, R⁶, R⁷, and R⁸ are independently selected from the group consisting of H, CN, OH, halo, NRR′, C₁-C₆-alkyl, C₂-C₆-alkenyl, C₂-C₆-alkynyl, C₁-C₆-haloalkyl, O(C₁-C₆-alkyl), O(C₁-C₆-haloalkyl), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(RR′), —CONRR′, —COOR′, —NRCOOR′, —(C₁-C₆-alkyl)C(O)N(RR′), C₆-C₁₀-aryl, C₃-C₈-cycloalkyl, O(C₃-C₈-cycloalkyl), —(C₁-C₆-alkyl)(C₆-C₁₀-aryl), —(C₁-C₆-alkyl)(C₃-C₈-cycloalkyl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), 5- to 10-membered heteroaryl (wherein 1-4 heteroaryl members are independently selected from N, O, and S), —(C₁-C₆-alkyl)(5- to 10-membered heteroaryl (wherein 1-4 heteroaryl members are independently selected from N, O, and S)).

R and R′ are independently selected from H and C₁-C₆-alkyl.

In Formula (I), any alkyl, aryl, cycloalkyl, heterocycloalkyl, and heteroaryl in R¹, R^{1a}, R², R³, R⁴, R⁵, R⁶, R⁷, and R⁸ is optionally substituted with 1 to 6 substituents independently selected from the group consisting of C₁-C₆-alkyl, halo, NO₂, OH, CN, and C₁-C₆-haloalkyl.

In additional embodiments, the present disclosure provides compound of Formula (II) or a pharmaceutically acceptable salt thereof:

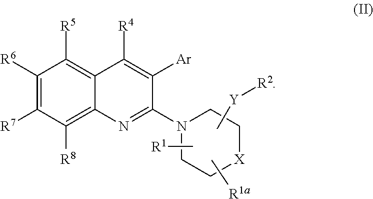

In Formula (II), Ar is a 5- or 6-membered heteroaryl (wherein 1-4 heteroaryl members are independently selected from N, O, and S) substituted with (R³).

In some embodiments, X is CH and Y is NH. In other embodiments, X is N and Y is selected from a bond, C(O), and —C(═N—CN)NR⁹. In still further embodiments, X is NH and —Y—R² is null (i.e., absent).

In embodiments, R¹ and R^{1a} are independently selected from the group consisting of H, C₁-C₆-alkyl, and halo.

In other embodiments. R¹ and R^{1a}, together with the carbon atoms to which they are bound, form a fused C₃-C₈-cycloalkyl or an optionally substituted 3- to 6-membered heterocycloalkyl (wherein 1-4 ring members are independently selected from N, O, and S).

In Formula (II), when X is CH, then: (i) at least one of R¹ and R^{1a} is not H and Ar is other than oxadiazolyl, thiadiazolyl, or triazolyl; or (ii) optionally R¹ or R^{1a}, together with Y and the carbon atoms to which they are bound, form a fused 5- to 6-membered heterocycloalkyl.

R² is selected from the group consisting of H, C₁-C₆-alkyl, C₃-C₈-cycloalkyl, —(C₁-C₆-alkyl)C₃-C₈-cycloalkyl, 3- to 6-membered heterocycloalkyl (wherein 1-4 ring members are independently selected from N, O, and S), and —(C₁-C₆-alkyl)(3- to 6-membered heterocycloalkyl (wherein 1-4 ring members are independently selected from N, O, and S)).

R⁹ is selected from the group consisting of H, C₁-C₆-alkyl, C₁-C₆-haloalkyl, C₃-C₈-cycloalkyl, and —(C₁-C₆-alkyl)(C₃-C₈-cycloalkyl).

R³ in each instance is independently C₁-C₆-alkyl or C₁-C₆-haloalkyl.

Subscript n is 0, 1, or 2.

R⁴, R⁵, R⁶, R⁷, and R⁸ are independently selected from the group consisting of H, CN, OH, halo, NRR′, C₁-C₆-alkyl. C₂-C₆-alkenyl, C₂-C₆-alkynyl, C₁-C₆-haloalkyl, O(C₁-C₆-alkyl), O(C₁-C₆-haloalkyl), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(RR′), —CONRR′, —COOR′, —NRCOOR′, —(C₁-C₆-alkyl)C(O)N(RR′), C₆-C₁₀-aryl, C₃-C₈-cycloalkyl. O(C₃-C₈-cycloalkyl), —(C₁-C₆-alkyl)(C₆-C₁₀-aryl), —(C₁-C₆-alkyl)(C₃-C₈-cycloalkyl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), 5- to 10-membered heteroaryl (wherein 1-4 heteroaryl members are independently selected from N, O, and S). —(C₁-C₆-alkyl)(5- to 10-membered heteroaryl (wherein 1-4 heteroaryl members are independently selected from N, O, and S)).

R and R′ are independently selected from H and C₁-C₆-alkyl.

In Formula (II), any alkyl, aryl, cycloalkyl, heterocycloalkyl, and heteroaryl in R¹, R^{1a}, R², R³, R⁴, R⁵, R⁶, R⁷, and R⁸ is optionally substituted with 1 to 6 substituents independently selected from the group consisting of C₁-C₆-alkyl, halo, NRR′, NO₂, OR, CN, and C₁-C₆-haloalkyl.

Notwithstanding the provisions for Formula (11), it should be understood that Formula (II) does not include the following compound:

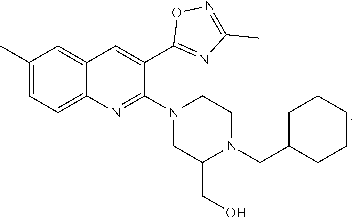 [SMILES: Cc1ccc2nc(N3CCN(CC4CCCCC4)C(CO)C3)c(-c3nc(C)no3)cc2c1]

In additional embodiments, the present disclosure provides a pharmaceutical composition comprising a compound or pharmaceutically acceptable salt thereof as disclosed herein and a pharmaceutically acceptable carrier.

In still further embodiments, the present disclosure provides a method for treating a disorder in a subject suffering therefrom, wherein the disorder is one for which antagonism of kappa-opioid receptor (KOR) is therapeutically indicated. The method comprises administering to the subject a compound or pharmaceutically acceptable salt thereof as disclosed herein.

In other embodiments, the present disclosure provides a method for treating a disorder in a subject suffering therefrom, wherein the disorder is selected from those disclosed herein, such as substance abuse or addiction, a psychiatric disorder, obesity and eating disorders, migraine, postnatal depression, a neurodegenerative disease or disorder, epilepsy, status epilepticus, and seizure. The method comprises administering to the subject a compound or pharmaceutically acceptable salt thereof as disclosed herein.

In an embodiment, the present disclosure provides a compound of Formula (1) or a pharmaceutically acceptable salt thereof for treating a disorder as disclosed herein in a subject suffering therefrom. In another embodiment, there is provided a compound of Formula (I) or a pharmaceutically acceptable salt thereof for use in the manufacture of a medicament for treating a disorder as disclosed herein.

### DETAILED DESCRIPTION

The present disclosure relates in part to compounds that antagonize the kappa opioid receptor (KOR). One advantage of the compounds resides in their high potencies, particularly in combination with their selectivity for KOR over mu-opioid receptor (MOR).

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂ CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂C H(CH₂CH₃)₂, —CH₂CH₂C(CH₃)₃. —CH₂CH₂C(CH₂CH₃)₃, —CH(CH₃)CH₂CH(CH₃)₂, —CH(CH₃) CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein, such as halogen(s), for example.

Each of the terms “halogen,” “halide,” and “halo” refers to —F or fluoro. —C₁ or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, I-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. The ring heteroatoms can also include oxidized S or N, such as sulfinyl, sulfonyl, and N-oxides of a tertiary ring nitrogen. A heterocycloalkyl can be fused to another ring system, such as with an aryl or heteroaryl of 5-6 ring members. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

The term “nitrile” or “cyano” can be used interchangeably and refers to a —CN group.

As used herein and in the appended claims, the singular forms “a,” “and,” and “the” include plural referents unless the context clearly dictates otherwise. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, the term “isotopologue” is an isotopically enriched compound. As used herein, and unless otherwise indicated, the term “isotopically enriched” refers to an atom having an isotopic composition other than the naturally abundant isotopic composition of that atom. “Isotopically enriched” can also refer to a compound containing at least one atom having an isotopic composition other than the natural isotopic composition of that atom. In an isotopologue, “isotopic enrichment” refers to the percentage of incorporation of an amount of a specific isotope of a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

Thus, as used herein, and unless otherwise indicated, the term “isotopic enrichment factor” refers to the ratio between the isotopic composition and the natural isotopic composition of a specified isotope.

With regard to the compounds provided herein, when a particular atom's position is designated as having deuterium or “D,” it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom. The isotopic enrichment and isotopic enrichment factor of the compounds provided herein can be determined using conventional analytical methods known to one of ordinary skill in the art, including mass spectrometry and nuclear magnetic resonance spectroscopy.

As used herein, and unless otherwise specified to the contrary, the term “compound” is inclusive in that it encompasses a compound or a pharmaceutically acceptable salt, stereoisomer, isotopologue, and/or tautomer thereof. Thus, for instance, a compound includes a pharmaceutically acceptable salt of a tautomer of the compound. Similarly, a compound of includes a pharmaceutically acceptable salt of an isotopologue of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing or slowing the spread, progression, or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Compounds

In various embodiments, the present disclosure provides a compound of Formula (I) or a pharmaceutically acceptable salt thereof:

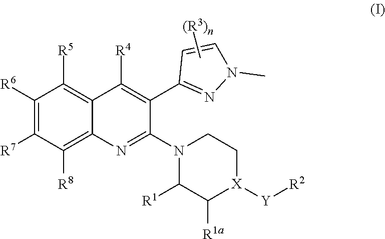

In Formula (I), according to an embodiment, X is CH and Y is NH. In another embodiment, X is N and Y is —C(═N—CN)NR⁹. In still another embodiment, X is NH and —Y—R² is null, i.e., absent.

In an embodiment, R¹ and R^{1a} are independently selected from the group consisting of H, C₁-C₆-alkyl, and halo. In another embodiment, R¹ and R^{1a}, together with the carbon atoms to which they are bound, form a fused C₃-C₈-cycloalkyl or a 3- to 6-membered heterocycloalkyl (wherein 1-4 ring members are independently selected from N, O, and S:

R² is selected from the group consisting of H, C₁-C₆-alkyl, C₃-C₈-cycloalkyl, —(C₁-C₆-alkyl)C₃-C₈-cycloalkyl, and 3- to 6-membered heterocycloalkyl (wherein 1-4 ring members are independently selected from N, O, and S).

R⁹ is selected from the group consisting of H, C₁-C₆-alkyl, C₁-C₆-haloalkyl, C₃-C₈-cycloalkyl, and —(C₁-C₆-alkyl)(C₃-C₈-cycloalkyl).

R³ in each instance is C₁-C₆-alkyl.

Subscript n is 0, 1, or 2.

The substituents R⁴, R⁵, R⁶, R⁷, and R⁸ are independently selected from the group consisting of H, CN, OH, halo, NRR′, C₁-C₆-alkyl, C₂-C₆-alkenyl, C₂-C₆-alkynyl, C₁-C₆-haloalkyl, O(C₁-C₆-alkyl), O(C₁-C₆-haloalkyl), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(RR′), —CONRR′, —COOR′, —NRCOOR′, —(C₁-C₆-alkyl)C(O)N(RR′), C₆-C₁₀-aryl, C₃-C₈-cycloalkyl, O(C₃-C₈-cycloalkyl), —(C₁-C₆-alkyl)(C₆-C₁₀-aryl), —(C₁-C₆-alkyl)(C₃-C₈-cycloalkyl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), 5- to 10-membered heteroaryl (wherein 1-4 heteroaryl members are independently selected from N, O, and S), —(C₁-C₆-alkyl)(5- to 10-membered heteroaryl (wherein 1-4 heteroaryl members are independently selected from N, O, and S)).

R and R′ are independently selected from H and C₁-C₆-alkyl.

In Formula (I), any alkyl, aryl, cycloalkyl, heterocycloalkyl, and heteroaryl in R¹, R^{1a}, R², R³, R⁴. R, R¹. R⁷, and R⁸ is optionally substituted with 1 to 6 substituents independently selected from the group consisting of C₁-C₆-alkyl, halo, NO₂, OH, CN, and C₁-C₆-haloalkyl.

In additional embodiments, the present disclosure provides compound of Formula (II) or a pharmaceutically acceptable salt thereof:

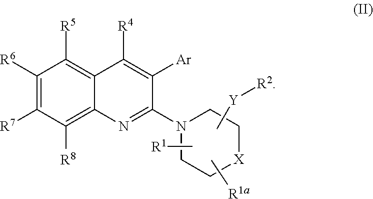

In Formula (11), Ar is a 5- or 6-membered heteroaryl (wherein 1-4 heteroaryl members are independently selected from N, O, and S) substituted with (R³)ₙ.

In some embodiments. X is CH and Y is NH. In other embodiments, X is N and Y is selected from a bond, C(O), and —C(═N—CN)NR⁹. In still further embodiments, X is NH and —Y—R² is null (i.e., absent).

In embodiments, R¹ and R^{1a} are independently selected from the group consisting of H, C₁-C₆-alkyl, and halo.

In other embodiments, R¹ and R^{1a}, together with the carbon atoms to which they are bound, form a fused C₃-C₈-cycloalkyl or an optionally substituted 3- to 6-membered heterocycloalkyl (wherein 1-4 ring members are independently selected from N, O, and S).

In Formula (11), when X is CH, then:
(i) at least one of R¹ and R^{1a} is not H and Ar is other than oxadiazolyl, thiadiazolyl, or triazolyl; or
(ii) optionally R¹ or R^{1a}, together with Y and the carbon atoms to which they are bound, form a fused 5- to 6-membered heterocycloalkyl.

R² is selected from the group consisting of H, C₁-C₆-alkyl, C₃-C₈-cycloalkyl, —(C₁-C₆-alkyl)C₃-C₈-cycloalkyl, 3- to 6-membered heterocycloalkyl (wherein 1-4 ring members are independently selected from N, O, and S), and —(C₁-C₆-alkyl)(3- to 6-membered heterocycloalkyl (wherein 1-4 ring members are independently selected from N, O, and S)).

R⁹ is selected from the group consisting of H, C₁-C₆-alkyl, C₁-C₆-haloalkyl, C₃-C₈-cycloalkyl, and —(C₁-C₆-alkyl)(C₃-C₈-cycloalkyl).

R³ in each instance is independently C₁-C₆-alkyl or C₁-C₆-haloalkyl.

Subscript n is 0, 1, or 2.

R, R⁵, R¹. R⁷, and R¹ are independently selected from the group consisting of H, CN, OH, halo, NRR′, C₁-C₆-alkyl, C₂-C₆-alkenyl, C₂-C₆-alkynyl, C₁-C₆-haloalkyl, O(C₁-C₆-alkyl), O(C₁-C₆-haloalkyl), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(RR′), —CONRR′, —COOR′, —NRCOOR′, —(C₁-C₆-alkyl)C(O)N(RR′), C₆-C₁₀-aryl, C₃-C₈-cycloalkyl, O(C₃-C₈-cycloalkyl), —(C₁-C₆-alkyl)(C₆-C₁₀-aryl), —(C₁-C₆-alkyl)(C₃-C₈-cycloalkyl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), 5- to 10-membered heteroaryl (wherein 1-4 heteroaryl members are independently selected from N, O, and S), —(C₁-C₆-alkyl)(5- to 10-membered heteroaryl (wherein 1-4 heteroaryl members are independently selected from N, O, and S)).

R and R′ are independently selected from H and C₁-C₆-alkyl.

In Formula (II), any alkyl, aryl, cycloalkyl, heterocycloalkyl, and heteroaryl in R¹, R^{a1}, R², R³, R⁴, R⁵, R⁴, R⁷, and R is optionally substituted with 1 to 6 substituents independently selected from the group consisting of C₁-C₆-alkyl, halo, NRR′, NO₂, OR, CN, and C₁-C₆-haloalkyl.

Notwithstanding the provisions for Formula (II), it should be understood that Formula (II) does not include the following compound;

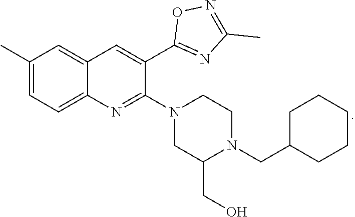 [SMILES: Cc1ccc2nc(N3CCN(CC4CCCCC4)C(CO)C3)c(-c3nc(C)no3)cc2c1]

In some embodiments, the compound or pharmaceutically acceptable salt thereof is of Formula (11A), wherein each substituent definition is as for Formula (II) disclosed herein:

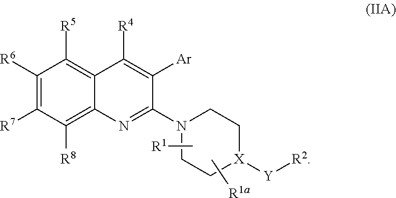

In various embodiments, Ar is a 5-membered heteroaryl (wherein 1-4 heteroaryl members are independently selected from N, O, and S). In illustrative embodiments, Ar is selected from the group consisting of pyrazolyl, imidazolyl, oxazolyl, isoxazolyl, oxadiazolyl, isoxadiazolyl, thiazolyl, isothiazolyl, and thiadiazolyl. In specific embodiments, Ar is selected from the group consisting of pyrazolyl, oxazolyl, and isoxazolyl.

In additional embodiments, X is N and Y is a bond.

In one embodiment, X is CH and Y is NH. In another embodiment, X is N and Y is —C(═N—CN)NR⁹. In an exemplary embodiment, R¹ is H.

In some embodiments, R² is an optionally substituted 3- to 6-membered heterocycloalkyl (wherein 1 ring member is O). For example, in various embodiments. R² is selected from the group consisting of optionally substituted:

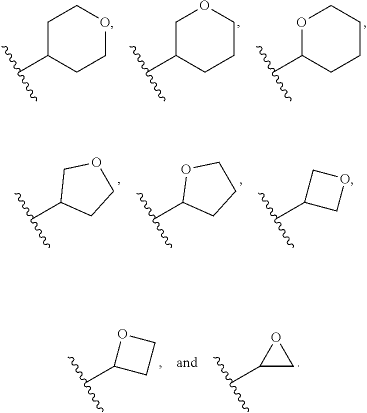

In an illustrative embodiment. R² is

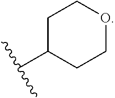 [SMILES: *C1CCOCC1]

In additional embodiments, R² is substituted by 1 to 3 substituents selected from halo and OH, and combinations thereof. In some embodiments, R² is substituted only with halo, such as 1, 2, or 3 halo. For example, in an embodiment, halo is F. In other embodiments, R² is substituted only with OH, such as 1, 2, or 3 OH.

In an embodiment, subscript n is 0. In another embodiment, n is 1.

In some embodiments, one of R¹ and R^{1a} is H and the other is halo. For example, R¹ is H and R^{1a} is halo, or R¹ is halo and R^{1a} is H. In additional embodiments, one of R¹ and R^{1a} is H and the other is F. In illustrative embodiments, R¹ is H and R^{1a} is F. or each of R¹ and R^{1a} is H. All these combinations of R¹ and R^{1a} are contemplated.

In some embodiments, R¹ and R^{1a}, together with the carbon atoms to which they are bound, form an optionally substituted fused C₃-C₈-cycloalkyl or an optionally substituted 3- to 6-membered heterocycloalkyl (wherein 1-4 ring members are independently selected from N, O, and S). In one embodiment, R¹ and R^{1a}, together with the carbon atoms to which they are bound, form an optionally substituted fused 3- to 6-membered heterocycloalkyl. In another embodiment, R¹ and R^{1a}, together with the carbon atoms to which they are bound, form an optionally substituted C₃-C₈-cycloalkyl. Exemplary cycloalkyl rings include cyclopropyl, cyclobutyl, cyclopentyl, and cyclohexyl.

In various embodiments, R¹ or R^{1a}, together with Y and the carbon atoms to which they are bound, form a fused 5- to 6-membered heterocycloalkyl. An illustrative heterocycloalkyl is pyrrolidinyl.

The present disclosure provides in additional embodiments a compound of Formula (1) or Formula (11) wherein R¹ is selected from the group consisting of H, CN, halo, and C₁-C₆-alkyl. In an embodiment, R⁴ is C₁-C₆-alkyl.

In still further embodiments, R⁵ and R⁷ are independently selected from the group consisting of H halo, and CN. For example, in an embodiment, at least one of R⁶ and R⁷ is H. In another embodiment, each of R⁵ and R⁷ is H.

Additional embodiments provide for Formula (1) compounds wherein R⁶ is selected from the group consisting of halo, CN, C₁-C₆-alkyl, C₂—C-alkenyl, C₂-C₆-alkynyl, and C₃-C₈-cycloalkyl. In an exemplary embodiment, R⁶ is C₁-C₆-alkyl.

R¹ in various embodiments is selected from the group consisting of H, CN, halo, C₁-C₆-alkyl, C₃-C₈-cycloalkyl, C₆-C₁₀-aryl, 5- to 10-membered heteroaryl (wherein 1-4 heteroaryl members are independently selected from N, O, and S), and —CONRR′. In some embodiments, R¹ is halo or 5- to 10-membered heteroaryl (wherein 1-4 heteroaryl members are independently selected from N, O, and S). An illustrative embodiment provides for Formula (I) compounds wherein R⁸ is halo, such as F.

The present disclosure provides in various embodiments a compound of Formula (I) wherein:
X is CH and Y is NH;
one of R¹ and R^{1a} is H and the other is F;
R² is an optionally substituted 3- to 6-membered heterocycloalkyl (wherein 1 ring member is 0);
n is 0 or 1;
R⁴ is selected from the group consisting of H, CN, halo, and C₁-C₆-alkyl;
R⁵ and R⁷ are independently selected from the group consisting of H, halo, and CN, wherein at least one of R⁵ and R⁷ is H;
R⁶ is selected from the group consisting of halo, CN, C₁-C₆-alkyl, C₂-C₆-alkenyl, C₂-C₆-alkynyl, and C₃-C₈-cycloalkyl; and
R⁸ is selected from the group consisting of H, CN, halo, C₁-C₆-alkyl, C₃-C₈-cycloalkyl, C₆-C₁₀-aryl, 5- to 10-membered heteroaryl (wherein 1-4 heteroaryl members are independently selected from N, O, and S), and —CONRR′.

Illustrative embodiments of Formula (1) and Formula (II) compounds and their pharmaceutically acceptable salts reside throughout the examples and Table 1 below.

TABLE 1
Representative Compounds of Formula (I).

| Compound | Structure |
|---|---|
| 1 | 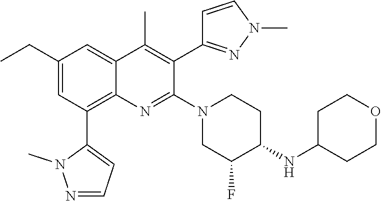 [SMILES: CCc1cc(-c2ccnn2C)c2nc(N3CC[C@H](NC4CCOCC4)[C@H](F)C3)c(-c3ccn(C)n3)c(C)c2c1] |
| 2 | 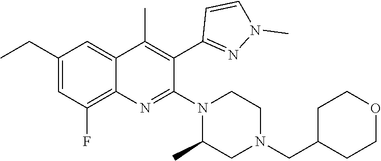 [SMILES: CCc1cc(F)c2nc(N3CCN(CC4CCOCC4)C[C@H]3C)c(-c3ccn(C)n3)c(C)c2c1] |
| 3 | 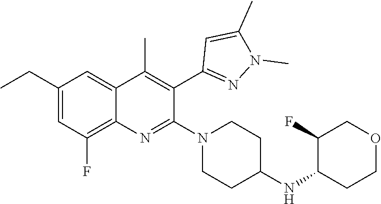 [SMILES: CCc1cc(F)c2nc(N3CCC(N[C@H]4CCOC[C@@H]4F)CC3)c(-c3cc(C)n(C)n3)c(C)c2c1] |
| 4 | 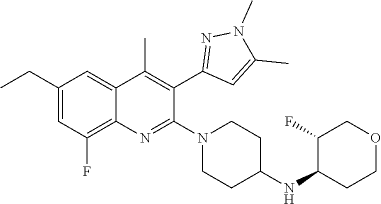 [SMILES: CCc1cc(F)c2nc(N3CCC(N[C@@H]4CCOC[C@H]4F)CC3)c(-c3cc(C)n(C)n3)c(C)c2c1] |
| 5 | 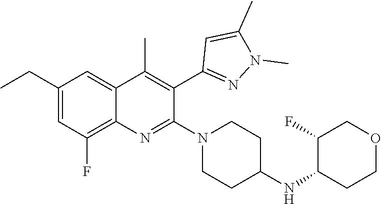 [SMILES: CCc1cc(F)c2nc(N3CCC(N[C@H]4CCOC[C@H]4F)CC3)c(-c3cc(C)n(C)n3)c(C)c2c1] |
| 6 | 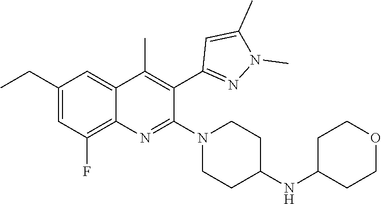 [SMILES: CCc1cc(F)c2nc(N3CCC(NC4CCOCC4)CC3)c(-c3cc(C)n(C)n3)c(C)c2c1] |
| 7 | 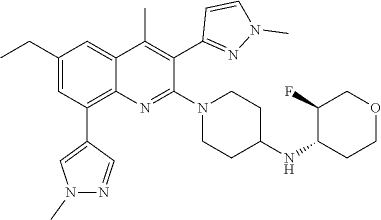 [SMILES: CCc1cc(-c2cnn(C)c2)c2nc(N3CCC(N[C@H]4CCOC[C@@H]4F)CC3)c(-c3ccn(C)n3)c(C)c2c1] |
| 8 | 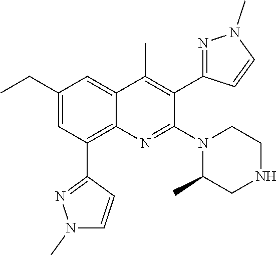 [SMILES: CCc1cc(-c2ccn(C)n2)c2nc(N3CCNC[C@H]3C)c(-c3ccn(C)n3)c(C)c2c1] |
| 9 | 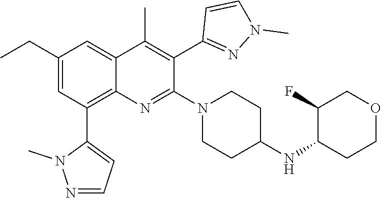 [SMILES: CCc1cc(-c2ccnn2C)c2nc(N3CCC(N[C@H]4CCOC[C@@H]4F)CC3)c(-c3ccn(C)n3)c(C)c2c1] |
| 10 | 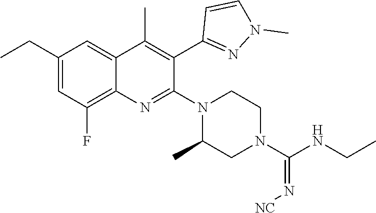 |
| 11 | 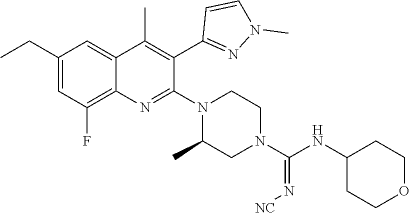 |
| 12 | 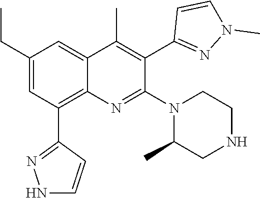 [SMILES: CCc1cc(-c2cc[nH]n2)c2nc(N3CCNC[C@H]3C)c(-c3ccn(C)n3)c(C)c2c1] |
| 13 | 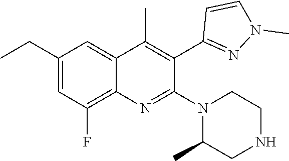 [SMILES: CCc1cc(F)c2nc(N3CCNC[C@H]3C)c(-c3ccn(C)n3)c(C)c2c1] |
| 14 | 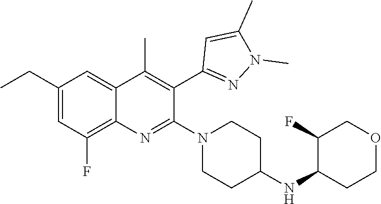 [SMILES: CCc1cc(F)c2nc(N3CCC(N[C@@H]4CCOC[C@@H]4F)CC3)c(-c3cc(C)n(C)n3)c(C)c2c1] |
| 15 | 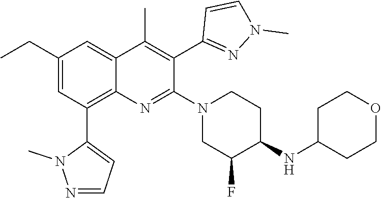 [SMILES: CCc1cc(-c2ccnn2C)c2nc(N3CC[C@@H](NC4CCOCC4)[C@@H](F)C3)c(-c3ccn(C)n3)c(C)c2c1] |
| 16 | 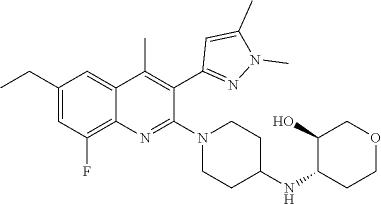 [SMILES: CCc1cc(F)c2nc(N3CCC(N[C@H]4CCOC[C@@H]4O)CC3)c(-c3cc(C)n(C)n3)c(C)c2c1] |
| 17 | 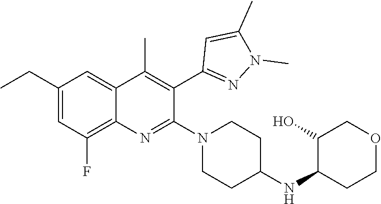 |
| 18 | 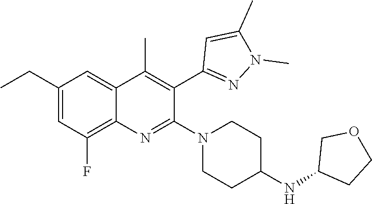 [SMILES: CCc1cc(F)c2nc(N3CCC(N[C@H]4CCOC4)CC3)c(-c3cc(C)n(C)n3)c(C)c2c1] |
| 19 | 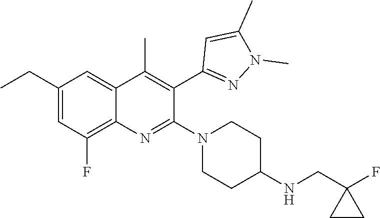 [SMILES: CCc1cc(F)c2nc(N3CCC(NCC4(F)CC4)CC3)c(-c3cc(C)n(C)n3)c(C)c2c1] |
| 20 | 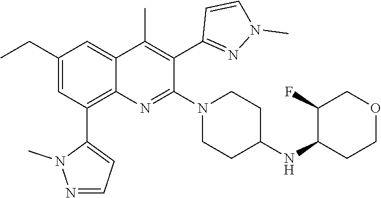 [SMILES: CCc1cc(-c2ccnn2C)c2nc(N3CCC(N[C@@H]4CCOC[C@@H]4F)CC3)c(-c3ccn(C)n3)c(C)c2c1] |
| 21 | 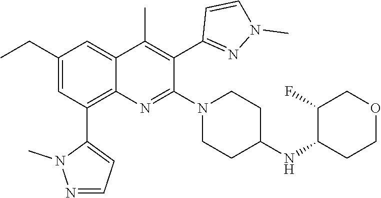 [SMILES: CCc1cc(-c2ccnn2C)c2nc(N3CCC(N[C@H]4CCOC[C@H]4F)CC3)c(-c3ccn(C)n3)c(C)c2c1] |
| 22 | 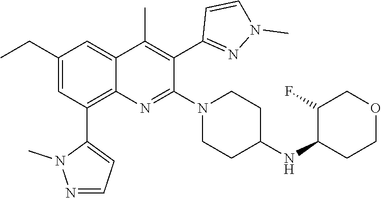 |
| 23 | 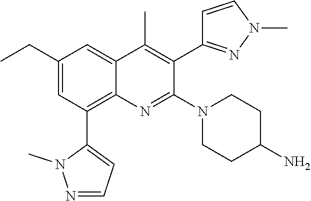 [SMILES: CCc1cc(-c2ccnn2C)c2nc(N3CCC(N)CC3)c(-c3ccn(C)n3)c(C)c2c1] |
| 24 | 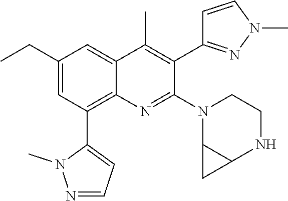 [SMILES: CCc1cc(-c2ccnn2C)c2nc(N3CCNC4CC43)c(-c3ccn(C)n3)c(C)c2c1] |
| 25 | 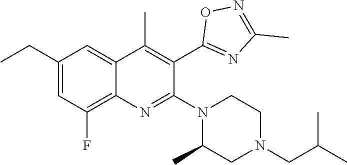 [SMILES: CCc1cc(F)c2nc(N3CCN(CC(C)C)C[C@H]3C)c(-c3nc(C)no3)c(C)c2c1] |
| 26 | 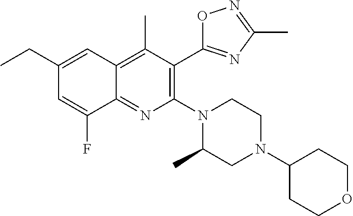 [SMILES: CCc1cc(F)c2nc(N3CCN(C4CCOCC4)C[C@H]3C)c(-c3nc(C)no3)c(C)c2c1] |
| 27 | 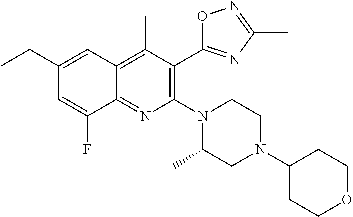 [SMILES: CCc1cc(F)c2nc(N3CCN(C4CCOCC4)C[C@@H]3C)c(-c3nc(C)no3)c(C)c2c1] |
| 28 | 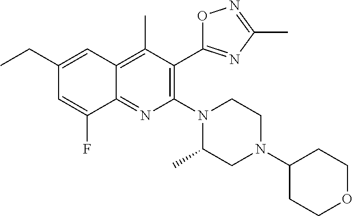 [SMILES: CCc1cc(F)c2nc(N3CCN(C4CCOCC4)C[C@@H]3C)c(-c3nc(C)no3)c(C)c2c1] |
| 29 | 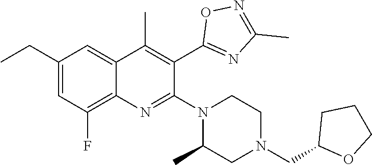 [SMILES: CCc1cc(F)c2nc(N3CCN(C[C@@H]4CCCO4)C[C@H]3C)c(-c3nc(C)no3)c(C)c2c1] |
| 30 | 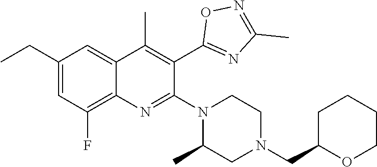 [SMILES: CCc1cc(F)c2nc(N3CCN(C[C@H]4CCCCO4)C[C@H]3C)c(-c3nc(C)no3)c(C)c2c1] |
| 31 | 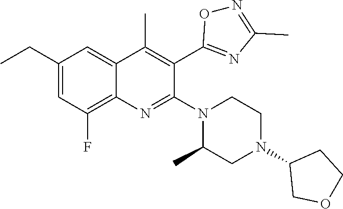 [SMILES: CCc1cc(F)c2nc(N3CCN([C@@H]4CCOC4)C[C@H]3C)c(-c3nc(C)no3)c(C)c2c1] |
| 32 | 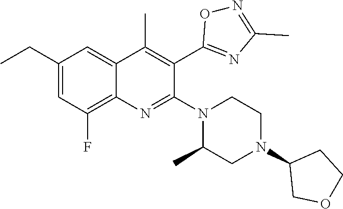 [SMILES: CCc1cc(F)c2nc(N3CCN([C@H]4CCOC4)C[C@H]3C)c(-c3nc(C)no3)c(C)c2c1] |
| 33 | 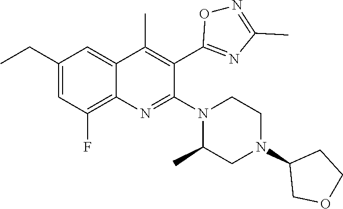 [SMILES: CCc1cc(F)c2nc(N3CCN([C@H]4CCOC4)C[C@H]3C)c(-c3nc(C)no3)c(C)c2c1] |
| 34 | 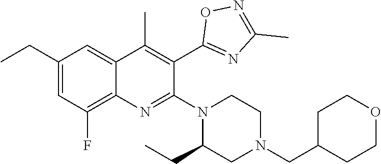 [SMILES: CCc1cc(F)c2nc(N3CCN(CC4CCOCC4)C[C@H]3CC)c(-c3nc(C)no3)c(C)c2c1] |
| 35 | 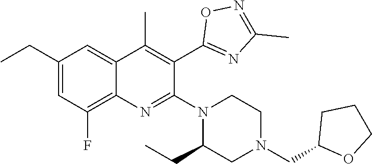 [SMILES: CCc1cc(F)c2nc(N3CCN(C[C@@H]4CCCO4)C[C@H]3CC)c(-c3nc(C)no3)c(C)c2c1] |
| 36 | 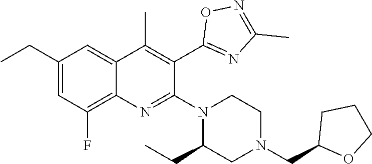 [SMILES: CCc1cc(F)c2nc(N3CCN(C[C@H]4CCCO4)C[C@H]3CC)c(-c3nc(C)no3)c(C)c2c1] |
| 37 | 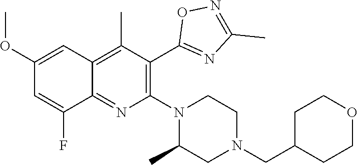 [SMILES: COc1cc(F)c2nc(N3CCN(CC4CCOCC4)C[C@H]3C)c(-c3nc(C)no3)c(C)c2c1] |
| 38 | 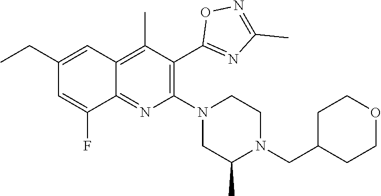 [SMILES: CCc1cc(F)c2nc(N3CCN(CC4CCOCC4)[C@@H](C)C3)c(-c3nc(C)no3)c(C)c2c1] |
| 39 | 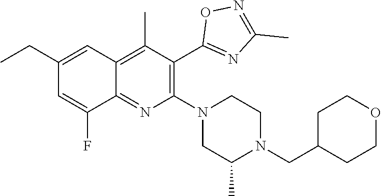 [SMILES: CCc1cc(F)c2nc(N3CCN(CC4CCOCC4)[C@H](C)C3)c(-c3nc(C)no3)c(C)c2c1] |
| 40 | 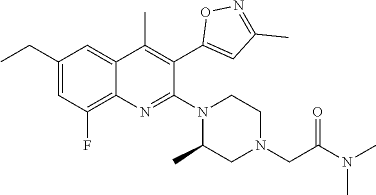 [SMILES: CCc1cc(F)c2nc(N3CCN(CC(=O)N(C)C)C[C@H]3C)c(-c3cc(C)no3)c(C)c2c1] |
| 41 | 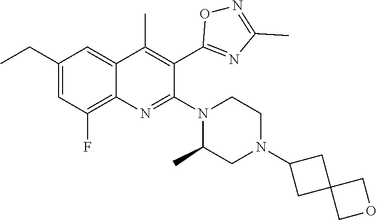 [SMILES: CCc1cc(F)c2nc(N3CCN(C4CC5(COC5)C4)C[C@H]3C)c(-c3nc(C)no3)c(C)c2c1] |
| 42 | 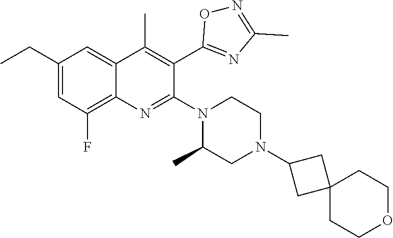 [SMILES: CCc1cc(F)c2nc(N3CCN(C4CC5(CCOCC5)C4)C[C@H]3C)c(-c3nc(C)no3)c(C)c2c1] |
| 43 | 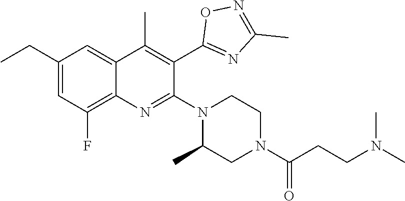 [SMILES: CCc1cc(F)c2nc(N3CCN(C(=O)CCN(C)C)C[C@H]3C)c(-c3nc(C)no3)c(C)c2c1] |
| 44 | 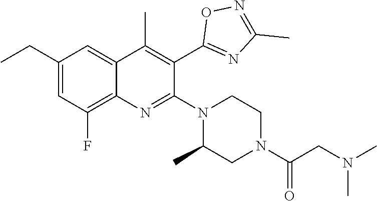 [SMILES: CCc1cc(F)c2nc(N3CCN(C(=O)CN(C)C)C[C@H]3C)c(-c3nc(C)no3)c(C)c2c1] |
| 45 | 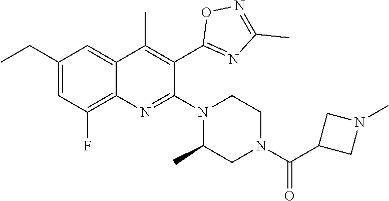 [SMILES: CCc1cc(F)c2nc(N3CCN(C(=O)C4CN(C)C4)C[C@H]3C)c(-c3nc(C)no3)c(C)c2c1] |
| 46 | 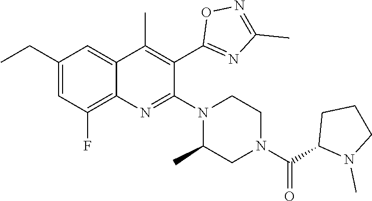 [SMILES: CCc1cc(F)c2nc(N3CCN(C(=O)[C@@H]4CCCN4C)C[C@H]3C)c(-c3nc(C)no3)c(C)c2c1] |
| 47 | 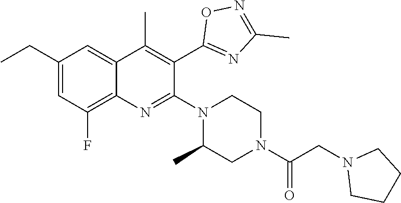 [SMILES: CCc1cc(F)c2nc(N3CCN(C(=O)CN4CCCC4)C[C@H]3C)c(-c3nc(C)no3)c(C)c2c1] |
| 48 | 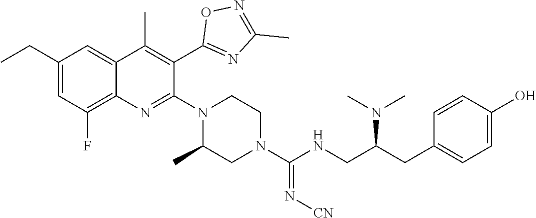 [SMILES: CCc1cc(F)c2nc(N3CCN(/C(=N/C#N)NC[C@H](Cc4ccc(O)cc4)N(C)C)C[C@H]3C)c(-c3nc(C)no3)c(C)c2c1] |
| 49 | 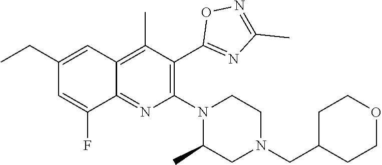 [SMILES: CCc1cc(F)c2nc(N3CCN(CC4CCOCC4)C[C@H]3C)c(-c3nc(C)no3)c(C)c2c1] |
| 50 | 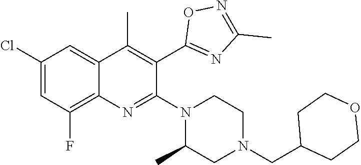 [SMILES: Cc1noc(-c2c(N3CCN(CC4CCOCC4)C[C@H]3C)nc3c(F)cc(Cl)cc3c2C)n1] |
| 51 | 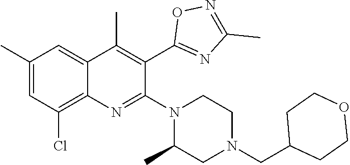 [SMILES: Cc1cc(Cl)c2nc(N3CCN(CC4CCOCC4)C[C@H]3C)c(-c3nc(C)no3)c(C)c2c1] |
| 52 | 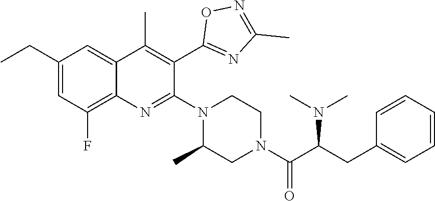 [SMILES: CCc1cc(F)c2nc(N3CCN(C(=O)[C@H](Cc4ccccc4)N(C)C)C[C@H]3C)c(-c3nc(C)no3)c(C)c2c1] |
| 53 | 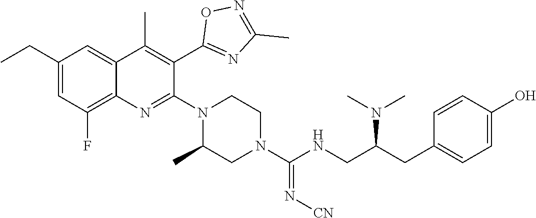 [SMILES: CCc1cc(F)c2nc(N3CCN(/C(=N/C#N)NC[C@H](Cc4ccc(O)cc4)N(C)C)C[C@H]3C)c(-c3nc(C)no3)c(C)c2c1] |
| 54 | 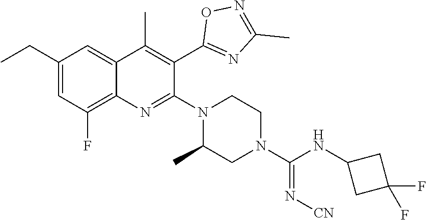 [SMILES: CCc1cc(F)c2nc(N3CCN(/C(=N/C#N)NC4CC(F)(F)C4)C[C@H]3C)c(-c3nc(C)no3)c(C)c2c1] |
| 55 | 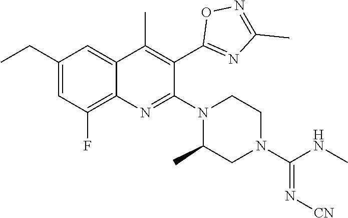 [SMILES: CCc1cc(F)c2nc(N3CCN(/C(=N/C#N)NC)C[C@H]3C)c(-c3nc(C)no3)c(C)c2c1] |
| 56 | 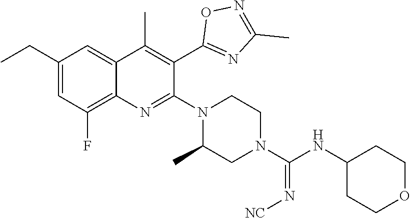 [SMILES: CCc1cc(F)c2nc(N3CCN(/C(=N\C#N)NC4CCOCC4)C[C@H]3C)c(-c3nc(C)no3)c(C)c2c1] |
| 57 | 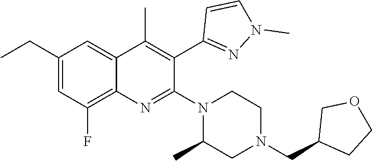 [SMILES: CCc1cc(F)c2nc(N3CCN(C[C@@H]4CCOC4)C[C@H]3C)c(-c3ccn(C)n3)c(C)c2c1] |
| 58 | 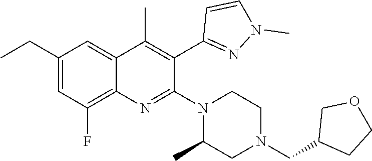 |
| 59 | 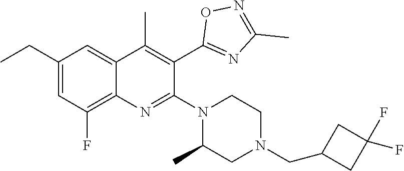 [SMILES: CCc1cc(F)c2nc(N3CCN(CC4CC(F)(F)C4)C[C@H]3C)c(-c3nc(C)no3)c(C)c2c1] |
| 60 | 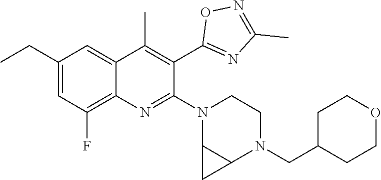 [SMILES: CCc1cc(F)c2nc(N3CCN(CC4CCOCC4)C4CC43)c(-c3nc(C)no3)c(C)c2c1] |
| 61 | 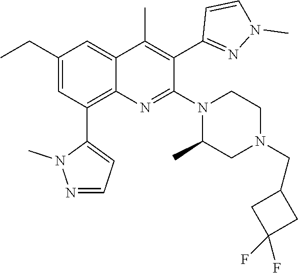 [SMILES: CCc1cc(-c2ccnn2C)c2nc(N3CCN(CC4CC(F)(F)C4)C[C@H]3C)c(-c3ccn(C)n3)c(C)c2c1] |
| 62 | 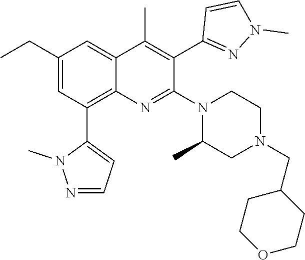 [SMILES: CCc1cc(-c2ccnn2C)c2nc(N3CCN(CC4CCOCC4)C[C@H]3C)c(-c3ccn(C)n3)c(C)c2c1] |
| 63 | 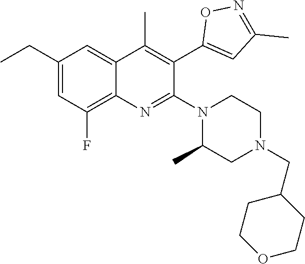 [SMILES: CCc1cc(F)c2nc(N3CCN(CC4CCOCC4)C[C@H]3C)c(-c3cc(C)no3)c(C)c2c1] |
| 64 | 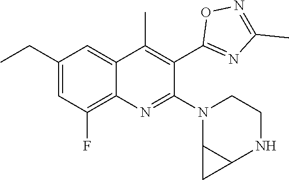 [SMILES: CCc1cc(F)c2nc(N3CCNC4CC43)c(-c3nc(C)no3)c(C)c2c1] |
| 65 | 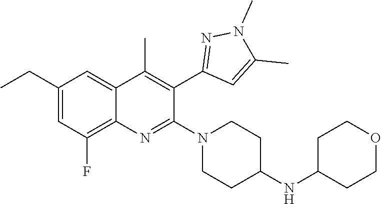 [SMILES: CCc1cc(F)c2nc(N3CCC(NC4CCOCC4)CC3)c(-c3cc(C)n(C)n3)c(C)c2c1] |
| 66 | 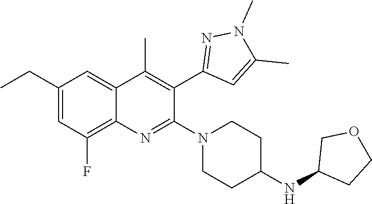 [SMILES: CCc1cc(F)c2nc(N3CCC(N[C@@H]4CCOC4)CC3)c(-c3cc(C)n(C)n3)c(C)c2c1] |
| 67 | 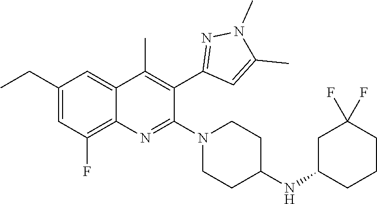 [SMILES: CCc1cc(F)c2nc(N3CCC(N[C@H]4CCCC(F)(F)C4)CC3)c(-c3cc(C)n(C)n3)c(C)c2c1] |
| 68 | 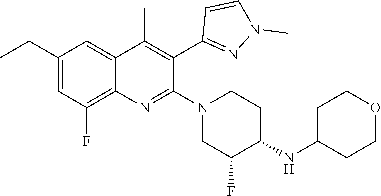 [SMILES: CCc1cc(F)c2nc(N3CC[C@H](NC4CCOCC4)[C@H](F)C3)c(-c3ccn(C)n3)c(C)c2c1] |
| 69 | 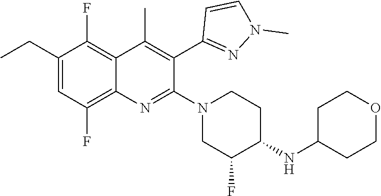 [SMILES: CCc1cc(F)c2nc(N3CC[C@H](NC4CCOCC4)[C@H](F)C3)c(-c3ccn(C)n3)c(C)c2c1F] |
| 70 | 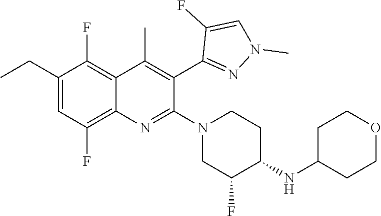 [SMILES: CCc1cc(F)c2nc(N3CC[C@H](NC4CCOCC4)[C@H](F)C3)c(-c3nn(C)cc3F)c(C)c2c1F] |
| 71 | 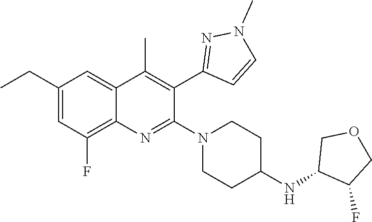 [SMILES: CCc1cc(F)c2nc(N3CCC(N[C@@H]4COC[C@@H]4F)CC3)c(-c3ccn(C)n3)c(C)c2c1] |
| 72 | 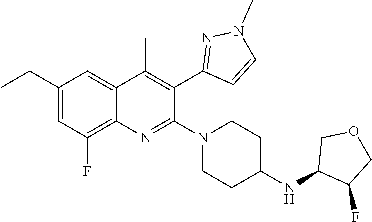 [SMILES: CCc1cc(F)c2nc(N3CCC(N[C@H]4COC[C@H]4F)CC3)c(-c3ccn(C)n3)c(C)c2c1] |
| 73 | 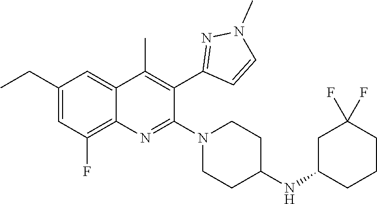 [SMILES: CCc1cc(F)c2nc(N3CCC(N[C@H]4CCCC(F)(F)C4)CC3)c(-c3ccn(C)n3)c(C)c2c1] |
| 74 | 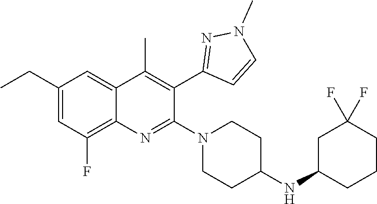 [SMILES: CCc1cc(F)c2nc(N3CCC(N[C@@H]4CCCC(F)(F)C4)CC3)c(-c3ccn(C)n3)c(C)c2c1] |
| 75 | 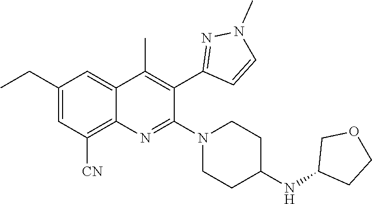 [SMILES: CCc1cc(C#N)c2nc(N3CCC(N[C@H]4CCOC4)CC3)c(-c3ccn(C)n3)c(C)c2c1] |
| 76 | 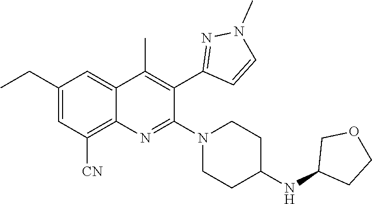 [SMILES: CCc1cc(C#N)c2nc(N3CCC(N[C@@H]4CCOC4)CC3)c(-c3ccn(C)n3)c(C)c2c1] |
| 77 | 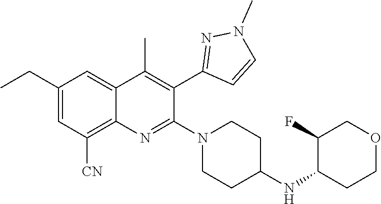 [SMILES: CCc1cc(C#N)c2nc(N3CCC(N[C@H]4CCOC[C@@H]4F)CC3)c(-c3ccn(C)n3)c(C)c2c1] |
| 78 | 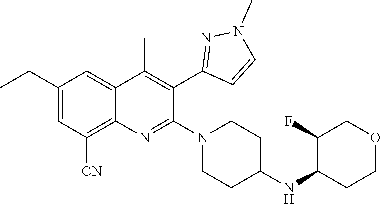 [SMILES: CCc1cc(C#N)c2nc(N3CCC(N[C@@H]4CCOC[C@@H]4F)CC3)c(-c3ccn(C)n3)c(C)c2c1] |
| 79 | 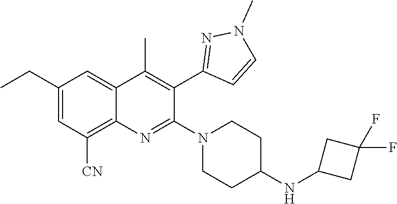 [SMILES: CCc1cc(C#N)c2nc(N3CCC(NC4CC(F)(F)C4)CC3)c(-c3ccn(C)n3)c(C)c2c1] |
| 80 | 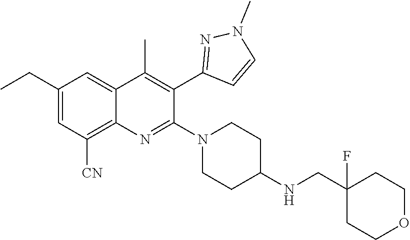 [SMILES: CCc1cc(C#N)c2nc(N3CCC(NCC4(F)CCOCC4)CC3)c(-c3ccn(C)n3)c(C)c2c1] |
| 81 | 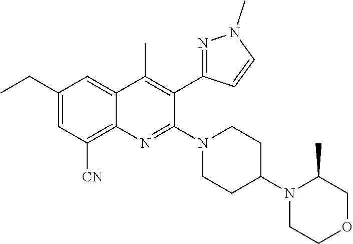 [SMILES: CCc1cc(C#N)c2nc(N3CCC(N4CCOC[C@@H]4C)CC3)c(-c3ccn(C)n3)c(C)c2c1] |
| 82 | 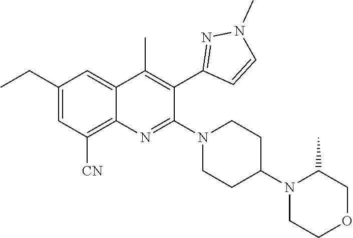 |
| 83 | 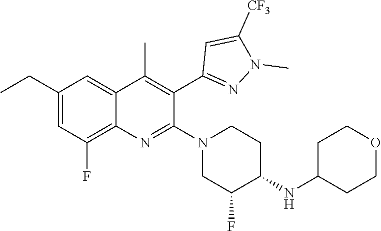 [SMILES: CCc1cc(F)c2nc(N3CC[C@H](NC4CCOCC4)[C@H](F)C3)c(-c3cc(C(F)(F)F)n(C)n3)c(C)c2c1] |
| 84 | 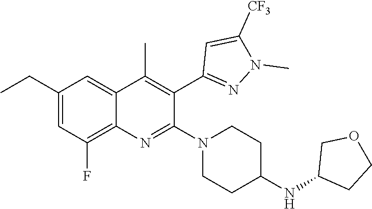 [SMILES: CCc1cc(F)c2nc(N3CCC(N[C@H]4CCOC4)CC3)c(-c3cc(C(F)(F)F)n(C)n3)c(C)c2c1] |
| 85 | 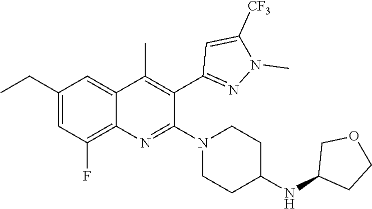 [SMILES: CCc1cc(F)c2nc(N3CCC(N[C@@H]4CCOC4)CC3)c(-c3cc(C(F)(F)F)n(C)n3)c(C)c2c1] |
| 86 | 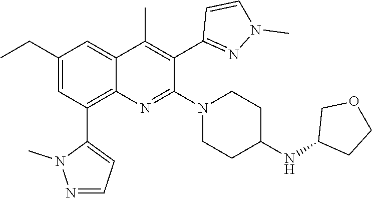 [SMILES: CCc1cc(-c2ccnn2C)c2nc(N3CCC(N[C@H]4CCOC4)CC3)c(-c3ccn(C)n3)c(C)c2c1] |
| 87 | 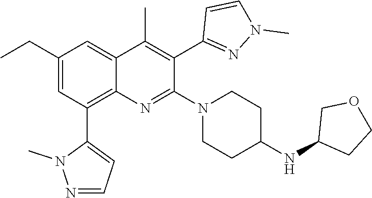 [SMILES: CCc1cc(-c2ccnn2C)c2nc(N3CCC(N[C@@H]4CCOC4)CC3)c(-c3ccn(C)n3)c(C)c2c1] |
| 88 | 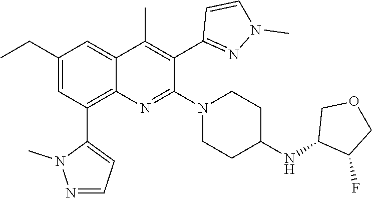 [SMILES: CCc1cc(-c2ccnn2C)c2nc(N3CCC(N[C@@H]4COC[C@@H]4F)CC3)c(-c3ccn(C)n3)c(C)c2c1] |
| 89 | 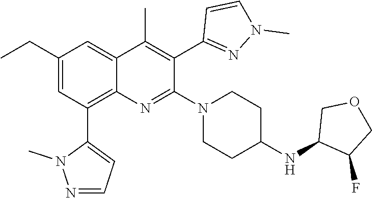 [SMILES: CCc1cc(-c2ccnn2C)c2nc(N3CCC(N[C@H]4COC[C@H]4F)CC3)c(-c3ccn(C)n3)c(C)c2c1] |
| 90 | 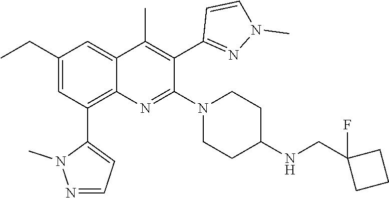 [SMILES: CCc1cc(-c2ccnn2C)c2nc(N3CCC(NCC4(F)CCC4)CC3)c(-c3ccn(C)n3)c(C)c2c1] |
| 91 | 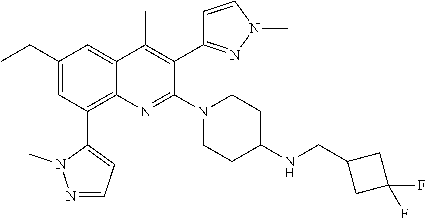 [SMILES: CCc1cc(-c2ccnn2C)c2nc(N3CCC(NCC4CC(F)(F)C4)CC3)c(-c3ccn(C)n3)c(C)c2c1] |
| 92 | 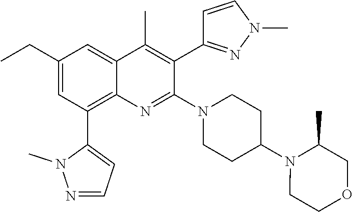 [SMILES: CCc1cc(-c2ccnn2C)c2nc(N3CCC(N4CCOC[C@@H]4C)CC3)c(-c3ccn(C)n3)c(C)c2c1] |
| 93 | 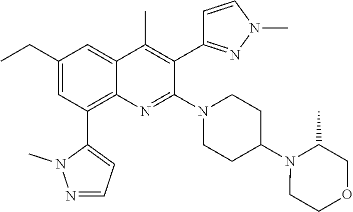 |
| 94 | 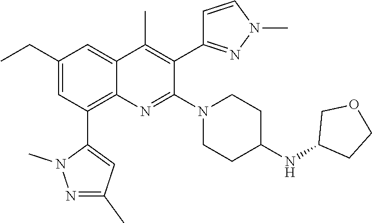 [SMILES: CCc1cc(-c2cc(C)nn2C)c2nc(N3CCC(N[C@H]4CCOC4)CC3)c(-c3ccn(C)n3)c(C)c2c1] |
| 95 | 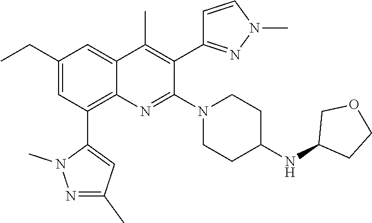 [SMILES: CCc1cc(-c2cc(C)nn2C)c2nc(N3CCC(N[C@@H]4CCOC4)CC3)c(-c3ccn(C)n3)c(C)c2c1] |
| 96 | 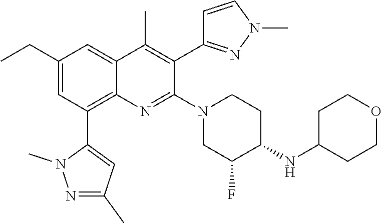 [SMILES: CCc1cc(-c2cc(C)nn2C)c2nc(N3CC[C@H](NC4CCOCC4)[C@H](F)C3)c(-c3ccn(C)n3)c(C)c2c1] |
| 97 | 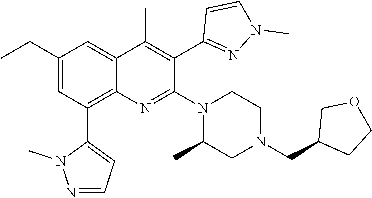 [SMILES: CCc1cc(-c2ccnn2C)c2nc(N3CCN(C[C@@H]4CCOC4)C[C@H]3C)c(-c3ccn(C)n3)c(C)c2c1] |
| 98 | 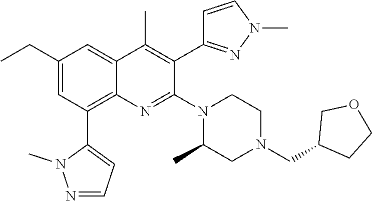 [SMILES: CCc1cc(-c2ccnn2C)c2nc(N3CCN(C[C@H]4CCOC4)C[C@H]3C)c(-c3ccn(C)n3)c(C)c2c1] |
| 99 | 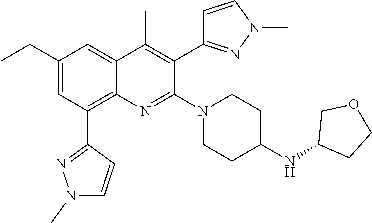 [SMILES: CCc1cc(-c2ccn(C)n2)c2nc(N3CCC(N[C@H]4CCOC4)CC3)c(-c3ccn(C)n3)c(C)c2c1] |
| 100 | 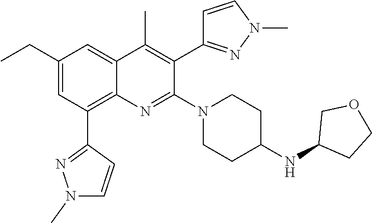 [SMILES: CCc1cc(-c2ccn(C)n2)c2nc(N3CCC(N[C@@H]4CCOC4)CC3)c(-c3ccn(C)n3)c(C)c2c1] |
| 101 | 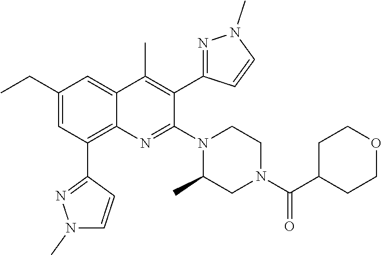 [SMILES: CCc1cc(-c2ccn(C)n2)c2nc(N3CCN(C(=O)C4CCOCC4)C[C@H]3C)c(-c3ccn(C)n3)c(C)c2c1] |
| 102 | 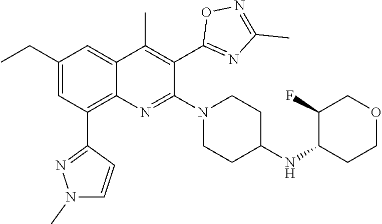 [SMILES: CCc1cc(-c2ccn(C)n2)c2nc(N3CCC(N[C@H]4CCOC[C@@H]4F)CC3)c(-c3nc(C)no3)c(C)c2c1] |
| 103 | 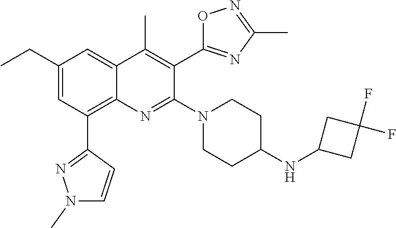 [SMILES: CCc1cc(-c2ccn(C)n2)c2nc(N3CCC(NC4CC(F)(F)C4)CC3)c(-c3nc(C)no3)c(C)c2c1] |
| 104 | 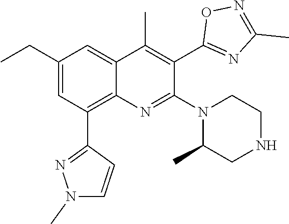 [SMILES: CCc1cc(-c2ccn(C)n2)c2nc(N3CCNC[C@H]3C)c(-c3nc(C)no3)c(C)c2c1] |
| 105 | 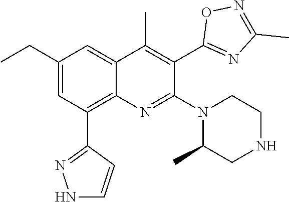 [SMILES: CCc1cc(-c2cc[nH]n2)c2nc(N3CCNC[C@H]3C)c(-c3nc(C)no3)c(C)c2c1] |
| 106 | 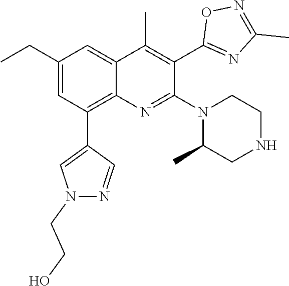 [SMILES: CCc1cc(-c2cnn(CCO)c2)c2nc(N3CCNC[C@H]3C)c(-c3nc(C)no3)c(C)c2c1] |
| 107 | 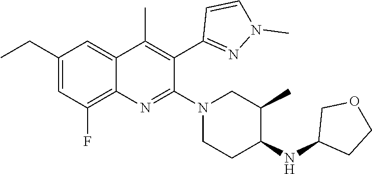 [SMILES: CCc1cc(F)c2nc(N3CC[C@H](N[C@@H]4CCOC4)[C@H](C)C3)c(-c3ccn(C)n3)c(C)c2c1] |
| 108 | 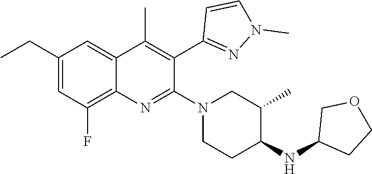 [SMILES: CCc1cc(F)c2nc(N3CC[C@H](N[C@@H]4CCOC4)[C@@H](C)C3)c(-c3ccn(C)n3)c(C)c2c1] |
| 109 | 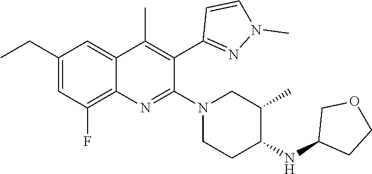 [SMILES: CCc1cc(F)c2nc(N3CC[C@@H](N[C@@H]4CCOC4)[C@@H](C)C3)c(-c3ccn(C)n3)c(C)c2c1] |
| 110 | 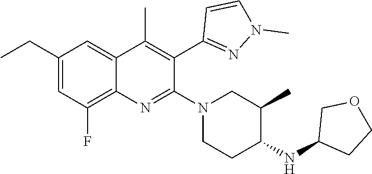 [SMILES: CCc1cc(F)c2nc(N3CC[C@@H](N[C@@H]4CCOC4)[C@H](C)C3)c(-c3ccn(C)n3)c(C)c2c1] |
| 111 | 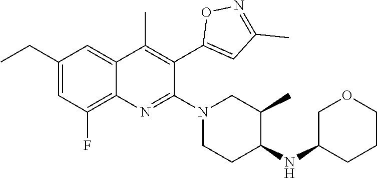 [SMILES: CCc1cc(F)c2nc(N3CC[C@H](N[C@@H]4CCCOC4)[C@H](C)C3)c(-c3cc(C)no3)c(C)c2c1] |
| 112 | 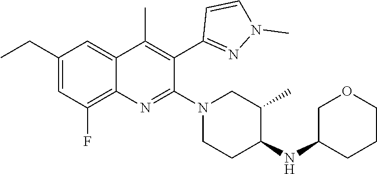 [SMILES: CCc1cc(F)c2nc(N3CC[C@H](N[C@@H]4CCCOC4)[C@@H](C)C3)c(-c3ccn(C)n3)c(C)c2c1] |
| 113 | 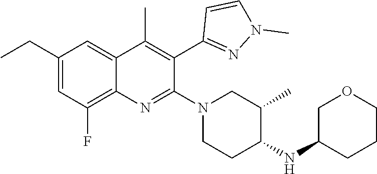 [SMILES: CCc1cc(F)c2nc(N3CC[C@@H](N[C@@H]4CCCOC4)[C@@H](C)C3)c(-c3ccn(C)n3)c(C)c2c1] |
| 114 | 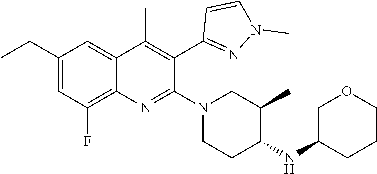 [SMILES: CCc1cc(F)c2nc(N3CC[C@@H](N[C@@H]4CCCOC4)[C@H](C)C3)c(-c3ccn(C)n3)c(C)c2c1] |
| 115 | 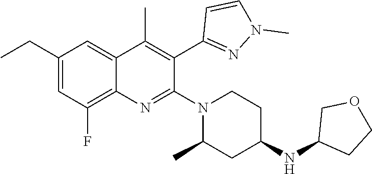 [SMILES: CCc1cc(F)c2nc(N3CC[C@@H](N[C@@H]4CCOC4)C[C@H]3C)c(-c3ccn(C)n3)c(C)c2c1] |
| 116 | 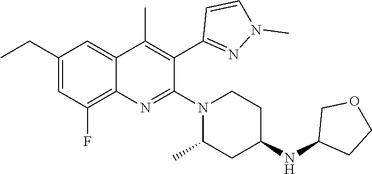 [SMILES: CCc1cc(F)c2nc(N3CC[C@@H](N[C@@H]4CCOC4)C[C@@H]3C)c(-c3ccn(C)n3)c(C)c2c1] |
| 117 | 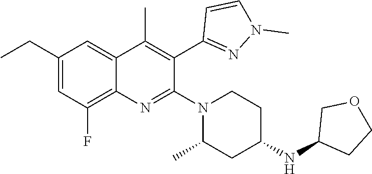 [SMILES: CCc1cc(F)c2nc(N3CC[C@H](N[C@@H]4CCOC4)C[C@@H]3C)c(-c3ccn(C)n3)c(C)c2c1] |
| 118 | 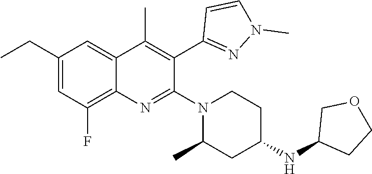 [SMILES: CCc1cc(F)c2nc(N3CC[C@H](N[C@@H]4CCOC4)C[C@H]3C)c(-c3ccn(C)n3)c(C)c2c1] |
| 119 | 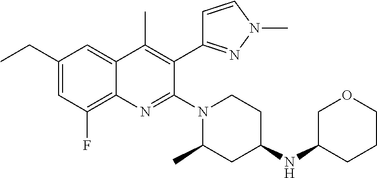 [SMILES: CCc1cc(F)c2nc(N3CC[C@@H](N[C@@H]4CCCOC4)C[C@H]3C)c(-c3ccn(C)n3)c(C)c2c1] |
| 120 | 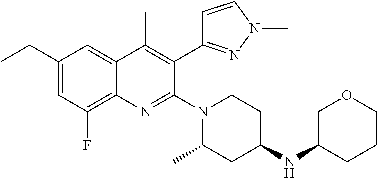 [SMILES: CCc1cc(F)c2nc(N3CC[C@@H](N[C@@H]4CCCOC4)C[C@@H]3C)c(-c3ccn(C)n3)c(C)c2c1] |
| 121 | 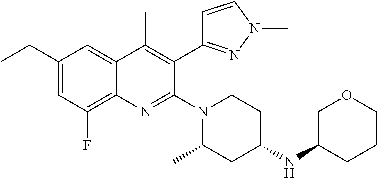 [SMILES: CCc1cc(F)c2nc(N3CC[C@H](N[C@@H]4CCCOC4)C[C@@H]3C)c(-c3ccn(C)n3)c(C)c2c1] |
| 122 | 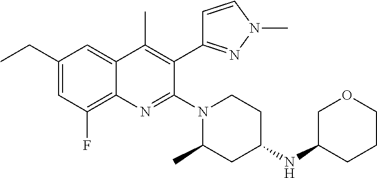 [SMILES: CCc1cc(F)c2nc(N3CC[C@H](N[C@@H]4CCCOC4)C[C@H]3C)c(-c3ccn(C)n3)c(C)c2c1] |
| 123 | 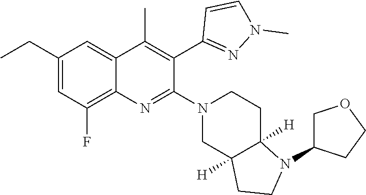 [SMILES: CCc1cc(F)c2nc(N3CC[C@@H]4[C@@H](CCN4[C@@H]4CCOC4)C3)c(-c3ccn(C)n3)c(C)c2c1] |
| 124 | 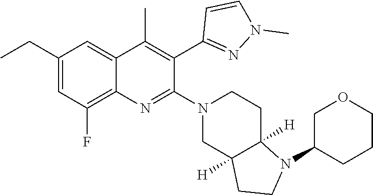 [SMILES: CCc1cc(F)c2nc(N3CC[C@@H]4[C@@H](CCN4[C@@H]4CCCOC4)C3)c(-c3ccn(C)n3)c(C)c2c1] |
| 125 | 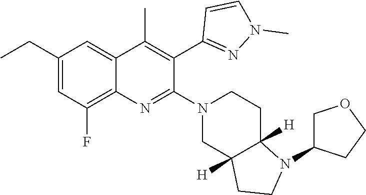 [SMILES: CCc1cc(F)c2nc(N3CC[C@H]4[C@H](CCN4[C@@H]4CCOC4)C3)c(-c3ccn(C)n3)c(C)c2c1] |
| 126 | 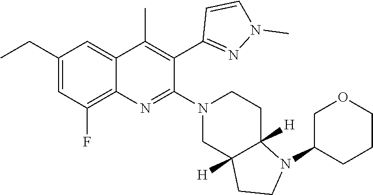 [SMILES: CCc1cc(F)c2nc(N3CC[C@H]4[C@H](CCN4[C@@H]4CCCOC4)C3)c(-c3ccn(C)n3)c(C)c2c1] |
| 127 | 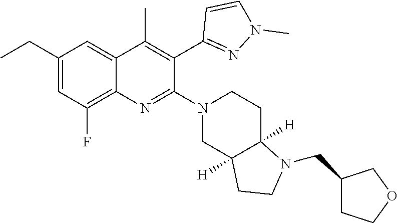 [SMILES: CCc1cc(F)c2nc(N3CC[C@@H]4[C@@H](CCN4C[C@@H]4CCOC4)C3)c(-c3ccn(C)n3)c(C)c2c1] |
| 128 | 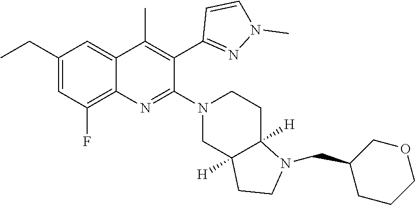 [SMILES: CCc1cc(F)c2nc(N3CC[C@@H]4[C@@H](CCN4C[C@@H]4CCCOC4)C3)c(-c3ccn(C)n3)c(C)c2c1] |
| 129 | 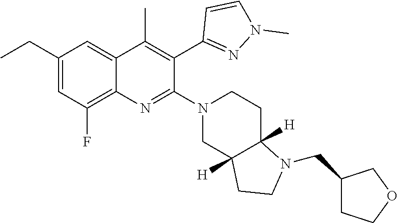 [SMILES: CCc1cc(F)c2nc(N3CC[C@H]4[C@H](CCN4C[C@@H]4CCOC4)C3)c(-c3ccn(C)n3)c(C)c2c1] |
| 130 | 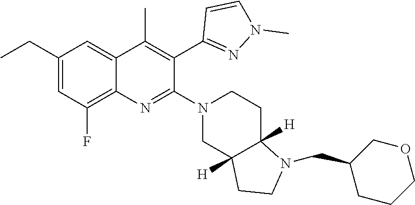 [SMILES: CCc1cc(F)c2nc(N3CC[C@H]4[C@H](CCN4C[C@@H]4CCCOC4)C3)c(-c3ccn(C)n3)c(C)c2c1] |
| 131 | 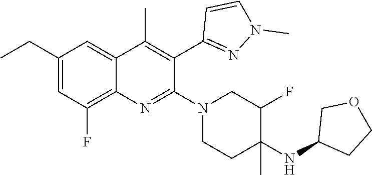 [SMILES: CCc1cc(F)c2nc(N3CCC(C)(N[C@@H]4CCOC4)C(F)C3)c(-c3ccn(C)n3)c(C)c2c1] |
| 132 | 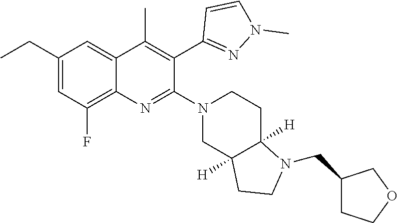 [SMILES: CCc1cc(F)c2nc(N3CC[C@@H]4[C@@H](CCN4C[C@@H]4CCOC4)C3)c(-c3ccn(C)n3)c(C)c2c1] |
| 133 | 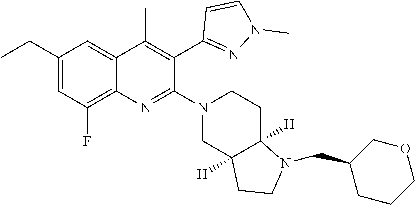 [SMILES: CCc1cc(F)c2nc(N3CC[C@@H]4[C@@H](CCN4C[C@@H]4CCCOC4)C3)c(-c3ccn(C)n3)c(C)c2c1] |
| 134 | 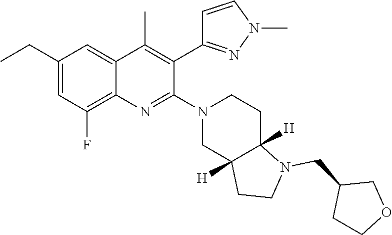 [SMILES: CCc1cc(F)c2nc(N3CC[C@H]4[C@H](CCN4C[C@@H]4CCOC4)C3)c(-c3ccn(C)n3)c(C)c2c1] |
| 135 | 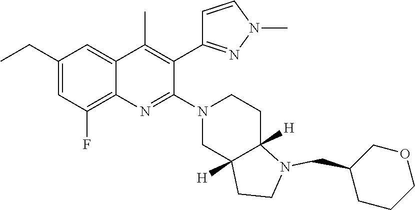 [SMILES: CCc1cc(F)c2nc(N3CC[C@H]4[C@H](CCN4C[C@@H]4CCCOC4)C3)c(-c3ccn(C)n3)c(C)c2c1] |
| 136 | 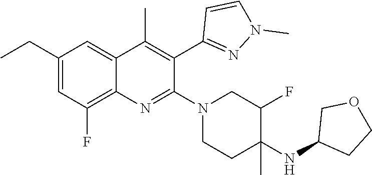 [SMILES: CCc1cc(F)c2nc(N3CCC(C)(N[C@@H]4CCOC4)C(F)C3)c(-c3ccn(C)n3)c(C)c2c1] |
| 137 | 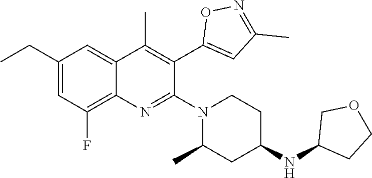 [SMILES: CCc1cc(F)c2nc(N3CC[C@@H](N[C@@H]4CCOC4)C[C@H]3C)c(-c3cc(C)no3)c(C)c2c1] |
| 138 | 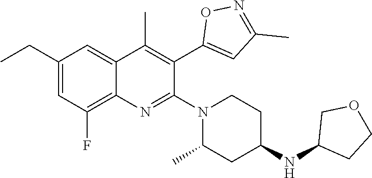 [SMILES: CCc1cc(F)c2nc(N3CC[C@@H](N[C@@H]4CCOC4)C[C@@H]3C)c(-c3cc(C)no3)c(C)c2c1] |
| 139 | 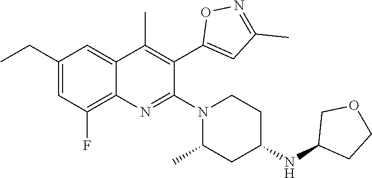 [SMILES: CCc1cc(F)c2nc(N3CC[C@H](N[C@@H]4CCOC4)C[C@@H]3C)c(-c3cc(C)no3)c(C)c2c1] |
| 140 | 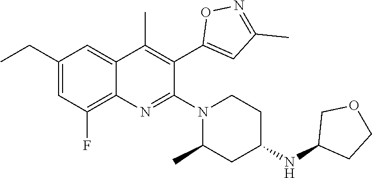 [SMILES: CCc1cc(F)c2nc(N3CC[C@H](N[C@@H]4CCOC4)C[C@H]3C)c(-c3cc(C)no3)c(C)c2c1] |
| 141 | 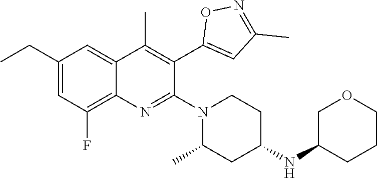 [SMILES: CCc1cc(F)c2nc(N3CC[C@H](N[C@@H]4CCCOC4)C[C@@H]3C)c(-c3cc(C)no3)c(C)c2c1] |
| 142 | 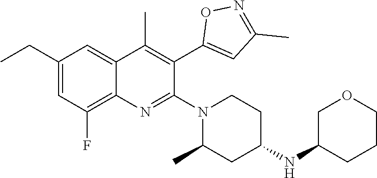 [SMILES: CCc1cc(F)c2nc(N3CC[C@H](N[C@@H]4CCCOC4)C[C@H]3C)c(-c3cc(C)no3)c(C)c2c1] |
| 143 | 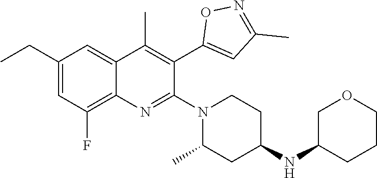 [SMILES: CCc1cc(F)c2nc(N3CC[C@@H](N[C@@H]4CCCOC4)C[C@@H]3C)c(-c3cc(C)no3)c(C)c2c1] |
| 144 | 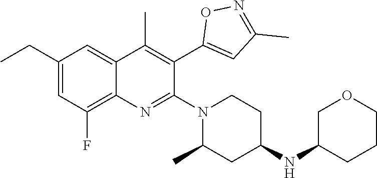 [SMILES: CCc1cc(F)c2nc(N3CC[C@@H](N[C@@H]4CCCOC4)C[C@H]3C)c(-c3cc(C)no3)c(C)c2c1] |
| 145 | 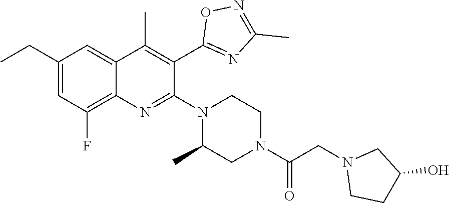 [SMILES: CCc1cc(F)c2nc(N3CCN(C(=O)CN4CC[C@@H](O)C4)C[C@H]3C)c(-c3nc(C)no3)c(C)c2c1] |
| 146 | 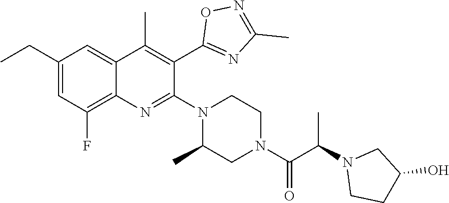 [SMILES: CCc1cc(F)c2nc(N3CCN(C(=O)[C@@H](C)N4CC[C@@H](O)C4)C[C@H]3C)c(-c3nc(C)no3)c(C)c2c1] |
| 147 | 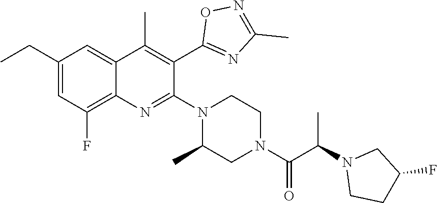 [SMILES: CCc1cc(F)c2nc(N3CCN(C(=O)[C@@H](C)N4CC[C@@H](F)C4)C[C@H]3C)c(-c3nc(C)no3)c(C)c2c1] |
| 148 | 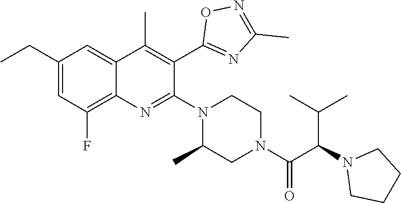 [SMILES: CCc1cc(F)c2nc(N3CCN(C(=O)[C@@H](C(C)C)N4CCCC4)C[C@H]3C)c(-c3nc(C)no3)c(C)c2c1] |
| 149 | 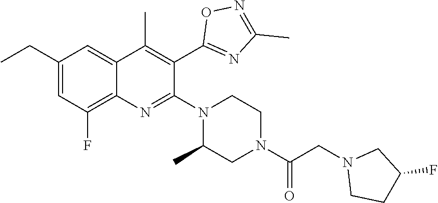 [SMILES: CCc1cc(F)c2nc(N3CCN(C(=O)CN4CC[C@@H](F)C4)C[C@H]3C)c(-c3nc(C)no3)c(C)c2c1] |
| 150 | 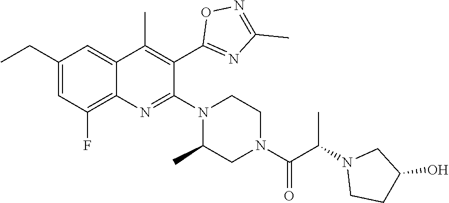 [SMILES: CCc1cc(F)c2nc(N3CCN(C(=O)[C@H](C)N4CC[C@@H](O)C4)C[C@H]3C)c(-c3nc(C)no3)c(C)c2c1] |
| 151 | 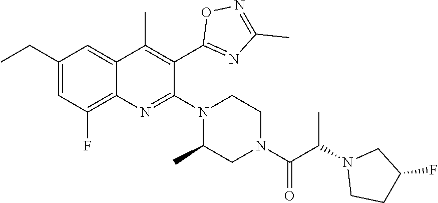 |
| 152 | 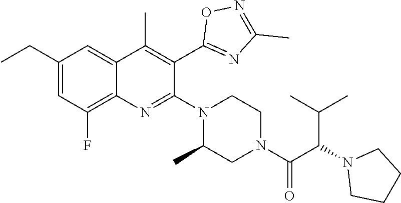 |
| 153 | 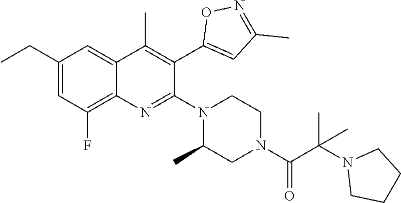 [SMILES: CCc1cc(F)c2nc(N3CCN(C(=O)C(C)(C)N4CCCC4)C[C@H]3C)c(-c3cc(C)no3)c(C)c2c1] |
| 154 | 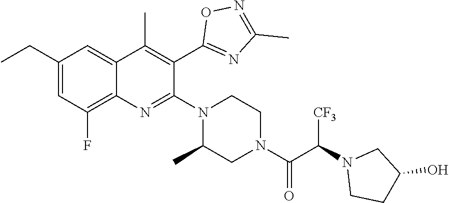 [SMILES: CCc1cc(F)c2nc(N3CCN(C(=O)[C@H](N4CC[C@@H](O)C4)C(F)(F)F)C[C@H]3C)c(-c3nc(C)no3)c(C)c2c1] |
| 155 | 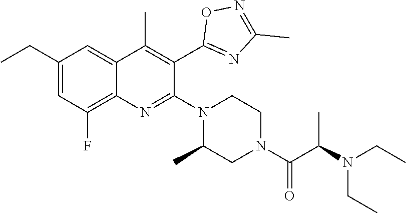 [SMILES: CCc1cc(F)c2nc(N3CCN(C(=O)[C@@H](C)N(CC)CC)C[C@H]3C)c(-c3nc(C)no3)c(C)c2c1] |
| 156 | 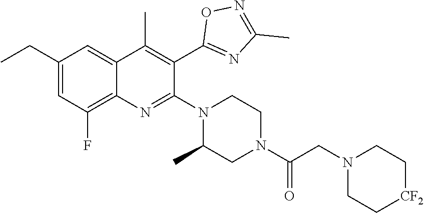 [SMILES: CCc1cc(F)c2nc(N3CCN(C(=O)CN4CCC(F)(F)CC4)C[C@H]3C)c(-c3nc(C)no3)c(C)c2c1] |
| 157 | 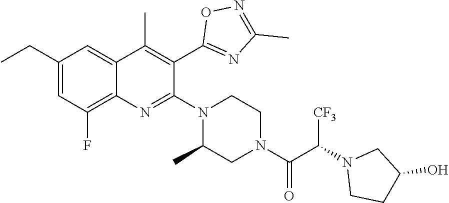 [SMILES: CCc1cc(F)c2nc(N3CCN(C(=O)[C@@H](N4CC[C@@H](O)C4)C(F)(F)F)C[C@H]3C)c(-c3nc(C)no3)c(C)c2c1] |
| 158 | 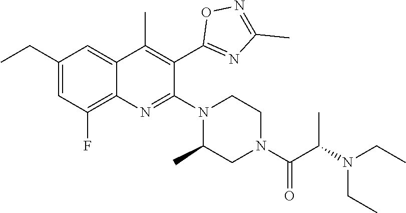 [SMILES: CCc1cc(F)c2nc(N3CCN(C(=O)[C@H](C)N(CC)CC)C[C@H]3C)c(-c3nc(C)no3)c(C)c2c1] |
| 159 | 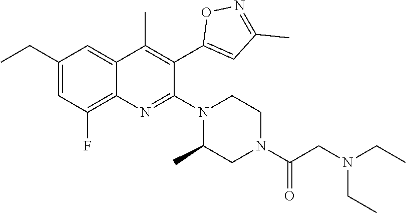 [SMILES: CCc1cc(F)c2nc(N3CCN(C(=O)CN(CC)CC)C[C@H]3C)c(-c3cc(C)no3)c(C)c2c1] |
| 160 | 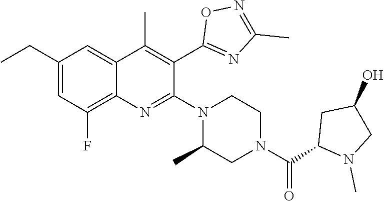 [SMILES: CCc1cc(F)c2nc(N3CCN(C(=O)[C@@H]4C[C@@H](O)CN4C)C[C@H]3C)c(-c3nc(C)no3)c(C)c2c1] |
| 161 | 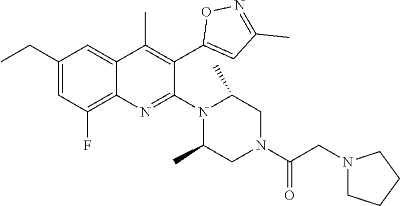 [SMILES: CCc1cc(F)c2nc(N3[C@H](C)CN(C(=O)CN4CCCC4)C[C@H]3C)c(-c3cc(C)no3)c(C)c2c1] |
| 162 | 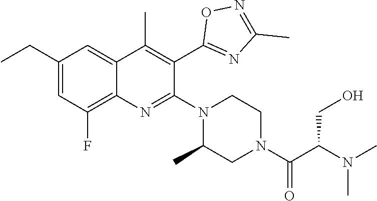 [SMILES: CCc1cc(F)c2nc(N3CCN(C(=O)[C@H](CO)N(C)C)C[C@H]3C)c(-c3nc(C)no3)c(C)c2c1] |
| 163 | 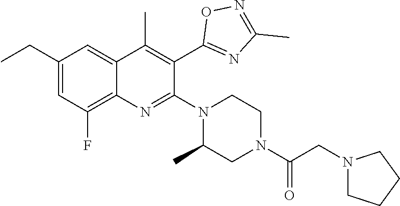 [SMILES: CCc1cc(F)c2nc(N3CCN(C(=O)CN4CCCC4)C[C@H]3C)c(-c3nc(C)no3)c(C)c2c1] |
| 164 | 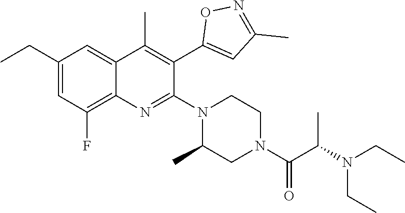 [SMILES: CCc1cc(F)c2nc(N3CCN(C(=O)[C@H](C)N(CC)CC)C[C@H]3C)c(-c3cc(C)no3)c(C)c2c1] |
| 165 | 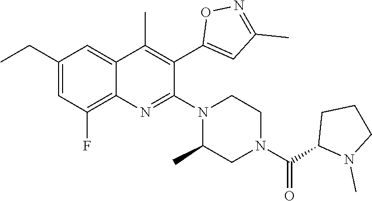 [SMILES: CCc1cc(F)c2nc(N3CCN(C(=O)[C@@H]4CCCN4C)C[C@H]3C)c(-c3cc(C)no3)c(C)c2c1] |
| 166 | 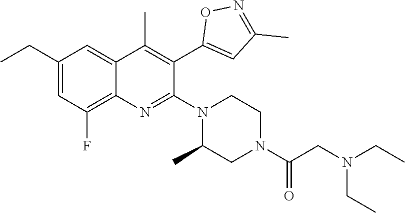 [SMILES: CCc1cc(F)c2nc(N3CCN(C(=O)CN(CC)CC)C[C@H]3C)c(-c3cc(C)no3)c(C)c2c1] |
| 167 | 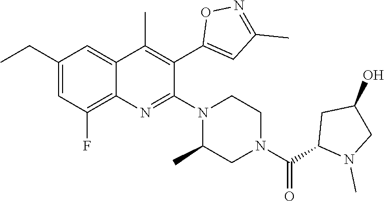 [SMILES: CCc1cc(F)c2nc(N3CCN(C(=O)[C@@H]4C[C@@H](O)CN4C)C[C@H]3C)c(-c3cc(C)no3)c(C)c2c1] |
| 168 | 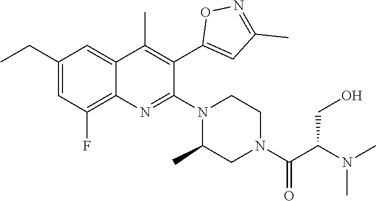 [SMILES: CCc1cc(F)c2nc(N3CCN(C(=O)[C@H](CO)N(C)C)C[C@H]3C)c(-c3cc(C)no3)c(C)c2c1] |
| 169 | 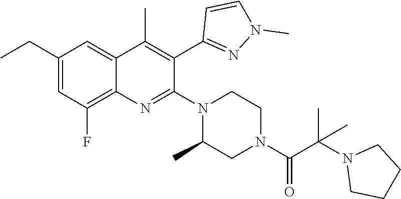 [SMILES: CCc1cc(F)c2nc(N3CCN(C(=O)C(C)(C)N4CCCC4)C[C@H]3C)c(-c3ccn(C)n3)c(C)c2c1] |
| 170 | 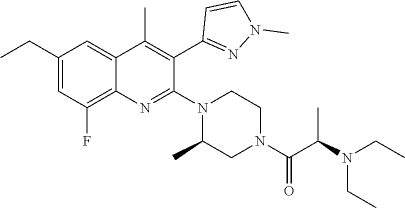 [SMILES: CCc1cc(F)c2nc(N3CCN(C(=O)[C@@H](C)N(CC)CC)C[C@H]3C)c(-c3ccn(C)n3)c(C)c2c1] |
| 171 | 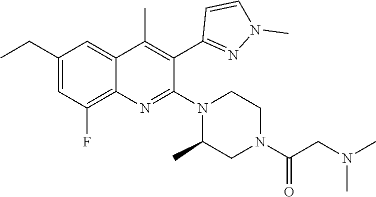 [SMILES: CCc1cc(F)c2nc(N3CCN(C(=O)CN(C)C)C[C@H]3C)c(-c3ccn(C)n3)c(C)c2c1] |
| 172 | 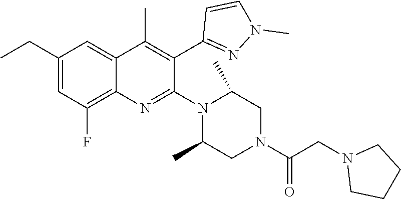 [SMILES: CCc1cc(F)c2nc(N3[C@H](C)CN(C(=O)CN4CCCC4)C[C@H]3C)c(-c3ccn(C)n3)c(C)c2c1] |
| 173 | 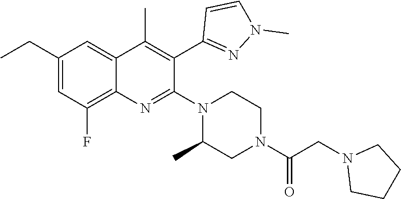 [SMILES: CCc1cc(F)c2nc(N3CCN(C(=O)CN4CCCC4)C[C@H]3C)c(-c3ccn(C)n3)c(C)c2c1] |
| 174 | 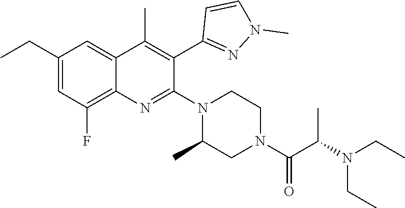 [SMILES: CCc1cc(F)c2nc(N3CCN(C(=O)[C@H](C)N(CC)CC)C[C@H]3C)c(-c3ccn(C)n3)c(C)c2c1] |
| 175 | 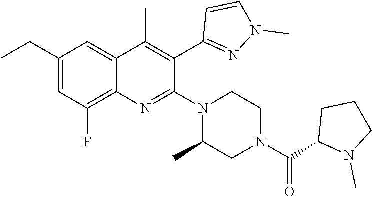 [SMILES: CCc1cc(F)c2nc(N3CCN(C(=O)[C@@H]4CCCN4C)C[C@H]3C)c(-c3ccn(C)n3)c(C)c2c1] |
| 176 | 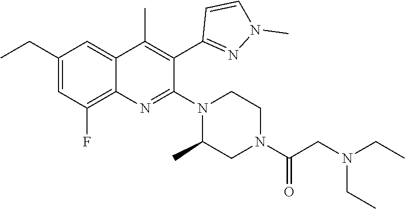 [SMILES: CCc1cc(F)c2nc(N3CCN(C(=O)CN(CC)CC)C[C@H]3C)c(-c3ccn(C)n3)c(C)c2c1] |
| 177 | 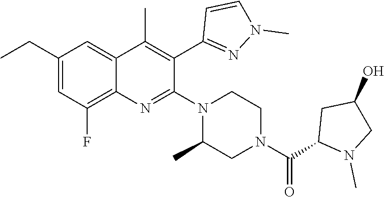 [SMILES: CCc1cc(F)c2nc(N3CCN(C(=O)[C@@H]4C[C@@H](O)CN4C)C[C@H]3C)c(-c3ccn(C)n3)c(C)c2c1] |
| 178 | 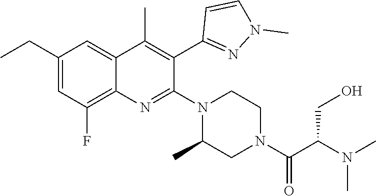 [SMILES: CCc1cc(F)c2nc(N3CCN(C(=O)[C@H](CO)N(C)C)C[C@H]3C)c(-c3ccn(C)n3)c(C)c2c1] |
| 179 | 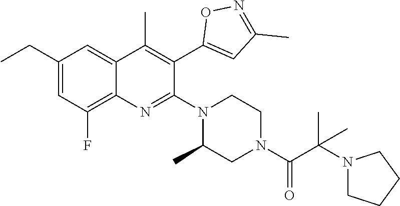 [SMILES: CCc1cc(F)c2nc(N3CCN(C(=O)C(C)(C)N4CCCC4)C[C@H]3C)c(-c3cc(C)no3)c(C)c2c1] |
| 180 | 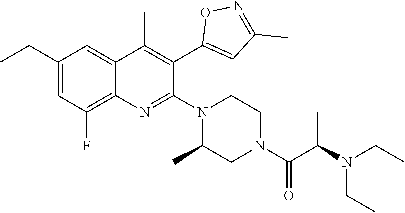 [SMILES: CCc1cc(F)c2nc(N3CCN(C(=O)[C@@H](C)N(CC)CC)C[C@H]3C)c(-c3cc(C)no3)c(C)c2c1] |
| 181 | 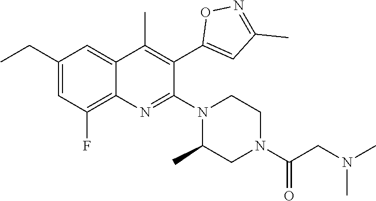 [SMILES: CCc1cc(F)c2nc(N3CCN(C(=O)CN(C)C)C[C@H]3C)c(-c3cc(C)no3)c(C)c2c1] |
| 182 | 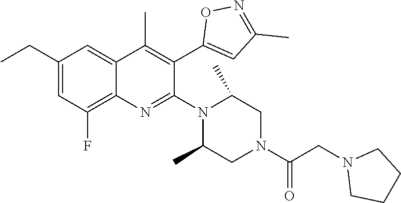 [SMILES: CCc1cc(F)c2nc(N3[C@H](C)CN(C(=O)CN4CCCC4)C[C@H]3C)c(-c3cc(C)no3)c(C)c2c1] |
| 183 | 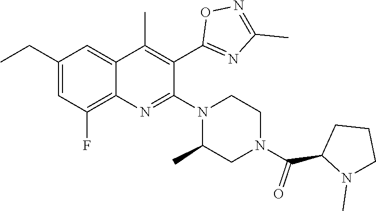 [SMILES: CCc1cc(F)c2nc(N3CCN(C(=O)[C@H]4CCCN4C)C[C@H]3C)c(-c3nc(C)no3)c(C)c2c1] |
| 184 | 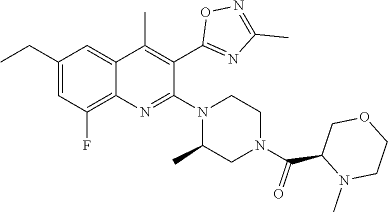 [SMILES: CCc1cc(F)c2nc(N3CCN(C(=O)[C@H]4COCCN4C)C[C@H]3C)c(-c3nc(C)no3)c(C)c2c1] |
| 185 | 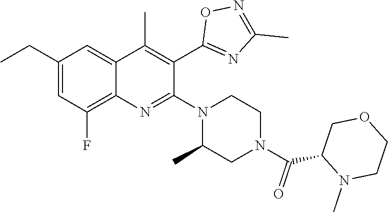 [SMILES: CCc1cc(F)c2nc(N3CCN(C(=O)[C@@H]4COCCN4C)C[C@H]3C)c(-c3nc(C)no3)c(C)c2c1] |
| 186 | 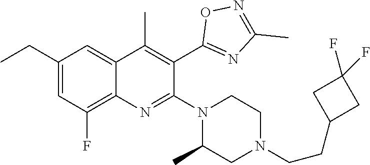 [SMILES: CCc1cc(F)c2nc(N3CCN(CCC4CC(F)(F)C4)C[C@H]3C)c(-c3nc(C)no3)c(C)c2c1] |
| 187 | 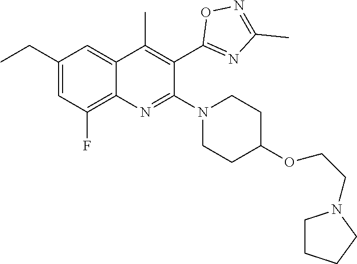 [SMILES: CCc1cc(F)c2nc(N3CCC(OCCN4CCCC4)CC3)c(-c3nc(C)no3)c(C)c2c1] |
| 188 | 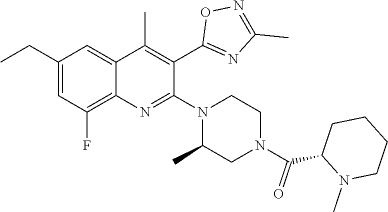 [SMILES: CCc1cc(F)c2nc(N3CCN(C(=O)[C@@H]4CCCCN4C)C[C@H]3C)c(-c3nc(C)no3)c(C)c2c1] |
| 189 | 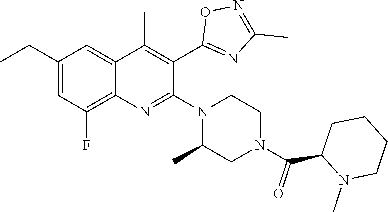 [SMILES: CCc1cc(F)c2nc(N3CCN(C(=O)[C@H]4CCCCN4C)C[C@H]3C)c(-c3nc(C)no3)c(C)c2c1] |
| 190 | 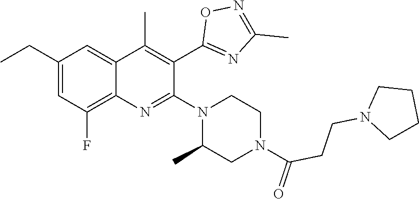 [SMILES: CCc1cc(F)c2nc(N3CCN(C(=O)CCN4CCCC4)C[C@H]3C)c(-c3nc(C)no3)c(C)c2c1] |
| 191 | 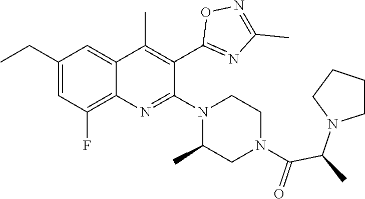 [SMILES: CCc1cc(F)c2nc(N3CCN(C(=O)[C@H](C)N4CCCC4)C[C@H]3C)c(-c3nc(C)no3)c(C)c2c1] |
| 192 | 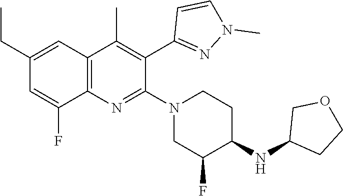 [SMILES: CCc1cc(F)c2nc(N3CC[C@@H](N[C@@H]4CCOC4)[C@@H](F)C3)c(-c3ccn(C)n3)c(C)c2c1] |
| 193 | 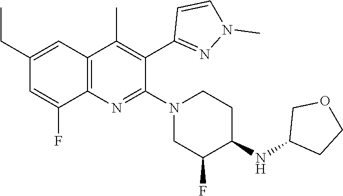 [SMILES: CCc1cc(F)c2nc(N3CC[C@@H](N[C@H]4CCOC4)[C@@H](F)C3)c(-c3ccn(C)n3)c(C)c2c1] |
| 194 | 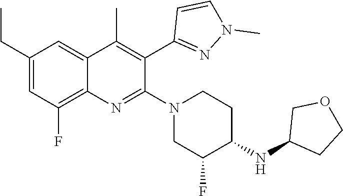 [SMILES: CCc1cc(F)c2nc(N3CC[C@H](N[C@@H]4CCOC4)[C@H](F)C3)c(-c3ccn(C)n3)c(C)c2c1] |
| 195 | 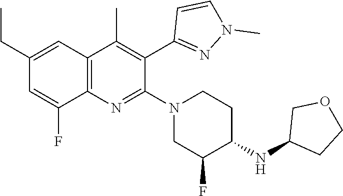 [SMILES: CCc1cc(F)c2nc(N3CC[C@H](N[C@@H]4CCOC4)[C@@H](F)C3)c(-c3ccn(C)n3)c(C)c2c1] |
| 196 | 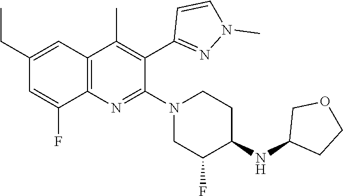 [SMILES: CCc1cc(F)c2nc(N3CC[C@@H](N[C@@H]4CCOC4)[C@H](F)C3)c(-c3ccn(C)n3)c(C)c2c1] |
| 197 | 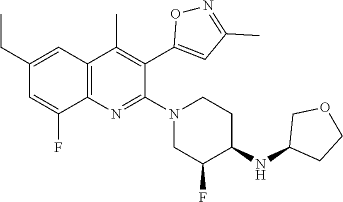 [SMILES: CCc1cc(F)c2nc(N3CC[C@@H](N[C@@H]4CCOC4)[C@@H](F)C3)c(-c3cc(C)no3)c(C)c2c1] |
| 198 | 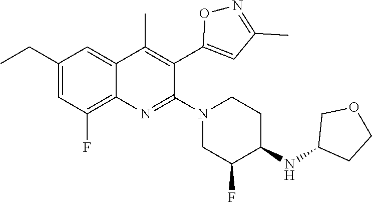 [SMILES: CCc1cc(F)c2nc(N3CC[C@@H](N[C@H]4CCOC4)[C@@H](F)C3)c(-c3cc(C)no3)c(C)c2c1] |
| 199 | 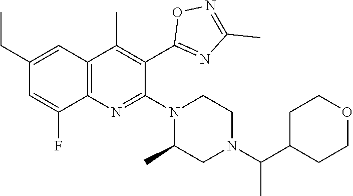 [SMILES: CCc1cc(F)c2nc(N3CCN(C(C)C4CCOCC4)C[C@H]3C)c(-c3nc(C)no3)c(C)c2c1] |
| 200 | 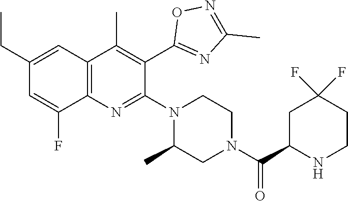 [SMILES: CCc1cc(F)c2nc(N3CCN(C(=O)[C@H]4CC(F)(F)CCN4)C[C@H]3C)c(-c3nc(C)no3)c(C)c2c1] |
| 201 | 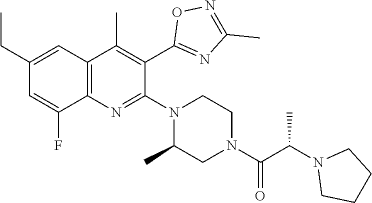 [SMILES: CCc1cc(F)c2nc(N3CCN(C(=O)[C@H](C)N4CCCC4)C[C@H]3C)c(-c3nc(C)no3)c(C)c2c1] |
| 202 | 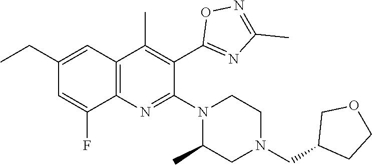 [SMILES: CCc1cc(F)c2nc(N3CCN(C[C@H]4CCOC4)C[C@H]3C)c(-c3nc(C)no3)c(C)c2c1] |
| 203 | 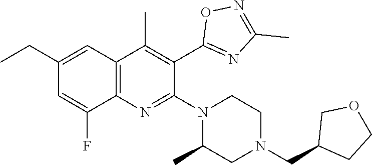 [SMILES: CCc1cc(F)c2nc(N3CCN(C[C@@H]4CCOC4)C[C@H]3C)c(-c3nc(C)no3)c(C)c2c1] |
| 204 | 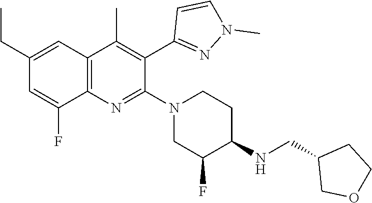 [SMILES: CCc1cc(F)c2nc(N3CC[C@@H](NC[C@@H]4CCOC4)[C@@H](F)C3)c(-c3ccn(C)n3)c(C)c2c1] |
| 205 | 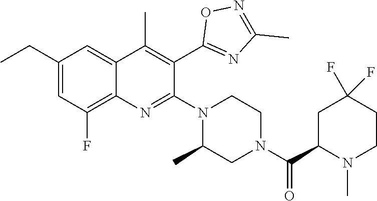 [SMILES: CCc1cc(F)c2nc(N3CCN(C(=O)[C@H]4CC(F)(F)CCN4C)C[C@H]3C)c(-c3nc(C)no3)c(C)c2c1] |
| 206 | 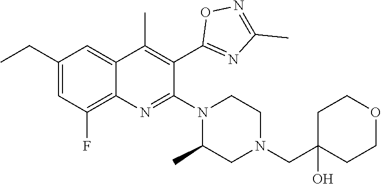 [SMILES: CCc1cc(F)c2nc(N3CCN(CC4(O)CCOCC4)C[C@H]3C)c(-c3nc(C)no3)c(C)c2c1] |
| 207 | 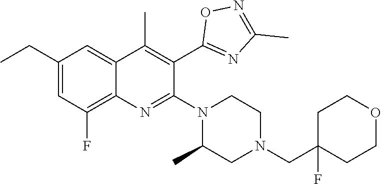 [SMILES: CCc1cc(F)c2nc(N3CCN(CC4(F)CCOCC4)C[C@H]3C)c(-c3nc(C)no3)c(C)c2c1] |
| 208 | 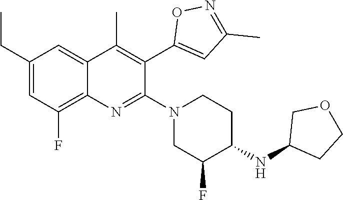 [SMILES: CCc1cc(F)c2nc(N3CC[C@H](N[C@@H]4CCOC4)[C@@H](F)C3)c(-c3cc(C)no3)c(C)c2c1] |

### Pharmaceutical Composition

The disclosure also provides a pharmaceutical composition comprising a therapeutically effective amount of one or more compounds disclosed herein, or a pharmaceutically acceptable salt, stereoisomer, isotopologue, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 or a pharmaceutically acceptable salt, stereoisomer, isotopologue, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, isotopologue, and/or tautomer thereof that is administered is governed by such considerations, and is the minimum amount necessary to exhibit antagonism against the kappa opioid receptor. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

In certain embodiments, the compound as described herein or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another embodiment, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

A compound of the present disclosure can be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

In another embodiment, the present disclosure provides a method for antagonizing the KOR. The method comprises contacting the receptor with an effective amount of a compound or a pharmaceutically acceptable salt thereof as described herein. The contacting can occur, for instance, in vivo or in vitro, in accordance with various embodiments.

The present disclosure also provides, in some embodiments, a method for treating a disorder in a subject suffering therefrom. The disorder is one for which antagonism of kappa-opioid receptor (KOR) is therapeutically indicated.

As described in summary above, the KOR is a member of the opioid receptor family which binds the opioid peptide dynorphin as the primary endogenous ligand. The term “antagonism” refers generally to molecules that interact with a receptor and thereby function as an antagonist, either by binding to the receptor at the binding site of its natural ligand or at locations other than the binding site. Thus, the expression “antagonism of KOR” or the like refers to antagonistic interaction with KOR either by binding to KOR at the site of dynorphin, or at a location other than the binding site (i.e., allosteric binding).

In another embodiment, the present disclosure provides a method for treating a disorder in a subject suffering therefrom, comprising administering to the subject a compound or pharmaceutically acceptable salt thereof as described herein. The disorder is one or more selected from a substance abuse or addiction, psychiatric disorder, obesity and eating disorders, migraine, postnatal depression, neurodegenerative disease or disorder, epilepsy, status epilepticus, and seizure.

In some embodiments, the disorder is disrupted sleep that is occasioned by, or is concomitant with, pain, a psychiatric disorder as described herein, or a drug therapy of a psychiatric disorder. The pain can be a chronic pain or a neuropathic pain. The disruption of sleep, in various embodiments, can be characterized as sleep disturbance, such as occurs by disorders of initiating and maintaining sleep (DIMS, insomnias), excessive somnolence, disorders of sleep-wake schedule, or partial arousals (parasomnias) [Cormier R E. Sleep Disturbances. In: Walker H K. Hall W D, Hurst J W, editors. Clinical Methods: The History, Physical, and Laboratory Examinations. 3rd edition. Boston: Butterworths; 1990. Chapter 77]. In other embodiments, the disruption of sleep is loss of sleep, such as occurs by abnormal frequency and/or duration of periods of wakefulness. In some embodiments, the disrupted sleep is characterized by a disrupted stage of sleep, such as rapid eye movement (REM) sleep. Treatment in this context, in accordance with the methods described herein, can result in the normalization of sleep, that is, a lessening or elimination of the disturbances of sleep. In various embodiments, normalized sleep includes restoration of REM sleep, extension of REM sleep duration, reduced frequency of interruption of REM sleep, and combinations thereof.

In some embodiments, the disorder is one of substance abuse or addiction. For example, the disorder can be chosen from gambling, drug addiction, drug abuse, alcohol dependence, alcohol abuse, and substance-induced depression or mood disorders.

In other embodiments, the disorder is a psychiatric disorder. Examples of psychiatric disorders amenable to treatment by the methods described herein include anxiety disorder, depressive disorder, mood disorder, schizophrenia spectrum disorders, stress-related disorder, obsessive-compulsive disorder, social phobia, generalized anxiety disorder (GAD), social anxiety disorder, post-traumatic stress disorder (PTSD), personality disorders, and autism spectrum disorders (ASD).

The term “anxiety disorder,” as understood in the art, refers generally to various forms of abnormal and pathological fear and anxiety. Current psychiatric diagnostic criteria recognize a wide variety of anxiety disorders, including generalized anxiety disorder, panic disorder, stress-related disorders, obsessive compulsive disorder, phobia, social anxiety disorder, separation anxiety disorder and post-traumatic stress disorder (PTSD). In one embodiment, the anxiety disorder is a social anxiety disorder. In another embodiment, the anxiety disorder is phobia

Generalized anxiety disorder is characterized by chronic and long-lasting anxiety whose focus is not any particular object or situation. A person suffering from generalized anxiety can experience non-specific persistent fear and worry and/or exhibit exaggerated concern with routine matters. Generalized anxiety disorder is the most common anxiety disorder to affect older adults.

A person suffering from panic disorder can unexpectedly experience brief attacks of intense terror and apprehension. Accompanying manifestations include trembling, shaking, confusion, dizziness, nausea, difficulty breathing. The APA defines the attacks as fear or discomfort that abruptly arises and peaks in less than ten minutes, that can last for several hours, and that can be triggered by stress, fear, or even exercise; the specific cause is not always apparent. A diagnosis of panic disorder additionally attaches chronic consequences to the attacks: these include worry over potential implications of the attack, persistent fear of future attacks, or significant behavioral changes occasioned by the attacks. Accordingly, those suffering from panic disorder can experience symptoms outside of specific panic episodes. For example, the panic sufferer can notice normal changes in heartbeat underlying a misplaced concern about cardiac health or onset of another panic attack. In some instances, a person may experience heightened awareness (hypervigilance) of body function during panic attacks, wherein any perceived physiological change is interpreted as a possible life threatening illness, i.e., extreme hypochondriasis).

Obsessive compulsive disorder (OCD) is an anxiety disorder primarily characterized by repetitive obsessions (distressing, persistent, and intrusive thoughts or images) and compulsions (urges to perform specific acts or rituals). The OCD thought pattern resides in a belief invoking a causative relationship where one does not actually exist. Compulsions can be entirely illogical, such as walking in a certain pattern to alleviate the obsession of impending harm. Compulsions can be outright inexplicable, often residing in an urge to complete a ritual triggered by nervousness. A minority of OCD sufferers may experience only obsessions with no overt compulsions; even fewer sufferers experience only compulsions.

Phobia is the single largest category of anxiety disorders, including all instances in which a specific stimulus or situation triggers fear or anxiety. Sufferers typically anticipate terrifying consequences from encountering the object of their fear: examples include social phobia, specific phobia, agoraphobia, and phobia of an animal, location, or a bodily fluid.

Post-traumatic stress disorder (PTSD) is an anxiety disorder resulting from a traumatic experience. Post-traumatic stress can follow an extreme situation, such as combat, rape, hostage situations, or even serious accident. It can also result from chronic exposure to a severe stressor; for example, soldiers may endure individual battles but suffer stress from protracted combat. Common PTSD symptoms include flashbacks, avoidant behaviors, and depression.

The methods described herein can be useful in the treatment of a depressive disorder, depression, or depressive illness. Examples include major depression, drug-resistant depression, dysthymia, and bipolar disorder.

In embodiments, the methods described herein are useful in treating a mood or affective disorder. Examples include major depressive disorder (MDD), bipolar disorder, anhedonia, dysthymia, major depression, psychotic major depression (PMD), psychotic depression, postpartum depression, seasonal affective disorder (SAD), and catatonic depression, which is a rare and severe form of major depression involving disturbances of motor behavior and other symptoms.

As used herein, the terms “anhedonia” and “anhedonic symptom” are interchangeable and are defined as the inability to experience pleasure from activities usually found enjoyable, such as exercise, hobbies, music, sexual activities, or social interactions. Anhedonia resembles the criterion of “depressive disorder with melancholic features” given by the DSM-5 as melancholic depression characterized by a loss of pleasure in most or all activities, a failure of reactivity to pleasurable stimuli, a quality of depressed mood more pronounced than that of grief or loss, a worsening of symptoms in the morning hours, early morning waking, psychomotor retardation, excessive weight loss, or excessive guilt. It should be understood that, in various embodiments, treatment of depressive disorder with melancholic features comprises treatment of both the depressive disorder and the melancholic features associated therewith. In an embodiment, the mood disorder is anhedonia. In another embodiment, the mood disorder is major depression. In yet another embodiment, the mood disorder is seasonal affective disorder (SAD).

In additional embodiments, the methods described herein are useful in the treatment of schizophrenia or a schizoaffective disorder, or obesity or an eating disorder, such as bulimia, anorexia nervosa, and the like.

In still further embodiments, the methods are use in treating migraine. Also contemplated is prophylactic therapy, by which administration of a KOR antagonist compound described herein prevents migraine in individuals who are at risk of or otherwise predisposed to reoccurrence of migraine.

In another embodiment, the methods described herein are useful for the treatment of postnatal depression (PND). Immediately after birth, significant decreases in progesterone levels can lead to the onset of PND. Symptoms of PND include mild depression to the more sever psychosis requiring hospitalization. PND also can be accompanied by, or manifested in, severe anxiety and irritability. Frustrating typical treatment regimens, PND is not amenable to treatment by classic antidepressants, and women suffering from PND show greater incidence of premenstrual syndrome (PMS).

In various embodiments, the methods described herein are useful for the treatment of a neurodegenerative disease or disorder, including disorders of mood and behavior associated with neurodegenerative diseases. The scope of neurodegenerative diseases contemplated herein includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; ataxia and convulsion; seizures that are caused by schizoaffective disorder or by drugs administered to treat schizophrenia; benign forgetfulness; brain edema; cerebellar ataxia, including McLeod neuroacanthocytosis syndrome (MLS); closed head injury; coma; contusive injuries, such as spinal cord injury and head injury; dementias including multi-infarct dementia and senile dementia; disturbances of consciousness; Down syndrome; drug-induced or medication-induced Parkinsonism, such as neuroleptic-induced acute akathisia, acute dystonia. Parkinsonism, tardive dyskinesia, neuroleptic malignant syndrome, and medication-induced postural tremor; epilepsy; fragile X syndrome; Gilles de la Tourette's syndrome; head trauma; hearing impairment and loss; Huntington's disease; Lennox syndrome; levodopa-induced dyskinesia; mental retardation; movement disorders including akinesias and akinetic (rigid) syndromes, including basal ganglia calcification, corticobasal degeneration, multiple system atrophy. Parkinsonism-ALS dementia complex, Parkinson's disease, postencephalitic parkinsonism, and progressively supranuclear palsy; muscular spasms and disorders associated with muscular spasticity or weakness including chorea (such as benign hereditary chorea, drug-induced chorea, hemiballism, Huntington's disease, neuroacanthocytosis. Sydenham's chorea, and symptomatic chorea), dyskinesia (such as tics including complex tics, simple tics, and symptomatic tics), myoclonus (including generalized myoclonus and focal cyloclonus), tremor (such as rest tremor, postural tremor, and intention tremor) and dystonia (including axial dystonia, dystonic writer's cramp, hemiplegic dystonia, paroxysmal dystonia, and focal dystonia such as blepharospasm, oromandibular dystonia, and spasmodic dysphonia and torticollis); neuronal damage including ocular damage, retinopathy, or macular degeneration of the eye; neurotoxic injury that follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; Parkinson's disease; seizure; status epilecticus; stroke; tinnitus; tubular sclerosis; and neurodegeneration induced by viral infection such as acquired immunodeficiency syndrome (AIDS) and encephalopathies. The methods also contemplate treatment of prevention of loss of neuronal function characteristic of neurodegenerative disorders.

In some embodiments, the methods described herein are useful in the treatment of epilepsy. Epilepsy is a brain disorder characterized by repeated seizures over time. Various types of epilepsy contemplated for treatment include generalized epilepsy, childhood absence epilepsy, juvenile myoclonic epilepsy, epilepsy with grand-mal seizures on awakening, West syndrome. Lennox-Gastaut syndrome, partial epilepsy, temporal lobe epilepsy, frontal lobe epilepsy, and benign focal epilepsy of childhood.

In embodiments, the methods described herein are useful in the treatment of status epilepticus. Status epilepticus (SE) can include convulsive status epilepticus, early status epilepticus, established status epilepticus, refractory status epilepticus, super-refractory status epilepticus; non-convulsive status epilepticus, generalized status epilepticus, complex partial status epilepticus; generalized periodic epileptiform discharges; and periodic lateralized epileptiform discharges.

Convulsive status epilepticus is characterized by the presence of convulsive status epileptic seizures, and can include early status epilepticus, established status epilepticus, refractory status epilepticus, or super-refractory status epilepticus. Early status epilepticus is treated with a first line therapy. Established status epilepticus is characterized by status epileptic seizures that persist despite treatment with a first line therapy; therefore, a second line therapy is administered. Refractory status epilepticus is characterized by status epileptic seizures which persist despite treatment with first- and second-line therapies; a general anesthetic is typically administered. Super refractory status epilepticus is characterized by status epileptic seizures that persist despite treatment with first- and second-line therapies and a general anesthetic for 24 hours or more.

Non-convulsive status epilepticus includes focal non-convulsive status epilepticus, such as complex partial non-convulsive status epilepticus, simple partial non-convulsive status epilepticus, and subtle non-convulsive status epilepticus; and generalized non-convulsive status epilepticus, such as late onset absence non-convulsive status epilepticus, atypical absence non-convulsive status epilepticus, or typical absence non-convulsive status epilepticus.

In embodiments, the methods described herein are useful in the treatment of a seizure. As used herein, the term “seizure” refers to physical manifestations or changes in behavior that occur after an episode of abnormal electrical activity in the brain. In addition, the term “seizure” is often used interchangeably with “convulsion,” referring to rapid and uncontrollable shaking of a person's body. During a convulsion, the person's muscles contract and relax repeatedly. The type of behavior and brain activity define two categories of seizures, specifically generalized and partial (also called local or focal), respectively. Classification of a seizure informs a diagnose of epilepsy.

Electrical impulses produced throughout the entire brain give rise to generalized seizures, whereas impulses localized to a part of the brain give rise to partial seizures. The part of the brain generating the seizures is sometimes called the focus.

Generalized seizures are categorized according to six types. The most common and dramatic, and therefore the best-known, is the generalized convulsion: it is also called the grand-mal seizure. In this type of seizure, a sufferer loses consciousness and usually collapses. The loss of consciousness is followed by generalized body stiffening for 30 to 60 seconds—the “tonic” phase—then by violent jerking for 30 to 60 seconds—the “clonic” phase—after which the sufferer progresses into a deep—the “postictal” or after-seizure phase. During grand-mal seizures, a person can suffer injuries and accidents, such as tongue biting and urinary incontinence.

Second, absence seizures cause brief loss of consciousness, typically a few seconds, with few or no symptoms. The sufferer, who is most often a child, typically interrupts an activity with a blank stare. These seizures begin and end abruptly and they can occur several times a day. Sufferers are usually unaware of the seizure aside from possible awareness of “losing time.” Third, myoclonic seizures consist of sporadic jerks, usually on both sides of the body. Patients sometimes describe the jerks as brief electrical shocks. When violent, these seizures can result in dropping or involuntarily throwing objects. Fourth, clonic seizures consist of repetitive and rhythmic jerks that simultaneously involve both sides of the body. Fifth, tonic seizures are characterized by stiffening of the muscles. Finally, atonic seizures consist of sudden and general loss of muscle tone, particularly in the arms and legs, often resulting in a fall.

In various embodiments, the seizures described herein include epileptic seizures; acute repetitive seizures; cluster seizures; continuous seizures; unremitting seizures; prolonged seizures; recurrent seizures; status epilepticus seizures, such as refractory convulsive status epilepticus and non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; and withdrawal seizures.

### EXAMPLES

The present disclosure is further illustrated by the following examples. The examples below are non-limiting and constitute additional embodiments of the present disclosure.

General Methods. Unless otherwise stated, commercially available reagents and solvents were used without purification. Solvents for extraction: ACS grade. Solvents for reaction: reagent grade. Reagents: unless otherwise noted, from Alfa Aesar, Fisher, Combi-Blocks, and Aldrich highest quality available. TLC: silica gel 60 F254 aluminum plates. (whatman, type A1 Sil G/UV, 250 μm layer); visualization by UV absorption. Flash chromatography was performed on silica gel 60 (0.40-0.63 mm, 230-440 mesh, EM Science). Biotage Flash+ systems were used for medium-pressure column chromatography. NMR: ¹H and ¹³C spectra were obtained at Bruker AV NEO 500 MHz spectrometer and Bruker AVIII 400 MHz spectrometers. ¹H, and ¹³C NMR data are reported with chemical shifts (S) in parts-per-million (ppm) relative to the residual signal of the deuterated solvent as follows: chemical shift, multiplicity (s=singlet, d=doublet, t=triplet, q=quartet, qn=quintet, m=multiplet, and br=broad), coupling constant in Hz. Reactions were monitored by Agilent 1260 Infinity with Agilent 6120 Quadrupole LC/MS detector. Purity was determined by LCMS using a Agilent SB—C₁₈ column (1.8 μm, 2.1×50 mm) and detection was performed with a UV at 254 and 230 nm wavelength. Elution was carried out with a 10-90% gradient over 5 min of CH₃CN in water containing 0.1% HCO₂H at a flow rate of 1.0 mL/min at 25° C. The purity of all test compounds is higher than 95%. High resolution mass spectra were obtained on an Agilent 6230 TOF LC/MS system using electrospray ionization (ESI) in positive mode.

### Synthesis of Compounds

The following exemplary procedures are offered to illustrate syntheses of specific compounds described in the present disclosure. The skilled person in the art can readily adapt the procedures, starting materials, and reagents to the syntheses of all compounds described herein.

### Example 1: (3R,4S)-1-(6-ethyl-4-methyl-3-(1-methyl-1H-pyrazol-3-yl)-8-(1-methyl-1H-pyrazol-5-yl)quinolin-2-yl)-3-fluoro-N-(tetrahydro-2H-pyran-4-yl)piperidin-4-amine (1)

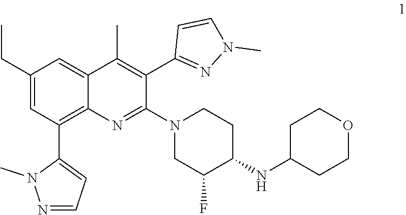 [SMILES: CCc1cc(-c2ccnn2C)c2nc(N3CC[C@H](NC4CCOCC4)[C@H](F)C3)c(-c3ccn(C)n3)c(C)c2c1]

### Step 1. 2-chloro-6-ethyl-4-methyl-3-(1-methyl-1H-pyrazol-3-yl)-8-(1-methyl-1H-pyrazol-5-yl)quinoline (int-5)

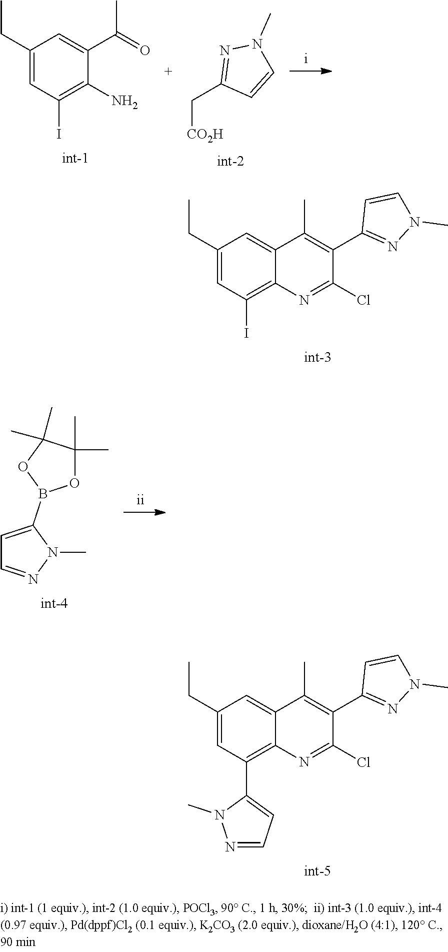

A mixture of (1-methyl-1H-pyrazol-3-yl) acetic acid (int-2; 121 mg, 0.86 mmol) and int-1 (250 mg, 0.86 mmol) in POCl₃ (3 mL) was stirred at 90° C. for 1 h. The POCl₃ was removed under reduced pressure. The residue was quenched with ice/water and basified with a saturated aqueous solution of NaHCO₃ to pH ˜7, and the product extracted with EtOAc. The organic phase was dried over Na₂SO₄ and concentrated under reduced pressure. The crude product was purified by column chromatography using hexanes/EtOAc to give 2-chloroquinoline int-3. ¹H NMR (500 MHz, CDCl₃): δ (ppm) 8.22 (d, J=1.7 Hz, 1H), 7.77-7.73 (m, 1H), 7.51 (d, J=2.2 Hz, 1H), 6.33 (d, J=2.2 Hz, 1H), 4.02 (s, 3H), 2.81 (q, J=7.6 Hz, 2H), 2.54 (s, 3H), 1.34 (t, J=7.6 Hz, 3H). LCMS: (M+1) m/z=412, 414.

A mixture of int-3 (50 mg, 0.12 mmol), int-4 (25 mg, 0.11 mmol) Pd(dppf)Cl₂ (8 mg, 0.012 mmol) and K₂CO₃ (33 mg, 0.24 mmol) in dioxane/H₂O (0.8/0.2 mL) was flushed with nitrogen. The mixture was heated at 120° C. for 2 h. After cooling to rt, the mixture was partitioned between brine (30 mL) and EtOAc (30 mL). The aqueous layer was extracted with EtOAc (2×30 mL) and the combined organic layers were dried over Na₂SO₄ and concentrated under reduced pressure. The residue was purified by column chromatography using hexanes/EtOAc to give int-5. ¹H NMR (500 MHz, CDCl₃) δ 7.86 (d, J=1.9 Hz, 1H), 7.61 (d, J=1.9 Hz, 1H), 7.60 (d, J=1.9 Hz, 1H), 7.51 (d, J=2.2 Hz, 1H), 6.41 (d, J=1.9 Hz, 1H), 6.34 (d, J=2.2 Hz, 1H), 4.02 (s, 3H), 3.76 (s, 3H), 2.89 (q, J=7.6 Hz, 1H), 2.58 (s, 3H), 1.37 (t, J=7.6 Hz, 3H). LCMS: (M+1) m/z=366, 368.

### Step 2. (3R,4S)-3-fluoro-N-(tetrahydro-2H-pyran-4-yl)piperidin-4-amine

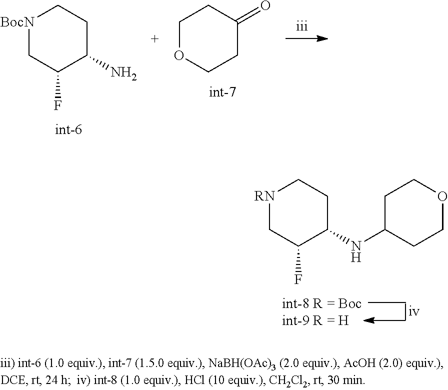

A mixture of ketone int-7 (275 mg, 2.75 mmol), chiral amine int-6 (400 mg, 1.83 mmol), NaBH(OAc)₃ (776 mg, 3.66 mmol), and AcOH (209 μL, 3.66 mmol) in 1,2-dichloroethane (6 mL) was stirred at room temperature for 24 hrs. The mixture was quenched with water and the product extracted with EtOAc (3×), the organic phase was concentrated under reduced pressure, and the product was purified by column chromatography using CH₂C₂:MeOH to give compound int-8. LCMS: (M+1) m/z=303.

To a solution of int-8 (470 mg, 1.15 mmol) in CH₂C₂ (I mL) was slowly added at room temperature HCl (4M dioxane) (2.9 mL, 11.5 mmol), and the resultant mixture was stirred at room temperature for 30 min. The solution was concentrated under reduced pressure. The mixture was redissolved in MeOH and quenched with PL-HCO₃ MPSPE resin (Agilent) and stirred for 2 min. The mixture was filtered and the resin washed with MeOH. The organic phase was concentrated under reduced pressure and the product int-9 used without further purification. LCMS (M+H) m/z=203

### Step 3. (3R,4S)-1-(6-ethyl-4-methyl-3-(1-methyl-1H-pyrazol-3-yl)-8-(1-methyl-1H-pyrazol-5-yl)quinolin-2-yl)-3-fluoro-N-(tetrahydro-2H-pyran-4-yl)piperidin-4-amine (1)

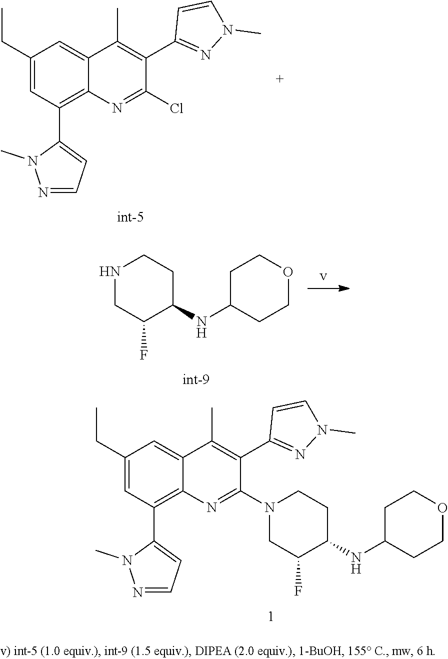

A suspension of int-5 (10 mg, 0.027 mmol), int-9 (8.3 mg, 0.04 mmol) and DIPEA (9.5 μL, 0.054 mmol) in I-BuOH (0.5 mL) was heated at 155° C. under microwave irradiation for 6 h. After cooling to room temperature, the mixture was concentrated under reduced pressure and purified by prep-TLC using CH₂Cl₂/MeOH to give 1. ¹H NMR (500 MHz, CDCl₃) δ 7.80-7.76 (m, 1H), 7.57 (d, J=1.8 Hz, 1H), 7.47 (d, J=2.0 Hz, 1H), 7.45 (d, J=2.2 Hz, 1H), 6.39 (d, J=2.2 Hz, 1H), 6.35 (d, J=1.8 Hz, 1H), 4.66 (d, J=48.6 Hz, 1H), 4.01 (s, 3H), 3.98-3.92 (m, 2H), 3.88-3.80 (m, 2H), 3.73 (s, 3H), 3.45-3.36 (m, 3H), 2.87-2.73 (m, 4H), 2.71-2.61 (m, 1H), 2.57 (s, 3H), 2.52-2.45 (m, 1H), 1.73-1.66 (m, 2H), 1.50-1.37 (m, 4H), 1.34 (t, J=7.6 Hz, 4H). HRMS (ESI-TOF) calcd. for C₃₀H₃₈FN₇O [M+H]⁺ 532.3195, found 532.3169.

### Example 2: (R)-6-ethyl-8-fluoro-4-methyl-3-(1-methyl-1H-pyrazol-3-yl)-2-(2-methyl-4-((tetrahydro-2H-pyran-4-yl)methyl)piperazin-1-yl)quinoline (2)

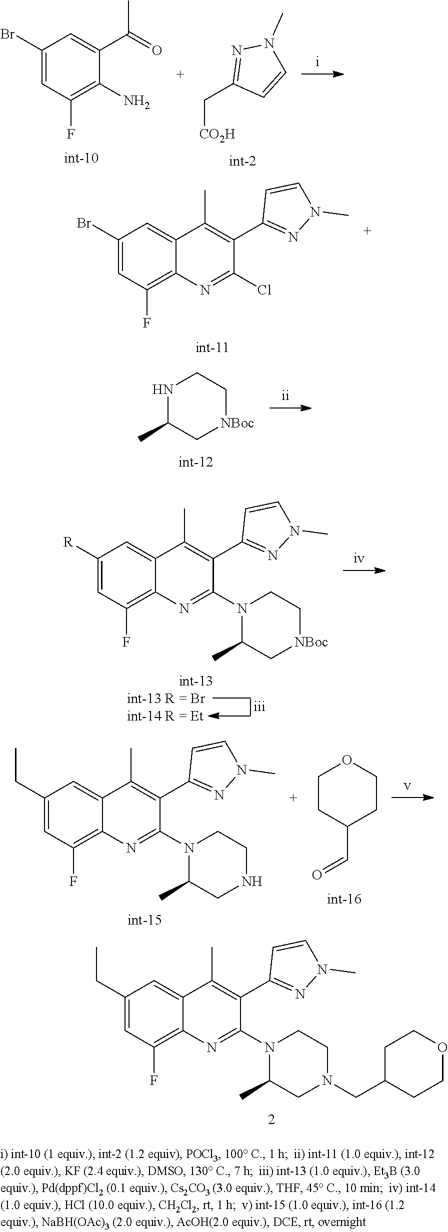

A mixture of (1-methyl-1H-pyrazol-3-yl) acetic acid (int-2; 350 mg, 2.5 mmol) and int-10 (480 mg, 2.08 mmol) in POCl₃ (5 mL) was stirred at 100° C. for 1 h. The POCl₃ was removed under reduced pressure. The residue was quenched with ice/water and basified with saturated NaHCO₃(aq) to pH ˜7, and the product extracted with EtOAc. The organic phase was dried over Na₂SO₄ and concentrated under reduced pressure. The crude product was purified by column chromatography using hexanes/EtOAc to give 2-chloroquinoline int-11. ¹H NMR (500 MHz, CDCl₃) δ 7.95 (dd, J=2.0, 1.4 Hz, 1H), 7.57 (dd, J=9.2, 2.0 Hz, 1H), 7.53 (d, J=2.3 Hz, 1H), 6.36 (d, J=2.2 Hz, 1H), 4.02 (s, 3H), 2.53 (s, 3H). LCMS: (M+1) m/z=353, 355.

A mixture of int-11 (180 mg, 0.507 mmol), int-12 (203 mg, 1.01 mmol) and KF (71 mg, 1.22 mmol) in DMSO was heated at 130 C for 7 h under microwave irradiation. The mixture was cooled to room temperature, diluted with EtOAc (60 mL) and washed with brine (3×). The organic phase was dried over Na₂SO₄, concentrated under reduced pressure and the product was purified by column chromatography using CH₂Cl₂/EtOAc. int-13 was obtained. LCMS: (M+1) m/z=518, 520.

To a suspension of int-13 (150 mg, 0.29 mmol), Cs₂CO₃ (283 mg, 0.87 mmol) and Pd(dppf)Cl₂ (21 mg, 0.029 mmol) in THF (3 mL) was added Et₃B (1M in THF) (868 μL, 0.87 mmol). The reaction was heated at 45° C. for 10 min. The mixture was diluted with EtOAc and washed with brine (2×). The organic phase was dried over Na₂SO₄, concentrated under reduced pressure and the product was purified by column chromatography using CH₂Cl₂/EtOAc to give the product int-14. ¹H NMR (500 MHz, CDCl₃) δ 7.46 (d, J=2.2 Hz, 1H), 7.45 (d, J=1.8 Hz, 1H), 7.17 (dd, J=11.5, 1.8 Hz, 1H), 6.31 (d, J=2.2 Hz, 1H), 4.01 (s, 3H), 3.87-3.68 (m, 2H), 3.50-3.30 (m, 2H), 3.27-2.97 (m, 3H), 2.77 (q, J=7.6 Hz, 2H), 2.49 (s, 3H), 1.44 (s, 9H), 1.30 (t, J=7.6 Hz, 3H). LCMS: (M+1) m/z=468.

To a solution of int-14 (95 mg, 0.2 mmol) in CH₂Cl₂ (1 mL) was slowly added at rt HCl (4M dioxane) (0.5 mL, 2.0 mmol) and the mixture was stirred at it for 1 h. The solution was concentrated under reduced pressure and the product int-15 was used without further purification. LCMS (M+H) m/z=368

A mixture of int-15 (11 mg, 0.027 mmol), int-16 (3.7 mg, 0.032 mmol), NaBH(OAc)₃ (11.4 mg, 0.054 mmol), DIPEA (4.7 μL, 0.027 mmol) and AcOH (3.1 μL, 0.054 mmol) in 1,2-dichloroethane (0.4 mL) was stirred at rt for overnight. The mixture was diluted with EtOAc and washed with brine, the organic phase was dried over Na₂SO₄ and concentrated under reduced pressure. The residue was purified by preparative-TLC (CH₂Cl₂:MeOH, 95:5) to give 2. ¹H NMR (500 MHz, CDCl₃) δ 7.44 (d, J=2.2 Hz, 1H), 7.43 (s, 1H), 7.16 (dd, J=11.6, 1.8 Hz, 1H), 6.32 (d, J=2.1 Hz, 1H), 4.00 (s, 3H), 3.97-3.92 (m, 2H), 3.78-3.72 (m, 1H), 3.40-3.32 (m, 2H), 3.30-3.27 (m, 1H), 2.76 (q, J=7.6 Hz, 2H), 2.50-2.46 (m, 4), 2.29-2.24 (m, 1H), 2.22-2.09 (m, 3H), 2.07-2.01 (m, 1H), 1.72-1.61 (m, 2H), 1.30 (t, J=7.6 Hz, 3H), 1.26-1.21 (m, 2H), 1.07 (d, J=6.5 Hz, 3H). HRMS (ESI-TOF) calcd. for C₂₇H₃₆FN₅O [M+H]⁺ 466.2977, found 466.2959.

### Example 3: 1-(3-(1,5-dimethyl-1H-pyrazol-3-yl)-6-ethyl-8-fluoro-4-methylquinolin-2-yl)-N-((3R,4S)-3-fluorotetrahydro-2H-pyran-4-yl)piperidin-4-amine

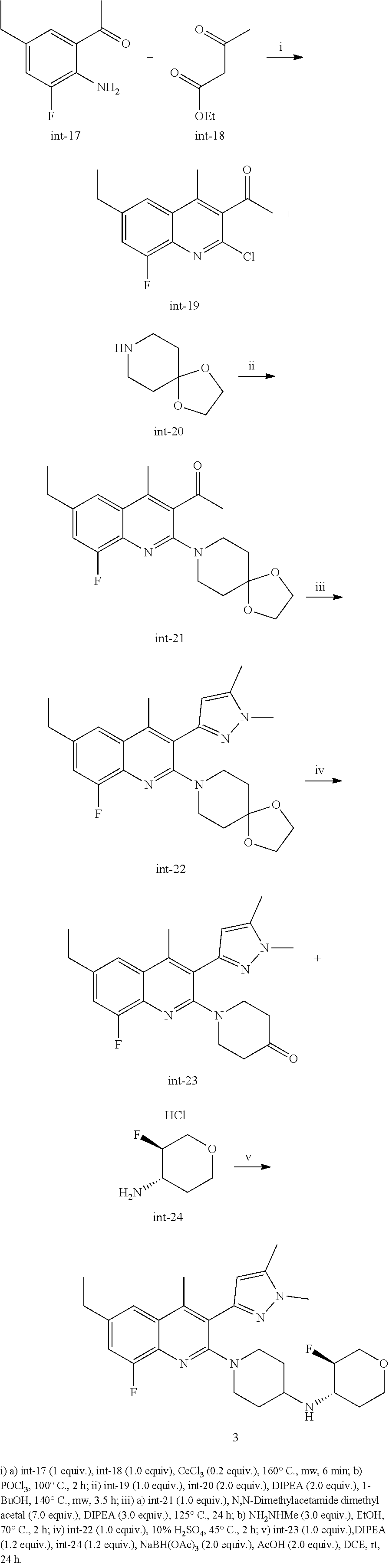

A mixture of int-17 (250 mg, 1.38 mmol), int-18 (175 IL, 1.38 mmol) and CeCl₃ (68 mg, 0.28 mmol) was heated in a microwave vial at 160 C under microwave irradiation for 6 min. The mixture was stirred with 1 mL of water for 5 min. The solid was filtrated and washed with water (3×) and with hexanes (2×). The quinolone product was. ¹H NMR (500 MHz, CDCl₃) δ 9.42 (br s, 1H), 7.34 (s, 1H), 7.18 (dd, J=11.0, 1.6 Hz, 1H), 2.73 (q, J=7.6 Hz, 2H), 2.60 (s, 3H), 2.44 (s, 3H), 1.28 (t, J=7.6 Hz, 3H). LCMS: (M+1) m/z=248.

A mixture of the quinolone product above (160 mg, 0.65 mmol) in POCl₃ (3 mL) was stirred at 90° C. for 1 h. The POCl₃ was removed under reduced pressure. The residue was quenched with ice/water and the product extracted with EtOAc (3×). The organic phase was dried over Na₂SO₄ and concentrated under reduced pressure. The crude was purified by column chromatography using hexanes/EtOAc to give 2-chloroquinoline int-19. ¹H NMR (500 MHz, CDCl₃) δ 7.54 (dd, J=1.8, 0.9 Hz, 1H), 7.34 (dd, J=10.8, 1.7 Hz, 1H), 2.84 (q, J=7.6 Hz, 2H), 2.65 (s, 3H), 2.59 (s, 31H), 1.33 (t, J=7.6 Hz, 3H).

A suspension of int-19 (840 mg, 3.16 mmol), int-20 (811 μL, 6.32 mmol) and DIPEA (1.1 mL, 6.32 mmol) in 1-BuOH (10 mL) was heated at 140° C. under microwave irradiation for 3.5 h. The mixture was concentrated under reduced pressure and purified by column chromatography using hexanes/EtOAc to give int-21. ¹H NMR (500 MHz, CDCl₃) δ 7.46 (dd, J=1.8, 0.9 Hz, 1H), 7.22 (dd, J=11.4, 1.8 Hz, 1H), 3.98 (s, 4H), 3.52-3.47 (m, 4H), 2.78 (q, J=7.6 Hz, 2H), 2.53 (s, 3H), 2.51 (s, 3H), 1.87-1.81 (m, 4H), 1.30 (t, J=7.6 Hz, 3H). LCMS: (M+1) m/z=373.

A mixture of int-21 (370 mg, 1.0 mmol), N,N-dimethylacetamide dimethyl acetal (1.02 mL, 7.0 equiv.), DIPEA (523 μL, 3.0 mmol) was heated at 125 C for 20 h. The mixture was concentrated under reduced pressure. The residue was dissolved in EtOAc and washed with brine (3×). The organic phase was dried over Na₂SO₄ and concentrated under reduced pressure. The residue was washed with hexanes (3×), and the resulting solid was used in the next step without further purification.

A mixture of the above intermediate (880 mg, 2.0 mmol) and N-methyl hydrazine in EtOH (6 ml) was heated at 70 C for 2 h. The mixture was concentrated under reduced pressure and the product purified by column chromatography using hexanes/EtOAc. Imidazole int-22 was obtained. ¹H NMR (500 MHz, CDCl₃) δ 7.43 (d, J=1.8 Hz, 1H), 7.21 (dd, J=11.5, 1.8 Hz, 1H), 6.08 (s, 1H), 3.93 (s, 411), 3.55 (s, 3H), 3.444-3.37 (m, 2H), 3.26-3.18 (m, 2H), 2.78 (q, J=7.6 Hz, 2H), 2.41 (s, 3H), 2.33 (s, 3H), 1.63 (t. J=5.7 Hz, 4H), 1.31 (t, J=7.6 Hz, 3H).

A mixture of int-22 (180 mg, 0.42 mmol) in 10% H₂SO₄ was stirred at 45° C. for 2 h. The mixture was basified with sat. aq. NaHCO₃ to pH 7 and the product was extracted with EtOAc (2×). The organic phase was dried over Na₂SO₄ and concentrated under reduced pressure. Product int-23 was obtained and used without further purification. ¹H NMR (500 MHz, CDCl₃) δ 7.48 (d, J=1.5 Hz, 1H), 7.25 (dd, J=11.2, 1.8 Hz, 1H), 6.13 (s, 1H), 3.68-3.60 (m, 2H), 3.58 (s, 3H), 3.48-3.42 (m, 2H), 2.80 (q, J=7.6 Hz, 2H), 2.47-2.35 (m, 7H), 2.34 (s, 3H), 1.32 (t, J=7.6 Hz, 3H). LCMS: (M+1) m/z=381.

A mixture of int-23 (40 mg, 0.105 mmol), int-24 (20 mg, 0.126 mmol), NaBH(OAc)₃ (46 mg, 0.21 mmol), DIPEA (16 μL, 0.126 mmol) and AcOH (12 μL, 0.21 mmol) in 1,2-dichloroethane (0.6 mL) was stirred at room temperature overnight. The mixture was diluted with EtOAc and washed with brine, the organic phase was dried over Na₂SO₄ and concentrated under reduced pressure. The residue was purified by preparative-TLC (DCM:MeOH, 95:5) to give 3. ¹H NMR (500 MHz, CDCl₃) δ 7.46-7.42 (m, 1H), 7.22 (dd, J=11.5, 1.7 Hz, 1H), 6.09 (d, J=1.3 Hz, 1H), 4.27 (dtd, J=49.3, 8.5, 4.6 Hz, 1H), 4.03 (ddd, J=11.7, 7.4, 4.6 Hz, 1H), 3.89-3.81 (m, 1H), 3.71-3.63 (m, 1H), 3.62-3.56 (m, 1H), 3.55 (s, 3H), 3.40 (tt, J=10.9, 2.3 Hz, 1H), 3.35-3.27 (m, 1H), 3.01-2.90 (m, 2H), 2.78 (q, J=7.6 Hz, 2H), 2.72-2.60 (m, 2H), 2.42 (s, 3H), 2.34 (s, 3H), 1.99-1.85 (m, 2H), 1.84-1.73 (m, 11), 1.50-1.39 (m, 1H), 1.31 (t, J=7.6 Hz, 3H), 1.28-1.18 (m, 1H), 1.16-1.01 (m, 1H). HRMS (ESI-TOF) calcd. for C₂₇H₃₅F₂N₅O [M+H]⁺ 484.2883, found 484.2859.

### Example 4: 1-(3-(1,5-dimethyl-1H-pyrazol-3-yl)-6-ethyl-8-fluoro-4-methylquinolin-2-yl)-N-((3S,4R)-3-fluorotetrahydro-2H-pyran-4-yl)piperidin-4-amine (4)

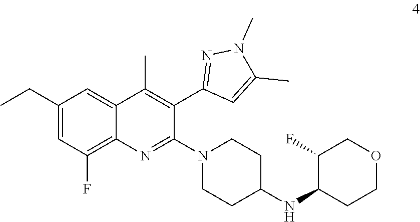 [SMILES: CCc1cc(F)c2nc(N3CCC(N[C@@H]4CCOC[C@H]4F)CC3)c(-c3cc(C)n(C)n3)c(C)c2c1]

Compound 4 was obtained by the procedure of Example 3 using the appropriate amine and int-23. HRMS (ESI-TOF) calcd. for C₂₇H₃₅F₂N₅O [M+H]⁺ 484.2883, found 484.2874.

### Example 5: 1-(3-(1,5-dimethyl-1H-pyrazol-3-yl)-6-ethyl-8-fluoro-4-methylquinolin-2-yl)-N-((3S,4S)-3-fluorotetrahydro-2H-pyran-4-yl)piperidin-4-amine (5)

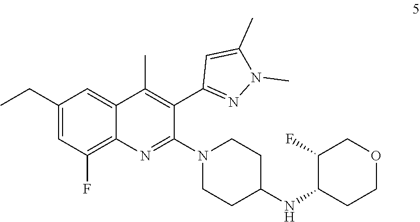 [SMILES: CCc1cc(F)c2nc(N3CCC(N[C@H]4CCOC[C@H]4F)CC3)c(-c3cc(C)n(C)n3)c(C)c2c1]

Compound 5 was obtained by the procedure of Example 3 using the appropriate amine and int-23. HRMS (ESI-TOF) calcd. for C₂₇H₃₅F₂N₅O [M+H]⁺ 484.2883, found 484.2866.

### Example 6: 1-(3-(1,5-dimethyl-1H-pyrazol-3-yl)-6-ethyl-8-fluoro-4-methylquinolin-2-yl)-N-(tetrahydro-2H-pyran-4-yl)piperidin-4-amine (6)

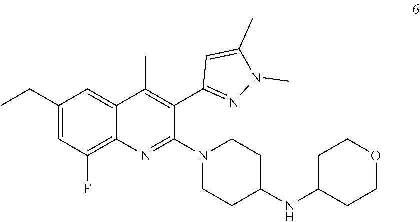 [SMILES: CCc1cc(F)c2nc(N3CCC(NC4CCOCC4)CC3)c(-c3cc(C)n(C)n3)c(C)c2c1]

Compound 6 was obtained by the procedure of Example 3 using the appropriate amine and int-23. HRMS (ESI-TOF) calcd. for C₂₇H₃₆FN₅O [M+H]⁺ 466.2977, found 466.2954.

### Example 7: 1-(6-ethyl-4-methyl-3,8-bis(1-methyl-1H-pyrazol-3-yl)quinolin-2-yl)-N-((3R,4S)-3-fluorotetrahydro-2H-pyran-4-yl)piperidin-4-amine (7)

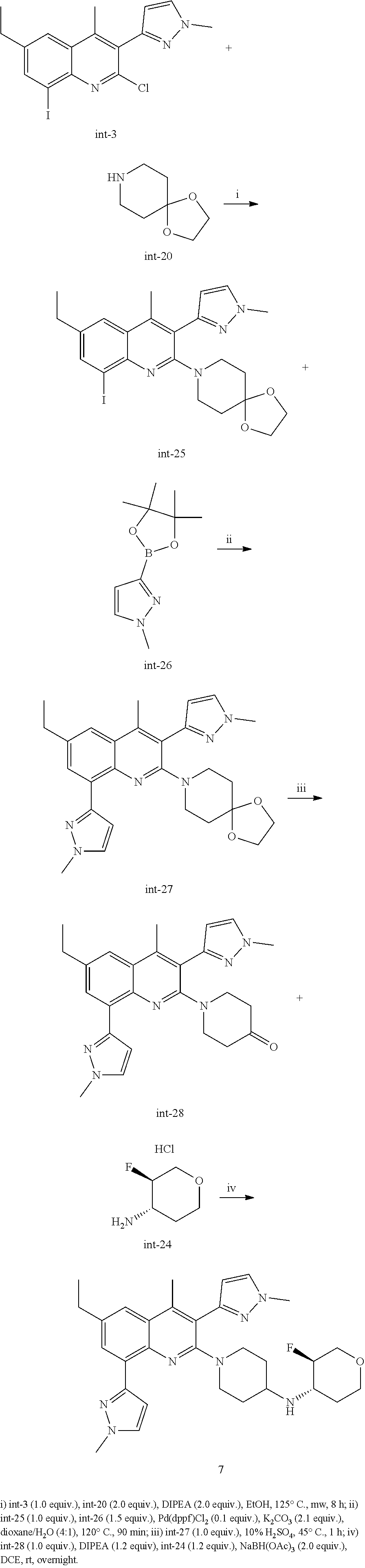

A mixture of int-3 (80 mg, 0.194 mmol), int-20 (50 μL, 0.39 mmol) and DIPEA (68 μL, 0.39 mmol) was heated at 125 C for 8 h under microwave irradiation. The mixture was concentrated under reduced pressure and the product purified by column chromatography using hexanes/EtOAc to give product int-25. ¹H NMR (500 MHz, CDCl₃) δ 8.05 (d, J=1.8 Hz, 1H), 7.64-7.61 (m, 1H), 7.44 (d, J=2.2 Hz, 1H), 6.32 (d, J=2.2 Hz, 1H), 4.00 (s, 3H), 3.94 (s, 4H), 3.41-3.35 (m, 4H), 2.74 (q, J=7.6 Hz, 2H), 2.49 (s, 3H), 1.69 (t, J=5.7 Hz, 4H), 1.30 (t, J=7.6 Hz, 3H). LCMS: (M+1) m/z=519.

A mixture of int-25 (60 mg, 0.116 mmol), int-26 (36 mg, 0.174 mmol) and Pd(dppf)Cl₂ (8.5 mg, 0.012 mmol) and K₂C₀₃ (32 mg, 0.23 mmol) in dioxane/H₂O (0.8/0.2 mL) was flushed with nitrogen. The mixture was heated at 120° C. for 2 h. After cooling to room temperature, the mixture was partitioned between brine (30 mL) and EtOAc (30 mL). The aqueous layer was extracted with EtOAc (2×30 mL) and the combined organic layers were dried over Na₂SO₄ and concentrated under reduced pressure. The residue was purified by column chromatography using hexanes/EtOAc to give int-27. ¹H NMR (500 MHz, CDCl₃) δ 8.16 (d, J=2.0 Hz, 1H), 7.67 (d, J=2.0 Hz, 1H), 7.47-7.41 (m, 3H), 6.35 (d, J=2.2 Hz, 1H), 4.01 (s, 3H), 4.00 (s, 3H), 3.94 (s, 4H), 3.30 (t, J=5.7 Hz, 4H), 2.83 (q, J=7.6 Hz, 2H), 2.54 (s, 3H), 1.64 (t, J=5.7 Hz, 4H), 1.34 (t, J=7.6 Hz, 3H).

A mixture of int-27 (29 mg, 0.061 mmol) in 10% H₂SO₄ was stirred at 45 C for 1 h. The mixture was basified with sat. aq. solution of NaHCO₃ to pH 7 and the product was extracted with EtOAc (2×). The organic phase was dried over Na₂SO₄ and concentrated under reduced pressure. int-28 was obtained and used without further purification. ¹H NMR (500 MHz, CDCl₃) δ 8.11 (s, 1H), 7.71 (d, J=2.0 Hz, 1H), 7.49 (d, J=2.2 Hz, 1H), 7.44 (d. J=2.2 Hz, 1H), 7.22 (d, J=2.2 Hz, 1H), 6.36 (d, J=2.2 Hz, 1H), 4.02 (s, 3H), 4.01 (s, 3H), 3.53 (t, J=5.9 Hz, 4H), 2.85 (q, J=7.6 Hz, 2H), 2.55 (s, 3H), 2.38 (t, J=5.9 Hz, 4H), 1.35 (t, J=7.6 Hz, 3H).

A mixture of int-28 (24 mg, 0.056 mmol), int-24 (10.6 mg, 0.067 mmol), NaBH(OAc)₃ (24 mg, 0.112 mmol), DIPEA (11.7 μL, 0.067 mmol) and AcOH (6.5 μL, 0.112 mmol) in 1,2-dichloroethane (0.6 mL) was stirred at rt overnight. The mixture was diluted with EtOAc and washed with brine, the organic phase was dried over Na₂SO₄ and concentrated under reduced pressure. The residue was purified by preparative-TLC (DCM:MeOH, 95:5) to give 7. ¹H NMR (500 MHz, CDCl₃) δ 8.16 (d, J=2.0 Hz, 1H), 7.68 (d, J=2.0 Hz, 1H), 7.45 (d, J=2.2 Hz, 2H), 7.40 (d, J=2.2 Hz, 1H), 6.35 (d, J=2.2 Hz, 1H), 4.41-4.23 (m, 1H), 4.07-4.02 (m, 1H), 4.02 (s, 3H), 4.01 (s, 3H), 3.89-3.83 (m, 1H), 3.68-3.62 (m, 2H), 3.43-3.34 (m, 1H), 3.32-3.26 (m, 1H), 3.02-2.95 (m, 1H), 2.84 (q, J=7.6 Hz, 2H), 2.79-2.66 (m, 3H), 2.54 (s, 3H), 2.01-1.94 (m, 1H), 1.87-1.65 (m, 2H), 1.54-1.43 (m, 1H), 1.34 (t, J=7.6 Hz, 3H), 1.30-1.18 (m, 2H). HRMS (ESI-TOF) calcd. for C₃₀H₃₈FN₇O [M+H]⁺ 532.3195, found 532.3171.

### Example 8: (R)-6-ethyl-4-methyl-3,8-bis(1-methyl-1H-pyrazol-3-yl)-2-(2-methylpiperazin-1-yl)quinoline (8)

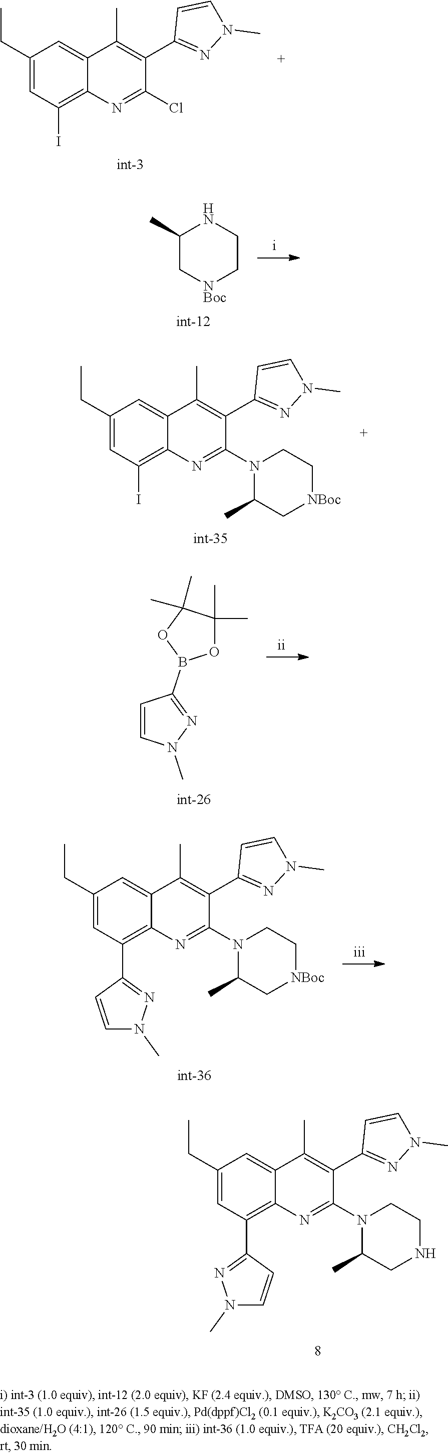 [SMILES: CCc1cc(I)c2nc(Cl)c(-c3ccn(C)n3)c(C)c2c1 ; C[C@@H]1CN(C(=O)OC(C)(C)C)CCN1 ; CCc1cc(I)c2nc(N3CCN(C(=O)OC(C)(C)C)C[C@H]3C)c(-c3ccn(C)n3)c(C)c2c1 ; Cn1ccc(B2OC(C)(C)C(C)(C)O2)n1 ; CCc1cc(-c2ccn(C)n2)c2nc(N3CCN(C(=O)OC(C)(C)C)C[C@H]3C)c(-c3ccn(C)n3)c(C)c2c1 ; CCc1cc(-c2ccn(C)n2)c2nc(N3CCNC[C@H]3C)c(-c3ccn(C)n3)c(C)c2c1]

A mixture of int-3 (150 mg, 0.36 mmol) and int-12 (144 mg, 0.72 mmol), KF (50 mg, 0.86 mmol) in DMSO (1.0 mL) was heated at 130 C for 7 h under microwave (mw) irradiation. The mixture was cooled to room temperature, diluted with EtOAc (60 mL) and washed with brine (3×). The organic phase was dried over Na₂SO₄, concentrated under reduced pressure and the product was purified by column chromatography using hexanes/EtOAc. int-35 was obtained. ¹H NMR (500 MHz, CDCl₃) δ 8.07 (d. J=1.8 Hz, 1H), 7.64 (d, J=1.8 Hz, 1H), 7.46 (d, J=2.2 Hz, 1H), 6.28 (d, J=2.2 Hz, 1H), 4.01 (s, 3H), 3.98-3.67 (m, 2H), 3.61-3.38 (m, 2H), 3.22-2.94 (m, 3H), 2.75 (q, J=7.6 Hz, 2H), 2.48 (s, 3H), 1.45 (s, 9H), 1.30 (t, J=7.6 Hz, 3H), 1.13-1.01 (m, 3H).

A mixture of int-35 (Example 8; 45 mg, 0.078 mmol), int-26 (24 mg, 0.117 mmol) and Pd(dppf)C₁₂ (5.7 mg, 0.0078 mmol) and K₂C₀₃ (23 mg, 0.164 mmol) in dioxane/H₂O (0.8/0.2 mL) was flushed with nitrogen. The mixture was heated at 120° C. for 1.5 h. After cooling to rt, the mixture was partitioned between brine (30 mL) and EtOAc (30 mL). The aqueous layer was extracted with EtOAc (2×20 mL) and the combined organic layers were dried over Na₂SO₄ and concentrated under reduced pressure. The residue was purified by column chromatography using hexanes/EtOAc to give int-36. ¹H NMR (500 MHz, CDCl₃) δ 8.15 (d, J=2.0 Hz, 1H), 7.69 (d, J=2.0 Hz, 1H), 7.45 (d, J=2.2 Hz, 1H), 7.44 (d, J=2.2 Hz, 1H), 7.34 (d, J=2.2 Hz, 1H), 6.32 (d, J=2.2 Hz, 1H), 4.02 (s, 3H), 4.01 (s, 3H), 3.94-3.61 (m, 2H), 3.46-2.96 (m, 5H), 2.84 (q, J=7.6 Hz, 2H), 2.54 (s, 3H), 1.44 (s, 9H), 1.35 (t, J=7.6 Hz, 3H), 0.99-0.89 (m, 3H).

A mixture of int-36 (30 mg, 0.057 mmol) and TFA (87 μL, 1.13 mmol) in CH₂Cl₂ (500 μL) was stirred at room temperature for 30 min. The mixture was concentrated under reduced pressure and the product purified by prep-TLC using CH₂Cl₂/MeOH to give 8. LCMS: (M+1) m/z=430.

### Example 9: 1-(6-ethyl-4-methyl-3-(1-methyl-1H-pyrazol-3-yl)-8-(1-methyl-1H-pyrazol-5-yl)quinolin-2-yl)-N-((3R,4S)-3-fluorotetrahydro-2H-pyran-4-yl)piperidin-4-amine (9)

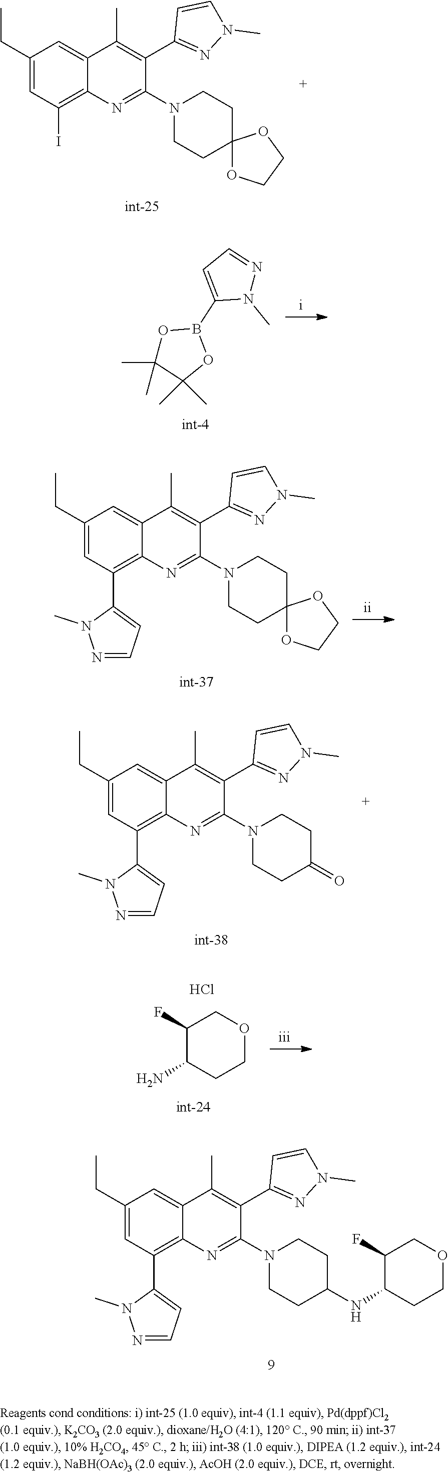

A mixture of int-25 (100 mg, 0.19 mmol), int-4 (44 mg, 0.21 mmol) and Pd(dppf)C₁₂ (14 mg, 0.019 mmol) and K₂CO₃ (53 mg, 0.38 mmol) in dioxane/H₂O (0.8/0.2 mL) was flushed with nitrogen. The mixture was heated at 120° C. for 1.5 h. After cooling to rt, the mixture was partitioned between brine (30 mL) and EtOAc (30 mL). The aqueous layer was extracted with EtOAc (2×20 mL) and the combined organic layers were dried over Na₂SO₄ and concentrated under reduced pressure. The residue was purified by column chromatography using CH₂Cl₂/MeOH to give int-37. ¹H NMR (500 MHz, CDCl₃) δ 7.76 (d, J=2.0 Hz, 1H), 7.56 (d, J=1.8 Hz, 1H), 7.45 (d, J=2.0 Hz, 1H), 7.44 (d, J=2.1 Hz, 1H), 6.34 (d, J=1.9 Hz, 1H), 6.32 (d, J=2.2 Hz, 1H), 4.00 (s, 3H), 3.89 (s, 4H), 3.74 (s, 3H), 3.16-3.10 (m, 4H), 2.82 (q, J=7.6 Hz, 2H), 2.54 (s, 3H), 1.53 (t, J=5.7 Hz, 4H), 1.34 (t, J=7.6 Hz, 3H).

A mixture of int-37 (78 mg, 0.165 mmol) and 10% H₂SO₄ (4 mL) was stirred at 45 C for 2 h. The mixture was basified with sat. aq. solution of NaHCO₃ to pH 7 and the product was extracted with EtOAc (2×). The organic phase was dried over Na₂SO₄ and concentrated under reduced pressure. The residue was purified by column chromatography using hexanes/EtOAc to give int-38. ¹H NMR (500 MHz, CDCl₃) δ 7.82-7.77 (m, 1H), 7.56 (d, J=1.9 Hz, 1H), 7.49 (d, J=2.1 Hz, 2H), 6.34 (d, J=2.2 Hz, 1H), 6.33 (d, J=1.9 Hz, 1H), 4.01 (s, 3H), 3.70 (s, 3H), 3.37 (t, J=6.0 Hz, 4H), 2.84 (q, J=7.6 Hz, 2H), 2.56 (s, 3H), 2.24 (t, J=5.9 Hz, 4H), 1.35 (t, J=7.6 Hz, 3H).

A mixture of int-38 (11 mg, 0.025 mmol), int-24 (5.0 mg, 0.031 mmol), NaBH(OAc)₃ (11 mg, 0.05 mmol), DIPEA (5.4 μL, 0.031 mmol) and AcOH (3 μL, 0.05 mmol) in 1,2-dichloroethane (0.4 mL) was stirred at rt overnight. The mixture was diluted with EtOAc and washed with brine, and the organic phase was dried over Na₂SO₄ and concentrated under reduced pressure. The residue was purified by preparative-TLC (DCM:MeOH, 95:5) to give 9. LCMS: (M+1) m/z=532.

### Example 10: (R,E)-N′-cyano-N-ethyl-4-(6-ethyl-8-fluoro-4-methyl-3-(1-methyl-1H-pyrazol-3-yl)quinolin-2-yl)-3-methylpiperazine-1-carboximidamide (10)

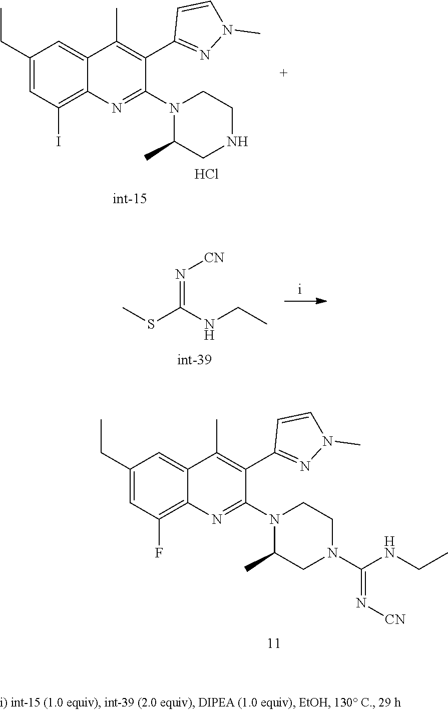

A mixture of int-15 (Example 2; 11 mg, 0.027 mmol) and int-39 (7.7 mg, 0.054 mmol), DIPEA (4.7 μL, 0.027 mmol) in EtOH (0.3 mL) was heated at 130 C for 29 h. The mixture was concentrated under reduced pressure and the product was purified by preparative-TLC using CH₂Cl₂/MeOH to give 10. ¹H NMR (500 MHz, CDCl₃) δ 7.47 (d, J=2.2 Hz, 1H), 7.47-7.44 (m, 1H), 7.19 (dd. J=11.5, 1.8 Hz, 1H), 6.28 (d, J=2.2 Hz, 1H), 4.68-4.61 (m, 1H), 4.01 (s, 3H), 3.92-3.86 (m, 1H), 3.59-3.53 (m, 1H), 3.52-3.39 (m, 4H), 3.36-3.26 (m, 3H), 2.78 (q, J=7.6 Hz, 2H), 2.48 (s, 3H), 1.31 (t, J=7.6 Hz, 3H), 1.22 (t, J=7.2 Hz, 3H), 1.04 (d, J=6.6 Hz, 3H). LCMS: (M+1) m/z=463.

### Example 11: (R,E)-N′-cyano-4-(6-ethyl-8-fluoro-4-methyl-3-(1-methyl-1H-pyrazol-3-yl)quinolin-2-yl)-3-methyl-N-(tetrahydro-2H-pyran-4-yl)piperazine-1-carboximidamide

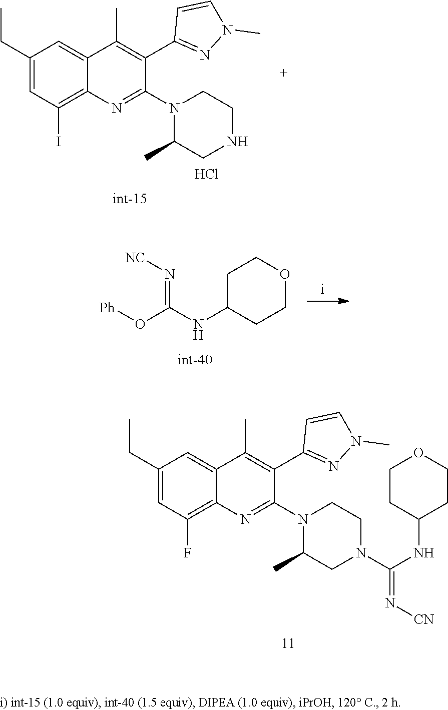 [SMILES: CCc1cc(I)c2nc(N3CCNC[C@H]3C)c(-c3ccn(C)n3)c(C)c2c1 ; Cl ; N#C/N=C(/NC1CCOCC1)Oc1ccccc1 ; CCc1cc(F)c2nc(N3CCN(/C(=N/C#N)NC4CCOCC4)C[C@H]3C)c(-c3ccn(C)n3)c(C)c2c1]

A mixture of int-15 (Example 2; 10 mg, 0.027 mmol) and int-40 (10 mg, 0.041 mmol), DIPEA (4.7 μL, 0.027 mmol) in iPrOH (0.3 mL) was heated at 120 C for 2 h. The mixture was concentrated under reduced pressure and the product was purified by preparative-TLC using CH₂C₂/MeOH to give 11. LCMS: (M+1) m/z=519.

### Example 12: (R)-6-ethyl-4-methyl-3-(1-methyl-1H-pyrazol-3-yl)-2-(2-methylpiperazin-1-yl)-8-(1H-pyrazol-3-yl)quinoline

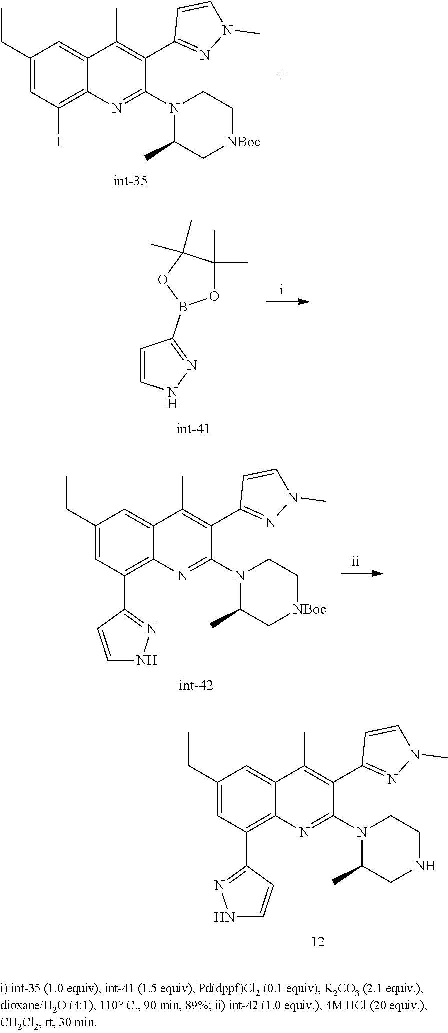 [SMILES: CCc1cc(I)c2nc(N3CCN(C(=O)OC(C)(C)C)C[C@H]3C)c(-c3ccn(C)n3)c(C)c2c1 ; CC1(C)OB(c2cc[nH]n2)OC1(C)C ; CCc1cc(-c2cc[nH]n2)c2nc(N3CCN(C(=O)OC(C)(C)C)C[C@H]3C)c(-c3ccn(C)n3)c(C)c2c1 ; CCc1cc(-c2cc[nH]n2)c2nc(N3CCNC[C@H]3C)c(-c3ccn(C)n3)c(C)c2c1]

A mixture of int-35 (Example 8; 30 mg, 0.052 mmol), int-41 (15 mg, 0.078 mmol) and Pd(dppf)Cl₂ (4 mg, 0.005 mmol) and K₂CO₃ (15 mg, 0.109 mmol) in dioxane/H₂O (0.8/0.2 mL) was flushed with nitrogen. The mixture was heated at 110° C. for 1.5 h. After cooling to rt, the mixture was partitioned between brine (30 mL) and EtOAc (30 mL). The aqueous layer was extracted with EtOAc (2×20 mL) and the combined organic layers were dried over Na₂SO₄ and concentrated under reduced pressure. The residue was purified by column chromatography using hexanes/EtOAc to give int-42 in 89% yield (24 mg). ¹H NMR (500 MHz, CDCl₃) δ 7.93 (d, J=1.9 Hz, 1H), 7.69 (d, J=1.9 Hz, 1H), 7.66 (d, J=1.9 Hz, 1H), 7.48 (d, J=2.2 Hz, 1H), 6.83 (d, J=2.0 Hz, 1H), 6.35 (d, J=2.1 Hz, 1H), 4.02 (s, 3H), 3.90-3.74 (m, 1H), 3.65-3.24 (m, 4H), 3.18-2.95 (m, 2H), 2.84 (q, J=7.6 Hz, 2H), 2.55 (s, 3H), 1.35 (t, J=7.6 Hz, 3H), 1.14-0.91 (m, 3H). LCMS: (M+1) m/z=516.

A mixture of int-42 (20 mg, 0.038 mmol) and 4.0 M HCl in dioxane (194 μL, 0.78 mmol) in CH₂Cl₂ (200 μL) was stirred at room temperature for 30 min. The mixture was concentrated under reduced pressure and the product purified by prep-TLC using CH₂Cl₂/MeOH to give 12. LCMS: (M+1) m/z=416.

### Example 13: (R)-6-ethyl-8-fluoro-4-methyl-3-(1-methyl-1H-pyrazol-3-yl)-2-(2-methylpiperazin-1-yl)quinoline (13)

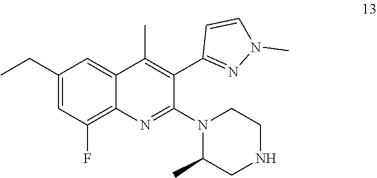 [SMILES: CCc1cc(F)c2nc(N3CCNC[C@H]3C)c(-c3ccn(C)n3)c(C)c2c1]

Compound 13 was prepared by the procedure of Example 2, and it is the same as int-15 shown in the scheme therein. HRMS (ESI-TOF) calcd. for C₂₂H₂₆FN₅ [M+H]⁺ 368.2245, found 368.2231.

### Example 14: 1-(3-(1,5-dimethyl-1H-pyrazol-3-yl)-6-ethyl-8-fluoro-4-methylquinolin-2-yl)-N-((3R,4R)-3-fluorotetrahydro-2H-pyran-4-yl)piperidin-4-amine (14)

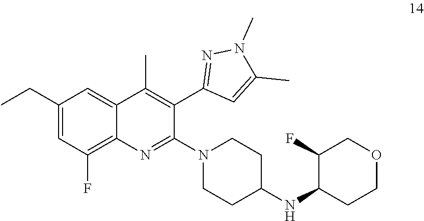 [SMILES: CCc1cc(F)c2nc(N3CCC(N[C@@H]4CCOC[C@@H]4F)CC3)c(-c3cc(C)n(C)n3)c(C)c2c1]

Compound 14 was obtained by the procedure of Example 3 using the appropriate amine and int-23. LCMS: (M+1) m/z=484.

### Example 15: (3S,4R)-1-(6-ethyl-4-methyl-3-(1-methyl-1H-pyrazol-3-yl)-8-(1-methyl-1H-pyrazol-5-yl)quinolin-2-yl)-3-fluoro-N-(tetrahydro-2H-pyran-4-yl)piperidin-4-amine (15)

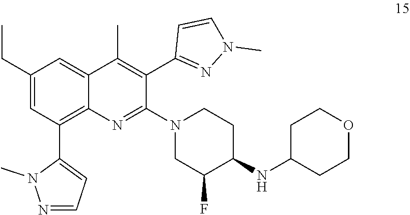 [SMILES: CCc1cc(-c2ccnn2C)c2nc(N3CC[C@@H](NC4CCOCC4)[C@@H](F)C3)c(-c3ccn(C)n3)c(C)c2c1]

Compound 15 was obtained by the procedure of Example 1 using the appropriate amine and int-5. LCMS: (M+1) m/z=532.

### Example 16: (3R,4S)-4-((1-(3-(1,5-dimethyl-1H-pyrazol-3-yl)-6-ethyl-8-fluoro-4-methylquinolin-2-yl)piperidin-4-yl)amino)tetrahydro-2H-pyran-3-ol (16)

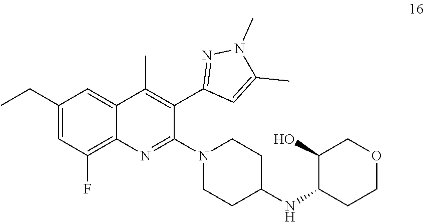 [SMILES: CCc1cc(F)c2nc(N3CCC(N[C@H]4CCOC[C@@H]4O)CC3)c(-c3cc(C)n(C)n3)c(C)c2c1]

Compound 16 was obtained by the procedure of Example 3 using the appropriate amine and int-23. LCMS: (M+1) m/z=482.

### Example 17: (3S,4R)-4-((1-(3-(1,5-dimethyl-1H-pyrazol-3-yl)-6-ethyl-8-fluoro-4-methylquinolin-2-yl)piperidin-4-yl)amino)tetrahydro-2H-pyran-3-ol (17)

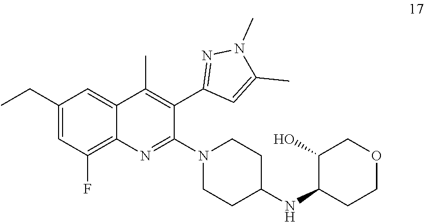 [SMILES: CCc1cc(F)c2nc(N3CCC(N[C@@H]4CCOC[C@H]4O)CC3)c(-c3cc(C)n(C)n3)c(C)c2c1]

Compound 17 was obtained by the procedure of Example 3 using the appropriate amine and int-23. LCMS: (M+1) m/z=482.

### Example 18: (S)-1-(3-(1,5-dimethyl-1H-pyrazol-3-yl)-6-ethyl-8-fluoro-4-methylquinolin-2-yl)-N-(tetrahydrofuran-3-yl)piperidin-4-amine (18)

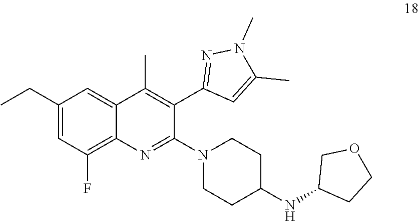 [SMILES: CCc1cc(F)c2nc(N3CCC(N[C@H]4CCOC4)CC3)c(-c3cc(C)n(C)n3)c(C)c2c1]

Compound 18 was obtained by the procedure of Example 3 using the appropriate amine and int-23. HRMS (ESI-TOF) calcd. for C₂₆H₃₄FN₅O [M+H]⁺ 452.2820, found 452.2812.

### Example 19: 1-(3-(1,5-dimethyl-1H-pyrazol-3-yl)-6-ethyl-8-fluoro-4-methylquinolin-2-yl)-N-((1-fluorocyclopropyl)methyl)piperidin-4-amine (19)

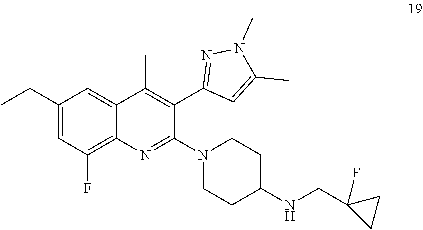 [SMILES: CCc1cc(F)c2nc(N3CCC(NCC4(F)CC4)CC3)c(-c3cc(C)n(C)n3)c(C)c2c1]

Compound 19 was obtained by the procedure of Example 3 using the appropriate amine and int-23. HRMS (ESI-TOF) calcd. for C₂₆H₃₃F₂N₅ [M+H]⁺ 454.2777. found 454.2767.

### Example 20: 1-(6-ethyl-4-methyl-3-(1-methyl-1H-pyrazol-3-yl)-8-(1-methyl-1H-pyrazol-5-yl)quinolin-2-yl)-N-((3R,4R)-3-fluorotetrahydro-2H-pyran-4-yl)piperidin-4-amine (20)

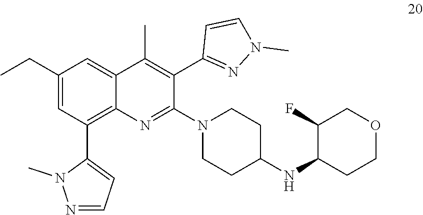 [SMILES: CCc1cc(-c2ccnn2C)c2nc(N3CCC(N[C@@H]4CCOC[C@@H]4F)CC3)c(-c3ccn(C)n3)c(C)c2c1]

Compound 20 was obtained by the procedure of Example 9 using the appropriate amine and int-38. LCMS: (M+1) m/z=532.

### Example 21: 1-(6-ethyl-4-methyl-3-(1-methyl-1H-pyrazol-3-yl)-8-(1-methyl-1H-pyrazol-5-yl)quinolin-2-yl)-N-((3S,4S)-3-fluorotetrahydro-2H-pyran-4-yl)piperidin-4-amine (21)

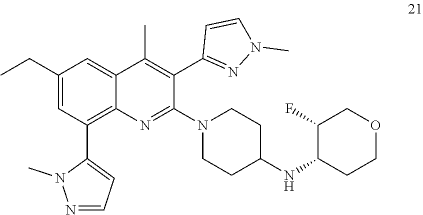 [SMILES: CCc1cc(-c2ccnn2C)c2nc(N3CCC(N[C@H]4CCOC[C@H]4F)CC3)c(-c3ccn(C)n3)c(C)c2c1]

Compound 21 was obtained by the procedure of Example 9 using the appropriate amine and int-38. LCMS: (M+1) m/z=532.

### Example 22: 1-(6-ethyl-4-methyl-3-(1-methyl-1H-pyrazol-3-yl)-8-(1-methyl-1H-pyrazol-5-yl)quinolin-2-yl)-N-((3S,4R)-3-fluorotetrahydro-2H-pyran-4-yl)piperidin-4-amine (22)

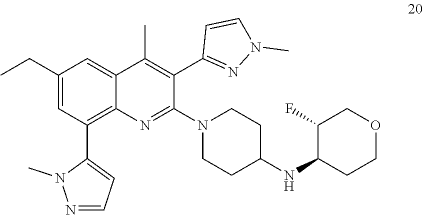 [SMILES: CCc1cc(-c2ccnn2C)c2nc(N3CCC(N[C@@H]4CCOC[C@H]4F)CC3)c(-c3ccn(C)n3)c(C)c2c1]

Compound 22 was obtained by the procedure of Example 9 using the appropriate amine and int-38. LCMS: (M+1) m/z=532.

### Example 23: 1-(6-ethyl-4-methyl-3-(1-methyl-1H-pyrazol-3-yl)-8-(1-methyl-1H-pyrazol-5-yl)quinolin-2-yl)piperidin-4-amine (23)

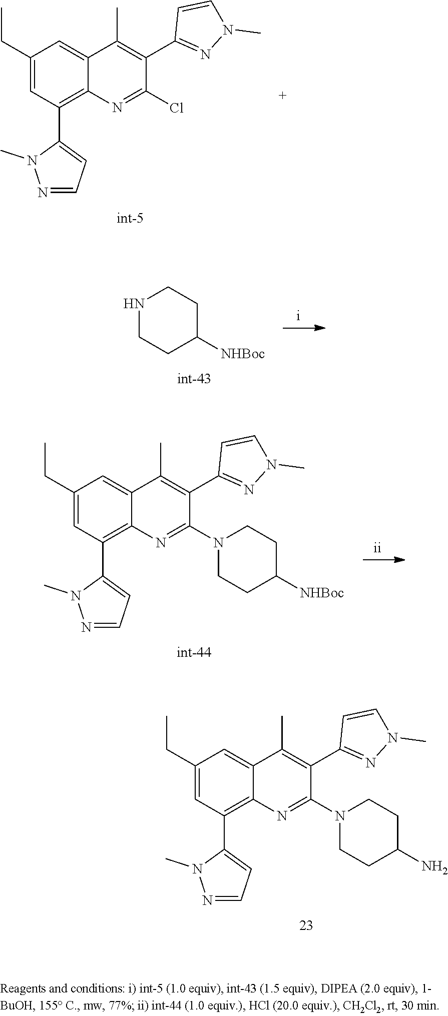

A mixture of int-5 (10 mg, 0.027 mmol) and int-43 (8.2 mg, 0.04 mmol), DIPEA (9 μL, 0.054 mmol) in n-BuOH (0.5 mL) was heated at 155 C for 6 h under microwave irradiation. The mixture was cooled to room temperature, concentrated under reduced pressure and the product was purified by prep-TLC using hexanes/EtOAc. int-44 was obtained in 77% yield (11 mg). LCMS: (M+1) m/z=530.

A mixture of int-44 (8 mg, 0.015 mmol) and 4.0 M HCl in dioxane (76 μL, 0.3 mmol) in CH₂C₂ (100 μL) was stirred at room temperature for 30 min. The mixture was concentrated under reduced pressure and the product purified by prep-TLC using CH₂Cl₂/MeOH to give compound 23. HRMS (ESI-TOF) calcd for C₂₅H₃₁N₇ [M+H]⁺ 430.2714, found 430.2717.

### Example 24: 2-(2,5-diazabicyclo[4.1.0]heptan-2-yl)-6-ethyl-4-methyl-3-(1-methyl-1H-pyrazol-3-yl)-8-(1-methyl-1H-pyrazol-5-yl)quinoline (24)

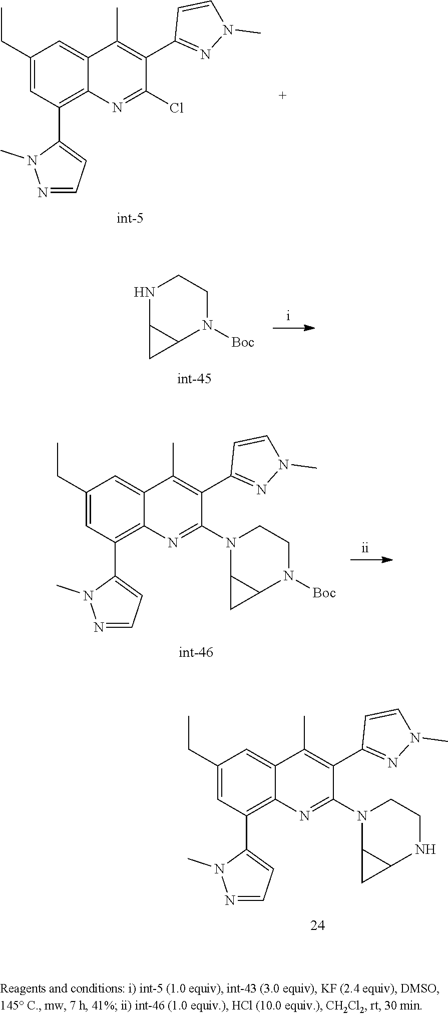

A mixture of int-5 (15 mg, 0.041 mmol) and int-45 (24 mg, 0.123 mmol), KF (5.7 mg, 0.098 mmol) in DMSO (0.5 mL) was heated at 145 C for 7 h under microwave irradiation. The mixture was cooled to room temperature, diluted with EtOAc (40 mL) and washed brine (3×). The organic phase was dried over Na₂SO₄, concentrated under reduced pressure and the product was purified by column chromatography using CH₂Cl₂/MeOH. Int-46 was obtained in 41% yield (9 mg). LCMS: (M+1) m/z=528.

A mixture of int-46 (8 mg, 0.015 mmol) and 4.0 M HCl in dioxane (38 μL, 0.152 mmol) in CH₂Cl₂ (150 μL) was stirred at room temperature for 30 min. The mixture was concentrated under reduced pressure and the product purified by prep-TLC using CH₂Cl₂/MeOH to give compound 24. HRMS (ESI-TOF) calcd for C₂₅H₂₉N₇ [M+H]⁺ 428.2557. found 428.2551.

### Example 25: (3S,4R)-1-(6-ethyl-8-fluoro-4-methyl-3-(1-methyl-1H-pyrazol-3-yl)quinolin-2-yl)-3-fluoro-N—((R)-tetrahydrofuran-3-yl)piperidin-4-amine (192)

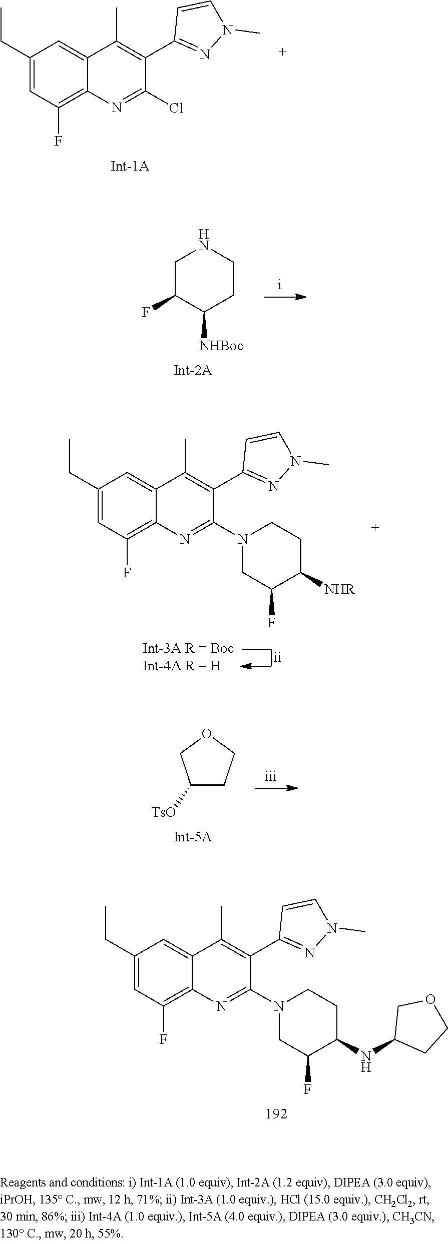

A mixture of Int-1A (200 mg, 0.66 mmol) and Int-2A (172 mg, 0.79 mmol), DIPEA (345 μL, 1.98 mmol) in iPrOH (345 μL) was heated at 135 C for 12 h under microwave irradiation. The mixture was concentrated under reduced pressure and the product was purified by column chromatography using hexanes/EtOAc. Int-3A was obtained in 71% yield (230 mg). ¹H NMR (400 MHz, CDCl₃) δ 7.48-7.42 (m, 2H), 7.18 (dd, J=11.6, 1.7 Hz, 1H), 6.37 (d, J=2.2 Hz, 1H), 4.83 (d, J=9.0 Hz, 1H), 4.70 (dd, J=49.0, 3.0 Hz, 1H), 4.14-4.04 (m, 1H), 4.01 (s, 3H), 3.68 (dd, J=23.0.9.1 Hz, 1H), 3.59-3.51 (m, 1H), 3.20-3.01 (m, 1H), 2.77 (q. J=7.5 Hz, 2H), 2.73-2.64 (m, 1H), 2.50 (s, 3H), 1.71-1.54 (m, 2H), 1.43 (s, 9H), 1.30 (t, J=7.6 Hz, 3H). LCMS: (M+1) m/z=486.

A mixture of Int-3A (220 mg, 0.45 mmol) and 4.0 M HCl in dioxane (1.7 mL, 6.79 mmol) in CH₂C₂ (2 mL) was stirred at room temperature for 30 min. The mixture was concentrated under reduced pressure, the crude dissolved in MeOH and filtered through PL-HCO₃ MP agilent cartridge and the cartridge washed with MeOH (3×). The organic phase was concentrated under reduced pressure. Int-4A was obtained in 86% yield (150 mg) and the product used without further purification. HRMS (ESI-TOF) calcd for C₂₁H₂₅F₂N₅ [M+H]⁺ 386.2151, found 386.2164.

A mixture of Int-4A (50 mg, 0.13 mmol), Int-5A (126 mg, 0.52 mmol) and DIPEA (68 μL, 0.39 mmol) in CH₃CN (500 μL) was stirred at 120 C for 20 h. The mixture was diluted with EtOAc and washed sequentially with brine and sat. aq. NaHCO₃. The organic phase was dried over Na₂SO₄ and concentrated under reduced pressure. The residue was purified by preparative-TLC (CH₂Cl₂:MeOH, 95:5) to give 192 in 55% yield (33 mg). HRMS (ESI-TOF) calcd for C₂₅H₃₁F₂N₅₀ [M+H]⁺ 456.2570, found 456.2580.

### Example 26: (3S,4R)-1-(6-ethyl-8-fluoro-4-methyl-3-(1-methyl-1H-pyrazol-3-yl)quinolin-2-yl)-3-fluoro-N—((S)-tetrahydrofuran-3-yl)piperidin-4-amine (193)

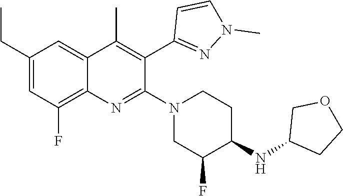 [SMILES: CCc1cc(F)c2nc(N3CC[C@@H](N[C@H]4CCOC4)[C@@H](F)C3)c(-c3ccn(C)n3)c(C)c2c1]

Compound 193 was obtained by the procedure of Example 25 using Int-4A and the appropriate tosylate. HRMS (ESI-TOF) calcd for C₂₅H₃₁F₂N₅O [M+H]⁺ 456.2570, found 456.2580.

### Example 27: (3R,4S)-1-(6-ethyl-8-fluoro-4-methyl-3-(1-methyl-1H-pyrazol-3-yl)quinolin-2-yl)-3-fluoro-N—((R)-tetrahydrofuran-3-yl)piperidin-4-amine (194)

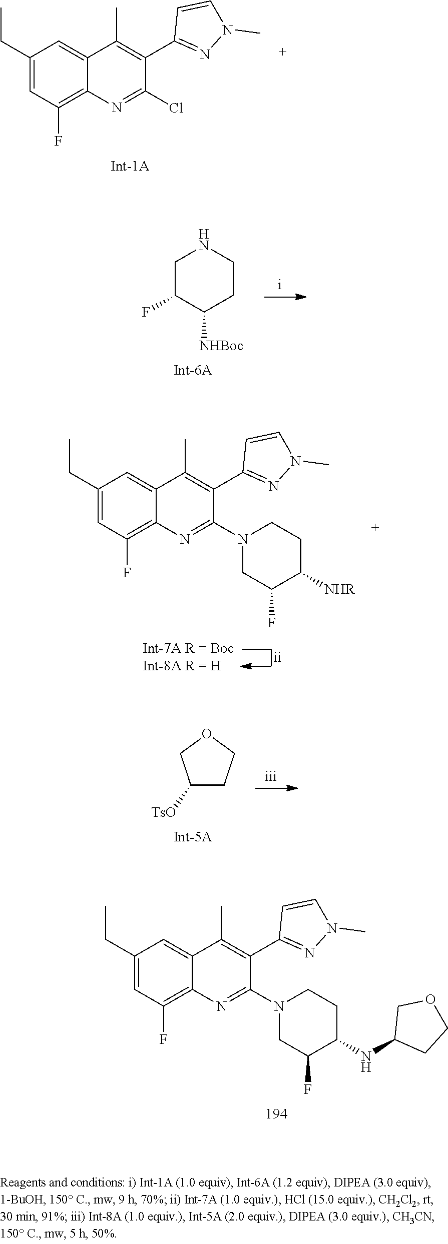

A mixture of Int-1A (100 mg, 0.33 mmol) and Int-6A (85 mg, 0.39 mmol), DIPEA (172 μL, 0.99 mmol) in 1-BuOH (345 μL) was heated at 150 C for 9 h under microwave irradiation. The mixture was concentrated under reduced pressure and the product was purified by column chromatography using hexanes/EtOAc. Int-7A was obtained in 70% yield (112 mg). ¹H NMR (600 MHz, CDCl₃) δ 7.47-7.43 (m, 2H), 7.18 (dd, J=11.5, 1.8 Hz, 1H), 6.37 (d, J=2.2 Hz, 1H), 4.82 (d, J=9.1 Hz, 1H), 4.71 (d, J=48.9 Hz, 1H), 4.13-4.06 (m, 1H), 4.01 (s, 3H), 3.74-3.63 (m, 1H), 3.58-3.52 (m, 1H), 3.16-3.06 (m, 1H), 2.77 (q, J=7.6 Hz, 2H), 2.69 (t, J=12.2 Hz, 1H), 2.50 (s, 3H), 1.67-1.53 (m, 2H), 1.44 (s, 9H), 1.30 (t, J=7.6 Hz, 3H). LCMS: (M+1) m/z=486.

A mixture of Int-7A (100 mg, 0.2 mmol) and 4.0 M HCl in dioxane (0.77 mL, 3.08 mmol) in CH₂Cl₂ (1 mL) was stirred at room temperature for 30 min. The mixture was concentrated under reduced pressure, the crude dissolved in MeOH and filtered through PL-HCO3 MP agilent cartridge, the cartridge washed with MeOH (3×). The organic phase was concentrated under reduced pressure and Int-8A was obtained in 90% yield (71 mg). The product was used without further purification. HRMS (ESI-TOF) calcd for C21H25F2N5 [M+H]⁺ 386.2151, found 386.2150.

A mixture of Int-8A (20 mg, 0.052 mmol), Int-5A (25 mg, 0.103 mmol) and DIPEA (27 μL, 0.156 mmol) in CH₃CN (500 μL) was stirred at 150 C for 5 h. The mixture was filtered through a PL-HCO₃ MP agilent cartridge and washed with MeOH (3×). The organic phase was concentrated under reduced pressure. The residue was purified by preparative-TLC (CH₂Cl₂:MeOH, 95:5) to give 194 in 50% yield (12 mg). HRMS (ESI-TOF) calcd for C25H31F2N5O [M+H]⁺ 456.2570, found 456.2579.

### Example 28: (3S,4S)-1-(6-ethyl-8-fluoro-4-methyl-3-(1-methyl-1H-pyrazol-3-yl)quinolin-2-yl)-3-fluoro-N—((R)-tetrahydrofuran-3-yl)piperidin-4-amine (195)

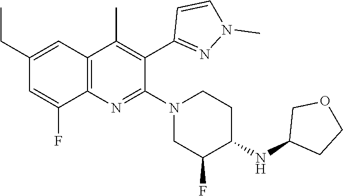 [SMILES: CCc1cc(F)c2nc(N3CC[C@H](N[C@@H]4CCOC4)[C@@H](F)C3)c(-c3ccn(C)n3)c(C)c2c1]

Compound 195 was obtained following the procedure used for the synthesis of 192 using the appropriate chiral amine and tosylate. HRMS (ESI-TOF) calcd for C₂₅H₃₁F₂N₅O [M+H]⁺ 456.2570, found 456.2582.

### Example 29: (3R,4R)-1-(6-ethyl-8-fluoro-4-methyl-3-(1-methyl-1H-pyrazol-3-yl)quinolin-2-yl)-3-fluoro-N—((R)-tetrahydrofuran-3-yl)piperidin-4-amine (196)

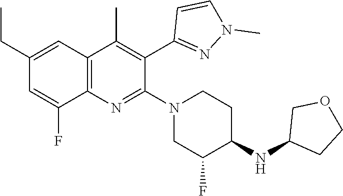 [SMILES: CCc1cc(F)c2nc(N3CC[C@@H](N[C@@H]4CCOC4)[C@H](F)C3)c(-c3ccn(C)n3)c(C)c2c1]

Compound 1% was obtained following the procedure used for the synthesis of 192 using the appropriate chiral amine and tosylate. HRMS (ESI-TOF) calcd for C25H31F2N5O [M+H]⁺ 456.2570, found 456.2587.

### Example 30: (3S,4R)-1-(6-ethyl-8-fluoro-4-methyl-3-(3-methylisoxazol-5-yl)quinolin-2-yl)-3-fluoro-N—((R)-tetrahydrofuran-3-yl)piperidin-4-amine (197)

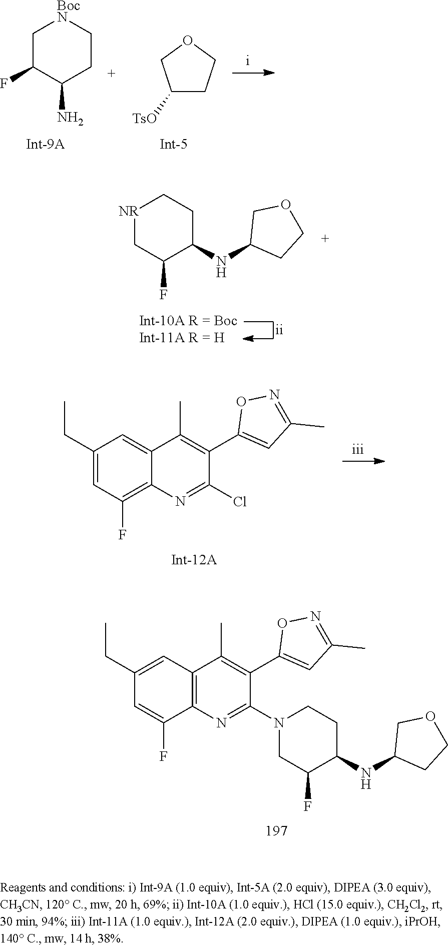

A mixture of Int-9A (1.0 g, 4.58 mmol), Int-5A (2.2 g, 9.16 mmol) and DIPEA (2.4 mL, 13.74 mmol) in CH₃CN (10 mL) was stirred at 120 C for 20 h under microwave irradiation. The mixture was filtered through a PL-HCO₃ MP agilent cartridge and the cartridge washed with MeOH (3×). The organic phase was concentrated under reduced pressure. The residue was purified by column chromatography using CH₂Cl₂/MeOH to give int-10 in 69% yield (915 mg). ¹H NMR (400 MHz, CDCl₃) δ 4.70 (d, J=47.7 Hz, 1H), 4.33 (bs, 1H), 4.18-3.99 (m, 1H), 3.98-3.89 (m, 1H), 3.88-3.74 (m, 2H), 3.58-3.47 (m, 2H), 3.06-2.94 (m, 2H), 2.72-2.60 (m, 1H), 2.14-2.03 (m, 1H), 1.78-1.57 (m, 3H), 1.45 (s, 9H).

A mixture of int-10A (940 mg, 3.26 mmol) and 4.0 M HCl in dioxane (12 mL, 48.9 mmol) in CH₂Cl₂ (10 mL) was stirred at room temperature for 30 min. The mixture was concentrated under reduced pressure, the product dissolved in MeOH and filtered through PL-HCO₃ MP agilent cartridge, the cartridge washed with MeOH (4×) and the organic phase concentrated under reduced pressure. Int-11A was obtained in 94% yield (580 mg) and the product used without further purification. HRMS (ESI-TOF) calcd for C9H17FN2O [M+H]⁺ 189.1398, found 189.1399.

A mixture of Int-12A (15 mg, 0.05 mmol), Int-11A (19 mg, 0.1 mmol) and DIPEA (9 μL, 0.05 mmol) in iPrOH (500 μL) was heated at 140 C for 14 h under microwave irradiation. The mixture was concentrated under reduced pressure and the product was purified by prep-TLC using CH₂Ch/MeOH. 197 was obtained in 52% yield (11.9 mg). HRMS (ESI-TOF) calcd for C₂₅H30F2N402 [M+H]⁺ 457.2410, found 457.2420.

### Example 31: (3S,4R)-1-(6-ethyl-8-fluoro-4-methyl-3-(3-methylisoxazol-5-yl)quinolin-2-yl)-3-fluoro-N—((S)-tetrahydrofuran-3-yl)piperidin-4-amine (198)

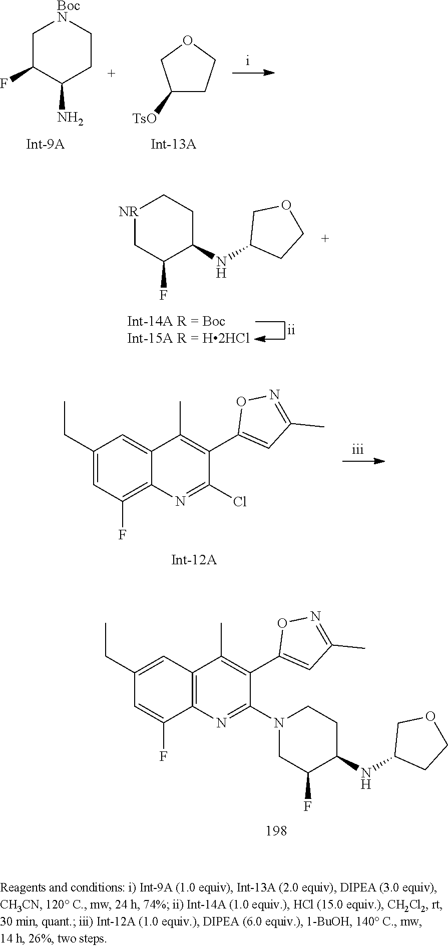

A mixture of Int-9A (500 mg, 2.29 mmol), Int-13A (1.11 g, 4.58 mmol) and DIPEA (1.2 mL, 6.87 mmol) in CH₃CN (10 mL) was stirred at 120 C for 24 h under microwave irradiation. The mixture was filtered through a PL-HCO₃ MP agilent cartridge and the cartridge washed with MeOH (3×). The organic phase was concentrated under reduced pressure. The residue was purified by column chromatography using CH₂Cl₂/MeOH to give Int-14A in 74% yield (490 mg). ¹H NMR (400 MHz, CDCl₃) δ 4.68 (d, J=48.5 Hz, 1H), 4.35 (bs, 1H), 4.20-4.00 (m, 1H), 3.97-3.90 (m, 1H), 3.88-3.74 (m, 2H), 3.59-3.46 (m, 2H), 3.09-2.74 (m, 2H), 2.73-2.59 (m, 1H), 2.15-2.02 (m, 1H), 1.78-1.55 (m, 3H), 1.46 (s, 9H).

A mixture of Int-14A (29 mg, 0.1 mmol) and 4.0 M HCl in dioxane (0.38 mL, 1.5 mmol) in CH₂Cl₂ (300 μL) was stirred at room temperature for 30 min. The mixture was concentrated under reduced pressure and the salt used without further purification. HRMS (ESI-TOF) calcd for C₉H17FN20 [M+H]⁺ 189.1398, found 189.1404. A mixture of Int-12A (15 mg, 0.05 mmol), Int-15A and DIPEA (52 μL, 0.3 mmol) in 1-BuOH (500 μL) was heated at 140 C for 14 h under microwave irradiation. The mixture was filtered through a PL-HCO3 MP agilent cartridge and the cartridge washed with MeOH (3×). The organic phase was concentrated under reduced pressure and the product was purified by prep-TLC using CH₂Cl₂/MeOH. 198 was obtained in 26% yield (two steps, 6.1 mg). HRMS (ESI-TOF) calcd for C25H30F2N4O2 [M+H]⁺ 457.2410, found 457.2408.

### Example 32: (3S,4S)-1-(6-ethyl-8-fluoro-4-methyl-3-(3-methylisoxazol-5-yl)quinolin-2-yl)-3-fluoro-N—((R)-tetrahydrofuran-3-yl)piperidin-4-amine (208)

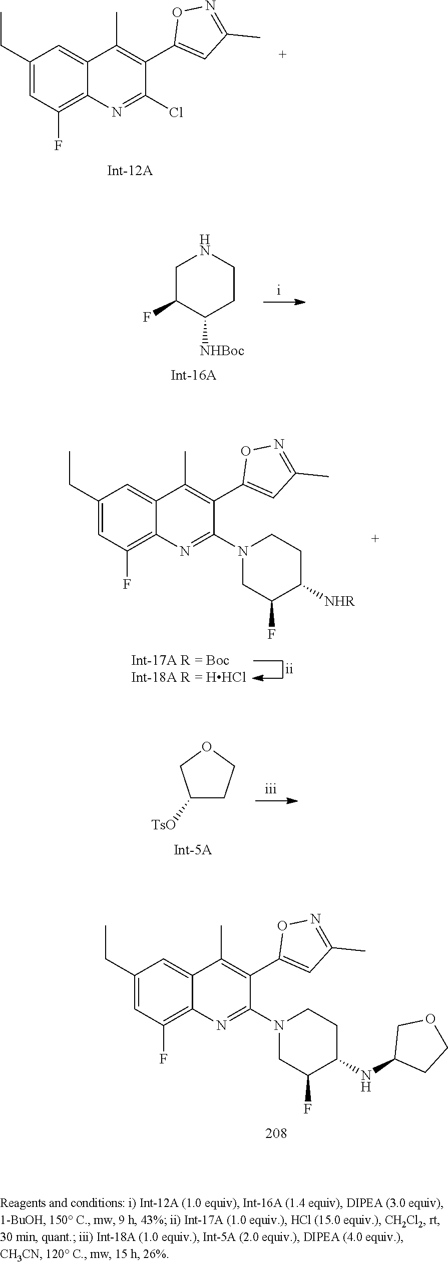

A mixture of Int-12A (50 mg, 0.164 mmol) and Int-16A (50 mg, 0.23 mmol), DIPEA (86 μL, 0.492 mmol) in 1-BuOH (500 μL) was heated at 150 C for 9 h under microwave irradiation. The mixture was concentrated under reduced pressure and the product was purified by column chromatography using hexanes/EtOAc. Int-17A was obtained in 43% yield (21 mg). ¹H NMR (500 MHz, CDCl₃) δ 7.49-7.45 (m, 1H), 7.25 (dd, J=11.5, 1.8 Hz, 1H), 6.25 (s, 1H), 4.57 (bs, 1H), 4.50-4.33 (m, 1H), 3.94-3.85 (m, 1H), 3.79-3.67 (m, 1H), 3.25 (d, J=13.7 Hz, 1H), 3.08-3.01 (m, 1H), 2.87 (ddd, J=13.5, 10.7, 2.9 Hz, 1H), 2.79 (q, J=7.6 Hz, 2H), 2.47 (s, 3H), 2.42 (s, 3H), 2.07-1.98 (m, 1H), 1.59-1.53 (m, 1H), 1.44 (s, 9H), 1.31 (t, J=7.6 Hz, 3H). LCMS: HRMS (ESI-TOF) calcd for C26H32F2N4O3 [M+H]⁺ 487.2515. found 487.2513.

A mixture of Int-17A (15 mg, 0.03 mmol) and 4.0 M HCl in dioxane (115 μL, 0.46 mmol) in CH₂Cl₂ (200 μL) was stirred at room temperature for 30 min. The mixture was concentrated under reduced pressure and the product was used without further purification. HRMS (ESI-TOF) calcd for C₂₁H24F2N40 [M+H]⁺ 387.1991, found 387.1995.

A mixture of Int-18A (10 mg, 0.023 mmol), Int-5A (11.5 mg, 0.047 mmol) and DIPEA (16 μL, 0.092 mmol) in CH₃CN (300 μL) was stirred at 120 C for 15 h. The mixture was filtered through a PL-HCO₃ MP agilent cartridge and the cartridge washed with MeOH (3×). The organic phase was concentrated under reduced pressure. The residue was purified by preparative-TLC (CH₂Cl₂:MeOH, 95:5) to give 208 in 20% yield (2.1 mg). HRMS (ESI-TOF) calcd for C₂₅H30F2N402 [M+H]⁺ 457.2410, found 457.2410.

### Example 33: ((R)-4,4-difluoro-1-methylpiperidin-2-yl)((R)-4-(6-ethyl-8-fluoro-4-methyl-3-(3-methyl-1,2,4-oxadiazol-5-yl)quinolin-2-yl)-3-methylpiperazin-1-yl)methanone (205)

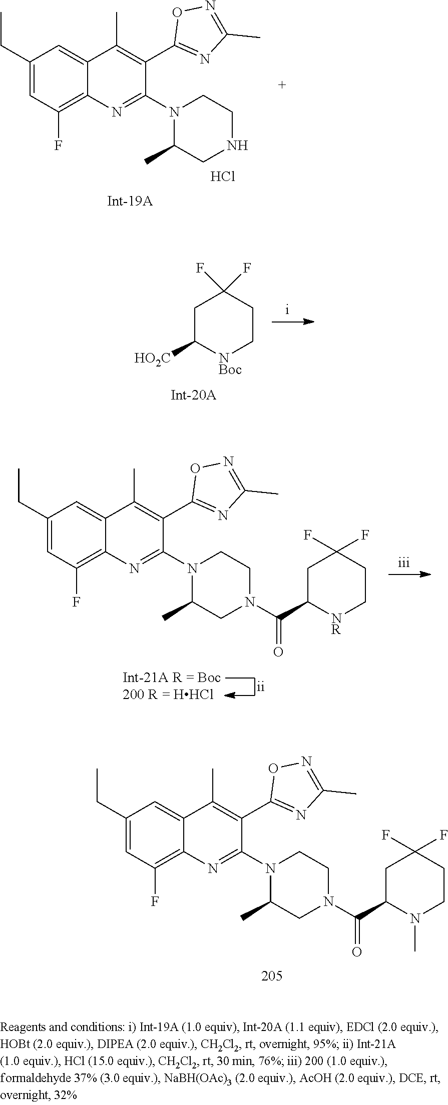

A mixture of Int-19A (25 mg, 0.062 mmol), Int-20A (18 mg, 0.067 mmol), EDCI (24 mg, 0.124 mmol), HOBt (17 mg, 0.124 mmol) and DIPEA (22PL, 0.124 mmol) in CH₂Cl₂ was stirred at room temperature overnight. The mixture was transferred to a separatory funnel with EtOAc and washed with brine (2×). The organic phase was dried over Na₂SO₄ and concentrated under reduced pressure. The crude product was purified by column chromatography using hexanes/EtOAc to give Int-21A in 95% yield (36 mg). HRMS (ESI-TOF) calcd for C31H39F3N6O4 [M+H]⁺ 617.3058, found 617.3065.

A mixture of Int-21A (30 mg, 0.048 mmol) and 4M HCl in dioxane (182 μL, 0.729 mmol) in CH₂C₂ (0.5 mL) was stirred at room temperature for 30 minutes. The mixture was concentrated under reduced pressure, the solid dissolved in MeOH and filtered through PL-HCO₃ MP agilent cartridge and washed with MeOH (3×). The organic phase was concentrated under reduced pressure and the product purified by prep-TLC using CH₂Cl₂/MeOH to yield 200 in 76% yield (19 mg). HRMS (ESI-TOF) calcd for C26H31F3N₆O₂ [M+H]⁺ 517.2533, found 517.2533.

A mixture of 200 (15 mg, 0.029 mmol), formaldehyde 37% aq. soln. (7 μl, 0.087 mmol), NaBH(OAc)₃ (11.5 mg, 0.054 mmol) and AcOH (3.1 μL, 0.054 mmol) in 1,2-dichloroethane (0.3 mL) was stirred at rt overnight. The mixture was diluted with EtOAc and washed with brine, the organic phase was dried over Na₂SO₄ and concentrated under reduced pressure. The crude material was purified by preparative-TLC (CH₂Cl₂:MeOH, 95:5) to give 205 in 52% yield (8 mg). HRMS (ESI-TOF) calcd for C27H33F3N6O2 [M+H]⁺ 531.2690, found 531.2679.

### Example 34: (R)-5-(6-ethyl-8-fluoro-2-(4-((4-fluorotetrahydro-2H-pyran-4-yl)methyl)-2-methylpiperazin-1-yl)-4-methylquinolin-3-yl)-3-methyl-1,2,4-oxadiazole (207)

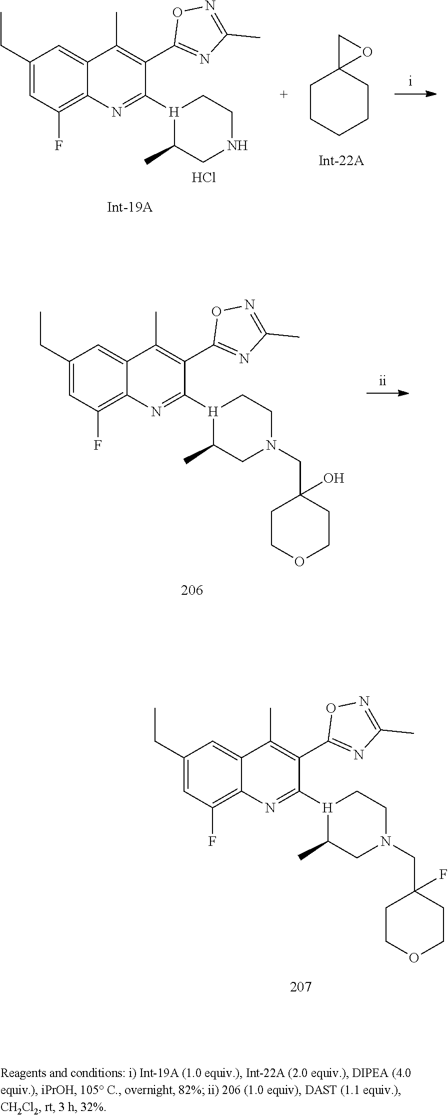

A mixture of Int-19A (26 mg, 0.06 mmol), Int-22A (15 mg, 0.13 mmol) and DIPEA (42 μL, 0.24 mmol) in iPrOH (300 μL) was stirred at 105 C overnight. The mixture was concentrated under reduced pressure. The product was purified by preparative-TLC (CH₂Cl₂; MeOH) to give 206 in 82% yield (24 mg). HRMS (ESI-TOF) calcd for C₂₆H34FN503 [M+H]⁺ 484.2719, found 484.2714.

A mixture of 206 (10 mg, 0.02 mmol) and DAST (3.7 mg, 0.022 mmol) in CH₂Cl₂ was attired at room temperature for 3 h. The mixture was quenched with 5% aq. Sodium bicarbonate and the product extracted with EtOAc (2×). The organic phase was dried over Na₂SO₄ and concentrated under reduced pressure. The crude material was purified by column chromatography using hexanes/EtOAc to give 207 in 32% yield (3.1 mg). HRMS (ESI-TOF) calcd for C₂₆H33F2N502 [M+H]⁺ 486.2675, found 486.2686.

### Example 35: (2R,4R)-1-(6-ethyl-8-fluoro-4-methyl-3-(1-methyl-1H-pyrazol-3-yl)quinolin-2-yl)-2-methyl-N—((R)-tetrahydrofuran-3-yl)piperidin-4-amine (115)

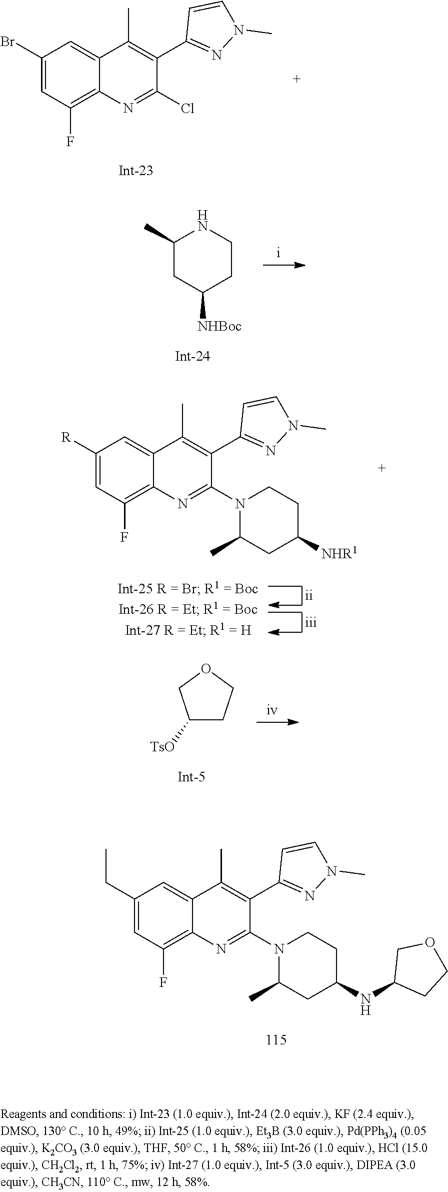

A mixture of Int-23 (50 mg, 0.14 mmol) and Int-24 (60 mg, 2.0 mmol), KF (20 mg, 0.336 mmol) in DMSO was heated at 130° C. for 10 h under microwave irradiation. The mixture was diluted with EtOAc (30 mL) and washed with brine (2×). The organic phase was dried over Na₂SO₄, concentrated under reduced pressure and the product was purified by column chromatography using hexanes/EtOAc. Int-25 was obtained in 49% yield (37 mg). ¹H NMR (400 MHz, CDCl₃) δ 7.85 (s, 1H), 7.45-7.41 (m, 2H), 6.33 (d, J=2.2 Hz, 1H), 4.37 (bs, 1H), 3.99 (s, 3H), 3.64-3.48 (m, 2H), 3.20-3.13 (m, 1H), 2.68-2.59 (m, 1H), 2.48 (s, 3H), 2.03-1.96 (m, 1H), 1.76-1.68 (m, 1H), 1.43 (s, 9H), 1.34-1.26 (m, 1H), 1.19 (d, J=6.0 Hz, 3H). LCMS: (M+1) m/z=532, 534.

To a mixture of Int-25 (50 mg, 0.094 mmol), K₂CO₃ (25.9 mg, 0.188 mmol) and Pd(PPh₃)₄ (5.4 mg, 0.005 mmol) in THF (0.5 mL) was added Et₃B (1M in THF) (282 μL, 0.282 mmol). The reaction was heated at 50° C. for 1 h. The mixture was diluted with EtOAc and washed with brine (2×). The organic phase was dried over Na₂SO₄, concentrated under reduced pressure and the product was purified by column chromatography using CH₂Cl₂/EtOAc to give Int-26 in 58% yield (26 mg). HRMS (ESI-TOF) calcd for C₃₇H₃₆FN₅O₂ [M+H]⁺ 482.2926, found 482.2927.

To a solution of Int-26 (185 mg, 0.384 mmol) in CH₂Cl₂ (1 mL) was slowly added at rt HCl (4M dioxane) (1.44 mL, 5.76 mmol) and the mixture was stirred at rt for 1 h. The solution was concentrated under reduced pressure. The crude was dissolved in MeOH and filtered through PL-HCO₃ MP agilent cartridge and washed with MeOH (3×). The organic phase was concentrated under reduced pressure to yield Int-27 in 75% yield (110 mg). The product was used without further purification. HRMS (ESI-TOF) calcd for C₂₂H₂₅FN₅ [M+H]⁺ 382.2402, found 382.2403.

A mixture of Int-27 (110 mg, 0.288 mmol), Int-5 (209.6 mg, 0.865 mmol) and DIPEA (151 μL, 0.865 mmol) in CH₃CN (2 mL) was stirred at 110° C. for 12 h under microwave irradiation. The mixture was filtered through a PL-HCO₃ MP agilent cartridge and washed with MeOH (3×). The organic phase was concentrated under reduced pressure. The residue was purified by column chromatography using CH₂Cl₂/MeOH to give 115 in 58% yield (75 mg). HRMS (ESI-TOF) calcd for C₂₆H34FN₅O [M+H]⁺ 452.2820, found 452.2825.

### Example 36: 1-(6-ethyl-8-fluoro-4-methyl-3-(1-methyl-1H-pyrazol-3-yl)quinolin-2-yl)-N-((3R,4R)-4-fluorotetrahydrofuran-3-yl)piperidin-4-amine (71)

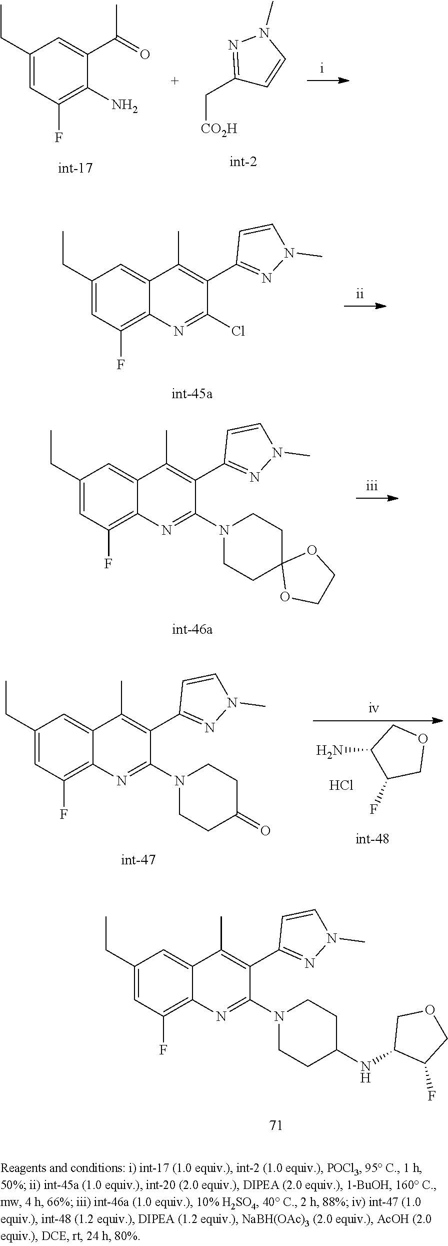

A mixture of (1-methyl-1H-pyrazol-3-yl)acetic acid int-2 (386 mg, 2.76 mmol) and int-17 (500 mg, 2.76 mmol) in POCl₃ (5 mL) was stirred at 95° C. for 1 h. The POCl₃ was removed under reduced pressure. The residue was quenched with ice/water and basified with sat. aq. solution of NaHCO₃ to pH ˜7, and the product extracted with EtOAc (2×). The organic phase was dried over Na₂SO₄ and concentrated under reduced pressure. The crude product was purified by column chromatography using hexanes/EtOAc to give 2-chloroquinoline int-45a as a pale brown solid (420 mg, 50% yield). ¹H NMR (500 MHz, CDCl₃) δ 7.57-7.53 (m, 1H), 7.51 (d, J=2.2 Hz, 1H), 7.30 (dd, J=11.0, 1.7 Hz, 1H), 6.34 (d, J=2.2 Hz, 1H), 4.02 (s, 3H), 2.83 (q, J=7.6 Hz, 2H), 2.54 (s, 3H), 1.33 (t, J=7.6 Hz, 3H).

A mixture of int-45a (80 mg, 0.26 mmol) and int-20 (75 mg, 0.527 mmol), DIPEA (92 μL, 0.12 mmol) in 1-BuOH (2 mL) was heated at 160° C. for 4 h under microwave irradiation. The mixture was concentrated under reduced pressure and the product was purified by column chromatography using Hexanes/EtOAc. int-46a was obtained in 66% yield (71 mg). ¹H NMR (500 MHz, CDCl₃) δ 7.45 (d, J=2.2 Hz, 1H), 7.43 (d, J=1.8, 1H), 7.16 (dd, J=11.6, 1.8 Hz, 1H), 6.34 (d, J=2.2 Hz, 1H), 4.00 (s, 3H), 3.93 (s, 4H), 3.37-3.29 (m, 4H), 2.77 (q, J=7.6 Hz, 2H), 2.48 (s, 3H), 1.66-1.60 (m, 4H), 1.30 (t, J=7.6 Hz, 3H).

A mixture of int-46a (60 mg, 0.146 mmol), 10% H2SO₄ (1 ml) in THF (1 mL) was stirred at 40° C. for 2 h. The mixture was basified with sat. aq. solution of NaHCO₃ to pH 7.5 and the product was extracted with EtOAc (2×). The organic phase was dried over Na₂SO₄ and concentrated under reduced pressure. The residue obtained in 88% yield (47 mg) and was used without further purification. ¹H NMR (500 MHz, CDCl₃) δ 7.50 (d, J=2.2 Hz, 1H), 7.47 (d, J=1.8, 1H), 7.20 (dd, J=11.5, 1.8 Hz, 1H), 6.35 (d, J=2.2 Hz, 1H), 4.02 (s, 3H), 3.55 (t, J=6.0 Hz, 4H), 2.79 (q, J=7.6 Hz, 2H), 2.50 (s, 3H), 2.41 (t, J=6.0 Hz, 4H), 1.31 (t, J=7.6 Hz, 3H).

A mixture of int-47 (10 mg, 0.027 mmol), int-48 (4.7 mg, 0.033 mmol), NaBH(OAc)₃ (11.5 mg, 0.05 mmol), DIPEA (5.7 μL, 0.033 mmol) and AcOH (3 μL, 0.05 mmol) in 1,2-dichloroethane (0.4 mL) was stirred at rt overnight. The mixture was diluted with EtOAc and washed with brine, the organic phase was dried over Na₂SO₄ and concentrated under reduced pressure. The residue was purified by preparative-TLC (DCM:MeOH, 95:5) to give 71 in 80% yield (9.9 mg). HRMS (ESI-TOF) calcd for C25H31F2N₅O [M+H]⁺ 456.2570, found 456.2557.

### Example 37: 1-(6-ethyl-8-fluoro-4-methyl-3-(1-methyl-1H-pyrazol-3-yl)quinolin-2-yl)-N-((3S,4S)-4-fluorotetrahydrofuran-3-yl)piperidin-4-amine (72)

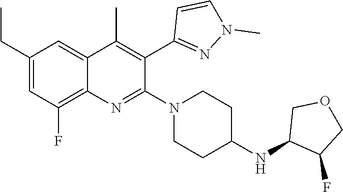 [SMILES: CCc1cc(F)c2nc(N3CCC(N[C@H]4COC[C@H]4F)CC3)c(-c3ccn(C)n3)c(C)c2c1]

Compound 72 was obtained following the procedure used for the synthesis of 71 using the appropriate chiral amine and ketone int-47. HRMS (ESI-TOF) calcd for C25H31F2N5O [M+H]⁺ 456.2570, found 456.2550.

### Example 38: (S)—N-(3,3-difluorocyclohexyl)-1-(6-ethyl-8-fluoro-4-methyl-3-(1-methyl-1H-pyrazol-3-yl)quinolin-2-yl)piperidin-4-amine (73)

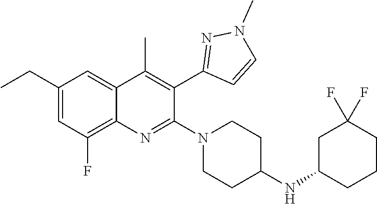 [SMILES: CCc1cc(F)c2nc(N3CCC(N[C@H]4CCCC(F)(F)C4)CC3)c(-c3ccn(C)n3)c(C)c2c1]

Compound 73 was obtained following the procedure used for the synthesis of 71 using the appropriate chiral amine and ketone int-47. HRMS (ESI-TOF) calcd for C27H34F3N5 [M+H]⁺ 486.2839, found 486.2836.

### Example 39: (R)-1-(4-(6-ethyl-8-fluoro-4-methyl-3-(3-methyl-1,2,4-oxadiazol-5-yl)quinolin-2-yl)-3-methylpiperazin-1-yl)-2-(pyrrolidin-1-yl)ethan-1-one (47)

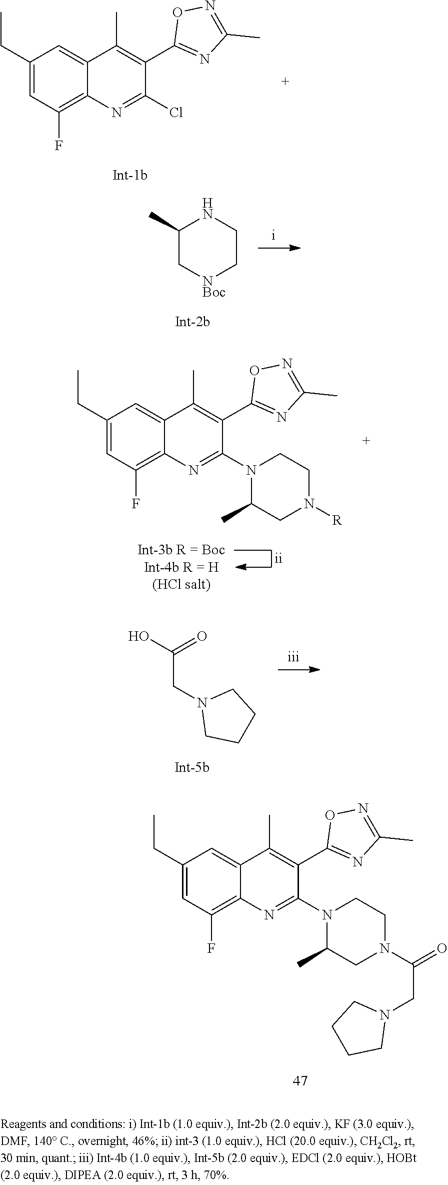

A mixture of Int-1b (350 mg, 1.14 mmol), Int-2b (458 mg, 2.29 mmol) and KF (200 mg, 3.42 mmol) in anhydrous DMF was attired at 140° C. overnight. The mixture was transferred to a separatory funnel with EtOAc (100 mL) and washed with brine (3×). The organic phase was dried over Na₂SO₄ and concentrated under reduced pressure. The crude product was purified by column chromatography using hexanes/EtOAc to give Int-3 in 46% yield (247 mg). ¹H NMR (500 MHz, CDCl₃) δ 7.51-7.47 (m, 1H), 7.29 (dd, J=11.2, 1.7 Hz, 1H), 3.70-3.53 (m, 2H), 3.46-3.36 (m, 1H), 3.30-3.16 (m, 4H), 2.80 (q, J=7.6 Hz, 2H), 2.55 (s, 3H), 2.51 (s, 3H), 1.45 (s, 9H), 1.32 (t, J=7.6 Hz, 3H), 1.07-0.98 (m, 3H). LCMS: (M+1) m/z=470.

A mixture of Int-3b (130 mg, 0.28 mmol) and 4M HCl in dioxane (715 μL, 5.6 mmol) in CH₂C₂ (5 mL) was stirred at room temperature for 30 minutes. The mixture was concentrated under reduced pressure and Int-4b was used without further purification. HRMS (ESI-TOF) calcd for C₂₀H24FN50 [M+H]⁺ 370.2038. found 370.2042.

[87-19] A mixture of Int-4b (25 mg, 0.062 mmol), Int-5b (16 mg, 0.124 mmol), EDCI (24 mg, 0.124 mmol), HOBt (17 mg, 0.124 mmol) and DIPEA (22 μL, 0.124 mmol) in CH₂Cl₂ was stirred at room temperature for 3 h. The mixture was transferred to a separatory funnel with EtOAc and washed with brine (2×). The organic phase was dried over Na₂SO₄ and concentrated under reduced pressure. The crude product was purified by column chromatography using CH₂Cl₂/MeOH to give 47 in 70% yield (21 mg). ¹H NMR (500 MHz, CDCl₃) δ 7.52-7.48 (m, 1H), 7.29 (dd, J=11.2, 1.8 Hz, 1H), 3.90-3.84 (m, 0.5H), 3.81-3.71 (m, 1.5H), 3.61-3.17 (m, 7H), 2.81 (t, J=7.6 Hz, 2H), 2.75-2.65 (m, 4H), 2.56 (s, 3H), 2.52 (s, 3H), 1.86-1.78 (m, 4H), 1.32 (t, J=7.6 Hz, 3H), 1.09 (d, J=6.5 Hz, 1.5H), 1.00 (d, J=6.5 Hz, 1.5H). HRMS (ESI-TOF) calcd for C26H33FN6O2 [M+H]⁺ 481.2722, found 481.2721.

### Example 40: (R)-5-(6-ethyl-8-fluoro-4-methyl-2-(2-methyl-4-((tetrahydro-2H-pyran-4-yl)methyl)piperazin-1-yl)quinolin-3-yl)-3-methyl-1,2,4-oxadiazole (49)

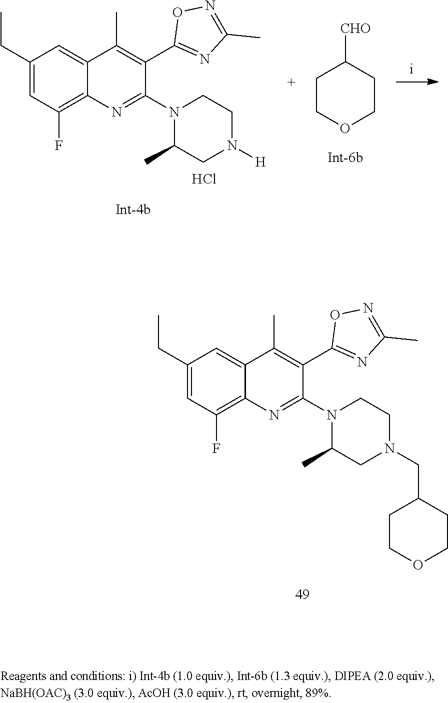 [SMILES: CCc1cc(F)c2nc(N3CCNC[C@H]3C)c(-c3nc(C)no3)c(C)c2c1 ; O=CC1CCOCC1 ; Cl ; CCc1cc(F)c2nc(N3CCN(CC4CCOCC4)C[C@H]3C)c(-c3nc(C)no3)c(C)c2c1]

A mixture of Int-4b (25 mg, 0.062 mmol), Int-6b (9 mg, 0.08 mmol), NaBH(OAc)₃ (39 mg, 0.186 mmol), DIPEA (21.6 s-L, 0.124 mmol) and AcOH (10.6 μL, 0.186 mmol) in 1,2-dichloroethane (0.5 mL) was stirred at rt overnight. The mixture was diluted with EtOAc and washed with brine, the organic phase was dried over Na₂SO₄ and concentrated under reduced pressure. The residue was purified by preparative-TLC (CH₂Cl₂:MeOH, 95:5) to give 49 in 89% yield (26 mg). HRMS (ESI-TOF) calcd for C26H36FN5O2 [M+H]⁺ 468.2769, found 468.2758.

### Example 41: (R)-5-(2-(4-((3,3-difluorocyclobutyl)methyl)-2-methylpiperazin-1-yl)-6-ethyl-8-fluoro-4-methylquinolin-3-yl)-3-methyl-1,2,4-oxadiazole (59)

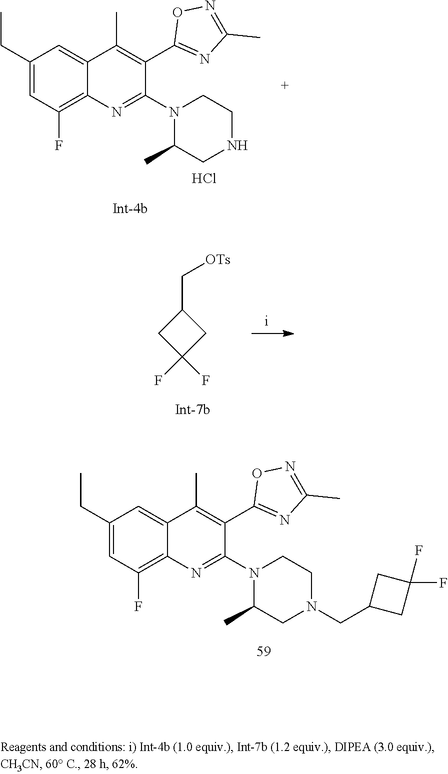 [SMILES: CCc1cc(F)c2nc(N3CCNC[C@H]3C)c(-c3nc(C)no3)c(C)c2c1 ; Cl ; Cc1ccc(S(=O)(=O)OCC2CC(F)(F)C2)cc1 ; CCc1cc(F)c2nc(N3CCN(CC4CC(F)(F)C4)C[C@H]3C)c(-c3nc(C)no3)c(C)c2c1]

A mixture of Int-4b (10 mg, 0.024 mmol), Int-7b (8.2 mg, 0.03 mmol) and DIPEA (12.6 μL, 0.072 mmol) in CH₃CN (200 μL) was stirred at 60° C. for 28 h. The mixture was diluted with EtOAc and washed sequentially with brine and sat. aq. NaHCO₃. The organic phase dried over Na₂SO₄ and concentrated under reduced pressure. The residue was purified by preparative-TLC (DCM:MeOH, 95:5) to give 59 in 62% yield (7.1 mg). HRMS (ESI-TOF) calcd for C₂₅H30F3N50 [M+H]⁺ 474.2475, found 474.2478.

### Example 42: 5-(6-ethyl-8-fluoro-4-methyl-2-((2R)-2-methyl-4-(1-(tetrahydro-2H-pyran-4-yl)ethyl)piperazin-1-yl)quinolin-3-yl)-3-methyl-1,2,4-oxadiazole (199)

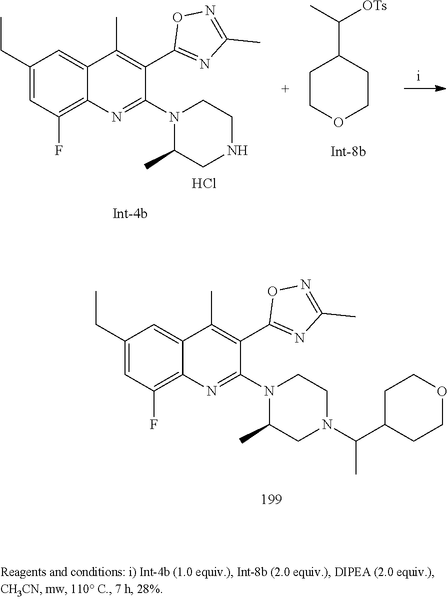 [SMILES: CCc1cc(F)c2nc(N3CCNC[C@H]3C)c(-c3nc(C)no3)c(C)c2c1 ; Cc1ccc(S(=O)(=O)OC(C)C2CCOCC2)cc1 ; Cl ; CCc1cc(F)c2nc(N3CCN(C(C)C4CCOCC4)C[C@H]3C)c(-c3nc(C)no3)c(C)c2c1]

A mixture of Int-4 (12 mg, 0.029 mmol), Int-8 (17 mg, 0.059 mmol) and DIPEA (10.3 μL, 0.072 mmol) in CH₃CN (500 μL) was stirred at 110° C. for 7 h under microwave irradiation. The mixture was diluted with EtOAc and washed sequentially with brine and sat. aq. NaHCO₃. The organic phase dried over Na₂SO₄ and concentrated under reduced pressure. The residue was purified by preparative-TLC (DCM:MeOH, 95:5) to give CYM-3252 in 28% yield (4 mg). HRMS (ESI-TOF) calcd for C₂₇H36FN502 [M+H]⁺ 482.2926, found 482.2921.

### Example 43: (R)-5-(6-chloro-8-fluoro-4-methyl-2-(2-methyl-4-((tetrahydro-2H-pyran-4-yl)methyl)piperazin-1-yl)quinolin-3-yl)-3-methyl-1,2,4-oxadiazole (50)

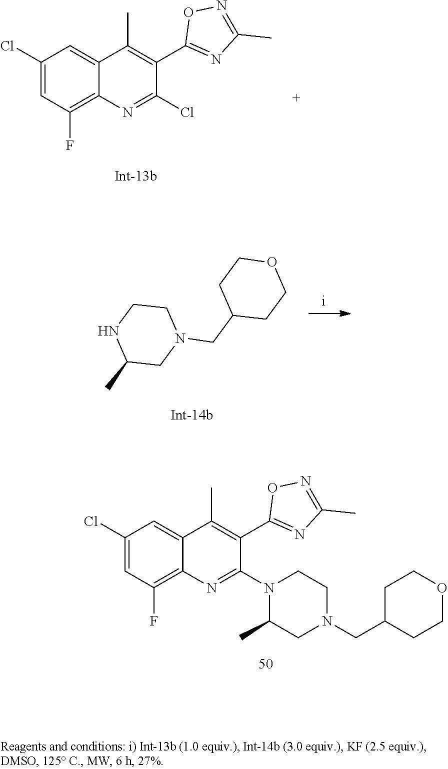

A mixture of Int-13b (8 mg, 0.26 mmol), Int-14b (15 mg, 0.77 mmol) and KF (3.7 mg, 0.64 mmol) in anhydrous DMSO was attired at 125° C. under microwave irradiation for 6 h. The mixture was transferred to a separatory funnel with EtOAc (30 mL) and washed with brine (3×). The organic phase was dried over Na₂SO₄ and concentrated under reduced pressure. The crude product was purified by preparative-TLC using CH₂Cl₂/MeOH to give cpd-5 in 27% yield (3.2 mg). LCMS: (M+1) m/z=474.

### Example 44: (R)-5-(8-chloro-4,6-dimethyl-2-(2-methyl-4-((tetrahydro-2H-pyran-4-yl)methyl)piperazin-1-yl)quinolin-3-yl)-3-methyl-1,2,4-oxadiazole (51)

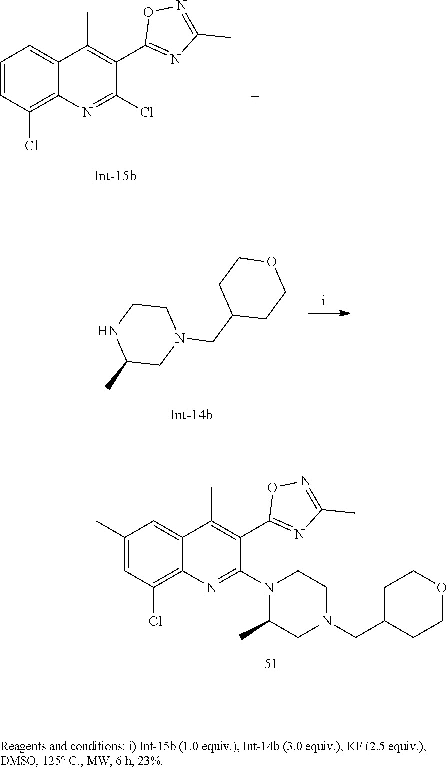 [SMILES: Cc1noc(-c2c(Cl)nc3c(Cl)cccc3c2C)n1 ; C[C@@H]1CN(CC2CCOCC2)CCN1 ; Cc1cc(Cl)c2nc(N3CCN(CC4CCOCC4)C[C@H]3C)c(-c3nc(C)no3)c(C)c2c1]

A mixture of Int-15b (8 mg, 0.26 mmol), Int-14b (15 mg, 0.77 mmol) and KF (3.7 mg, 0.64 mmol) in anhydrous DMSO was attired at 125° C. under microwave irradiation for 6 h. The mixture was transferred to a separatory funnel with EtOAc (30 mL) and washed with brine (3×). The organic phase was dried over Na₂SO₄ and concentrated under reduced pressure. The crude product was purified by preparative-TLC using CH₂Cl₂/MeOH to give 51 in 23% yield (2.8 mg). LCMS: (M+1) m/z=470.

### Example 45: (R)-5-(8-fluoro-6-methoxy-4-methyl-2-(2-methyl-4-((tetrahydro-2H-pyran-4-yl)methyl)piperazin-1-yl)quinolin-3-yl)-3-methyl-1,2,4-oxadiazole (37)

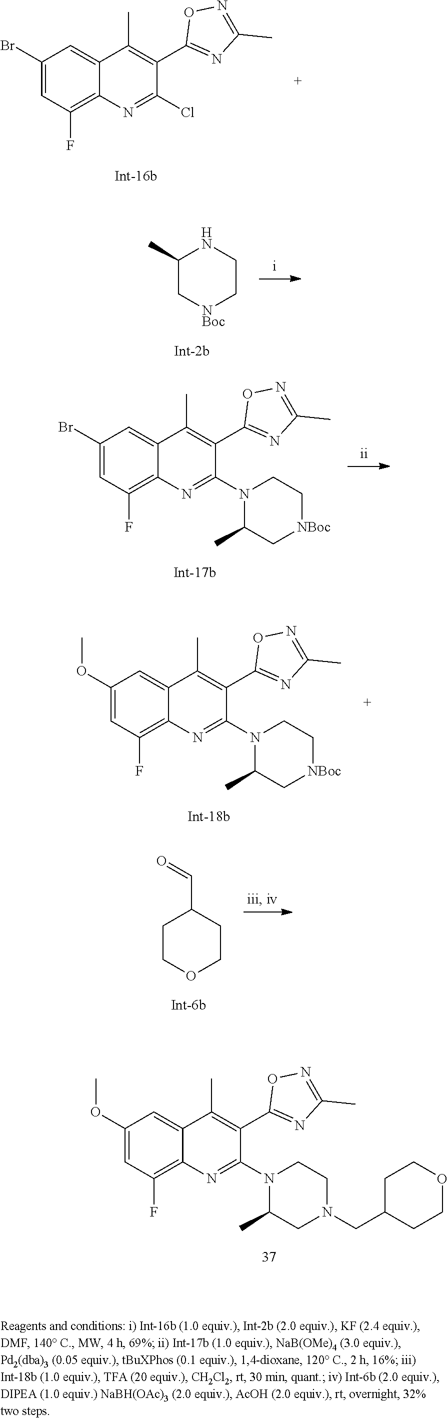

A mixture of Int-16b (100 mg, 0.28 mmol). Int-2b (112 mg, 0.56 mmol) and KF (39 mg, 0.67 mmol) in anhydrous DMF was attired at 140° C. under microwave irradiation for 4 h. The mixture was transferred to a separatory funnel with EtOAc (50 mL) and washed with brine (3×). The organic phase was dried over Na₂SO₄ and concentrated under reduced pressure. The crude product was purified by column chromatography using hexanes/EtOAc to give Int-17b in 69% yield (100 mg). ¹H NMR (400 MHz, CDCl₃) δ 7.85 (s, 1H), 7.51 (dd, J=9.4, 2.0 Hz, 1H), 3.83-3.67 (m, 2H), 3.60-3.51 (m, 1H), 3.36-3.21 (m, 2H), 3.20-3.00 (m, 2H), 2.56 (s, 3H), 2.49 (s, 3H), 1.45 (s, 9H), 1.08 (d. J=6.5 Hz, 3H). LCMS: (M+1) m/z=520, 522.

A mixture of Int-17b (100 mg, 0.172 mmol), sodium tetramethoxyborate (91 mg, 0.576 mmol), Pd₂(dba)₃ (9 mg, 0.009 mmol), tBuXPhos (8 mg, 0.192 mmol) in 1,4-dioxane (2 ml) was purged with nitrogen and heated at 120° C. for 2 h. The mixture was transferred to a separatory funnel with EtOAc (50 mL) and washed with brine (3×). The organic phase was dried over Na₂SO₄ and concentrated under reduced pressure. The crude product was purified by column chromatography using hexanes/EtOAc to give Int-18 in 16% yield (15 mg). LCMS: (M+1) m/z=472.

A mixture of Int-18b (9 mg, 0.019 mmol) and TFA (29 μL, 0.38 mmol) in CH₂Cl₂ (0.5 mL) was stirred at room temperature for 30 minutes. The mixture was concentrated under reduced pressure and the product used without further purification. LCMS: (M+1) m/z=372.

A mixture of the intermediate obtained above, Int-6b (4.3 mg, 0.038 mmol), NaBH(OAc)₃ (8 mg, 0.038 mmol), DIPEA (2.5 μL, 0.019 mmol) and AcOH (2.1 μL, 0.038 mmol) in 1,2-dichloroethane (0.4 mL) was stirred at rt overnight. The mixture was diluted with EtOAc and washed with brine, the organic phase was dried over Na₂SO₄ and concentrated under reduced pressure. The residue was purified by preparative-TLC using CH₂Cl₂/MeOH to give 37 in 32% yield (2.9 mg) (two steeps). LCMS: (M+1) m/z=470.

### Example 46: (R)-5-(6-ethyl-2-(2-ethyl-4-((tetrahydro-2H-pyran-4-yl)methyl)piperazin-1-yl)-8-fluoro-4-methylquinolin-3-yl)-3-methyl-1,2,4-oxadiazole (34)

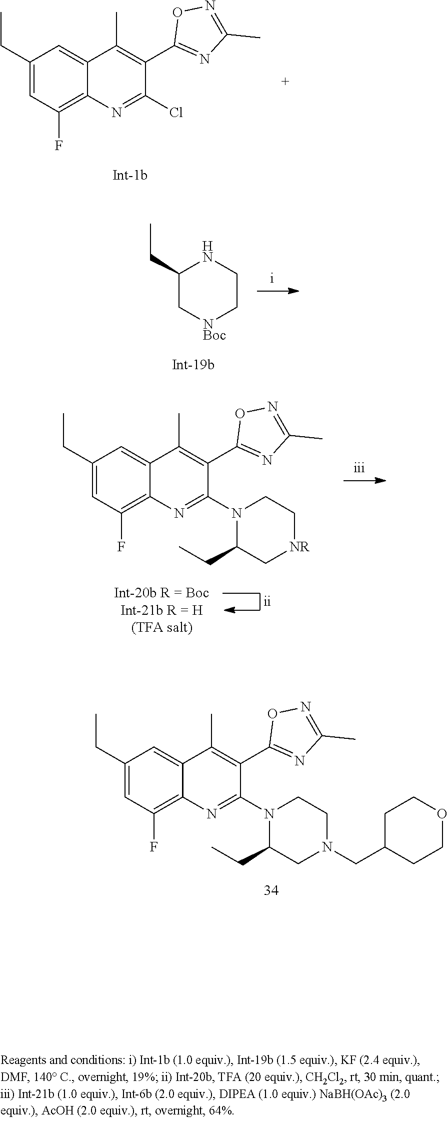

A mixture of Int-1b (100 mg, 0.327 mmol), Int-19b (105 mg, 0.49 mmol) and KF (46 mg, 0.784 mmol) in anhydrous DMF was attired at 140° C. overnight. The mixture was transferred to a separatory funnel with EtOAc (50 mL) and washed with brine (3×). The organic phase was dried over Na₂SO₄ and concentrated under reduced pressure. The crude product was purified by column chromatography using hexanes/EtOAc to give Int-20b in 19% yield (30 mg). LCMS: (M+1) m/z=484.

A mixture of Int-20b (30 mg, 0.062 mmol) and TFA (95 μL, 1.24 mmol) in CH₂Cl₂ (0.5 mL) was stirred at room temperature for 30 minutes. The mixture was concentrated under reduced pressure and the product used without further purification. LCMS: (M+1) m/z=384

A mixture Int-21b (8 mg, 0.016 mmol). Int-6b (3.7 mg, 0.032 mmol), NaBH(OAc)₃ (6.8 mg, 0.032 mmol), DIPEA (2.8 μL, 0.016 mmol) and AcOH (1.8 μL, 0.032 mmol) in 1,2-dichloroethane (0.3 mL) was stirred at rt overnight. The mixture was diluted with EtOAc and washed with brine, the organic phase was dried over Na₂SO₄ and concentrated under reduced pressure. The residue was purified by preparative-TLC using CH₂C₂/MeOH to give 34 in 64% yield (5.0 mg) (two steps). LCMS: (M+1) m/z=482.

### Example 47: (R)-5-(6-ethyl-8-fluoro-4-methyl-2-(2-methyl-4-(tetrahydro-2H-pyran-4-yl)piperazin-1-yl)quinolin-3-yl)-3-methyl-1,2,4-oxadiazole (26) and (S)-5-(6-ethyl-8-fluoro-4-methyl-2-(2-methyl-4-(tetrahydro-2H-pyran-4-yl)piperazin-1-yl)quinolin-3-yl)-3-methyl-1,2,4-oxadiazole (27)

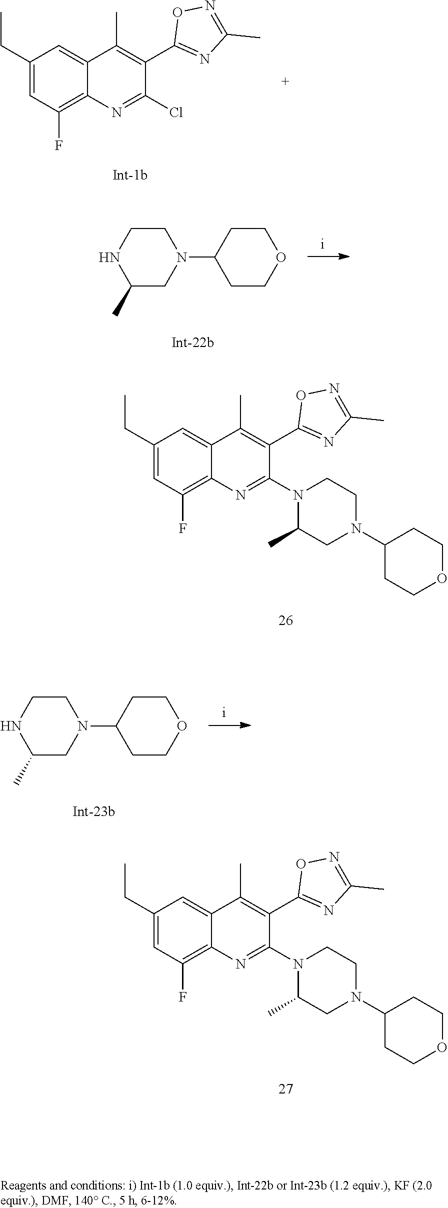 [SMILES: CCc1cc(F)c2nc(Cl)c(-c3nc(C)no3)c(C)c2c1 ; C[C@@H]1CN(C2CCOCC2)CCN1 ; CCc1cc(F)c2nc(N3CCN(C4CCOCC4)C[C@H]3C)c(-c3nc(C)no3)c(C)c2c1 ; C[C@H]1CN(C2CCOCC2)CCN1 ; CCc1cc(F)c2nc(N3CCN(C4CCOCC4)C[C@@H]3C)c(-c3nc(C)no3)c(C)c2c1]

A mixture of Int-1b (15 mg, 0.049 mmol), Int-22b or Int-23b (22 mg, 0.0.059 mmol), and KF (6 mg, 0.098 mmol) in anhydrous DMF was attired at 140° C. for 5 h. The mixture was transferred to a separatory funnel with EtOAc (30 mL) and washed with brine (3×). The organic phase was dried over Na₂SO₄ and concentrated under reduced pressure. The crude product was purified by preparative-TLC using CH₂Cha/MeOH. Compound 26 was obtained in 12% yield (2.7 mg). LCMS: (M+1) m/z=454. Compound 27 was obtained in 6% yield (1.4 mg). LCMS: (M+1) m/z=454.

### Example 48: (S)-5-(6-ethyl-8-fluoro-4-methyl-2-(3-methyl-4-((tetrahydro-2H-pyran-4-yl)methyl)piperazin-1-yl)quinolin-3-yl)-3-methyl-1,2,4-oxadiazole (38)

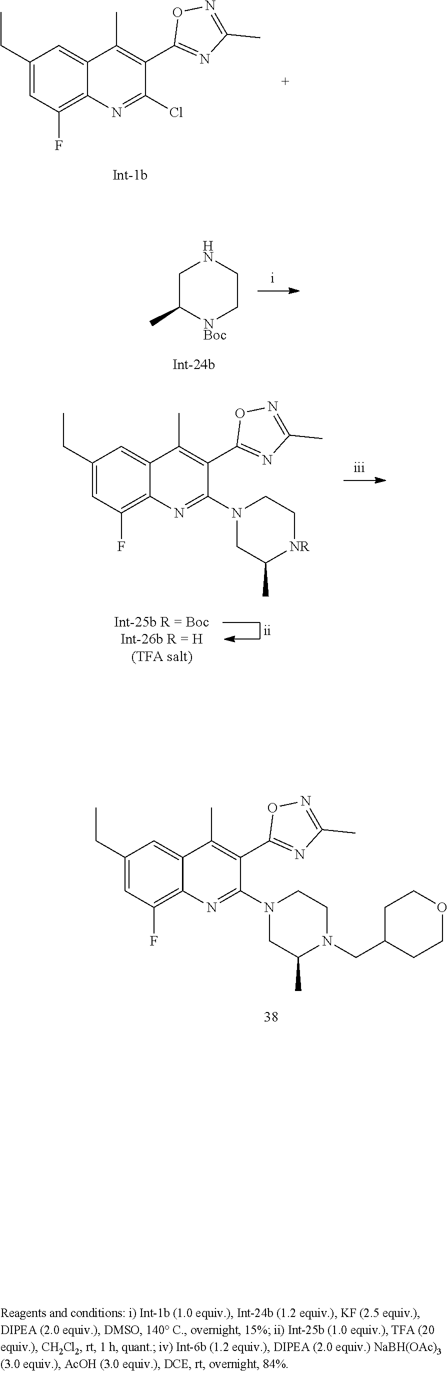

A mixture of Int-1b (75 mg, 0.245 mmol), Int-24b (60 mg, 0.294 mmol), DIPEA (86 μL, 0.49 mmol) and KF (36 mg, 0.613 mmol) in anhydrous DMF was attired at 140° C. for 8 h. The mixture was transferred to a separatory funnel with EtOAc (40 mL) and washed with brine (3×). The organic phase was dried over Na₂SO₄ and concentrated under reduced pressure. The product was purified by column chromatography using hexanes/EtOAc in 15% yield (18 mg). LCMS: (M+1) m/z=470.

A mixture of Int-25b (18 mg, 0.038 mmol) and TFA (60 μL, 0.76 mmol) in CH₂Cl₂ (0.5 mL) was stirred at room temperature for 30 minutes. The mixture was concentrated under reduced pressure and the product used without further purification. LCMS: (M+1) m/z=370.

A mixture Int-26b (21.5 mg, 0.038 mmol), Int-6b (5.2 mg, 0.0456 mmol), NaBH(OAc)₃ (24 mg, 0.114 mmol), DIPEA (13 μL, 0.076 mmol) and AcOH (6.5 AL, 0.114 mmol) in 1,2-dichloroethane (0.4 mL) was stirred at rt overnight. The mixture was diluted with EtOAc and washed with brine, the organic phase was dried over Na₂SO₄ and concentrated under reduced pressure. The residue was purified by preparative-TLC using CH₂Cl₂/MeOH to give 38 in 84% yield (15 mg). LCMS: (M+1) m/z=468.

### Example 49: (R)-5-(6-ethyl-8-fluoro-4-methyl-2-(3-methyl-4-((tetrahydro-2H-pyran-4-yl)methyl)piperazin-1-yl)quinolin-3-yl)-3-methyl-1,2,4-oxadiazole (39)

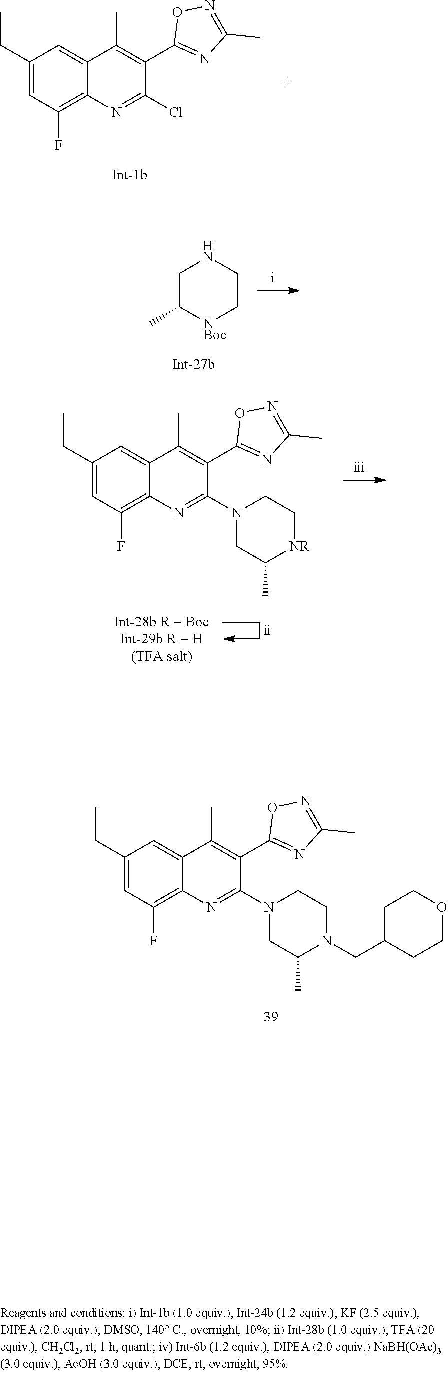

A mixture of int-1b (75 mg, 0.245 mmol), Int-24b (60 mg, 0.294 mmol), DIPEA (86 μL, 0.49 mmol) and KF (36 mg, 0.613 mmol) in anhydrous DMF was attired at 140° C. for 8 h. The mixture was transferred to a separatory funnel with EtOAc (40 mL) and washed with brine (3×). The organic phase was dried over Na₂SO₄ and concentrated under reduced pressure. The product was purified by column chromatography using hexanes/EtOAc in 10% yield (12 mg). LCMS: (M+1) m/z=470.

A mixture of Int-28b (12 mg, 0.025 mmol) and TFA (40 μL, 0.511 mmol) in CH₂Cl₂ (0.4 mL) was stirred at room temperature for 30 minutes. The mixture was concentrated under reduced pressure and the product used without further purification. LCMS: (M+1) m/z=370.

A mixture Int-29b (12.0 mg, 0.025 mmol), Int-6b (3.4 mg, 0.03 mmol), NaBH(OAc)₃ (16 mg, 0.075 mmol), DIPEA (9 μL, 0.05 mmol) and AcOH (4.3 μL, 0.075 mmol) in 1,2-dichloroethane (0.4 mL) was stirred at rt overnight. The mixture was diluted with EtOAc and washed with brine, the organic phase was dried over Na₂SO₄ and concentrated under reduced pressure. The residue was purified by preparative-TLC using CH₂Cl₂/MeOH to give 39 in 95% yield (11.1 mg). LCMS: (M+1) m/z=468.

### Example 50: (R)-5-(6-ethyl-8-fluoro-4-methyl-2-(2-methyl-4-((tetrahydro-2H-pyran-4-yl)methyl)piperazin-1-yl)quinolin-3-yl)-3-methylisoxazole (63)

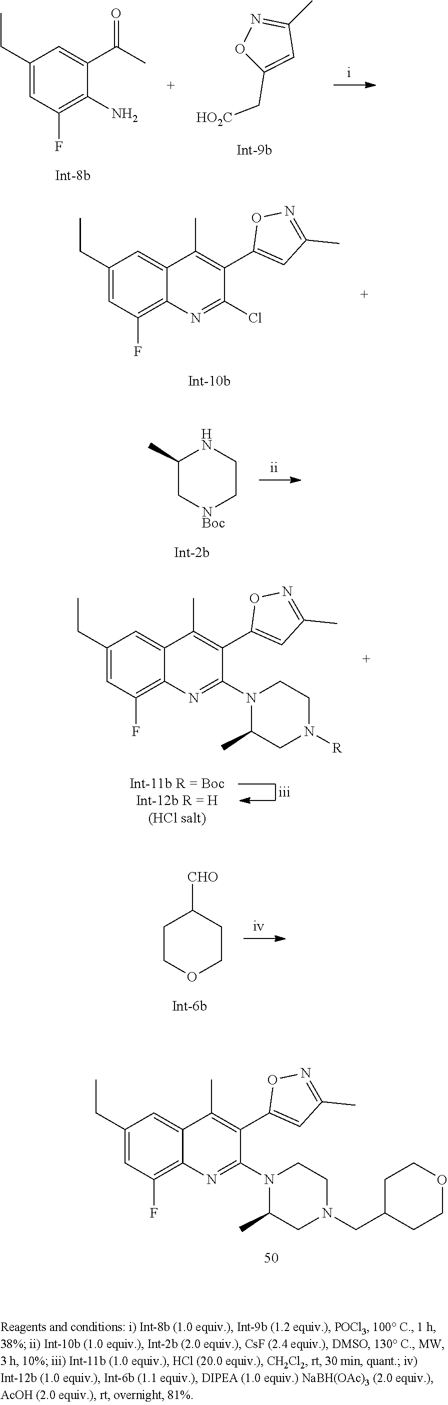

A mixture of Int-9b (187 mg, 1.32 mmol) and Int-8b (200 mg, 1.1 mmol) in POCl₃ (2 mL) was stirred at 100° C. for 1 h. The POCl₃ was removed under reduced pressure. The residue was quenched with ice/water and basified with sat. aq. solution of NaHCO₃ to pH ˜7, and the product extracted with EtOAc (2×). The organic phase was dried over Na₂SO₄ and concentrated under reduced pressure. The product was purified by column chromatography using hexanes/EtOAc to give Int-10b in 38% yield (130 mg). ¹H NMR (500 MHz, CDCl₃) δ 7.58 (s, 1H), 7.38 (dd, J=10.7, 1.7 Hz, 1H), 6.35 (s, 1H), 2.85 (q, J=7.6 Hz, 2H), 2.56 (s, 3H), 2.45 (s, 3H), 1.34 (t, J=7.6 Hz, 3H). LCMS: (M+1) m/z=305.

A mixture of Int-10b (65 mg, 0.213 mmol), Int-2b (86 mg, 0.427 mmol) and CsF (78 mg, 0.51 mmol) in anhydrous DMSO was attired at 130° C. under microwave irradiation for 3 h. The mixture was transferred to a separatory funnel with EtOAc (100 mL) and washed with brine (4×). The organic phase was dried over Na₂SO₄ and concentrated under reduced pressure. The crude product was purified by column chromatography using hexanes/EtOAc to give Int-11b in 16% yield (16.2 mg). ¹H NMR (500 MHz, CDCl₃) δ 7.46 (d, J=1.7 Hz, 1H), 7.24 (dd, J=11.3, 1.8 Hz, 1H), 6.24 (s, 1H), 3.84-3.68 (m, 1H), 3.64-3.58 (m, 1H), 3.55-3.45 (m, 1H), 3.30-3.18 (m, 2H), 3.16-3.03 (m, 2H), 2.78 (q, J=7.6 Hz, 2H), 2.45 (s, 3H), 2.42 (s, 3H), 1.45 (s, 9H), 1.31 (t, J=7.6 Hz, 3H), 1.04 (d, J=6.5 Hz, 3H). LCMS: (M+1) m/z=469.

A mixture of Int-11b (15 mg, 0.032 mmol) and 4M HCl in dioxane (40 μL, 0.16 mmol) in CH₂Cl₂ (0.5 mL) was stirred at room temperature for 30 minutes. The mixture was concentrated under reduced pressure and Int-12b was used without further purification. LCMS: (M+1) m/z=369.

A mixture of Int-12b (15 mg, 0.037 mmol), Int-6b (5 mg, 0.041 mmol), NaBH(OAc)₃ (16 mg, 0.074 mmol), DIPEA (6.5 μL, 0.037 mmol) and AcOH (4.2 μL, 0.074 mmol) in 1,2-dichloroethane (0.5 mL) was stirred at rt overnight. The mixture was diluted with EtOAc and washed with brine, the organic phase was dried over Na₂SO₄ and concentrated under reduced pressure. The residue was purified by preparative-TLC using CH₂Cl₂/MeOH to give 50 in 81% yield (14 mg). HRMS (ESI-TOF) calcd for C₂₇H35FN402 [M+H]⁺ 467.2817, found 467.2831.

### Biological Activity

### General Methods and Materials

OPRKappa 1 Tango B-Arrestin Assay Protocol-Antagonist Mode. The purpose of this assay is to confirm the potency of the present compounds as OPRK1 antagonists. The assay uses Tango OPRK1-bla U2OS cells which express OPRK1 linked to a GAL4-VP16 transcription factor via a TEV protease site. The cells also express a β-arrestin/TEV protease fusion protein and a β-lactamase (BLA) reporter gene under the control of a UAS response element. Stimulation of the OPRK1 receptor by agonist causes migration of the β-arrestin fusion protein to the GPCR, and through proteolysis liberates GAL4-VP16 from the receptor. The liberated VP16-GAL4 migrates to the nucleus, where it induces transcription of the BLA gene. BLA expression is monitored by measuring fluorescence resonance energy transfer (FRET) of a cleavable, fluorogenic, cell-permeable BLA substrate. As designed, test compounds that are OPRK1 antagonists inhibit agonist activation and migration of the fusion protein, thus preventing proteolysis of GAL4-VP16 and BLA transcription, leading to no increase in well FRET. Compounds were tested in quadruplicate using a 10-point, 1:3 dilution series starting at a nominal concentration of 10 micromolar.

The Tango OPRK1-U20S Dividing cell line was routinely cultured in 150 mm dishes at 37C, 5% CO₂ and 95% relative humidity (RH). The growth media consisted of McCoys 5A Media supplemented with 10% v/v dialyzed fetal bovine serum, 25 mM HEPES, 0.1 mM non-essential amino acids, 1 mM Sodium Pyruvate, and 1× antibiotic mix (penicillin streptomycin).

On Day 1 of the assay, 16,000 cells in 10 μL of assay media (DMEM-Glutamax—with Sodium Pyruvate, 10% fetal bovine serum stripped with charcoal-dextran (CDS), 25 mM HEPES, 0.1 mM non-essential amino acids, antibiotic mix (penicillin streptomycin)) were seeded into each well of a 384 Greiner 788092 black low profile, clear bottom, low volume plate, and incubated 16-24 hours at 37° C., 5% CO₂ and 95% (RH).

On Day 2, 50 nL of test compound in DMSO was added to the appropriate wells and plates were incubated for 30 min at 37° C. 5% CO₂ and 95% (RH). Next was added 0.65 μL of commercially available U50488 OPRK1 agonist or DMSO in assay media: this EC80 Challenge consists of 0.6 μL of 111 nM U50488 to yield a final assay concentration of 6 nM). After incubation for 4 hours at 37° C., 5% CO₂ and 95% (RH), 2.5 μL of LiveBLazer™FRET B/G (CCF4-AM) Mix (Soln.'s A,B,C,&D) was added to each well and incubated at room temperature in the dark for 2 hours. Well fluorescence was measured on Perkin Elmer's Envision using an Excitation filter 409 nm, Emission filters at 460 nm and 590 nm, bottom read.

The Percent Inhibition was calculated from the median ratio as follows:

$$\% Inhibition = \{ 1 - [ \frac{{FI}_{test compound} - Median {FI}_{high control}}{Median {FI}_{low control} - Median {FI}_{high control}} ] \} \times 100$$

where:
   test compound is defined as wells containing test compound;
   low control is defined as wells containing U50488 challenge (6 nM final)=0% inhibition; and
   high control is defined as wells containing DMSO=100% inhibition.

### List of Reagents:

Tango™OPRK1-bla U20S Cells (Invitrogen K1576)
McCoy's 5A Medium, (Invitrogen 16600-082)
Dialyzed Fetal Bovine Serum, (Invitrogen 26400-036)
Non Essential Amino Acids 100× (Invitrogen part 11140-050)
HEPES (pH 7.3) 1M, (Invitrogen 15630-080)
Sodium Pyruvate 100× (Invitrogen 11360-070)
Penicillin Streptomycin, (Invitrogen 15640)
Trypsin 0.25% EDTA (Invitrogen 25200056)
DPBS without Calcium/Magnesium (Invitrogen 14190-136)
DMEM, High Glucose, GlutaMAX (Invitrogen 10569-010)
Fetal Bovine Serum, Charcoal Stripped (Invitrogen 12676-011)
DMSO Dry (Sigma D2650)
U50488 OPRK1 Agonist MW410.29 (Tocris 67198-19-0)
GNTI dihydrochloride OPRK1 Antagonist MW571.5 (Tocris 1282)
Nor-Binaltorphimine dihydrochloridc MW770.75 (Tocris 0347)

LiveBLAzere™-FRET/BG Loading Mix: (Invitrogen K₁₀₃₀ (5 mg)), composed of Solutions A, B, C, and D:
Solution A (6 μL): LiveBLAzer™-FRET/BG Substrate (CCF4-AM)
Solution B (60 μL)
Solution C (904 μL)
Solution C (must add 250 μl of 1N NaOH to 45 mL of Solution C prior to use)
Solution D (30 μL) [Probinicid] (Sigma P8161) make 200 mM stock in NaOH—H₂O.

OPRMu1 Discover X—β-arrestin Assay—Antagonist Mode. The purpose of this assay is to confirm the potency and specificity of compounds synthesized to be OPRK1 antagonists. This assay evaluates the OPRMu1 activation in membrane recruitment of β-arrestin. In addition, the assay evaluates GPCR-β-arrestin proximity using low affinity fragment complementation of beta-galactosidase (beta-gal). The assay employs U20S cells, which express OPRMu1 fused to the complementary beta-gal fragment (enzyme acceptor). As designed, compounds that act as antagonists will prevent receptor activation resulting in reduced well luminescence. Compounds were tested in quadruplicate using a 10-point, 1:3 dilution series starting at a nominal concentration of 10 micromolar.

The Discover X OPRMu1-U20S cell line was cultured in 150 mm dishes at 37° C., 5% CO₂ and 95% relative humidity (RH). The growth media consisted of DMEM/F12 1:1 Media supplemented with 10% (v/v) heat inactivated fetal bovine serum, 25 mM HEPES, 0.1 mM non-essential amino acids, 1 mM Sodium Pyruvate, and 1× antibiotic mix (penicillin streptomycin).

On Day 1 of the assay, 5000 cells in 20 μL of assay buffer (Discover X's Cell Plating Reagent 5) were seeded into each well of a 384 Corning 3570 standard white plate, and incubated 16-24 hours at 37° C., 5% CO₂ and 95% RH.

On Day 2, 100 nL of test compound in DMSO were added to the appropriate wells and the plates were then incubated for 30 min at 37° C. 5% CO₂ and 95% RH. Next, 2.2 μL of DAMGO OPRMu1 agonist (commercially available) or DMSO in assay media. (The EC80 Challenge consists of 1.8 μL of 3.7 μM DAMGO, 0.4 μL of Assay Buffer, with a final assay concentration of 303 nM). After incubation for 3 hours at 37° C. 5% CO₂, and 95% RH, 10 μL of Path Hunter Detection Mix was added to each well, and the plate was then incubated at room temperature in the dark for 1 hour. Well luminescence was measured on Perkin Elmer's Envision.

The Percent Inhibition was calculated from the median ratio as follows:

$$\% Inhibition = \{ 1 - [ \frac{{FI}_{test compound} - Median {FI}_{high control}}{Median {FI}_{low control} - Median {FI}_{high control}} ] \} \times 100$$

where:
   test compound is defined as wells containing test compound;
   low control is defined as wells containing DAMGO challenge (200 nM final)=0% inhibition; and
   high control is defined as wells containing DMSO=100% inhibition.

### List of Reagents:

DMEM Medium, (Invitrogen 11965)
F12 Medium, (Invitrogen 11765)
Heat Inactivated Fetal Bovine Serum, (Invitrogen 10082147)
Non Essential Amino Acids 100× (Invitrogen 11140-050)
HEPES (pH 7.3) 1M, (Invitrogen 15630-080)
Sodium Pyruvate 100× (Invitrogen 11360-070)
Penicillin Streptomycin, (Invitrogen 15640)
Trypsin 0.25% EDTA (Invitrogen 25200056)
DPBS without Calcium/Magnesium (Invitrogen 14190-136)
DMSO Dry (Sigma D2650)
DAMGO OPRMu1 Agonist MW513.19 (Sigma E7384-5MG)
B-Funaltrexamine Hydrochloride OPRM1 Antagonist MW (SIGMA 0003-2MG)
PathHunter Cell Plating 5 Reagent (Discover X 93-0563R5A)
Corning 3750 Standard 384 well white plate with lid.

PathHunter Detection Mix (DiscoverX 93-0001): 1 part Galacton Star/5 parts Emerald II/19 parts PH Cell Assay Buffer.

OPRDelta 1 Tango B-Arrestin Assay Protocol—Antagonist Mode. The Tango OPRDelta1-U20S Dividing cell line was cultured in 150 mm dishes at 37° C., 5% CO₂ and 95% relative humidity (RH). The growth media consisted of McCoys 5A Media supplemented with 10% (v/v) dialyzed fetal bovine serum, 25 mM HEPES, 0.1 mM non-essential amino acids, 1 mM Sodium Pyruvate, and 1× antibiotic mix (penicillin streptomycin).

On Day 1 of the assay, 16,000 cells in 10 μL of assay media (DMEM-Glutamax—with Sodium Pyruvate, 10% fetal bovine serum stripped with charcoal-dextran (CDS), 25 mM HEPES, 0.1 mM non-essential amino acids, antibiotic mix (penicillin streptomycin)) were seeded into each well of a 384 Greiner 788092 black low profile, clear bottom, low volume plate. 50 nL of test compound in DMSO was added to the appropriate wells and then the plates were incubated for 30 min at 37° C., 5% CO₂ and 95% RH. Next, 1.1 μL of SNC80 OPRD1 agonist (commercially available) or DMSO in assay media (EC80 Challenge consists of 1.1 μL of 3.7 μM SNC80, final assay concentration=370 nM) was added to appropriate wells and incubated 16-24 hours at 37° C., 5% CO₂ and 95% RH.

On Day 2, 2.5 μL of LiveBLazer™FRET B/G (CCF4-AM) Mix (Soln.'s A, B, C, and D) was added to each well, and the plate was then incubated at room temperature in the dark for 2 hours. Well fluorescence was measured on Perkin Elmer's Envision using an Excitation filter 405 nm, Emission filters at 460 nm and 590 nm, bottom read.

The Percent Inhibition was calculated from the median ratio as follows:

$$\% Inhibition = \{ 1 - [ \frac{{FI}_{test compound} - Median {FI}_{high control}}{Median {FI}_{low control} - Median {FI}_{high control}} ] \} \times 100$$

where:
   test compound is defined as wells containing test compound;
   low control is defined as wells containing SNC80 challenge (370 nM final)=0% inhibition; and
   high control is defined as wells containing DMSO=100% inhibition.

### List of Reagents:

Tango™OPRD1-bla U20S (Invitrogen K1778)
McCoy's 5A Medium, (Invitrogen 16600-082)
Dialyzed Fetal Bovine Serum, (Invitrogen 26400-036)
Non Essential Amino Acids 100× (Invitrogen 11140-050)
HEPES (pH 7.3) 1M, (Invitrogen 15630-080)
Sodium Pyruvate 100× (Invitrogen 11360-070)
Penicillin Streptomycin, (Invitrogen 15640)
Trypsin 0.25% EDTA (Invitrogen 25200056)
DPBS without Calcium/Magnesium (Invitrogen 14190-136)
DMEM, High Glucose, GlutaMAX (Invitrogen 10569-010)
Fetal Bovine Serum, Charcoal Stripped (Invitrogen 12676-011)
DMSO Dry (Sigma D2650)
SNC80 OPRD1 Agonist MW449.63 (Sigma S2812)
SDM25N hydrochloride OPRD1 Antagonist MW468.98 (Tocris 1410)

LiveBLAzer™-FRET/BG Loading Mix: (Invitrogen K1030 (5 mg)), composed of Solutions A, B, C, and D:
Solution A (6 μL): LiveBLAzer™-FRET/BG Substrate (CCF4-AM)
Solution B (60 μL)
Solution C (904 μL)
Solution C (must add 250 μl of 1N NaOH to 45 mL of Solution C prior to use)
Solution D (30 μL) [Probinicid](Sigma P8161) make 200 mM stock in NaOH—H₂O.

### Biological Examples

Results of the assays are provided in Table 2 below. Activities of representative compounds are expressed as IC₅₀ against the kappa opioid receptor (KOR) and mu opioid receptor (MOR). Table 2 also presents selectivity of representative compounds for KOR.

TABLE 2
Activity of Representative Compounds

| | | KOR | MOR | |
|---|---|---|---|---|
| Compound | | IC₅₀ | IC₅₀ | Selectivity |
| No. | Structure | (nM) | (nM) | (MOR/KOR) |
| 1 | 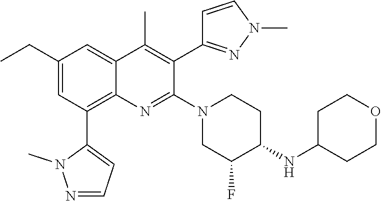 [SMILES: CCc1cc(-c2ccnn2C)c2nc(N3CC[C@H](NC4CCOCC4)[C@H](F)C3)c(-c3ccn(C)n3)c(C)c2c1] | +++ | − | +++ |
| 2 | 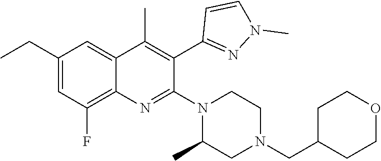 [SMILES: CCc1cc(F)c2nc(N3CCN(CC4CCOCC4)C[C@H]3C)c(-c3ccn(C)n3)c(C)c2c1] | +++ | − | +++ |
| 3 | 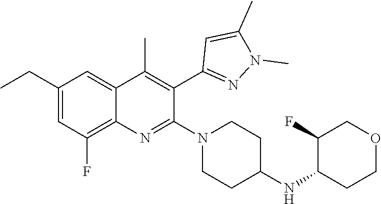 [SMILES: CCc1cc(F)c2nc(N3CCC(N[C@H]4CCOC[C@@H]4F)CC3)c(-c3cc(C)n(C)n3)c(C)c2c1] | +++ | + | +++ |
| 4 | 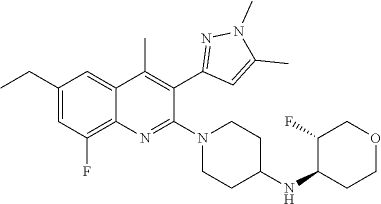 [SMILES: CCc1cc(F)c2nc(N3CCC(N[C@@H]4CCOC[C@H]4F)CC3)c(-c3cc(C)n(C)n3)c(C)c2c1] | ++ | + | ++ |
| 5 | 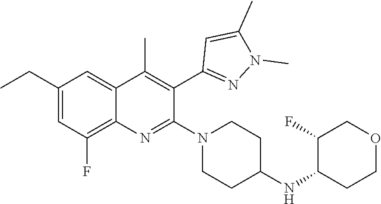 [SMILES: CCc1cc(F)c2nc(N3CCC(N[C@H]4CCOC[C@H]4F)CC3)c(-c3cc(C)n(C)n3)c(C)c2c1] | +++ | + | +++ |
| 6 | 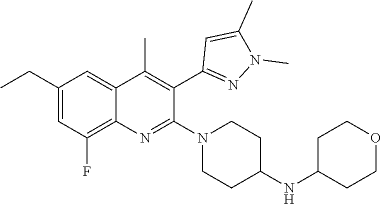 [SMILES: CCc1cc(F)c2nc(N3CCC(NC4CCOCC4)CC3)c(-c3cc(C)n(C)n3)c(C)c2c1] | +++ | + | ++ |
| 7 | 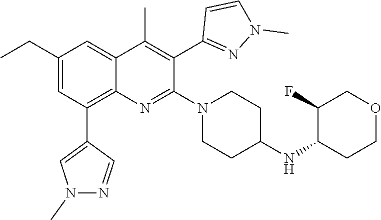 [SMILES: CCc1cc(-c2cnn(C)c2)c2nc(N3CCC(N[C@H]4CCOC[C@@H]4F)CC3)c(-c3ccn(C)n3)c(C)c2c1] | +++ | − | +++ |
| 8 | 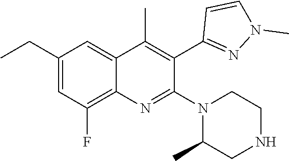 [SMILES: CCc1cc(F)c2nc(N3CCNC[C@H]3C)c(-c3ccn(C)n3)c(C)c2c1] | + | − | ++ |
| 9 | 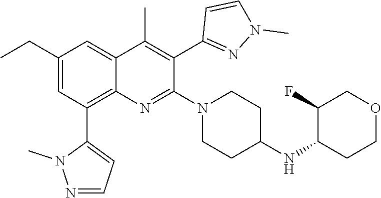 [SMILES: CCc1cc(-c2ccnn2C)c2nc(N3CCC(N[C@H]4CCOC[C@@H]4F)CC3)c(-c3ccn(C)n3)c(C)c2c1] | ++++ | + | +++ |
| 10 | 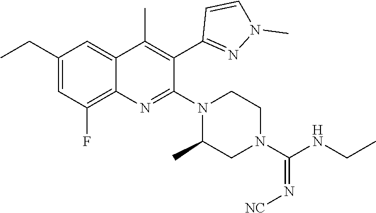 [SMILES: CCN/C(=N/C#N)N1CCN(c2nc3c(F)cc(CC)cc3c(C)c2-c2ccn(C)n2)[C@H](C)C1] | + | − | ++ |
| 11 | 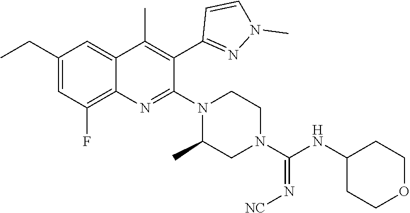 [SMILES: CCc1cc(F)c2nc(N3CCN(/C(=N\C#N)NC4CCOCC4)C[C@H]3C)c(-c3ccn(C)n3)c(C)c2c1] | ++ | +++ | ++ |
| 12 | 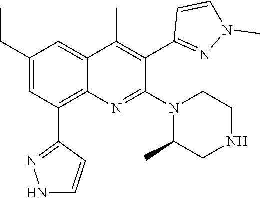 [SMILES: CCc1cc(-c2cc[nH]n2)c2nc(N3CCNC[C@H]3C)c(-c3ccn(C)n3)c(C)c2c1] | + | − | + |
| 13 | 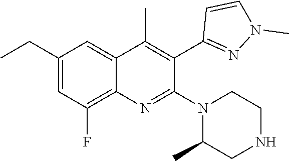 [SMILES: CCc1cc(F)c2nc(N3CCNC[C@H]3C)c(-c3ccn(C)n3)c(C)c2c1] | + | − | ++ |
| 14 | 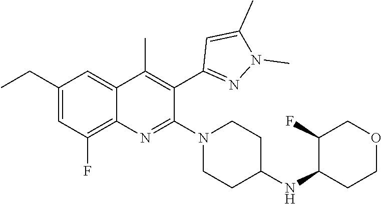 [SMILES: CCc1cc(F)c2nc(N3CCC(N[C@@H]4CCOC[C@@H]4F)CC3)c(-c3cc(C)n(C)n3)c(C)c2c1] | +++ | + | +++ |
| 15 | 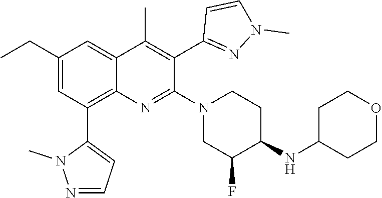 [SMILES: CCc1cc(-c2ccnn2C)c2nc(N3CC[C@@H](NC4CCOCC4)[C@@H](F)C3)c(-c3ccn(C)n3)c(C)c2c1] | +++ | ++ | ++ |
| 16 | 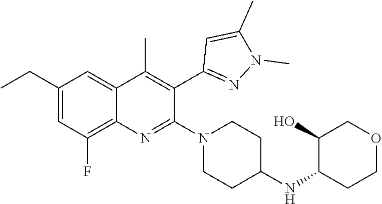 [SMILES: CCc1cc(F)c2nc(N3CCC(N[C@H]4CCOC[C@@H]4O)CC3)c(-c3cc(C)n(C)n3)c(C)c2c1] | +++ | + | ++ |
| 17 | 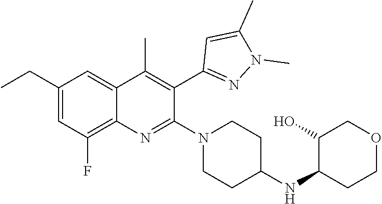 | +++ | + | ++ |
| 18 | 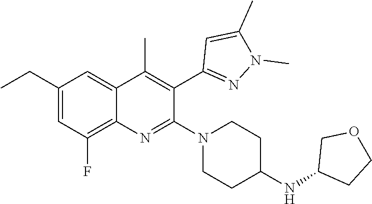 [SMILES: CCc1cc(F)c2nc(N3CCC(N[C@H]4CCOC4)CC3)c(-c3cc(C)n(C)n3)c(C)c2c1] | +++ | − | +++ |
| 19 | 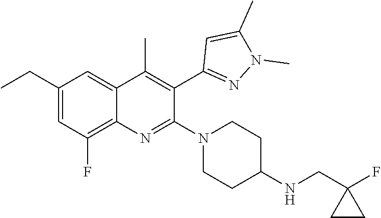 [SMILES: CCc1cc(F)c2nc(N3CCC(NCC4(F)CC4)CC3)c(-c3cc(C)n(C)n3)c(C)c2c1] | +++ | + | +++ |
| 20 | 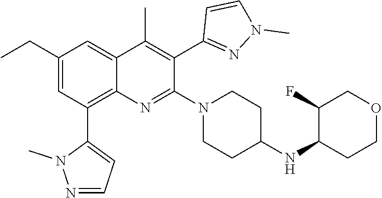 [SMILES: CCc1cc(-c2ccnn2C)c2nc(N3CCC(N[C@@H]4CCOC[C@@H]4F)CC3)c(-c3ccn(C)n3)c(C)c2c1] | ++++ | ++ | ++ |
| 21 | 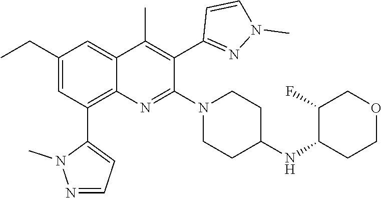 [SMILES: CCc1cc(-c2ccnn2C)c2nc(N3CCC(N[C@H]4CCOC[C@H]4F)CC3)c(-c3ccn(C)n3)c(C)c2c1] | +++ | ++ | ++ |
| 22 | 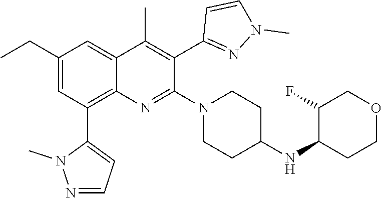 | +++ | + | ++ |
| 23 | 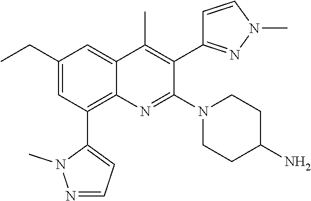 [SMILES: CCc1cc(-c2ccnn2C)c2nc(N3CCC(N)CC3)c(-c3ccn(C)n3)c(C)c2c1] | + | − | + |
| 24 | 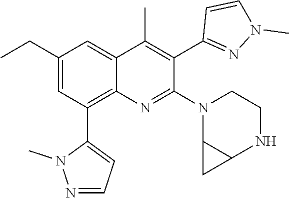 [SMILES: CCc1cc(-c2ccnn2C)c2nc(N3CCNC4CC43)c(-c3ccn(C)n3)c(C)c2c1] | + | − | + |
| 25 | 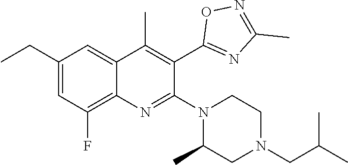 [SMILES: CCc1cc(F)c2nc(N3CCN(CC(C)C)C[C@H]3C)c(-c3nc(C)no3)c(C)c2c1] | ++++ | − | +++ |
| 26 | 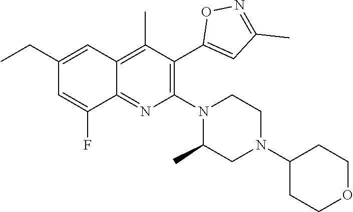 [SMILES: CCc1cc(F)c2nc(N3CCN(C4CCOCC4)C[C@H]3C)c(-c3cc(C)no3)c(C)c2c1] | ++++ | − | +++ |
| 27 | 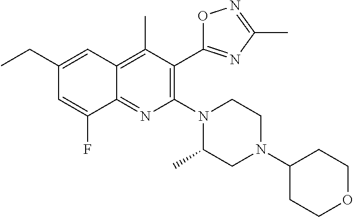 [SMILES: CCc1cc(F)c2nc(N3CCN(C4CCOCC4)C[C@@H]3C)c(-c3nc(C)no3)c(C)c2c1] | + | − | ++ |
| 28 | 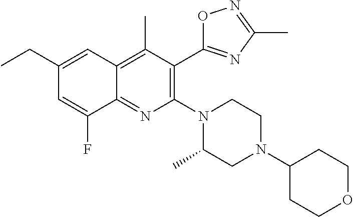 [SMILES: CCc1cc(F)c2nc(N3CCN(C4CCOCC4)C[C@@H]3C)c(-c3nc(C)no3)c(C)c2c1] | +++ | − | ++++ |
| 29 | 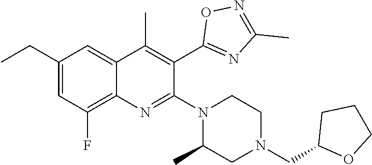 [SMILES: CCc1cc(F)c2nc(N3CCN(C[C@@H]4CCCO4)C[C@H]3C)c(-c3nc(C)no3)c(C)c2c1] | +++ | − | +++ |
| 30 | 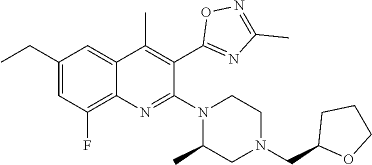 [SMILES: CCc1cc(F)c2nc(N3CCN(C[C@H]4CCCO4)C[C@H]3C)c(-c3nc(C)no3)c(C)c2c1] | +++ | − | +++ |
| 31 | 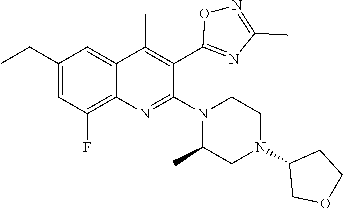 [SMILES: CCc1cc(F)c2nc(N3CCN([C@@H]4CCOC4)C[C@H]3C)c(-c3nc(C)no3)c(C)c2c1] | + | − | ++ |
| 32 | 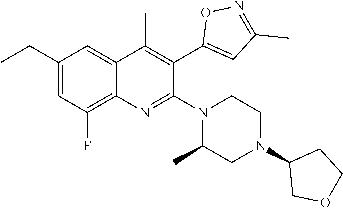 [SMILES: CCc1cc(F)c2nc(N3CCN([C@H]4CCOC4)C[C@H]3C)c(-c3cc(C)no3)c(C)c2c1] | ++ | − | +++ |
| 33 | 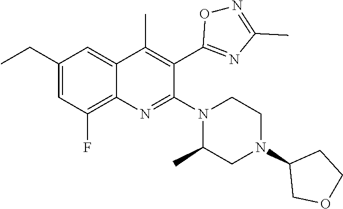 [SMILES: CCc1cc(F)c2nc(N3CCN([C@H]4CCOC4)C[C@H]3C)c(-c3nc(C)no3)c(C)c2c1] | +++ | − | +++ |
| 34 | 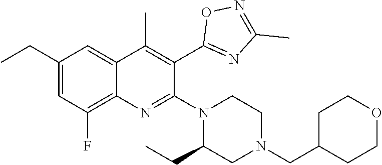 [SMILES: CCc1cc(F)c2nc(N3CCN(CC4CCOCC4)C[C@H]3CC)c(-c3nc(C)no3)c(C)c2c1] | ++ | + | + |
| 35 | 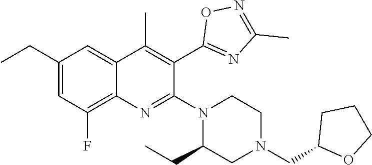 [SMILES: CCc1cc(F)c2nc(N3CCN(C[C@@H]4CCCO4)C[C@H]3CC)c(-c3nc(C)no3)c(C)c2c1] | ++ | − | ++ |
| 36 | 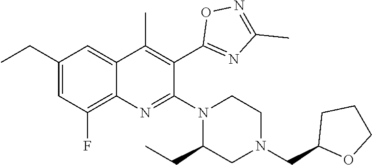 [SMILES: CCc1cc(F)c2nc(N3CCN(C[C@H]4CCCO4)C[C@H]3CC)c(-c3nc(C)no3)c(C)c2c1] | ++ | − | ++ |
| 37 | 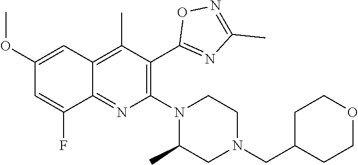 [SMILES: COc1cc(F)c2nc(N3CCN(CC4CCOCC4)C[C@H]3C)c(-c3nc(C)no3)c(C)c2c1] | + | − | ++ |
| 38 | 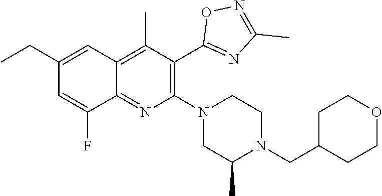 [SMILES: CCc1cc(F)c2nc(N3CCN(CC4CCOCC4)[C@@H](C)C3)c(-c3nc(C)no3)c(C)c2c1] | ++ | − | ++ |
| 39 | 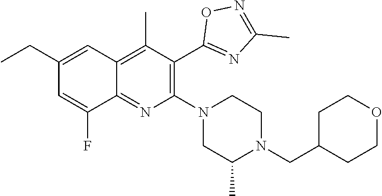 [SMILES: CCc1cc(F)c2nc(N3CCN(CC4CCOCC4)[C@H](C)C3)c(-c3nc(C)no3)c(C)c2c1] | + | − | ++ |
| 40 | 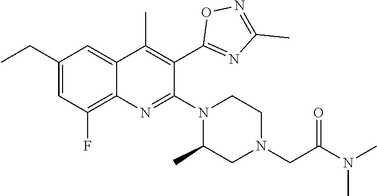 [SMILES: CCc1cc(F)c2nc(N3CCN(CC(=O)N(C)C)C[C@H]3C)c(-c3nc(C)no3)c(C)c2c1] | ++ | − | ++ |
| 41 | 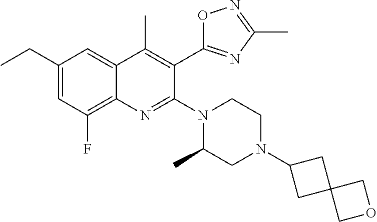 [SMILES: CCc1cc(F)c2nc(N3CCN(C4CC5(COC5)C4)C[C@H]3C)c(-c3nc(C)no3)c(C)c2c1] | ++ | − | +++ |
| 42 | 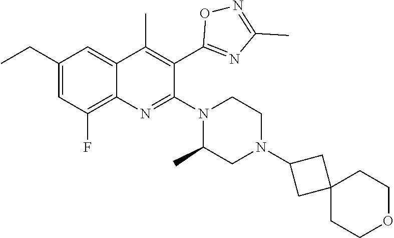 [SMILES: CCc1cc(F)c2nc(N3CCN(C4CC5(CCOCC5)C4)C[C@H]3C)c(-c3nc(C)no3)c(C)c2c1] | ++ | − | +++ |
| 43 | 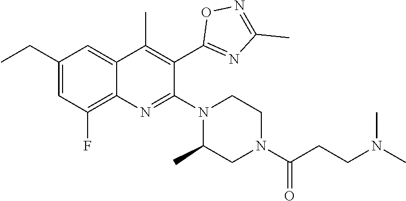 [SMILES: CCc1cc(F)c2nc(N3CCN(C(=O)CCN(C)C)C[C@H]3C)c(-c3nc(C)no3)c(C)c2c1] | ++ | − | ++ |
| 44 | 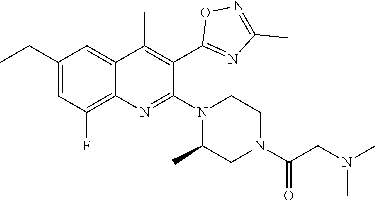 [SMILES: CCc1cc(F)c2nc(N3CCN(C(=O)CN(C)C)C[C@H]3C)c(-c3nc(C)no3)c(C)c2c1] | ++ | − | +++ |
| 45 | 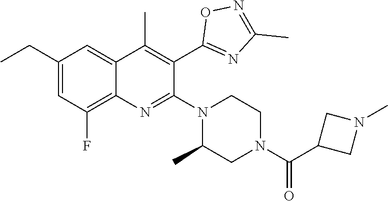 [SMILES: CCc1cc(F)c2nc(N3CCN(C(=O)C4CN(C)C4)C[C@H]3C)c(-c3nc(C)no3)c(C)c2c1] | ++ | − | ++ |
| 46 | 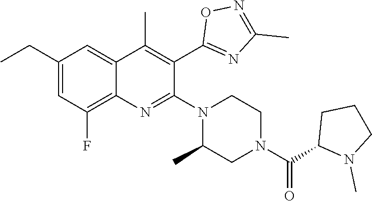 [SMILES: CCc1cc(F)c2nc(N3CCN(C(=O)[C@@H]4CCCN4C)C[C@H]3C)c(-c3nc(C)no3)c(C)c2c1] | ++ | − | +++ |
| 47 | 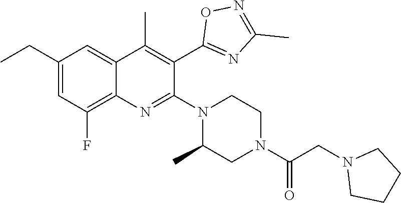 [SMILES: CCc1cc(F)c2nc(N3CCN(C(=O)CN4CCCC4)C[C@H]3C)c(-c3nc(C)no3)c(C)c2c1] | +++ | + | +++ |
| 48 | 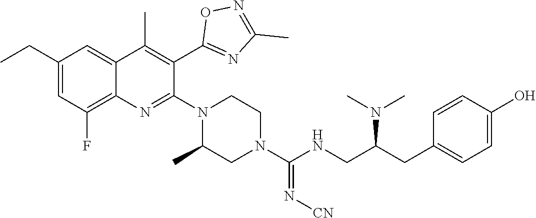 [SMILES: CCc1cc(F)c2nc(N3CCN(/C(=N/C#N)NC[C@H](Cc4ccc(O)cc4)N(C)C)C[C@H]3C)c(-c3nc(C)no3)c(C)c2c1] | ++ | + | + |
| 49 | 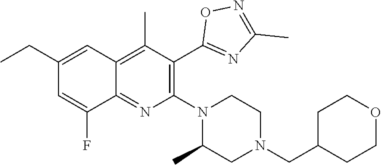 [SMILES: CCc1cc(F)c2nc(N3CCN(CC4CCOCC4)C[C@H]3C)c(-c3nc(C)no3)c(C)c2c1] | ++ | − | +++ |
| 50 | 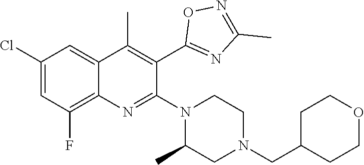 [SMILES: Cc1noc(-c2c(N3CCN(CC4CCOCC4)C[C@H]3C)nc3c(F)cc(Cl)cc3c2C)n1] | + | − | ++ |
| 51 | 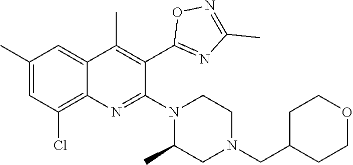 [SMILES: Cc1cc(Cl)c2nc(N3CCN(CC4CCOCC4)C[C@H]3C)c(-c3nc(C)no3)c(C)c2c1] | +++ | + | +++ |
| 52 | 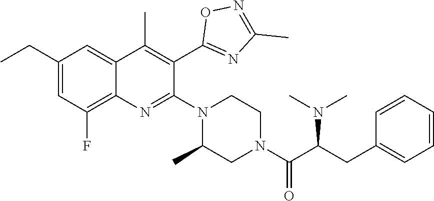 [SMILES: CCc1cc(F)c2nc(N3CCN(C(=O)[C@H](Cc4ccccc4)N(C)C)C[C@H]3C)c(-c3nc(C)no3)c(C)c2c1] | + | − | ++ |
| 53 | 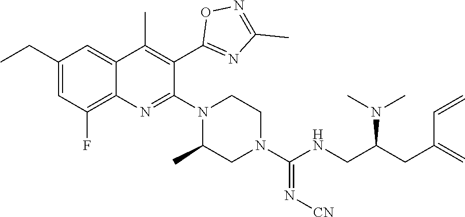 [SMILES: C=CC(=C)C[C@@H](CN/C(=N\C#N)N1CCN(c2nc3c(F)cc(CC)cc3c(C)c2-c2nc(C)no2)[C@H](C)C1)N(C)C] | ++ | + | + |
| 54 | 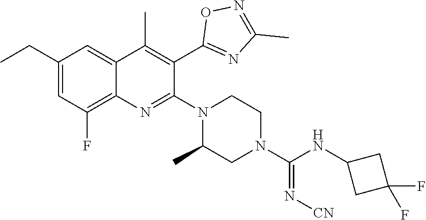 [SMILES: CCc1cc(F)c2nc(N3CCN(/C(=N/C#N)NC4CC(F)(F)C4)C[C@H]3C)c(-c3nc(C)no3)c(C)c2c1] | ++++ | ++ | +++ |
| 55 | 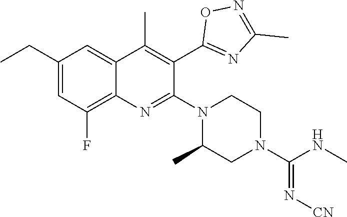 [SMILES: CCc1cc(F)c2nc(N3CCN(/C(=N/C#N)NC)C[C@H]3C)c(-c3nc(C)no3)c(C)c2c1] | ++ | − | ++ |
| 56 | 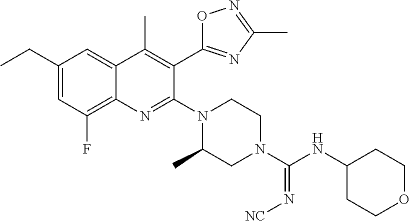 [SMILES: CCc1cc(F)c2nc(N3CCN(/C(=N\C#N)NC4CCOCC4)C[C@H]3C)c(-c3nc(C)no3)c(C)c2c1] | ++ | + | ++ |
| 57 | 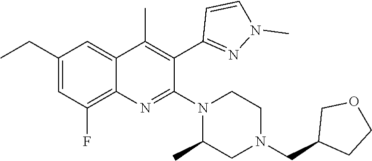 [SMILES: CCc1cc(F)c2nc(N3CCN(C[C@@H]4CCOC4)C[C@H]3C)c(-c3ccn(C)n3)c(C)c2c1] | + | − | ++ |
| 58 | 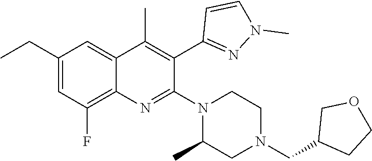 | ++ | − | +++ |
| 59 | 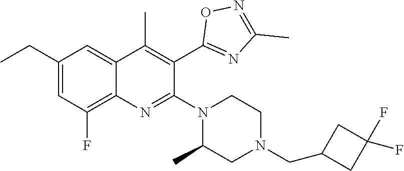 [SMILES: CCc1cc(F)c2nc(N3CCN(CC4CC(F)(F)C4)C[C@H]3C)c(-c3nc(C)no3)c(C)c2c1] | ++ | − | +++ |
| 60 | 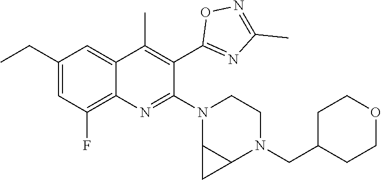 [SMILES: CCc1cc(F)c2nc(N3CCN(CC4CCOCC4)C4CC43)c(-c3nc(C)no3)c(C)c2c1] | + | − | |
| 61 | 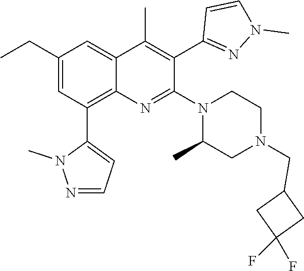 [SMILES: CCc1cc(-c2ccnn2C)c2nc(N3CCN(CC4CC(F)(F)C4)C[C@H]3C)c(-c3ccn(C)n3)c(C)c2c1] | + | − | ++ |
| 62 | 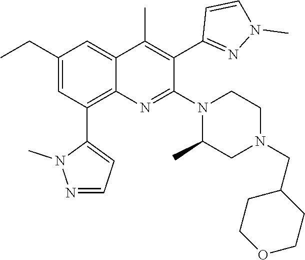 [SMILES: CCc1cc(-c2ccnn2C)c2nc(N3CCN(CC4CCOCC4)C[C@H]3C)c(-c3ccn(C)n3)c(C)c2c1] | +++ | + | +++ |
| 63 | 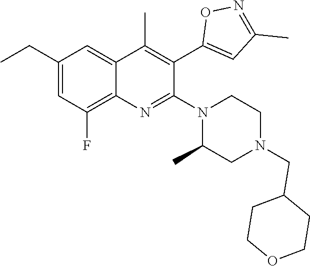 [SMILES: CCc1cc(F)c2nc(N3CCN(CC4CCOCC4)C[C@H]3C)c(-c3cc(C)no3)c(C)c2c1] | ++ | − | +++ |
| 64 | 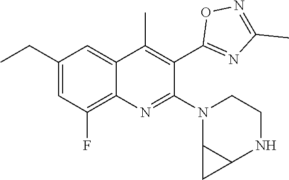 [SMILES: CCc1cc(F)c2nc(N3CCNC4CC43)c(-c3nc(C)no3)c(C)c2c1] | + | − | ++ |
| 65 | 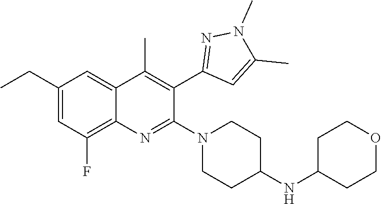 [SMILES: CCc1cc(F)c2nc(N3CCC(NC4CCOCC4)CC3)c(-c3cc(C)n(C)n3)c(C)c2c1] | +++ | − | +++ |
| 66 | 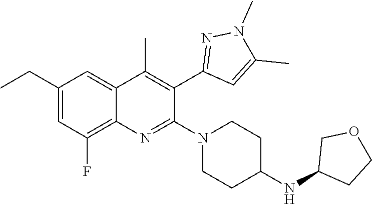 [SMILES: CCc1cc(F)c2nc(N3CCC(N[C@@H]4CCOC4)CC3)c(-c3cc(C)n(C)n3)c(C)c2c1] | +++ | + | +++ |
| 67 | 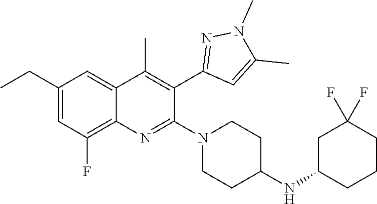 [SMILES: CCc1cc(F)c2nc(N3CCC(N[C@H]4CCCC(F)(F)C4)CC3)c(-c3cc(C)n(C)n3)c(C)c2c1] | +++ | ++ | ++ |
| 68 | 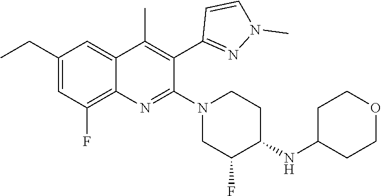 [SMILES: CCc1cc(F)c2nc(N3CC[C@H](NC4CCOCC4)[C@H](F)C3)c(-c3ccn(C)n3)c(C)c2c1] | ++ | − | +++ |
| 69 | 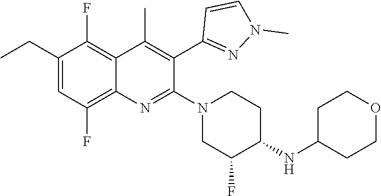 [SMILES: CCc1cc(F)c2nc(N3CC[C@H](NC4CCOCC4)[C@H](F)C3)c(-c3ccn(C)n3)c(C)c2c1F] | ++ | − | +++ |
| 70 | 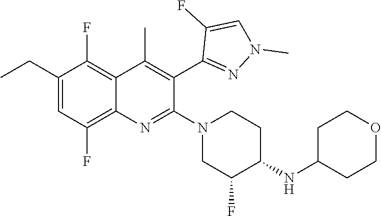 [SMILES: CCc1cc(F)c2nc(N3CC[C@H](NC4CCOCC4)[C@H](F)C3)c(-c3nn(C)cc3F)c(C)c2c1F] | +++ | − | +++ |
| 71 | 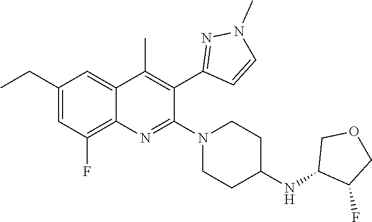 [SMILES: CCc1cc(F)c2nc(N3CCC(N[C@@H]4COC[C@@H]4F)CC3)c(-c3ccn(C)n3)c(C)c2c1] | +++ | − | +++ |
| 72 | 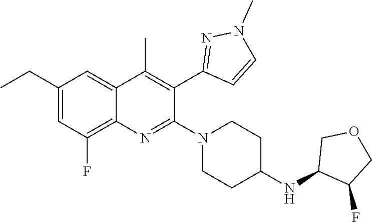 [SMILES: CCc1cc(F)c2nc(N3CCC(N[C@H]4COC[C@H]4F)CC3)c(-c3ccn(C)n3)c(C)c2c1] | ++ | − | ++ |
| 73 | 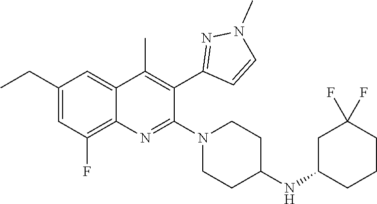 [SMILES: CCc1cc(F)c2nc(N3CCC(N[C@H]4CCCC(F)(F)C4)CC3)c(-c3ccn(C)n3)c(C)c2c1] | +++ | + | ++ |
| 74 | 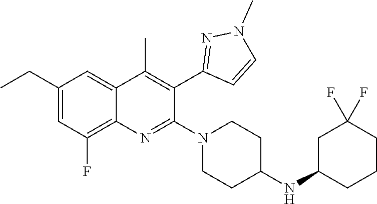 [SMILES: CCc1cc(F)c2nc(N3CCC(N[C@@H]4CCCC(F)(F)C4)CC3)c(-c3ccn(C)n3)c(C)c2c1] | +++ | + | ++ |
| 75 | 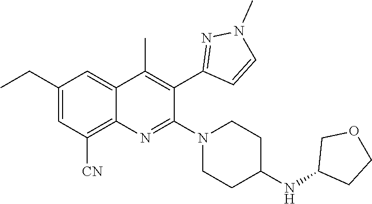 [SMILES: CCc1cc(C#N)c2nc(N3CCC(N[C@H]4CCOC4)CC3)c(-c3ccn(C)n3)c(C)c2c1] | +++ | + | +++ |
| 76 | 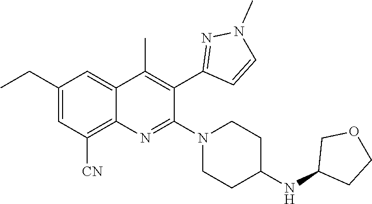 [SMILES: CCc1cc(C#N)c2nc(N3CCC(N[C@@H]4CCOC4)CC3)c(-c3ccn(C)n3)c(C)c2c1] | ++++ | + | +++ |
| 77 | 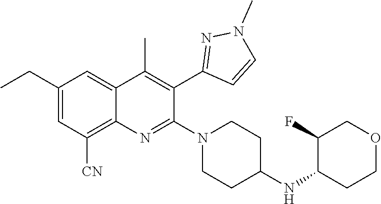 [SMILES: CCc1cc(C#N)c2nc(N3CCC(N[C@H]4CCOC[C@@H]4F)CC3)c(-c3ccn(C)n3)c(C)c2c1] | ++++ | + | +++ |
| 78 | 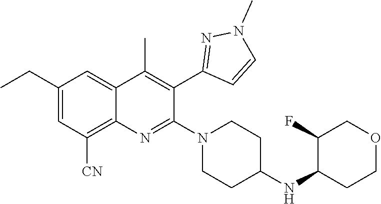 [SMILES: CCc1cc(C#N)c2nc(N3CCC(N[C@@H]4CCOC[C@@H]4F)CC3)c(-c3ccn(C)n3)c(C)c2c1] | ++++ | ++ | ++ |
| 79 | 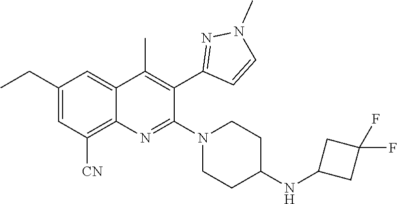 [SMILES: CCc1cc(C#N)c2nc(N3CCC(NC4CC(F)(F)C4)CC3)c(-c3ccn(C)n3)c(C)c2c1] | +++ | − | +++ |
| 80 | 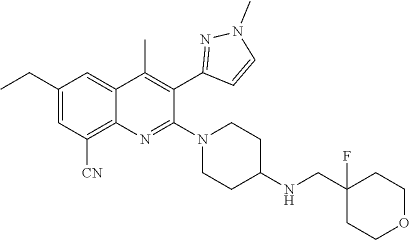 [SMILES: CCc1cc(C#N)c2nc(N3CCC(NCC4(F)CCOCC4)CC3)c(-c3ccn(C)n3)c(C)c2c1] | +++ | + | ++ |
| 81 | 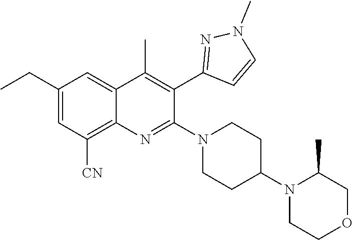 [SMILES: CCc1cc(C#N)c2nc(N3CCC(N4CCOC[C@@H]4C)CC3)c(-c3ccn(C)n3)c(C)c2c1] | ++ | − | ++ |
| 82 | 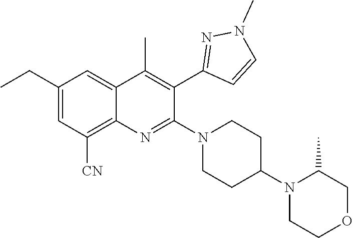 | ++ | − | ++ |
| 83 | 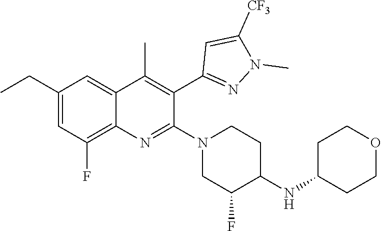 | + | − | +++ |
| 84 | 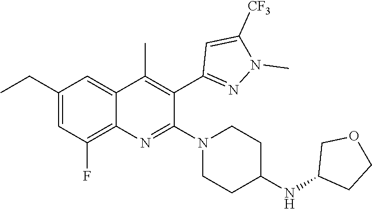 [SMILES: CCc1cc(F)c2nc(N3CCC(N[C@H]4CCOC4)CC3)c(-c3cc(C(F)(F)F)n(C)n3)c(C)c2c1] | ++ | − | +++ |
| 85 | 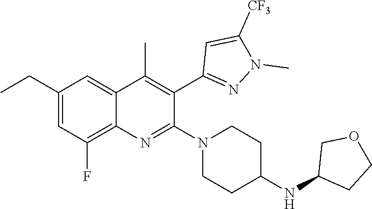 [SMILES: CCc1cc(F)c2nc(N3CCC(N[C@@H]4CCOC4)CC3)c(-c3cc(C(F)(F)F)n(C)n3)c(C)c2c1] | ++ | − | +++ |
| 86 | 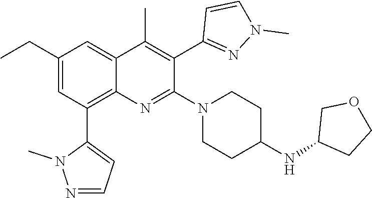 [SMILES: CCc1cc(-c2ccnn2C)c2nc(N3CCC(N[C@H]4CCOC4)CC3)c(-c3ccn(C)n3)c(C)c2c1] | ++++ | − | +++ |
| 87 | 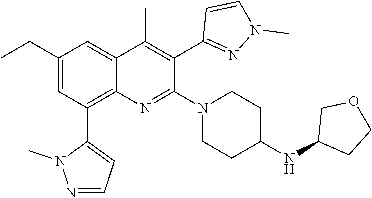 [SMILES: CCc1cc(-c2ccnn2C)c2nc(N3CCC(N[C@@H]4CCOC4)CC3)c(-c3ccn(C)n3)c(C)c2c1] | ++++ | + | +++ |
| 88 | 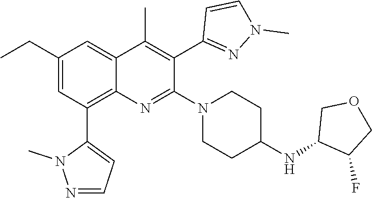 [SMILES: CCc1cc(-c2ccnn2C)c2nc(N3CCC(N[C@@H]4COC[C@@H]4F)CC3)c(-c3ccn(C)n3)c(C)c2c1] | +++ | + | ++ |
| 89 | 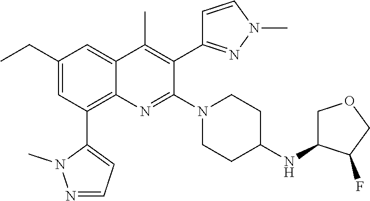 [SMILES: CCc1cc(-c2ccnn2C)c2nc(N3CCC(N[C@H]4COC[C@H]4F)CC3)c(-c3ccn(C)n3)c(C)c2c1] | +++ | − | ++ |
| 90 | 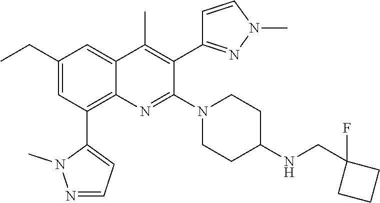 [SMILES: CCc1cc(-c2ccnn2C)c2nc(N3CCC(NCC4(F)CCC4)CC3)c(-c3ccn(C)n3)c(C)c2c1] | ++++ | ++ | +++ |
| 91 | 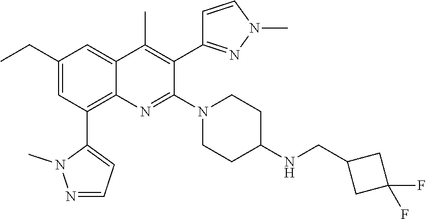 [SMILES: CCc1cc(-c2ccnn2C)c2nc(N3CCC(NCC4CC(F)(F)C4)CC3)c(-c3ccn(C)n3)c(C)c2c1] | +++ | ++ | ++ |
| 92 | 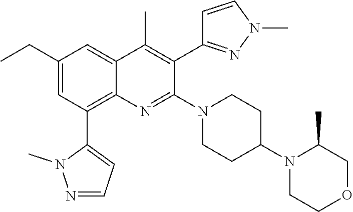 [SMILES: CCc1cc(-c2ccnn2C)c2nc(N3CCC(N4CCOC[C@@H]4C)CC3)c(-c3ccn(C)n3)c(C)c2c1] | − | − | |
| 93 | 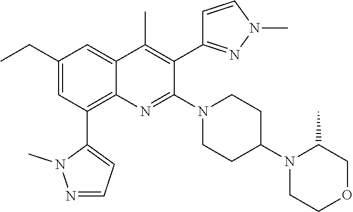 | − | − | |
| 94 | 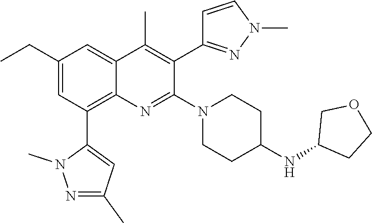 [SMILES: CCc1cc(-c2cc(C)nn2C)c2nc(N3CCC(N[C@H]4CCOC4)CC3)c(-c3ccn(C)n3)c(C)c2c1] | ++++ | ++ | +++ |
| 95 | 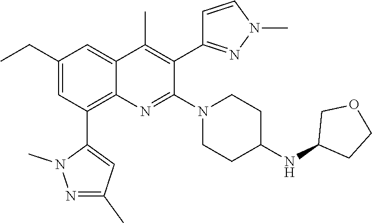 [SMILES: CCc1cc(-c2cc(C)nn2C)c2nc(N3CCC(N[C@@H]4CCOC4)CC3)c(-c3ccn(C)n3)c(C)c2c1] | ++++ | ++ | +++ |
| 96 | 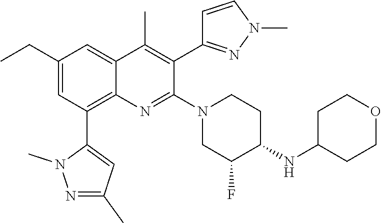 [SMILES: CCc1cc(-c2cc(C)nn2C)c2nc(N3CC[C@H](NC4CCOCC4)[C@H](F)C3)c(-c3ccn(C)n3)c(C)c2c1] | +++ | − | +++ |
| 97 | 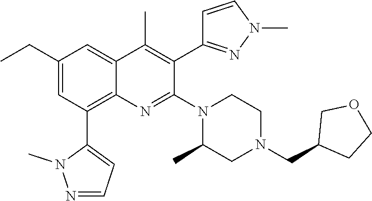 [SMILES: CCc1cc(-c2ccnn2C)c2nc(N3CCN(C[C@@H]4CCOC4)C[C@H]3C)c(-c3ccn(C)n3)c(C)c2c1] | +++ | − | +++ |
| 98 | 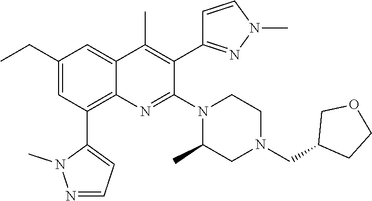 [SMILES: CCc1cc(-c2ccnn2C)c2nc(N3CCN(C[C@H]4CCOC4)C[C@H]3C)c(-c3ccn(C)n3)c(C)c2c1] | ++ | − | +++ |
| 99 | 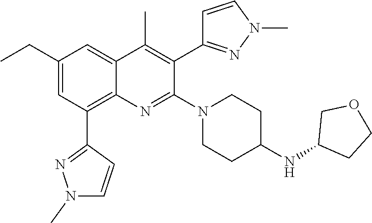 [SMILES: CCc1cc(-c2ccn(C)n2)c2nc(N3CCC(N[C@H]4CCOC4)CC3)c(-c3ccn(C)n3)c(C)c2c1] | ++++ | − | ++++ |
| 100 | 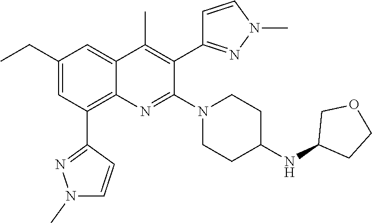 [SMILES: CCc1cc(-c2ccn(C)n2)c2nc(N3CCC(N[C@@H]4CCOC4)CC3)c(-c3ccn(C)n3)c(C)c2c1] | ++++ | − | ++++ |
| 101 | 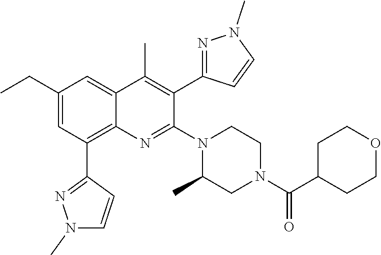 [SMILES: CCc1cc(-c2ccn(C)n2)c2nc(N3CCN(C(=O)C4CCOCC4)C[C@H]3C)c(-c3ccn(C)n3)c(C)c2c1] | − | − | |
| 102 | 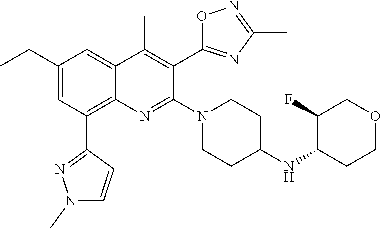 [SMILES: CCc1cc(-c2ccn(C)n2)c2nc(N3CCC(N[C@H]4CCOC[C@@H]4F)CC3)c(-c3nc(C)no3)c(C)c2c1] | ++++ | − | ++++ |
| 103 | 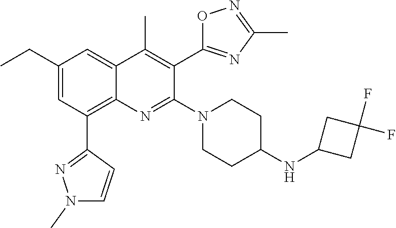 [SMILES: CCc1cc(-c2ccn(C)n2)c2nc(N3CCC(NC4CC(F)(F)C4)CC3)c(-c3nc(C)no3)c(C)c2c1] | +++ | − | +++ |
| 104 | 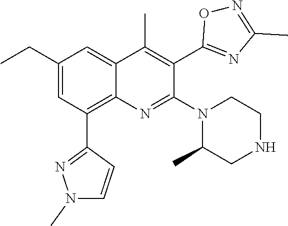 [SMILES: CCc1cc(-c2ccn(C)n2)c2nc(N3CCNC[C@H]3C)c(-c3nc(C)no3)c(C)c2c1] | ++ | − | +++ |
| 105 | 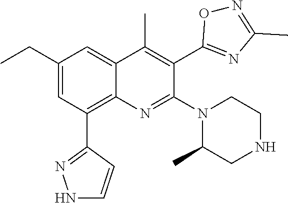 [SMILES: CCc1cc(-c2cc[nH]n2)c2nc(N3CCNC[C@H]3C)c(-c3nc(C)no3)c(C)c2c1] | ++ | − | ++ |
| 106 | 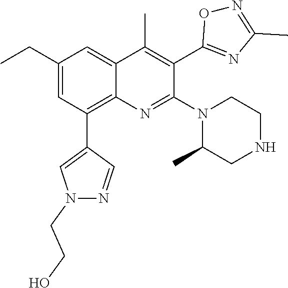 [SMILES: CCc1cc(-c2cnn(CCO)c2)c2nc(N3CCNC[C@H]3C)c(-c3nc(C)no3)c(C)c2c1] | + | − | ++ |
| 107 | 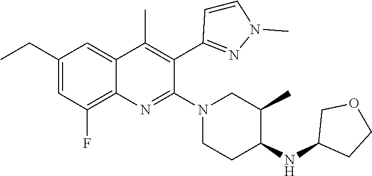 [SMILES: CCc1cc(F)c2nc(N3CC[C@H](N[C@@H]4CCOC4)[C@H](C)C3)c(-c3ccn(C)n3)c(C)c2c1] | ++ | − | +++ |
| 108 | 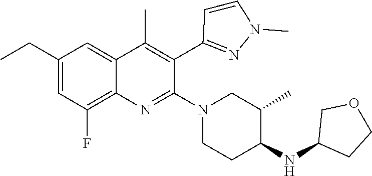 [SMILES: CCc1cc(F)c2nc(N3CC[C@H](N[C@@H]4CCOC4)[C@@H](C)C3)c(-c3ccn(C)n3)c(C)c2c1] | +++ | + | +++ |
| 109 | 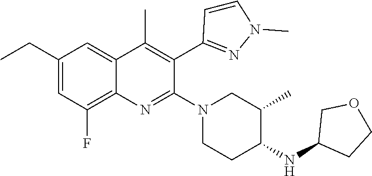 [SMILES: CCc1cc(F)c2nc(N3CC[C@@H](N[C@@H]4CCOC4)[C@@H](C)C3)c(-c3ccn(C)n3)c(C)c2c1] | | | |
| 110 | 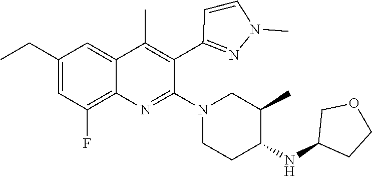 [SMILES: CCc1cc(F)c2nc(N3CC[C@@H](N[C@@H]4CCOC4)[C@H](C)C3)c(-c3ccn(C)n3)c(C)c2c1] | +++ | − | +++ |
| 111 | 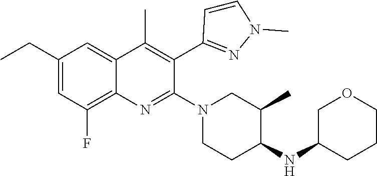 [SMILES: CCc1cc(F)c2nc(N3CC[C@H](N[C@@H]4CCCOC4)[C@H](C)C3)c(-c3ccn(C)n3)c(C)c2c1] | ++ | − | +++ |
| 112 | 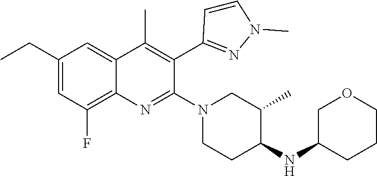 [SMILES: CCc1cc(F)c2nc(N3CC[C@H](N[C@@H]4CCCOC4)[C@@H](C)C3)c(-c3ccn(C)n3)c(C)c2c1] | +++ | − | +++ |
| 113 | 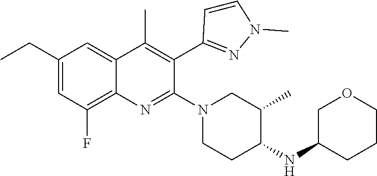 [SMILES: CCc1cc(F)c2nc(N3CC[C@@H](N[C@@H]4CCCOC4)[C@@H](C)C3)c(-c3ccn(C)n3)c(C)c2c1] | ++++ | − | ++++ |
| 114 | 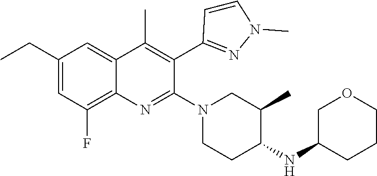 [SMILES: CCc1cc(F)c2nc(N3CC[C@@H](N[C@@H]4CCCOC4)[C@H](C)C3)c(-c3ccn(C)n3)c(C)c2c1] | +++ | + | ++ |
| 115 | 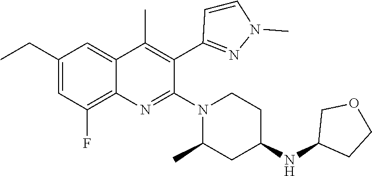 [SMILES: CCc1cc(F)c2nc(N3CC[C@@H](N[C@@H]4CCOC4)C[C@H]3C)c(-c3ccn(C)n3)c(C)c2c1] | | | |
| 116 | 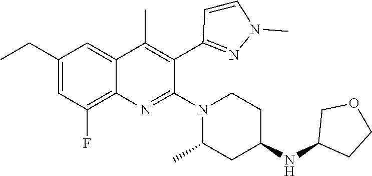 [SMILES: CCc1cc(F)c2nc(N3CC[C@@H](N[C@@H]4CCOC4)C[C@@H]3C)c(-c3ccn(C)n3)c(C)c2c1] | +++ | − | ++++ |
| 117 | 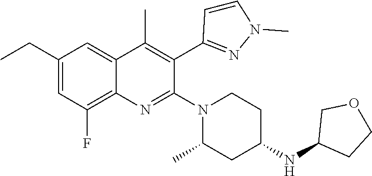 [SMILES: CCc1cc(F)c2nc(N3CC[C@H](N[C@@H]4CCOC4)C[C@@H]3C)c(-c3ccn(C)n3)c(C)c2c1] | +++ | + | +++ |
| 118 | 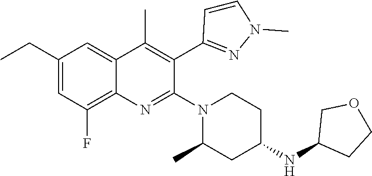 [SMILES: CCc1cc(F)c2nc(N3CC[C@H](N[C@@H]4CCOC4)C[C@H]3C)c(-c3ccn(C)n3)c(C)c2c1] | ++++ | + | ++++ |
| 119 | 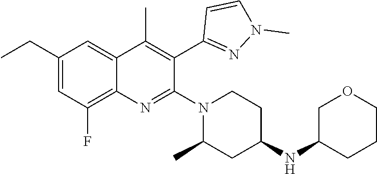 [SMILES: CCc1cc(F)c2nc(N3CC[C@@H](N[C@@H]4CCCOC4)C[C@H]3C)c(-c3ccn(C)n3)c(C)c2c1] | ++++ | + | ++++ |
| 120 | 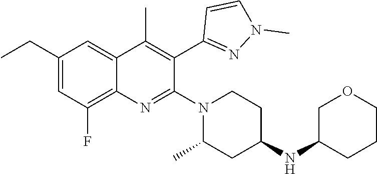 [SMILES: CCc1cc(F)c2nc(N3CC[C@@H](N[C@@H]4CCCOC4)C[C@@H]3C)c(-c3ccn(C)n3)c(C)c2c1] | ++++ | − | ++++ |
| 121 | 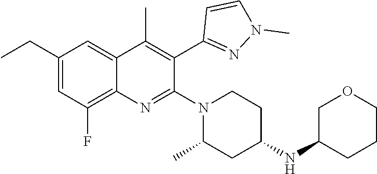 [SMILES: CCc1cc(F)c2nc(N3CC[C@H](N[C@@H]4CCCOC4)C[C@@H]3C)c(-c3ccn(C)n3)c(C)c2c1] | ++ | − | +++ |
| 122 | 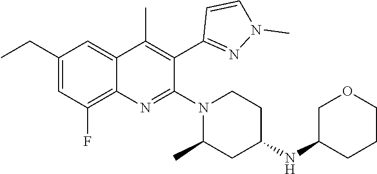 [SMILES: CCc1cc(F)c2nc(N3CC[C@H](N[C@@H]4CCCOC4)C[C@H]3C)c(-c3ccn(C)n3)c(C)c2c1] | ++++ | ++ | +++ |
| 123 | 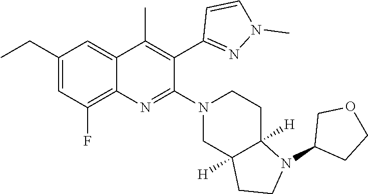 [SMILES: CCc1cc(F)c2nc(N3CC[C@@H]4[C@@H](CCN4[C@@H]4CCOC4)C3)c(-c3ccn(C)n3)c(C)c2c1] | ++ | − | +++ |
| 124 | 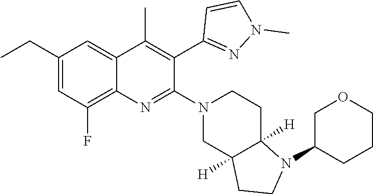 [SMILES: CCc1cc(F)c2nc(N3CC[C@@H]4[C@@H](CCN4[C@@H]4CCCOC4)C3)c(-c3ccn(C)n3)c(C)c2c1] | ++ | − | +++ |
| 125 | 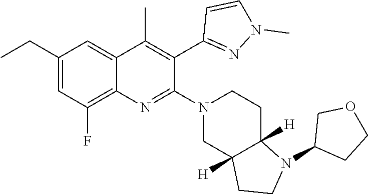 [SMILES: CCc1cc(F)c2nc(N3CC[C@H]4[C@H](CCN4[C@@H]4CCOC4)C3)c(-c3ccn(C)n3)c(C)c2c1] | +++ | − | ++++ |
| 126 | 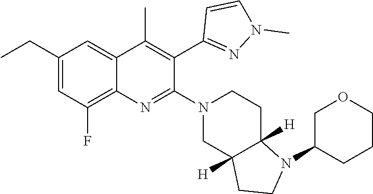 [SMILES: CCc1cc(F)c2nc(N3CC[C@H]4[C@H](CCN4[C@@H]4CCCOC4)C3)c(-c3ccn(C)n3)c(C)c2c1] | ++ | − | ++ |
| 127 | 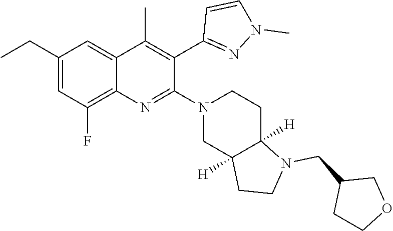 | + | − | +++ |
| 128 | 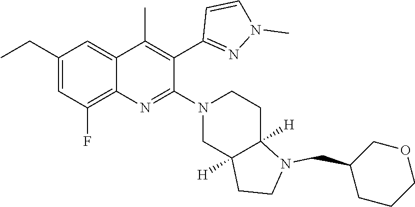 [SMILES: CCc1cc(F)c2nc(N3CC[C@@H]4[C@@H](CCN4C[C@@H]4CCCOC4)C3)c(-c3ccn(C)n3)c(C)c2c1] | + | − | ++ |
| 129 | 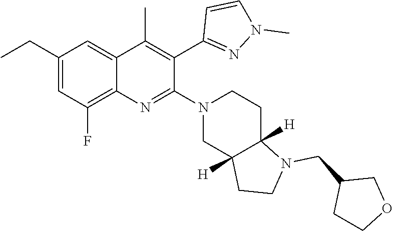 | ++ | − | ++ |
| 130 | 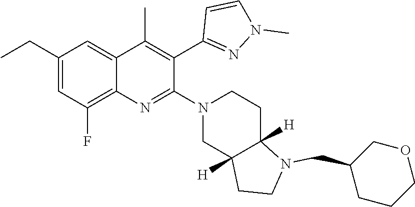 [SMILES: CCc1cc(F)c2nc(N3CC[C@H]4[C@H](CCN4C[C@@H]4CCCOC4)C3)c(-c3ccn(C)n3)c(C)c2c1] | ++ | − | ++ |
| 131 | 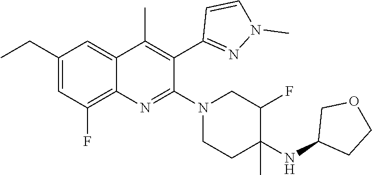 [SMILES: CCc1cc(F)c2nc(N3CCC(C)(N[C@@H]4CCOC4)C(F)C3)c(-c3ccn(C)n3)c(C)c2c1] | | | |
| 132 | 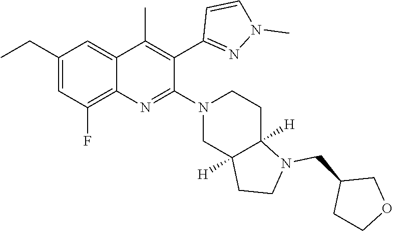 [SMILES: CCc1cc(F)c2nc(N3CC[C@@H]4[C@@H](CCN4C[C@@H]4CCOC4)C3)c(-c3ccn(C)n3)c(C)c2c1] | | | |
| 133 | 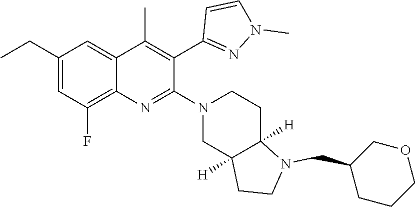 [SMILES: CCc1cc(F)c2nc(N3CC[C@@H]4[C@@H](CCN4C[C@@H]4CCCOC4)C3)c(-c3ccn(C)n3)c(C)c2c1] | ++ | − | +++ |
| 134 | 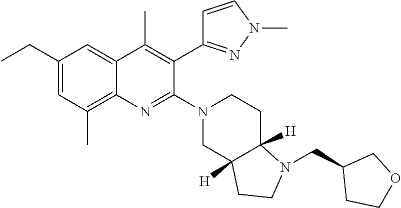 [SMILES: CCc1cc(C)c2nc(N3CC[C@H]4[C@H](CCN4C[C@@H]4CCOC4)C3)c(-c3ccn(C)n3)c(C)c2c1] | + | − | +++ |
| 135 | 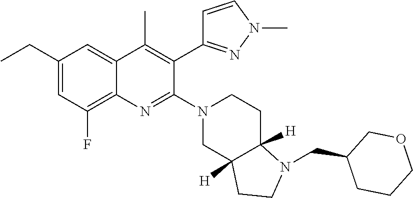 [SMILES: CCc1cc(F)c2nc(N3CC[C@H]4[C@H](CCN4C[C@@H]4CCCOC4)C3)c(-c3ccn(C)n3)c(C)c2c1] | | | |
| 136 | 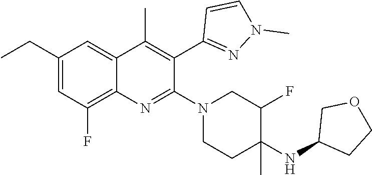 [SMILES: CCc1cc(F)c2nc(N3CCC(C)(N[C@@H]4CCOC4)C(F)C3)c(-c3ccn(C)n3)c(C)c2c1] | | | |
| 137 | 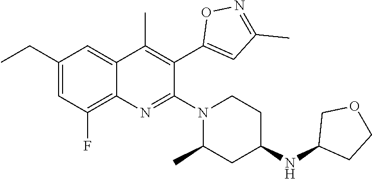 [SMILES: CCc1cc(F)c2nc(N3CC[C@@H](N[C@@H]4CCOC4)C[C@H]3C)c(-c3cc(C)no3)c(C)c2c1] | ++++ | + | ++++ |
| 138 | 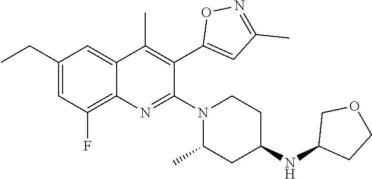 [SMILES: CCc1cc(F)c2nc(N3CC[C@@H](N[C@@H]4CCOC4)C[C@@H]3C)c(-c3cc(C)no3)c(C)c2c1] | ++ | − | +++ |
| 139 | 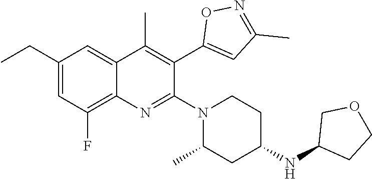 [SMILES: CCc1cc(F)c2nc(N3CC[C@H](N[C@@H]4CCOC4)C[C@@H]3C)c(-c3cc(C)no3)c(C)c2c1] | | | |
| 140 | 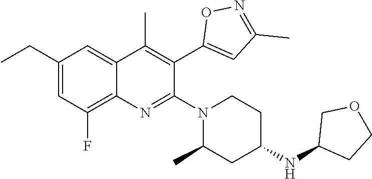 [SMILES: CCc1cc(F)c2nc(N3CC[C@H](N[C@@H]4CCOC4)C[C@H]3C)c(-c3cc(C)no3)c(C)c2c1] | ++++ | + | +++ |
| 141 | 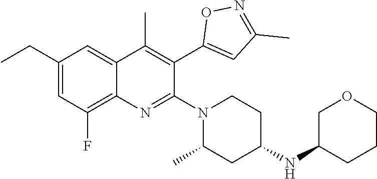 [SMILES: CCc1cc(F)c2nc(N3CC[C@H](N[C@@H]4CCCOC4)C[C@@H]3C)c(-c3cc(C)no3)c(C)c2c1] | | | |
| 142 | 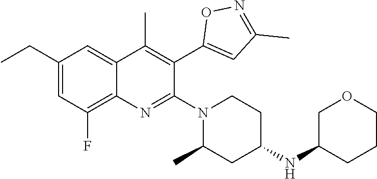 [SMILES: CCc1cc(F)c2nc(N3CC[C@H](N[C@@H]4CCCOC4)C[C@H]3C)c(-c3cc(C)no3)c(C)c2c1] | ++++ | ++ | +++ |
| 143 | 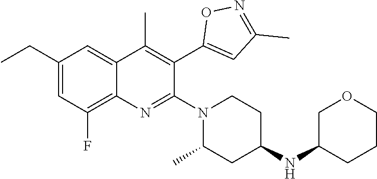 [SMILES: CCc1cc(F)c2nc(N3CC[C@@H](N[C@@H]4CCCOC4)C[C@@H]3C)c(-c3cc(C)no3)c(C)c2c1] | ++ | − | ++++ |
| 144 | 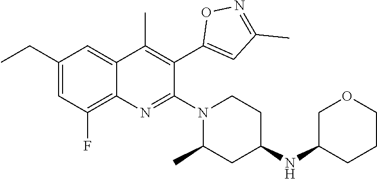 [SMILES: CCc1cc(F)c2nc(N3CC[C@@H](N[C@@H]4CCCOC4)C[C@H]3C)c(-c3cc(C)no3)c(C)c2c1] | ++++ | + | +++ |
| 145 | 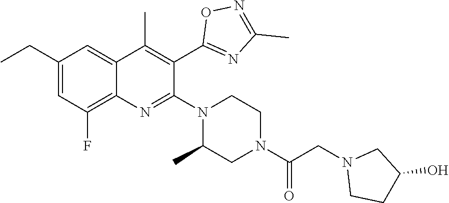 [SMILES: CCc1cc(F)c2nc(N3CCN(C(=O)CN4CC[C@@H](O)C4)C[C@H]3C)c(-c3nc(C)no3)c(C)c2c1] | +++ | − | ++++ |
| 146 | 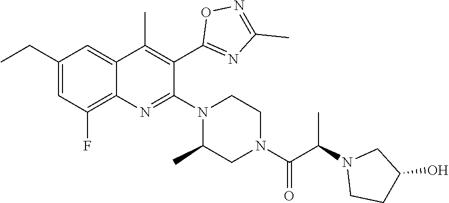 [SMILES: CCc1cc(F)c2nc(N3CCN(C(=O)[C@@H](C)N4CC[C@@H](O)C4)C[C@H]3C)c(-c3nc(C)no3)c(C)c2c1] | ++ | − | +++ |
| 147 | 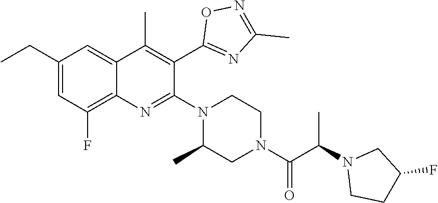 [SMILES: CCc1cc(F)c2nc(N3CCN(C(=O)[C@@H](C)N4CC[C@@H](F)C4)C[C@H]3C)c(-c3nc(C)no3)c(C)c2c1] | +++ | + | +++ |
| 148 | 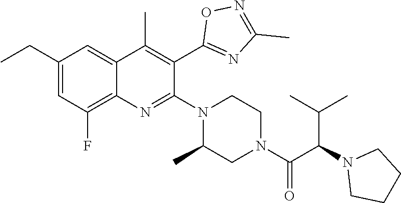 [SMILES: CCc1cc(F)c2nc(N3CCN(C(=O)[C@@H](C(C)C)N4CCCC4)C[C@H]3C)c(-c3nc(C)no3)c(C)c2c1] | +++ | − | +++ |
| 149 | 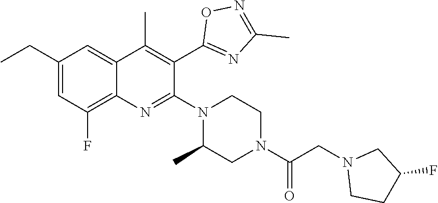 [SMILES: CCc1cc(F)c2nc(N3CCN(C(=O)CN4CC[C@@H](F)C4)C[C@H]3C)c(-c3nc(C)no3)c(C)c2c1] | +++ | + | +++ |
| 150 | 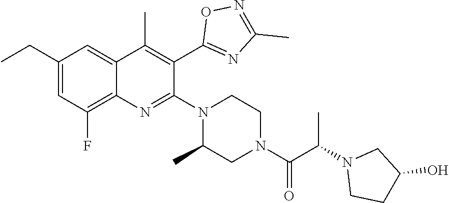 [SMILES: CCc1cc(F)c2nc(N3CCN(C(=O)[C@H](C)N4CC[C@@H](O)C4)C[C@H]3C)c(-c3nc(C)no3)c(C)c2c1] | + | − | +++ |
| 151 | 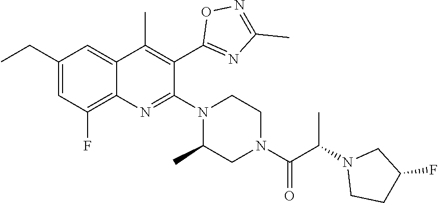 [SMILES: CCc1cc(F)c2nc(N3CCN(C(=O)[C@H](C)N4CC[C@@H](F)C4)C[C@H]3C)c(-c3nc(C)no3)c(C)c2c1] | +++ | − | +++ |
| 152 | 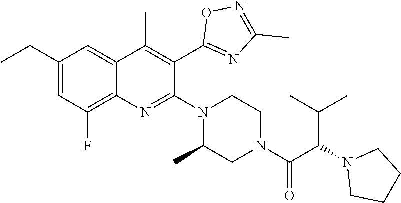 | +++ | − | +++ |
| 153 | 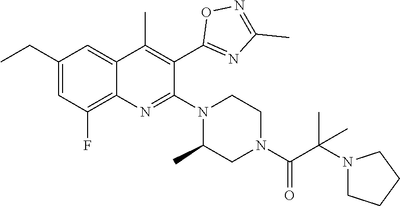 [SMILES: CCc1cc(F)c2nc(N3CCN(C(=O)C(C)(C)N4CCCC4)C[C@H]3C)c(-c3nc(C)no3)c(C)c2c1] | +++ | − | +++ |
| 154 | 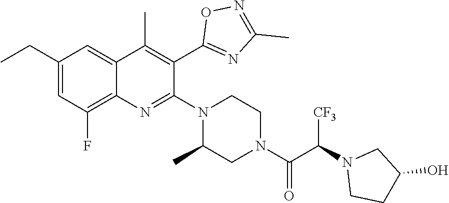 [SMILES: CCc1cc(F)c2nc(N3CCN(C(=O)[C@H](N4CC[C@@H](O)C4)C(F)(F)F)C[C@H]3C)c(-c3nc(C)no3)c(C)c2c1] | | | |
| 155 | 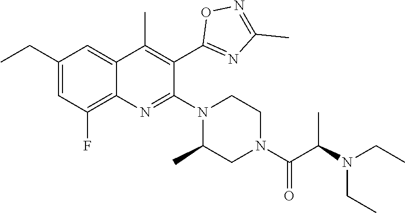 [SMILES: CCc1cc(F)c2nc(N3CCN(C(=O)[C@@H](C)N(CC)CC)C[C@H]3C)c(-c3nc(C)no3)c(C)c2c1] | +++ | − | +++ |
| 156 | 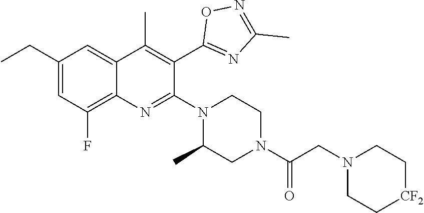 [SMILES: CCc1cc(F)c2nc(N3CCN(C(=O)CN4CCC(F)(F)CC4)C[C@H]3C)c(-c3nc(C)no3)c(C)c2c1] | +++ | + | ++ |
| 157 | 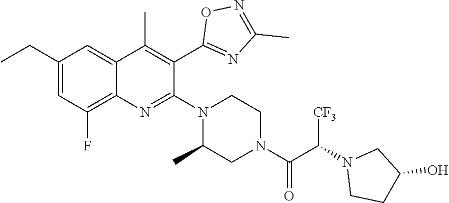 [SMILES: CCc1cc(F)c2nc(N3CCN(C(=O)[C@@H](N4CC[C@@H](O)C4)C(F)(F)F)C[C@H]3C)c(-c3nc(C)no3)c(C)c2c1] | | | |
| 158 | 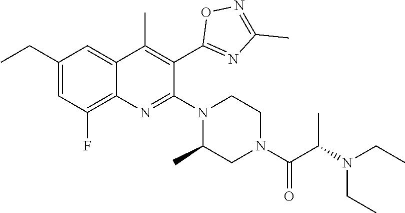 [SMILES: CCc1cc(F)c2nc(N3CCN(C(=O)[C@H](C)N(CC)CC)C[C@H]3C)c(-c3nc(C)no3)c(C)c2c1] | +++ | − | ++++ |
| 159 | 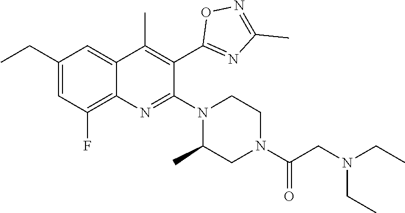 [SMILES: CCc1cc(F)c2nc(N3CCN(C(=O)CN(CC)CC)C[C@H]3C)c(-c3nc(C)no3)c(C)c2c1] | +++ | − | +++ |
| 160 | 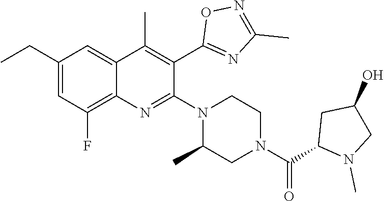 [SMILES: CCc1cc(F)c2nc(N3CCN(C(=O)[C@@H]4C[C@@H](O)CN4C)C[C@H]3C)c(-c3nc(C)no3)c(C)c2c1] | ++ | − | +++ |
| 161 | 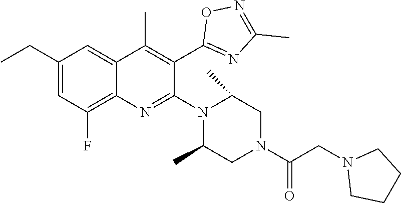 [SMILES: CCc1cc(F)c2nc(N3[C@H](C)CN(C(=O)CN4CCCC4)C[C@H]3C)c(-c3nc(C)no3)c(C)c2c1] | ++++ | + | +++ |
| 162 | 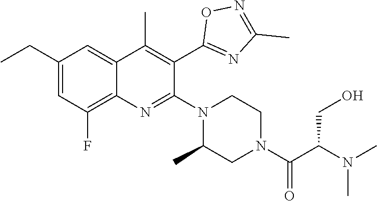 [SMILES: CCc1cc(F)c2nc(N3CCN(C(=O)[C@H](CO)N(C)C)C[C@H]3C)c(-c3nc(C)no3)c(C)c2c1] | +++ | − | +++ |
| 163 | 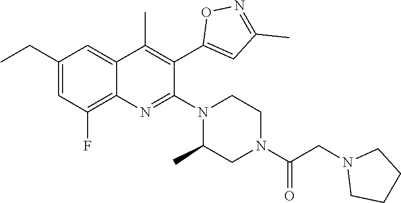 [SMILES: CCc1cc(F)c2nc(N3CCN(C(=O)CN4CCCC4)C[C@H]3C)c(-c3cc(C)no3)c(C)c2c1] | +++ | − | +++ |
| 164 | 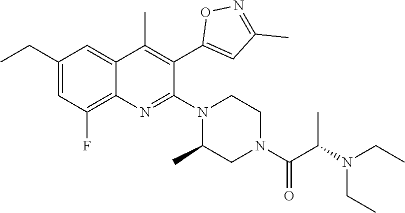 [SMILES: CCc1cc(F)c2nc(N3CCN(C(=O)[C@H](C)N(CC)CC)C[C@H]3C)c(-c3cc(C)no3)c(C)c2c1] | ++ | − | +++ |
| 165 | 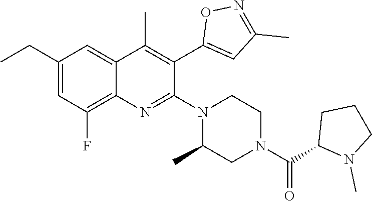 [SMILES: CCc1cc(F)c2nc(N3CCN(C(=O)[C@@H]4CCCN4C)C[C@H]3C)c(-c3cc(C)no3)c(C)c2c1] | ++ | − | +++ |
| 166 | 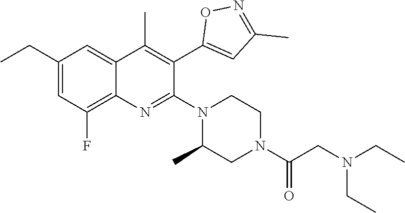 [SMILES: CCc1cc(F)c2nc(N3CCN(C(=O)CN(CC)CC)C[C@H]3C)c(-c3cc(C)no3)c(C)c2c1] | +++ | − | +++ |
| 167 | 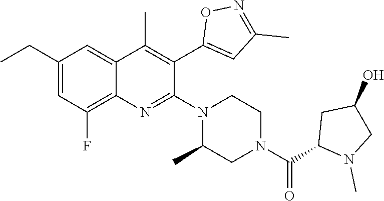 [SMILES: CCc1cc(F)c2nc(N3CCN(C(=O)[C@@H]4C[C@@H](O)CN4C)C[C@H]3C)c(-c3cc(C)no3)c(C)c2c1] | +++ | − | +++ |
| 168 | 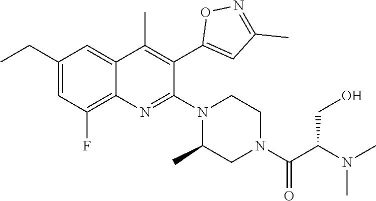 [SMILES: CCc1cc(F)c2nc(N3CCN(C(=O)[C@H](CO)N(C)C)C[C@H]3C)c(-c3cc(C)no3)c(C)c2c1] | +++ | − | +++ |
| 169 | 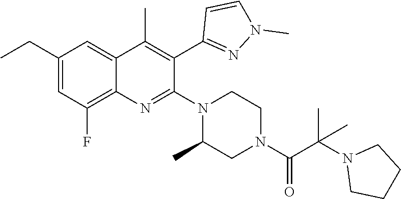 [SMILES: CCc1cc(F)c2nc(N3CCN(C(=O)C(C)(C)N4CCCC4)C[C@H]3C)c(-c3ccn(C)n3)c(C)c2c1] | +++ | − | ++ |
| 170 | 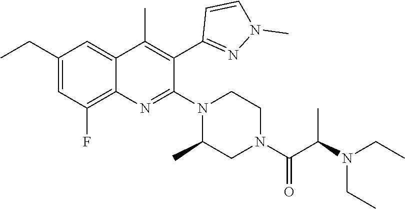 [SMILES: CCc1cc(F)c2nc(N3CCN(C(=O)[C@@H](C)N(CC)CC)C[C@H]3C)c(-c3ccn(C)n3)c(C)c2c1] | ++ | − | +++ |
| 171 | 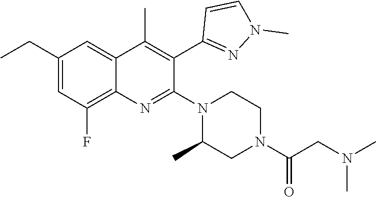 [SMILES: CCc1cc(F)c2nc(N3CCN(C(=O)CN(C)C)C[C@H]3C)c(-c3ccn(C)n3)c(C)c2c1] | ++ | − | +++ |
| 172 | 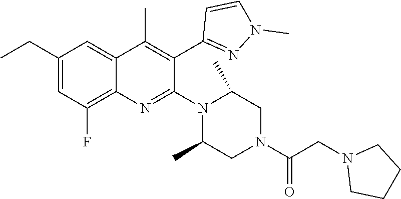 [SMILES: CCc1cc(F)c2nc(N3[C@H](C)CN(C(=O)CN4CCCC4)C[C@H]3C)c(-c3ccn(C)n3)c(C)c2c1] | | | |
| 173 | 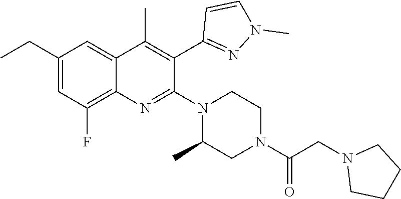 [SMILES: CCc1cc(F)c2nc(N3CCN(C(=O)CN4CCCC4)C[C@H]3C)c(-c3ccn(C)n3)c(C)c2c1] | +++ | + | +++ |
| 174 | 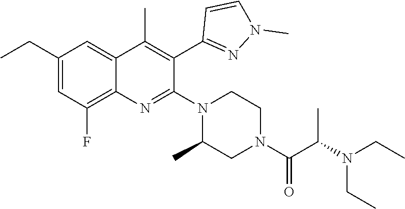 [SMILES: CCc1cc(F)c2nc(N3CCN(C(=O)[C@H](C)N(CC)CC)C[C@H]3C)c(-c3ccn(C)n3)c(C)c2c1] | ++ | − | +++ |
| 175 | 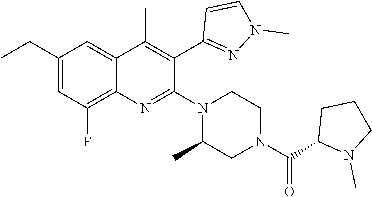 [SMILES: CCc1cc(F)c2nc(N3CCN(C(=O)[C@@H]4CCCN4C)C[C@H]3C)c(-c3ccn(C)n3)c(C)c2c1] | ++ | − | +++ |
| 176 | 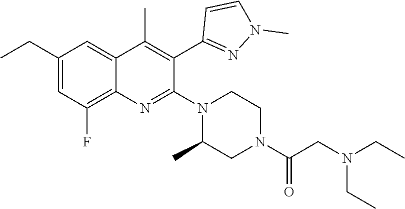 [SMILES: CCc1cc(F)c2nc(N3CCN(C(=O)CN(CC)CC)C[C@H]3C)c(-c3ccn(C)n3)c(C)c2c1] | +++ | − | +++ |
| 177 | 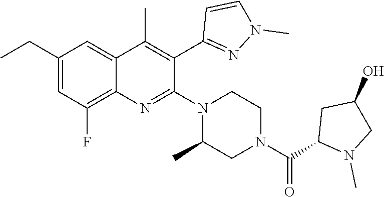 [SMILES: CCc1cc(F)c2nc(N3CCN(C(=O)[C@@H]4C[C@@H](O)CN4C)C[C@H]3C)c(-c3ccn(C)n3)c(C)c2c1] | + | − | +++ |
| 178 | 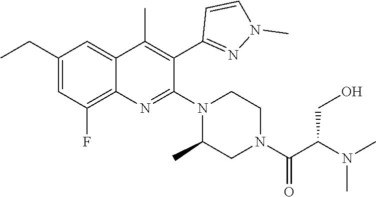 [SMILES: CCc1cc(F)c2nc(N3CCN(C(=O)[C@H](CO)N(C)C)C[C@H]3C)c(-c3ccn(C)n3)c(C)c2c1] | ++ | − | +++ |
| 179 | 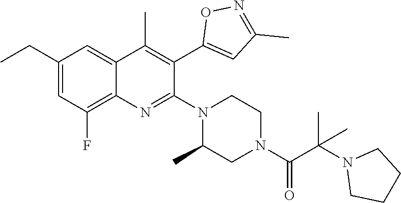 [SMILES: CCc1cc(F)c2nc(N3CCN(C(=O)C(C)(C)N4CCCC4)C[C@H]3C)c(-c3cc(C)no3)c(C)c2c1] | ++ | − | ++ |
| 180 | 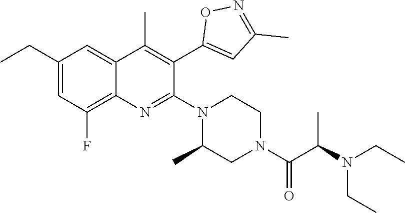 [SMILES: CCc1cc(F)c2nc(N3CCN(C(=O)[C@@H](C)N(CC)CC)C[C@H]3C)c(-c3cc(C)no3)c(C)c2c1] | +++ | − | +++ |
| 181 | 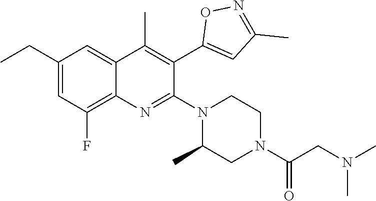 [SMILES: CCc1cc(F)c2nc(N3CCN(C(=O)CN(C)C)C[C@H]3C)c(-c3cc(C)no3)c(C)c2c1] | ++ | − | +++ |
| 182 | 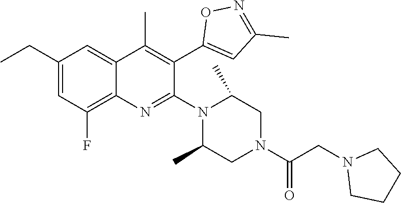 [SMILES: CCc1cc(F)c2nc(N3[C@H](C)CN(C(=O)CN4CCCC4)C[C@H]3C)c(-c3cc(C)no3)c(C)c2c1] | | | |
| 183 | 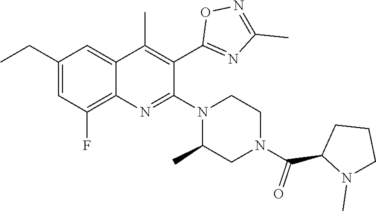 [SMILES: CCc1cc(F)c2nc(N3CCN(C(=O)[C@H]4CCCN4C)C[C@H]3C)c(-c3nc(C)no3)c(C)c2c1] | +++ | − | ++++ |
| 184 | 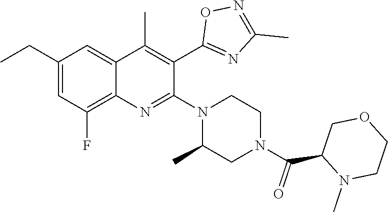 [SMILES: CCc1cc(F)c2nc(N3CCN(C(=O)[C@H]4COCCN4C)C[C@H]3C)c(-c3nc(C)no3)c(C)c2c1] | ++ | − | +++ |
| 185 | 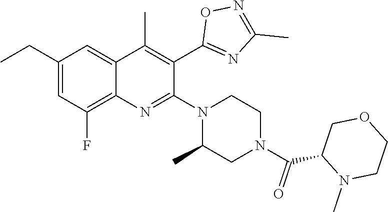 [SMILES: CCc1cc(F)c2nc(N3CCN(C(=O)[C@@H]4COCCN4C)C[C@H]3C)c(-c3nc(C)no3)c(C)c2c1] | ++ | − | +++ |
| 186 | 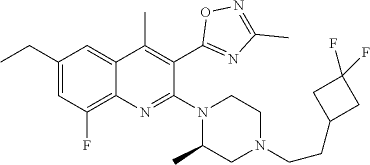 [SMILES: CCc1cc(F)c2nc(N3CCN(CCC4CC(F)(F)C4)C[C@H]3C)c(-c3nc(C)no3)c(C)c2c1] | +++ | + | +++ |
| 187 | 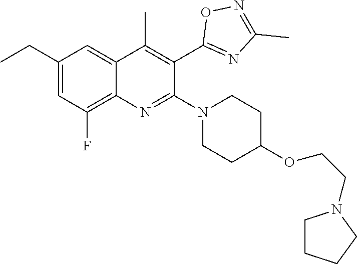 [SMILES: CCc1cc(F)c2nc(N3CCC(OCCN4CCCC4)CC3)c(-c3nc(C)no3)c(C)c2c1] | ++ | − | +++ |
| 188 | 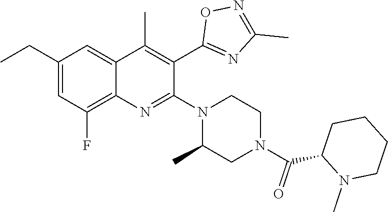 [SMILES: CCc1cc(F)c2nc(N3CCN(C(=O)[C@@H]4CCCCN4C)C[C@H]3C)c(-c3nc(C)no3)c(C)c2c1] | ++ | − | +++ |
| 189 | 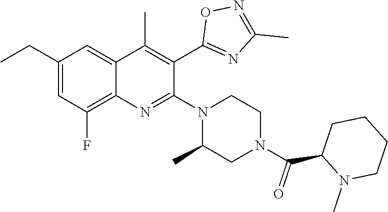 [SMILES: CCc1cc(F)c2nc(N3CCN(C(=O)[C@H]4CCCCN4C)C[C@H]3C)c(-c3nc(C)no3)c(C)c2c1] | +++ | − | ++++ |
| 190 | 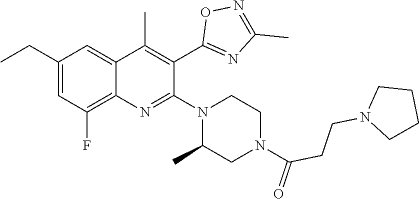 [SMILES: CCc1cc(F)c2nc(N3CCN(C(=O)CCN4CCCC4)C[C@H]3C)c(-c3nc(C)no3)c(C)c2c1] | +++ | + | ++ |
| 191 | 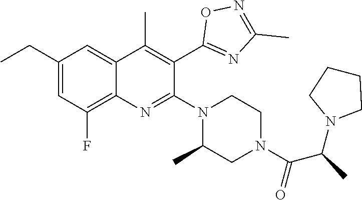 [SMILES: CCc1cc(F)c2nc(N3CCN(C(=O)[C@H](C)N4CCCC4)C[C@H]3C)c(-c3nc(C)no3)c(C)c2c1] | +++ | − | ++++ |
| 192 | 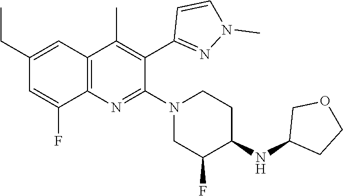 [SMILES: CCc1cc(F)c2nc(N3CC[C@@H](N[C@@H]4CCOC4)[C@@H](F)C3)c(-c3ccn(C)n3)c(C)c2c1] | ++++ | − | ++++ |
| 193 | 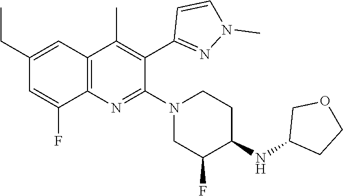 [SMILES: CCc1cc(F)c2nc(N3CC[C@@H](N[C@H]4CCOC4)[C@@H](F)C3)c(-c3ccn(C)n3)c(C)c2c1] | ++++ | − | ++++ |
| 194 | 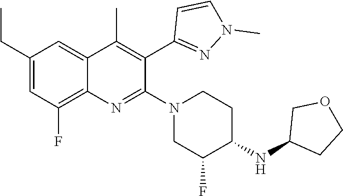 [SMILES: CCc1cc(F)c2nc(N3CC[C@H](N[C@@H]4CCOC4)[C@H](F)C3)c(-c3ccn(C)n3)c(C)c2c1] | +++ | − | +++ |
| 195 | 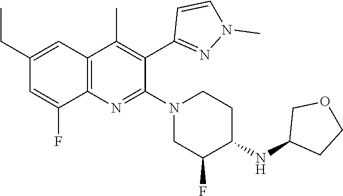 [SMILES: CCc1cc(F)c2nc(N3CC[C@H](N[C@@H]4CCOC4)[C@@H](F)C3)c(-c3ccn(C)n3)c(C)c2c1] | +++ | − | ++++ |
| 196 | 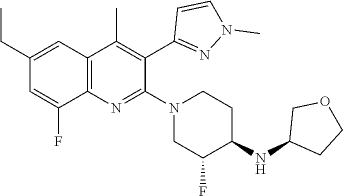 [SMILES: CCc1cc(F)c2nc(N3CC[C@@H](N[C@@H]4CCOC4)[C@H](F)C3)c(-c3ccn(C)n3)c(C)c2c1] | +++ | − | +++ |
| 197 | 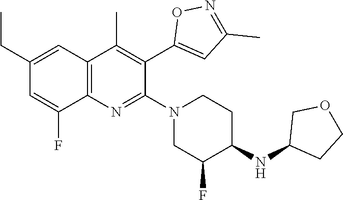 [SMILES: CCc1cc(F)c2nc(N3CC[C@@H](N[C@@H]4CCOC4)[C@@H](F)C3)c(-c3cc(C)no3)c(C)c2c1] | +++ | − | ++++ |
| 198 | 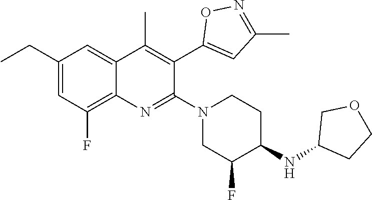 [SMILES: CCc1cc(F)c2nc(N3CC[C@@H](N[C@H]4CCOC4)[C@@H](F)C3)c(-c3cc(C)no3)c(C)c2c1] | +++ | − | ++++ |
| 199 | 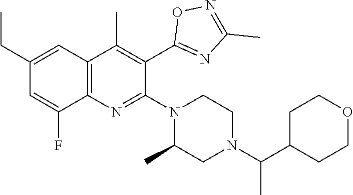 [SMILES: CCc1cc(F)c2nc(N3CCN(C(C)C4CCOCC4)C[C@H]3C)c(-c3nc(C)no3)c(C)c2c1] | ++ | − | ++++ |
| 200 | 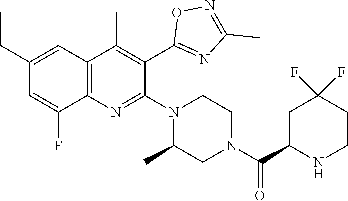 [SMILES: CCc1cc(F)c2nc(N3CCN(C(=O)[C@H]4CC(F)(F)CCN4)C[C@H]3C)c(-c3nc(C)no3)c(C)c2c1] | +++ | − | +++ |
| 201 | 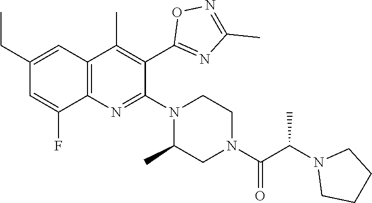 [SMILES: CCc1cc(F)c2nc(N3CCN(C(=O)[C@H](C)N4CCCC4)C[C@H]3C)c(-c3nc(C)no3)c(C)c2c1] | +++ | + | +++ |
| 202 | 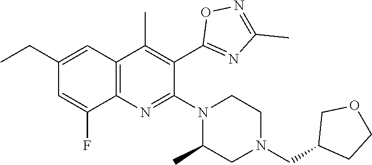 [SMILES: CCc1cc(F)c2nc(N3CCN(C[C@H]4CCOC4)C[C@H]3C)c(-c3nc(C)no3)c(C)c2c1] | +++ | − | ++++ |
| 203 | 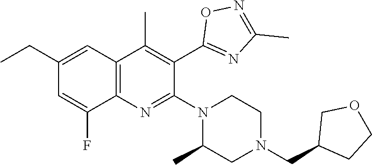 [SMILES: CCc1cc(F)c2nc(N3CCN(C[C@@H]4CCOC4)C[C@H]3C)c(-c3nc(C)no3)c(C)c2c1] | ++ | − | +++ |
| 204 | 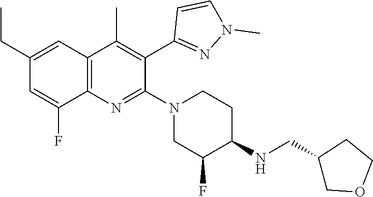 [SMILES: CCc1cc(F)c2nc(N3CC[C@@H](NC[C@@H]4CCOC4)[C@@H](F)C3)c(-c3ccn(C)n3)c(C)c2c1] | ++++ | + | ++++ |
| 205 | 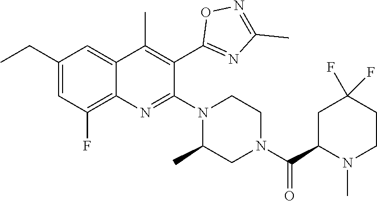 [SMILES: CCc1cc(F)c2nc(N3CCN(C(=O)[C@H]4CC(F)(F)CCN4C)C[C@H]3C)c(-c3nc(C)no3)c(C)c2c1] | +++ | − | +++ |
| 206 | 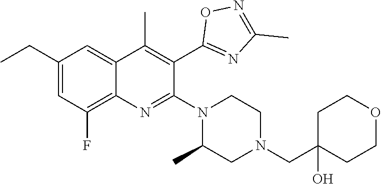 [SMILES: CCc1cc(F)c2nc(N3CCN(CC4(O)CCOCC4)C[C@H]3C)c(-c3nc(C)no3)c(C)c2c1] | +++ | − | ++++ |
| 207 | 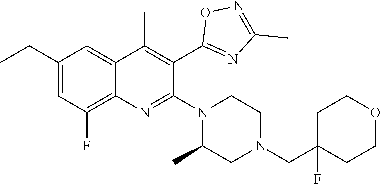 [SMILES: CCc1cc(F)c2nc(N3CCN(CC4(F)CCOCC4)C[C@H]3C)c(-c3nc(C)no3)c(C)c2c1] | +++ | − | ++++ |
| 208 | 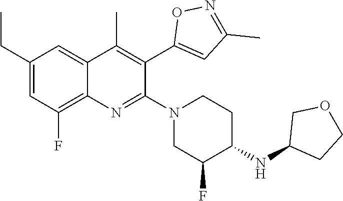 [SMILES: CCc1cc(F)c2nc(N3CC[C@H](N[C@@H]4CCOC4)[C@@H](F)C3)c(-c3cc(C)no3)c(C)c2c1] | ++ | − | +++ |

KOR (IC₅₀)
++++ (less than 1 nM)
+++ (1 nM to less than 10 nM)
++ (10 nM to less than 100 nM)
+ (100 nM or more)
MOR (IC₅₀)
+++ (1 nM to less than 10 nM)
++ (10 nM to less than 100 nM)
+ (100 nM to less than 1000 nM)
− (more than 1000 nM)
Kappa selectivity (MORIC₅₀ /KOR IC₅₀)
++++ (more than 1000-fold selectivity over MOR
+++ (between 100- to 1000-fold selective)
++ (between 10- and 100-fold selectivity)
+ (less than 10-fold selectivity)

1. A compound of Formula (I) or a pharmaceutically acceptable salt thereof: 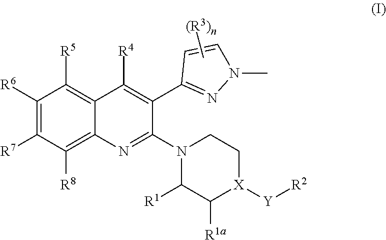
wherein
X is CH and Y is NH,
or X is N and Y is —C(═N—CN)NR⁹;
or X is NH and —Y—R² is null;
R¹ and R^{1a} are independently selected from the group consisting of H, C₁-C₆-alkyl, and halo;
or R¹ and R^{1a}, together with the carbon atoms to which they are bound, form a fused C₃-C₅-cycloalkyl or an optionally substituted 3- to 6-membered heterocycloalkyl (wherein 1-4 ring members are independently selected from N, O, and S);
R² is selected from the group consisting of H, C₁-C₆-alkyl, C₃-C₈-cycloalkyl, —(C₁-C₆-alkyl)C₃-C₈-cycloalkyl, and 3- to 6-membered heterocycloalkyl (wherein 1-4 ring members are independently selected from N, O, and S);
R⁹ is selected from the group consisting of H, C₁-C₆-alkyl, C₁-C₆-haloalkyl, C₃-C₅-cycloalkyl, and —(C₁-C₆-alkyl)(C₃-C₈-cycloalkyl);
R³ in each instance is C₁-C₆-alkyl;
n is 0, 1, or 2;
R⁴, R⁵, R⁶, R⁷, and R⁸ are independently selected from the group consisting of H, CN, OH, halo, NRR′, C₁-C₆-alkyl, C₂-C₆-alkenyl, C₂-C₆-alkynyl, C₁-C₆-haloalkyl, O(C₁-C₆-alkyl), O(C₁-C₆-haloalkyl), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(RR′), —CONRR′, —COOR′, —NRCOOR′, —(C₁-C₆-alkyl)C(O)N(RR′), C₆-C₁₀-aryl, C₃-C₈-cycloalkyl, O(C₃-C₈-cycloalkyl), —(C₁-C₆-alkyl)(C₆-C₁₀-aryl), —(C₁-C₆-alkyl)(C₃-C₈-cycloalkyl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), 5- to 10-membered heteroaryl (wherein 1-4 heteroaryl members are independently selected from N, O, and S), —(C₁-C₆-alkyl)(5- to 10-membered heteroaryl (wherein 1-4 heteroaryl members are independently selected from N, O, and S)), wherein
R and R′ are independently selected from H and C₁-C₆-alkyl, and
any alkyl, aryl, cycloalkyl, heterocycloalkyl, and heteroaryl in R¹, R^{1a}, R², R³, R⁴, R⁵, R⁶, R⁷, and R⁸ is optionally substituted with 1 to 6 substituents independently selected from the group consisting of C₁-C₆-alkyl, halo, NO₂, OH, CN, and C₁-C₆-haloalkyl.

2. A compound of Formula (II) or a pharmaceutically acceptable salt thereof: 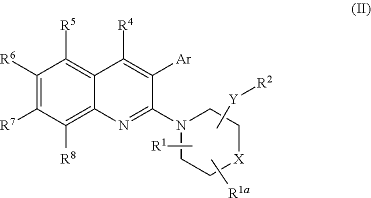
wherein
Ar is a 5- or 6-membered heteroaryl (wherein 1-4 heteroaryl members are independently selected from N, O, and S) substituted with (R³)ₙ;
X is CH and Y is NH;
or X is N and Y is selected from a bond, C(O), and —C(═N—CN)NR⁹;
or X is NH and —Y—R² is null;
R¹ and R^{1a} are independently selected from the group consisting of H, C₁-C₆-alkyl, and halo;
or R¹ and R^{1a}, together with the carbon atoms to which they are bound, form a fused C₃-C₅-cycloalkyl or an optionally substituted 3- to 6-membered heterocycloalkyl (wherein 1-4 ring members are independently selected from N, O, and S);
wherein when X is CH, then:
(i) at least one of R¹ and Ria is not H and Ar is other than oxadiazolyl, thiadiazolyl, or triazolyl; or
(ii) optionally R¹ or R^{1a}, together with Y and the carbon atoms to which they are bound, form a fused 5- to 6-membered heterocycloalkyl;
R² is selected from the group consisting of H, C₁-C₆-alkyl, C₃-C₈-cycloalkyl, —(C₁-C₆-alkyl)C₃-C₈-cycloalkyl, 3- to 6-membered heterocycloalkyl (wherein 1-4 ring members are independently selected from N, O, and S), and —(C₁-C₆-alkyl)(3- to 6-membered heterocycloalkyl (wherein 1-4 ring members are independently selected from N, O, and S));
R⁹ is selected from the group consisting of H, C₁-C₆-alkyl, C₁-C₆-haloalkyl, C₃-C₅-cycloalkyl, and —(C₁-C₆-alkyl)(C₃-C₈-cycloalkyl);
R³ in each instance is independently C₁-C₆-alkyl or C₁-C₆-haloalkyl;
n is 0, 1, or 2;
R⁴, R⁵, R⁶, R⁷, and R⁸ are independently selected from the group consisting of H, CN, OH, halo, NRR′, C₁-C₆-alkyl, C₂-C₆-alkenyl, C₂-C₆-alkynyl, C₁-C₆-haloalkyl, O(C₁-C₆-alkyl), O(C₁-C₆-haloalkyl), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(RR′), —CONRR′, —COOR′, —NRCOOR′, —(C₁-C₆-alkyl)C(O)N(RR′), C₆-C₁₀-aryl, C₃-C₈-cycloalkyl, O(C₃-C₈-cycloalkyl), —(C₁-C₆-alkyl)(C₁-C₁₀-aryl), —(C₁-C₆-alkyl)(C₃-C₈-cycloalkyl), 3- to 6-membered heterocycloalkyl (wherein 1-4 ring members are independently selected from N, O, and S), —(C₁-C₆-alkyl)(3- to 6-membered heterocycloalkyl (wherein 1-4 ring members are independently selected from N, O, and S), 5- to 10-membered heteroaryl (wherein 1-4 heteroaryl members are independently selected from N, O, and S), —(C₁-C₆-alkyl)(5- to 10-membered heteroaryl (wherein 1-4 heteroaryl members are independently selected from N, O, and S));
R and R′ are independently selected from H and C₁-C₆-alkyl, any alkyl, aryl, cycloalkyl, heterocycloalkyl, and heteroaryl in R¹, R^{1a}, R², R³, R⁴, R⁵, R⁶, R⁷, and R⁸ is optionally substituted with 1 to 6 substituents independently selected from the group consisting of C₁-C₆-alkyl, halo, NRR′, NO₂, OR, CN, and C₁-C₆-haloalkyl;
and wherein the compound is not:

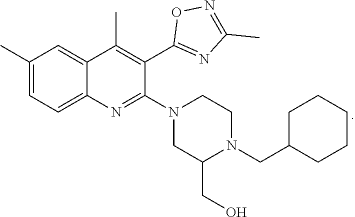 [SMILES: Cc1ccc2nc(N3CCN(CC4CCCCC4)C(CO)C3)c(-c3nc(C)no3)c(C)c2c1]

3. The compound or pharmaceutically acceptable salt thereof according to claim 2, wherein the compound is of Formula (11A):

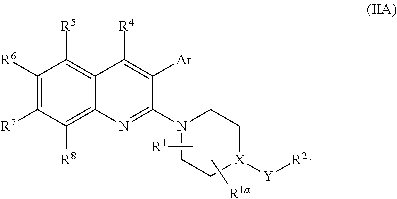

4. The compound or pharmaceutically acceptable salt thereof according to claim 2, wherein Ar is a 5-membered heteroaryl (wherein 1-4 heteroaryl members are independently selected from N, O, and S).

5. The compound or pharmaceutically acceptable salt thereof according to claim 4, wherein Ar is selected from the group consisting of pyrazolyl, imidazolyl, oxazolyl, isoxazolyl, oxadiazolyl, isoxadiazolyl, thiazolyl, isothiazolyl, and thiadiazolyl.

6. The compound or pharmaceutically acceptable salt thereof according claim 5, wherein Ar is selected from the group consisting of pyrazolyl, oxazolyl, and isoxazolyl.

7. The compound or pharmaceutically acceptable salt thereof according to claim 2, wherein X is N and Y is a bond.

8. The compound or pharmaceutically acceptable salt thereof according claim 2, wherein X is CH and Y is NH.

9. The compound or pharmaceutically acceptable salt thereof according to claim 2, wherein X is N and Y is —C(═N—CN)NR⁹.

10. The compound or pharmaceutically acceptable salt thereof according to claim 9, wherein R¹ is H.

11. The compound or pharmaceutically acceptable salt thereof according to claim 2, wherein R² is an optionally substituted 3- to 6-membered heterocycloalkyl (wherein 1 ring member is 0).

12. The compound or pharmaceutically acceptable salt thereof according to claim 11, wherein R² is selected from the group consisting of optionally substituted:

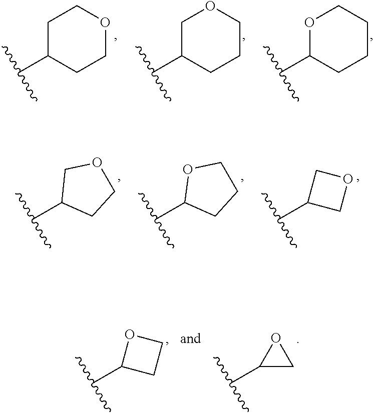

13. The compound or pharmaceutically acceptable salt thereof according to claim 12, wherein R² is

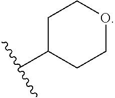 [SMILES: *C1CCOCC1]

14. The compound or pharmaceutically acceptable salt thereof according to claim 2, wherein R² is substituted by 1 to 3 substituents selected from halo and OH.

15. The compound or pharmaceutically acceptable salt thereof according to claim 13, wherein R² is substituted with halo.

16. The compound or pharmaceutically acceptable salt thereof according to claim 15, wherein halo is F.

17. The compound or pharmaceutically acceptable salt thereof according to claim 14, wherein R² is substituted with OH.

18. The compound or pharmaceutically acceptable salt thereof according to claim 2, wherein n is 0.

19. The compound or pharmaceutically acceptable salt thereof according to claim 2, wherein n is 1.

20. The compound or pharmaceutically acceptable salt thereof according to claim 2, wherein one of R¹ and R^{1a} is H and the other is halo.

21. The compound or pharmaceutically acceptable salt thereof according to claim 20, wherein one of R¹ and R^{1a} is H and the other is F.

22. The compound or pharmaceutically acceptable salt thereof according to claim 21, wherein R¹ is H and R^{1a} is F.

23. The compound or pharmaceutically acceptable salt thereof according to claim 19, wherein each of R¹ and R^{1a} is H.

24. The compound or pharmaceutically acceptable salt thereof according to claim 2, wherein R¹ and R^{1a}, together with the carbon atoms to which they are bound, form an optionally substituted fused C₃-C₈-cycloalkyl or an optionally substituted 3- to 6-membered heterocycloalkyl (wherein 1-4 ring members are independently selected from N, O, and S).

25. The compound or pharmaceutically acceptable salt thereof according to claim 24, wherein R¹ and R^{1a}, together with the carbon atoms to which they are bound, form an optionally substituted fused C₃-C₈-cycloalkyl.

26. The compound or pharmaceutically acceptable salt thereof according to claim 25, wherein the fused C₃-C₈-cycloalkyl is cyclopropyl.

27. The compound or pharmaceutically acceptable salt thereof according to claim 2, wherein R¹ or R^{1a}, together with Y and the carbon atoms to which they are bound, form a fused 5- to 6-membered heterocycloalkyl.

28. The compound or pharmaceutically acceptable salt thereof according to claim 27, wherein the fused 5- to 6-membered heterocycloalkyl is pyrrolidinyl.

29. The compound or pharmaceutically acceptable salt thereof according to claim 2, wherein R⁴ is selected from the group consisting of H, CN, halo, and C₁-C₆-alkyl.

30. The compound or pharmaceutically acceptable salt thereof according to claim 29, wherein R⁴ is C₁-C₆-alkyl.

31. The compound or pharmaceutically acceptable salt thereof according to claim 2, wherein R⁵ and R⁷ are independently selected from the group consisting of H, halo, and CN.

32. The compound or pharmaceutically acceptable salt thereof according to claim 31, wherein at least one of R⁵ and R⁷ is H.

33. The compound or pharmaceutically acceptable salt thereof according to claim 32, wherein each of R⁵ and R⁷ is H.

34. The compound or pharmaceutically acceptable salt thereof according to claim 2, wherein R⁶ is selected from the group consisting of halo, CN, C₁-C₆-alkyl, C₂-C₆-alkenyl, C₂-C₆-alkynyl, and C₃-C₈-cycloalkyl.

35. The compound or pharmaceutically acceptable salt thereof according to claim 34, wherein R⁶ is C₁-C₆-alkyl.

36. The compound or pharmaceutically acceptable salt thereof according to claim 2, wherein R⁸ is selected from the group consisting of H, CN, halo, C₁-C₆-alkyl, C₃-C₈-cycloalkyl, C₆-C₁₀-aryl, 5- to 10-membered heteroaryl (wherein 1-4 heteroaryl members are independently selected from N, O, and S), and —CONRR′.

37. The compound or pharmaceutically acceptable salt thereof according to claim 36, wherein R⁸ is halo or 5- to 10-membered heteroaryl (wherein 1-4 heteroaryl members are independently selected from N, O, and S).

38. The compound or pharmaceutically acceptable salt thereof according to claim 37, wherein R⁸ is F.

39. The compound or pharmaceutically acceptable salt thereof according to claim 1, wherein:
X is CH and Y is NH,
one of R¹ and R^{1a} is H and the other is F;
R² is an optionally substituted 3- to 6-membered heterocycloalkyl (wherein 1 ring member is O);
n is 0 or 1;
R⁴ is selected from the group consisting of H, CN, halo, and C₁-C₆-alkyl;
R⁵ and R⁷ are independently selected from the group consisting of H, halo, and CN, wherein at least one of R⁵ and R⁷ is H;
R⁶ is selected from the group consisting of halo, CN, C₁-C₆-alkyl, C₂-C₆-alkenyl, C₂-C₆-alkynyl, and C₃-C₅-cycloalkyl; and
R⁸ is selected from the group consisting of H, CN, halo, C₁-C₆-alkyl, C₃-C₈-cycloalkyl, C₆-C₁₀-aryl, 5- to 10-membered heteroaryl (wherein 1-4 heteroaryl members are independently selected from N, O, and S), and —CONRR′.

40. The compound or pharmaceutically acceptable salt thereof according to claim 1, wherein the compound is selected from the following table:

| | |
|---|---|
| 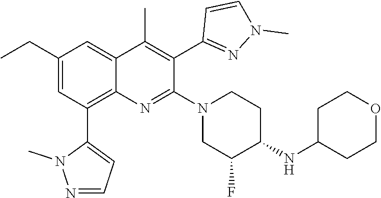 [SMILES: CCc1cc(-c2ccnn2C)c2nc(N3CC[C@H](NC4CCOCC4)[C@H](F)C3)c(-c3ccn(C)n3)c(C)c2c1] | 1 |
| 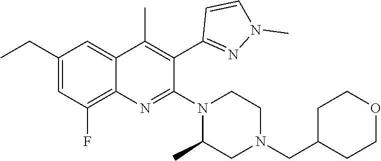 [SMILES: CCc1cc(F)c2nc(N3CCN(CC4CCOCC4)C[C@H]3C)c(-c3ccn(C)n3)c(C)c2c1] | 2 |
| 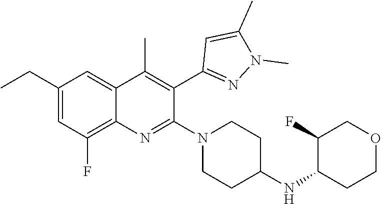 [SMILES: CCc1cc(F)c2nc(N3CCC(N[C@H]4CCOC[C@@H]4F)CC3)c(-c3cc(C)n(C)n3)c(C)c2c1] | 3 |
| 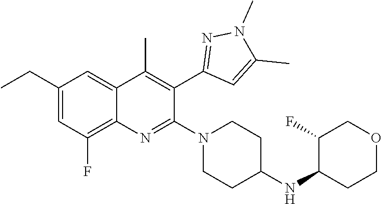 [SMILES: CCc1cc(F)c2nc(N3CCC(N[C@@H]4CCOC[C@H]4F)CC3)c(-c3cc(C)n(C)n3)c(C)c2c1] | 4 |
| 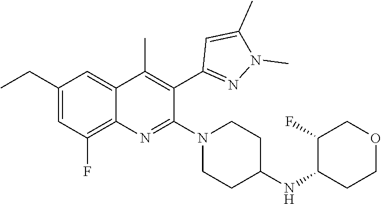 [SMILES: CCc1cc(F)c2nc(N3CCC(N[C@H]4CCOC[C@H]4F)CC3)c(-c3cc(C)n(C)n3)c(C)c2c1] | 5 |
| 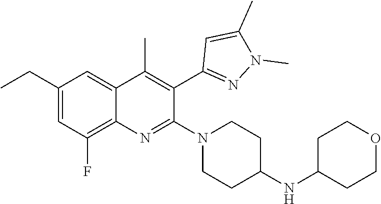 [SMILES: CCc1cc(F)c2nc(N3CCC(NC4CCOCC4)CC3)c(-c3cc(C)n(C)n3)c(C)c2c1] | 6 |
| 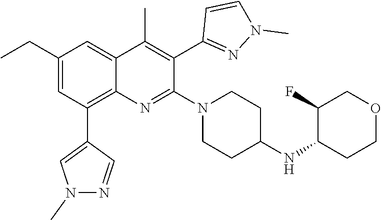 [SMILES: CCc1cc(-c2cnn(C)c2)c2nc(N3CCC(N[C@H]4CCOC[C@@H]4F)CC3)c(-c3ccn(C)n3)c(C)c2c1] | 7 |
| 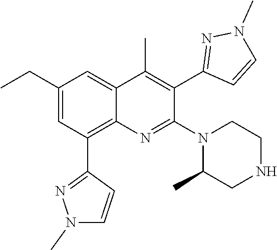 [SMILES: CCc1cc(-c2ccn(C)n2)c2nc(N3CCNC[C@H]3C)c(-c3ccn(C)n3)c(C)c2c1] | 8 |
| 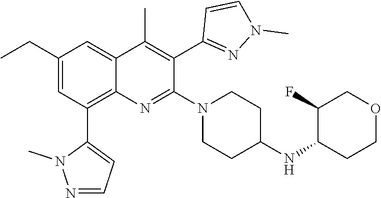 [SMILES: CCc1cc(-c2ccnn2C)c2nc(N3CCC(N[C@H]4CCOC[C@@H]4F)CC3)c(-c3ccn(C)n3)c(C)c2c1] | 9 |
| 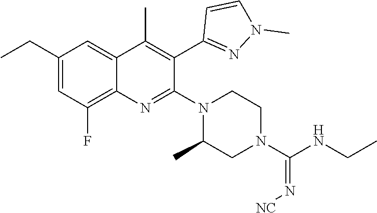 | 10 |
| 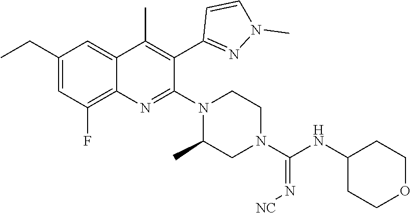 | 11 |
| 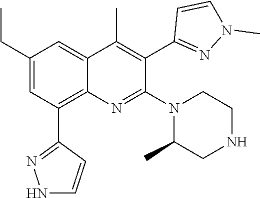 [SMILES: CCc1cc(-c2cc[nH]n2)c2nc(N3CCNC[C@H]3C)c(-c3ccn(C)n3)c(C)c2c1] | 12 |
| 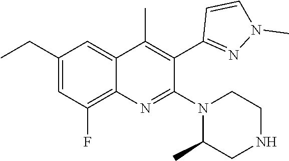 [SMILES: CCc1cc(F)c2nc(N3CCNC[C@H]3C)c(-c3ccn(C)n3)c(C)c2c1] | 13 |
| 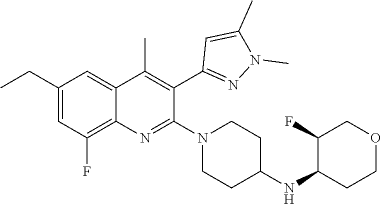 [SMILES: CCc1cc(F)c2nc(N3CCC(N[C@@H]4CCOC[C@@H]4F)CC3)c(-c3cc(C)n(C)n3)c(C)c2c1] | 14 |
| 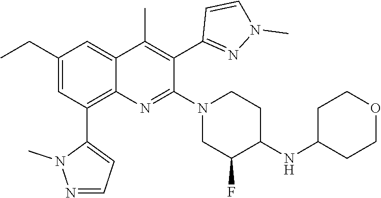 [SMILES: CCc1cc(-c2ccnn2C)c2nc(N3CCC(NC4CCOCC4)[C@@H](F)C3)c(-c3ccn(C)n3)c(C)c2c1] | 15 |
| 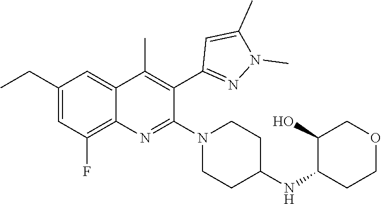 [SMILES: CCc1cc(F)c2nc(N3CCC(N[C@H]4CCOC[C@@H]4O)CC3)c(-c3cc(C)n(C)n3)c(C)c2c1] | 16 |
| 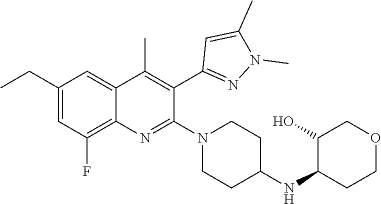 | 17 |
| 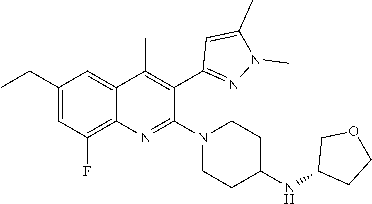 [SMILES: CCc1cc(F)c2nc(N3CCC(N[C@H]4CCOC4)CC3)c(-c3cc(C)n(C)n3)c(C)c2c1] | 18 |
| 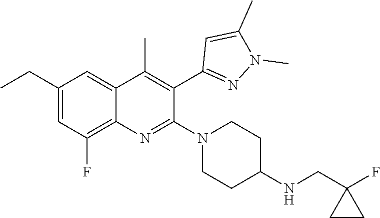 [SMILES: CCc1cc(F)c2nc(N3CCC(NCC4(F)CC4)CC3)c(-c3cc(C)n(C)n3)c(C)c2c1] | 19 |
| 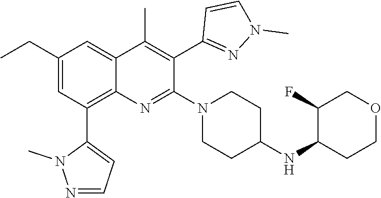 [SMILES: CCc1cc(-c2ccnn2C)c2nc(N3CCC(N[C@@H]4CCOC[C@@H]4F)CC3)c(-c3ccn(C)n3)c(C)c2c1] | 20 |
| 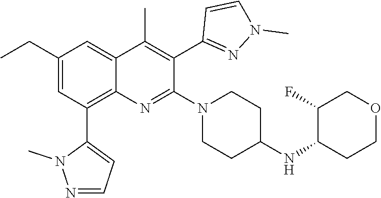 [SMILES: CCc1cc(-c2ccnn2C)c2nc(N3CCC(N[C@H]4CCOC[C@H]4F)CC3)c(-c3ccn(C)n3)c(C)c2c1] | 21 |
| 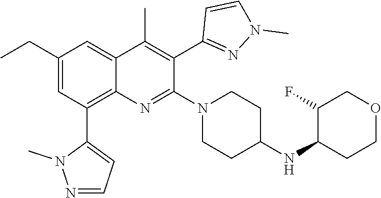 | 22 |
| 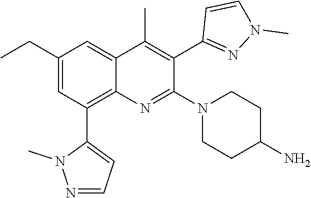 [SMILES: CCc1cc(-c2ccnn2C)c2nc(N3CCC(N)CC3)c(-c3ccn(C)n3)c(C)c2c1] | 23 |
| 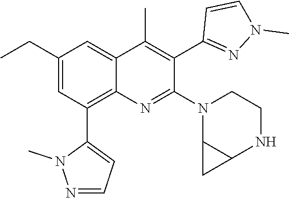 [SMILES: CCc1cc(-c2ccnn2C)c2nc(N3CCNC4CC43)c(-c3ccn(C)n3)c(C)c2c1] | 24 |
| 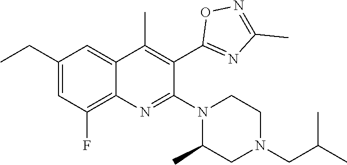 [SMILES: CCc1cc(F)c2nc(N3CCN(CC(C)C)C[C@H]3C)c(-c3nc(C)no3)c(C)c2c1] | 25 |
| 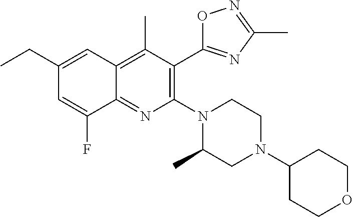 [SMILES: CCc1cc(F)c2nc(N3CCN(C4CCOCC4)C[C@H]3C)c(-c3nc(C)no3)c(C)c2c1] | 26 |
| 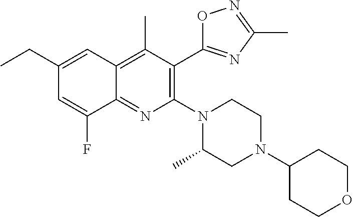 [SMILES: CCc1cc(F)c2nc(N3CCN(C4CCOCC4)C[C@@H]3C)c(-c3nc(C)no3)c(C)c2c1] | 27 |
| 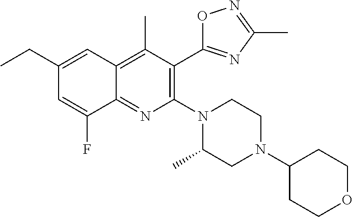 [SMILES: CCc1cc(F)c2nc(N3CCN(C4CCOCC4)C[C@@H]3C)c(-c3nc(C)no3)c(C)c2c1] | 28 |
| 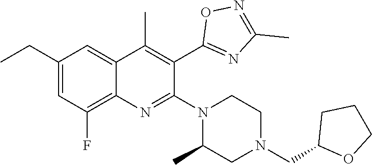 [SMILES: CCc1cc(F)c2nc(N3CCN(C[C@@H]4CCCO4)C[C@H]3C)c(-c3nc(C)no3)c(C)c2c1] | 29 |
| 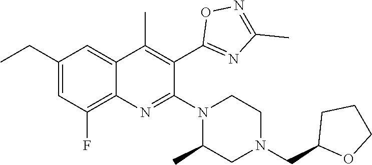 [SMILES: CCc1cc(F)c2nc(N3CCN(C[C@H]4CCCO4)C[C@H]3C)c(-c3nc(C)no3)c(C)c2c1] | 30 |
| 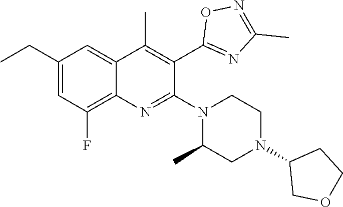 [SMILES: CCc1cc(F)c2nc(N3CCN([C@@H]4CCOC4)C[C@H]3C)c(-c3nc(C)no3)c(C)c2c1] | 31 |
| 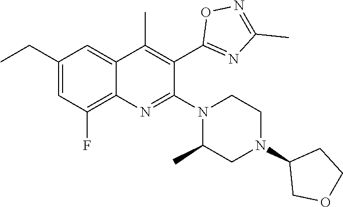 [SMILES: CCc1cc(F)c2nc(N3CCN([C@H]4CCOC4)C[C@H]3C)c(-c3nc(C)no3)c(C)c2c1] | 32 |
| 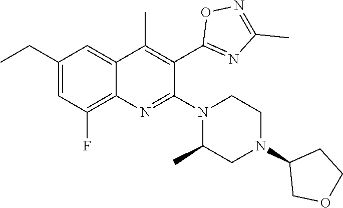 [SMILES: CCc1cc(F)c2nc(N3CCN([C@H]4CCOC4)C[C@H]3C)c(-c3nc(C)no3)c(C)c2c1] | 33 |
| 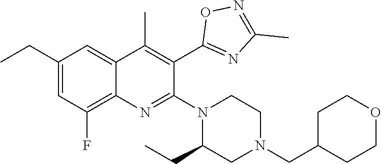 [SMILES: CCc1cc(F)c2nc(N3CCN(CC4CCOCC4)C[C@H]3CC)c(-c3nc(C)no3)c(C)c2c1] | 34 |
| 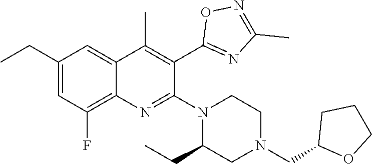 [SMILES: CCc1cc(F)c2nc(N3CCN(C[C@@H]4CCCO4)C[C@H]3CC)c(-c3nc(C)no3)c(C)c2c1] | 35 |
| 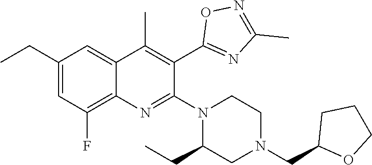 [SMILES: CCc1cc(F)c2nc(N3CCN(C[C@H]4CCCO4)C[C@H]3CC)c(-c3nc(C)no3)c(C)c2c1] | 36 |
| 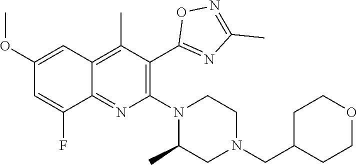 [SMILES: COc1cc(F)c2nc(N3CCN(CC4CCOCC4)C[C@H]3C)c(-c3nc(C)no3)c(C)c2c1] | 37 |
| 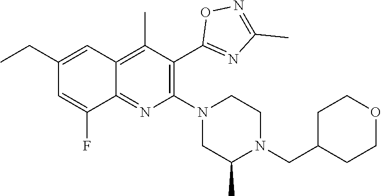 [SMILES: CCc1cc(F)c2nc(N3CCN(CC4CCOCC4)[C@@H](C)C3)c(-c3nc(C)no3)c(C)c2c1] | 38 |
| 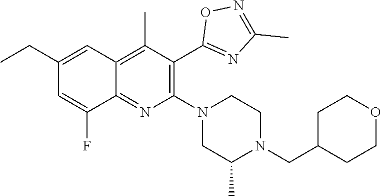 [SMILES: CCc1cc(F)c2nc(N3CCN(CC4CCOCC4)[C@H](C)C3)c(-c3nc(C)no3)c(C)c2c1] | 39 |
| 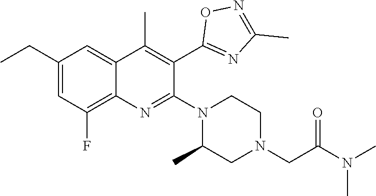 [SMILES: CCc1cc(F)c2nc(N3CCN(CC(=O)N(C)C)C[C@H]3C)c(-c3nc(C)no3)c(C)c2c1] | 40 |
| 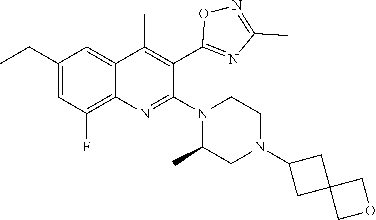 [SMILES: CCc1cc(F)c2nc(N3CCN(C4CC5(COC5)C4)C[C@H]3C)c(-c3nc(C)no3)c(C)c2c1] | 41 |
| 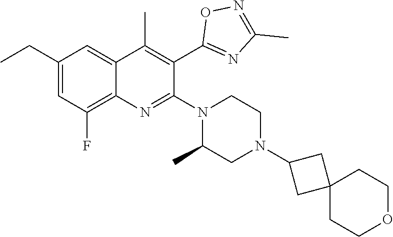 [SMILES: CCc1cc(F)c2nc(N3CCN(C4CC5(CCOCC5)C4)C[C@H]3C)c(-c3nc(C)no3)c(C)c2c1] | 42 |
| 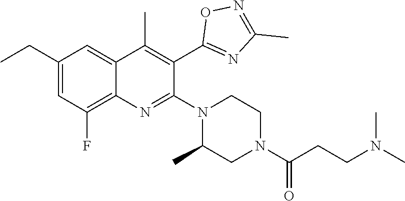 [SMILES: CCc1cc(F)c2nc(N3CCN(C(=O)CCN(C)C)C[C@H]3C)c(-c3nc(C)no3)c(C)c2c1] | 43 |
| 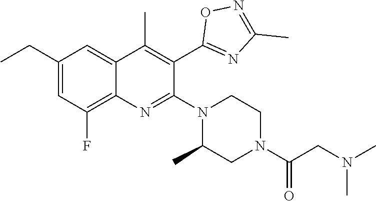 [SMILES: CCc1cc(F)c2nc(N3CCN(C(=O)CN(C)C)C[C@H]3C)c(-c3nc(C)no3)c(C)c2c1] | 44 |
| 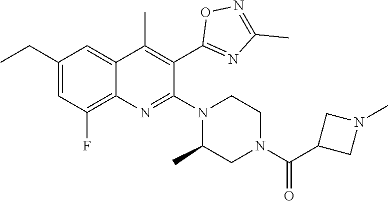 [SMILES: CCc1cc(F)c2nc(N3CCN(C(=O)C4CN(C)C4)C[C@H]3C)c(-c3nc(C)no3)c(C)c2c1] | 45 |
| 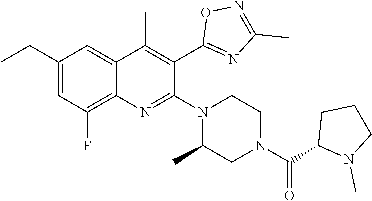 [SMILES: CCc1cc(F)c2nc(N3CCN(C(=O)[C@@H]4CCCN4C)C[C@H]3C)c(-c3nc(C)no3)c(C)c2c1] | 46 |
| 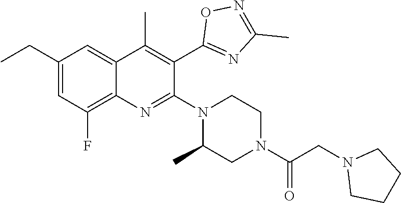 [SMILES: CCc1cc(F)c2nc(N3CCN(C(=O)CN4CCCC4)C[C@H]3C)c(-c3nc(C)no3)c(C)c2c1] | 47 |
| 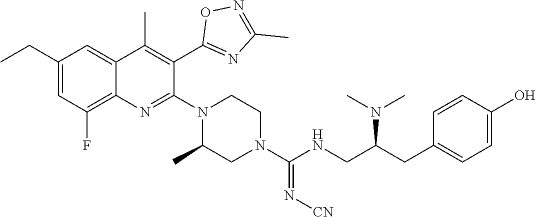 [SMILES: CCc1cc(F)c2nc(N3CCN(/C(=N/C#N)NC[C@H](Cc4ccc(O)cc4)N(C)C)C[C@H]3C)c(-c3nc(C)no3)c(C)c2c1] | 48 |
| 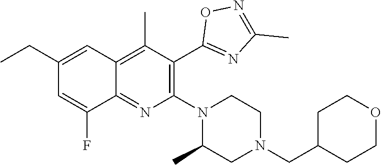 [SMILES: CCc1cc(F)c2nc(N3CCN(CC4CCOCC4)C[C@H]3C)c(-c3nc(C)no3)c(C)c2c1] | 49 |
| 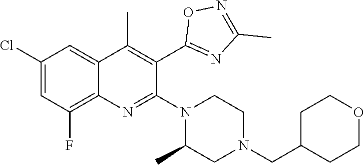 [SMILES: Cc1noc(-c2c(N3CCN(CC4CCOCC4)C[C@H]3C)nc3c(F)cc(Cl)cc3c2C)n1] | 50 |
| 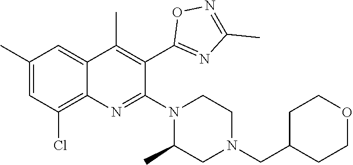 [SMILES: Cc1cc(Cl)c2nc(N3CCN(CC4CCOCC4)C[C@H]3C)c(-c3nc(C)no3)c(C)c2c1] | 51 |
| 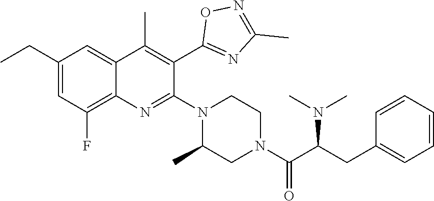 [SMILES: CCc1cc(F)c2nc(N3CCN(C(=O)[C@H](Cc4ccccc4)N(C)C)C[C@H]3C)c(-c3nc(C)no3)c(C)c2c1] | 52 |
| 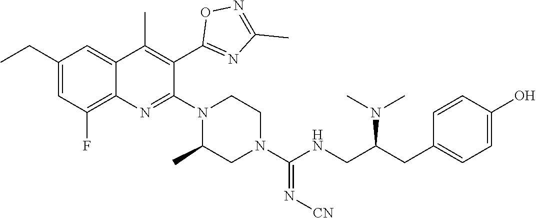 [SMILES: CCc1cc(F)c2nc(N3CCN(/C(=N/C#N)NC[C@H](Cc4ccc(O)cc4)N(C)C)C[C@H]3C)c(-c3nc(C)no3)c(C)c2c1] | 53 |
| 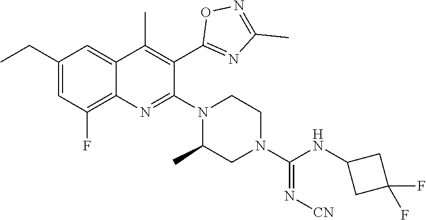 [SMILES: CCc1cc(F)c2nc(N3CCN(/C(=N/C#N)NC4CC(F)(F)C4)C[C@H]3C)c(-c3nc(C)no3)c(C)c2c1] | 54 |
| 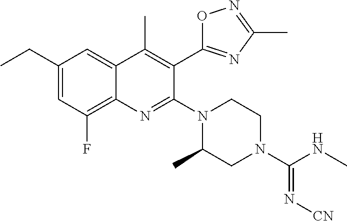 [SMILES: CCc1cc(F)c2nc(N3CCN(/C(=N/C#N)NC)C[C@H]3C)c(-c3nc(C)no3)c(C)c2c1] | 55 |
| 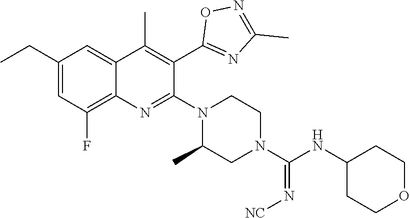 [SMILES: CCc1cc(F)c2nc(N3CCN(/C(=N\C#N)NC4CCOCC4)C[C@H]3C)c(-c3nc(C)no3)c(C)c2c1] | 56 |
| 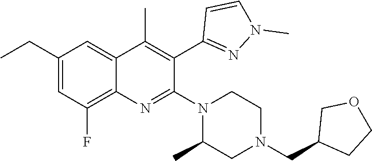 [SMILES: CCc1cc(F)c2nc(N3CCN(C[C@@H]4CCOC4)C[C@H]3C)c(-c3ccn(C)n3)c(C)c2c1] | 57 |
| 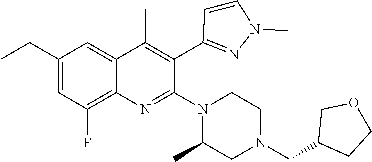 | 58 |
| 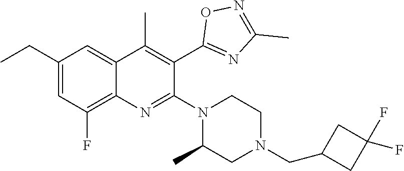 [SMILES: CCc1cc(F)c2nc(N3CCN(CC4CC(F)(F)C4)C[C@H]3C)c(-c3nc(C)no3)c(C)c2c1] | 59 |
| 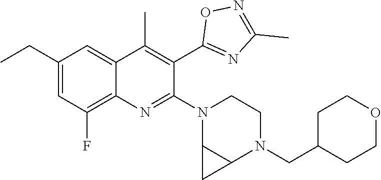 [SMILES: CCc1cc(F)c2nc(N3CCN(CC4CCOCC4)C4CC43)c(-c3nc(C)no3)c(C)c2c1] | 60 |
| 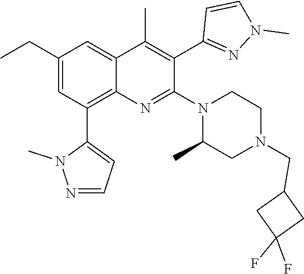 [SMILES: CCc1cc(-c2ccnn2C)c2nc(N3CCN(CC4CC(F)(F)C4)C[C@H]3C)c(-c3ccn(C)n3)c(C)c2c1] | 61 |
| 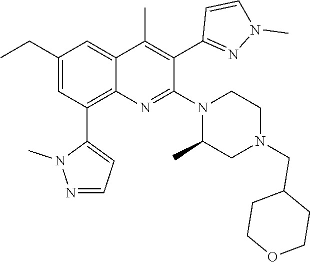 [SMILES: CCc1cc(-c2ccnn2C)c2nc(N3CCN(CC4CCOCC4)C[C@H]3C)c(-c3ccn(C)n3)c(C)c2c1] | 62 |
| 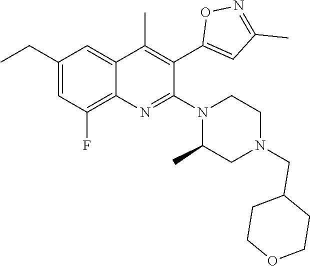 [SMILES: CCc1cc(F)c2nc(N3CCN(CC4CCOCC4)C[C@H]3C)c(-c3cc(C)no3)c(C)c2c1] | 63 |
| 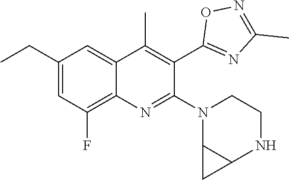 [SMILES: CCc1cc(F)c2nc(N3CCNC4CC43)c(-c3nc(C)no3)c(C)c2c1] | 64 |
| 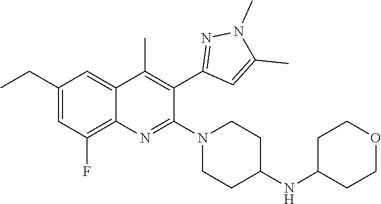 [SMILES: CCc1cc(F)c2nc(N3CCC(NC4CCOCC4)CC3)c(-c3cc(C)n(C)n3)c(C)c2c1] | 65 |
| 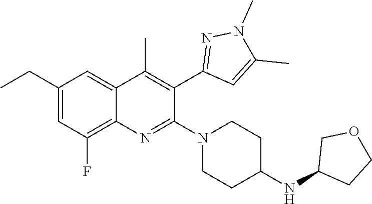 [SMILES: CCc1cc(F)c2nc(N3CCC(N[C@@H]4CCOC4)CC3)c(-c3cc(C)n(C)n3)c(C)c2c1] | 66 |
| 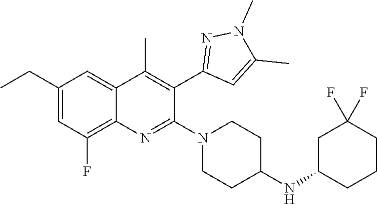 [SMILES: CCc1cc(F)c2nc(N3CCC(N[C@H]4CCCC(F)(F)C4)CC3)c(-c3cc(C)n(C)n3)c(C)c2c1] | 67 |
| 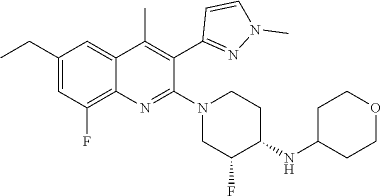 [SMILES: CCc1cc(F)c2nc(N3CC[C@H](NC4CCOCC4)[C@H](F)C3)c(-c3ccn(C)n3)c(C)c2c1] | 68 |
| 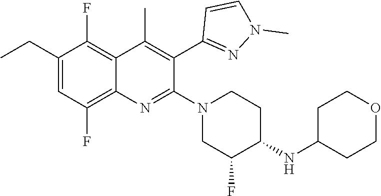 [SMILES: CCc1cc(F)c2nc(N3CC[C@H](NC4CCOCC4)[C@H](F)C3)c(-c3ccn(C)n3)c(C)c2c1F] | 69 |
| 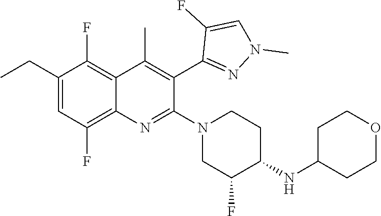 [SMILES: CCc1cc(F)c2nc(N3CC[C@H](NC4CCOCC4)[C@H](F)C3)c(-c3nn(C)cc3F)c(C)c2c1F] | 70 |
| 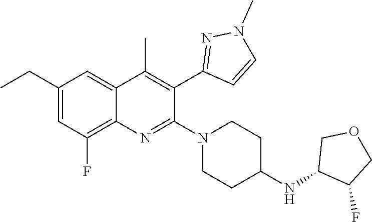 [SMILES: CCc1cc(F)c2nc(N3CCC(N[C@@H]4COC[C@@H]4F)CC3)c(-c3ccn(C)n3)c(C)c2c1] | 71 |
| 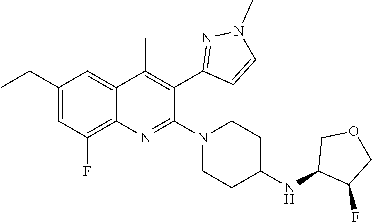 [SMILES: CCc1cc(F)c2nc(N3CCC(N[C@H]4COC[C@H]4F)CC3)c(-c3ccn(C)n3)c(C)c2c1] | 72 |
| 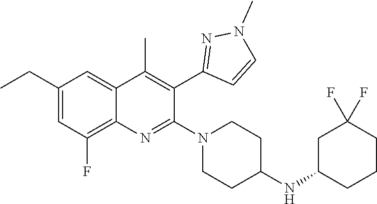 [SMILES: CCc1cc(F)c2nc(N3CCC(N[C@H]4CCCC(F)(F)C4)CC3)c(-c3ccn(C)n3)c(C)c2c1] | 73 |
| 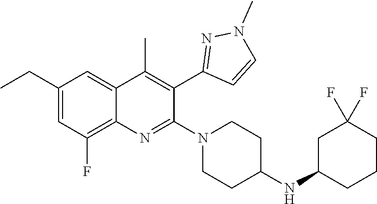 [SMILES: CCc1cc(F)c2nc(N3CCC(N[C@@H]4CCCC(F)(F)C4)CC3)c(-c3ccn(C)n3)c(C)c2c1] | 74 |
| 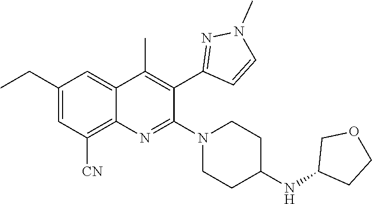 [SMILES: CCc1cc(C#N)c2nc(N3CCC(N[C@H]4CCOC4)CC3)c(-c3ccn(C)n3)c(C)c2c1] | 75 |
| 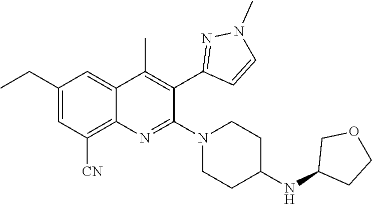 [SMILES: CCc1cc(C#N)c2nc(N3CCC(N[C@@H]4CCOC4)CC3)c(-c3ccn(C)n3)c(C)c2c1] | 76 |
| 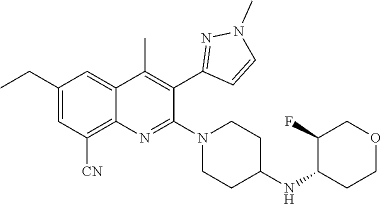 [SMILES: CCc1cc(C#N)c2nc(N3CCC(N[C@H]4CCOC[C@@H]4F)CC3)c(-c3ccn(C)n3)c(C)c2c1] | 77 |
| 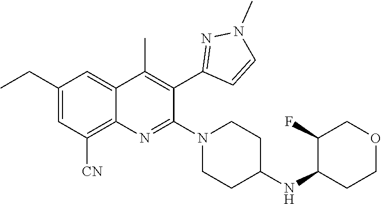 [SMILES: CCc1cc(C#N)c2nc(N3CCC(N[C@@H]4CCOC[C@@H]4F)CC3)c(-c3ccn(C)n3)c(C)c2c1] | 78 |
| 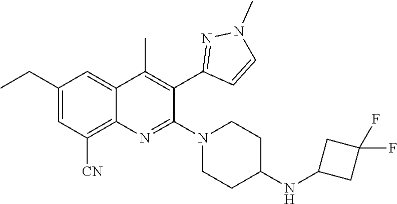 [SMILES: CCc1cc(C#N)c2nc(N3CCC(NC4CC(F)(F)C4)CC3)c(-c3ccn(C)n3)c(C)c2c1] | 79 |
| 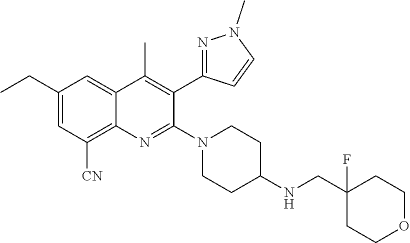 [SMILES: CCc1cc(C#N)c2nc(N3CCC(NCC4(F)CCOCC4)CC3)c(-c3ccn(C)n3)c(C)c2c1] | 80 |
| 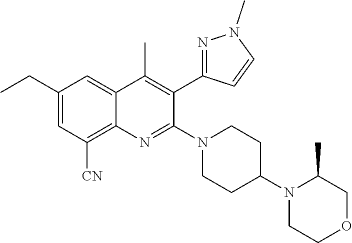 [SMILES: CCc1cc(C#N)c2nc(N3CCC(N4CCOC[C@@H]4C)CC3)c(-c3ccn(C)n3)c(C)c2c1] | 81 |
| 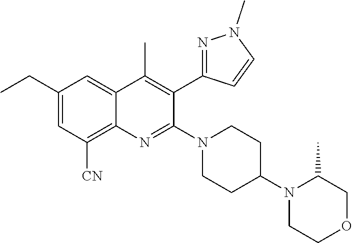 | 82 |
| 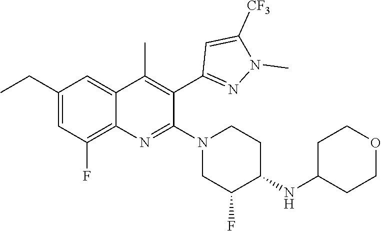 [SMILES: CCc1cc(F)c2nc(N3CC[C@H](NC4CCOCC4)[C@H](F)C3)c(-c3cc(C(F)(F)F)n(C)n3)c(C)c2c1] | 83 |
| 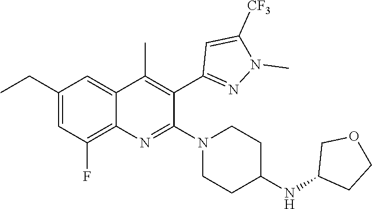 [SMILES: CCc1cc(F)c2nc(N3CCC(N[C@H]4CCOC4)CC3)c(-c3cc(C(F)(F)F)n(C)n3)c(C)c2c1] | 84 |
| 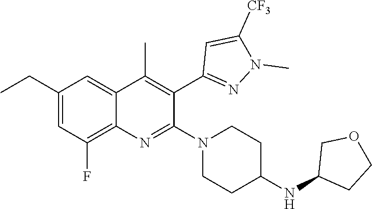 [SMILES: CCc1cc(F)c2nc(N3CCC(N[C@@H]4CCOC4)CC3)c(-c3cc(C(F)(F)F)n(C)n3)c(C)c2c1] | 85 |
| 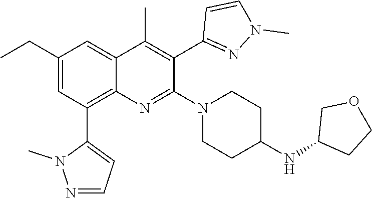 [SMILES: CCc1cc(-c2ccnn2C)c2nc(N3CCC(N[C@H]4CCOC4)CC3)c(-c3ccn(C)n3)c(C)c2c1] | 86 |
| 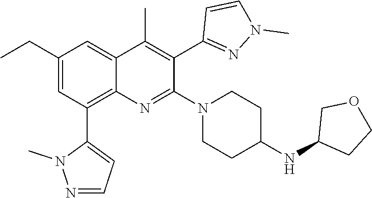 [SMILES: CCc1cc(-c2ccnn2C)c2nc(N3CCC(N[C@@H]4CCOC4)CC3)c(-c3ccn(C)n3)c(C)c2c1] | 87 |
| 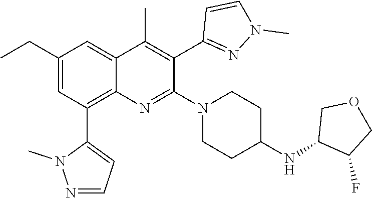 [SMILES: CCc1cc(-c2ccnn2C)c2nc(N3CCC(N[C@@H]4COC[C@@H]4F)CC3)c(-c3ccn(C)n3)c(C)c2c1] | 88 |
| 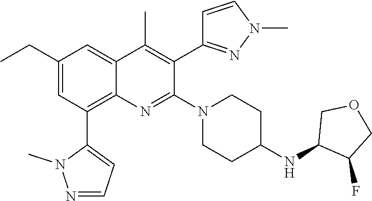 [SMILES: CCc1cc(-c2ccnn2C)c2nc(N3CCC(N[C@H]4COC[C@H]4F)CC3)c(-c3ccn(C)n3)c(C)c2c1] | 89 |
| 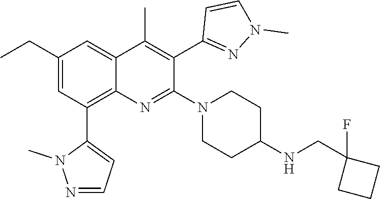 [SMILES: CCc1cc(-c2ccnn2C)c2nc(N3CCC(NCC4(F)CCC4)CC3)c(-c3ccn(C)n3)c(C)c2c1] | 90 |
| 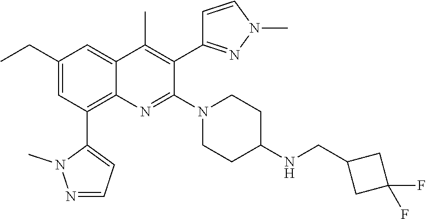 [SMILES: CCc1cc(-c2ccnn2C)c2nc(N3CCC(NCC4CC(F)(F)C4)CC3)c(-c3ccn(C)n3)c(C)c2c1] | 91 |
| 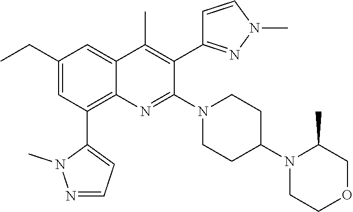 [SMILES: CCc1cc(-c2ccnn2C)c2nc(N3CCC(N4CCOC[C@@H]4C)CC3)c(-c3ccn(C)n3)c(C)c2c1] | 92 |
| 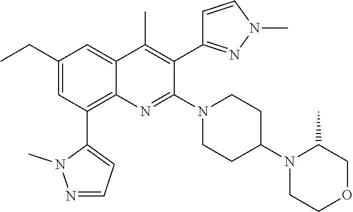 | 93 |
| 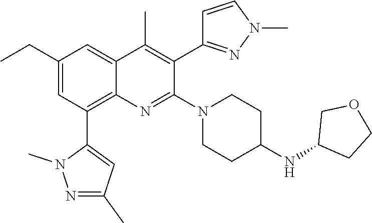 [SMILES: CCc1cc(-c2cc(C)nn2C)c2nc(N3CCC(N[C@H]4CCOC4)CC3)c(-c3ccn(C)n3)c(C)c2c1] | 94 |
| 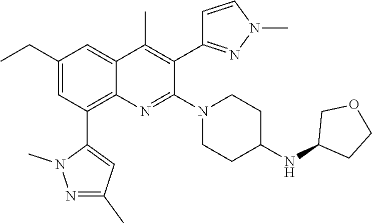 [SMILES: CCc1cc(-c2cc(C)nn2C)c2nc(N3CCC(N[C@@H]4CCOC4)CC3)c(-c3ccn(C)n3)c(C)c2c1] | 95 |
| 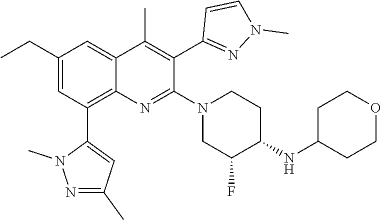 [SMILES: CCc1cc(-c2cc(C)nn2C)c2nc(N3CC[C@H](NC4CCOCC4)[C@H](F)C3)c(-c3ccn(C)n3)c(C)c2c1] | 96 |
| 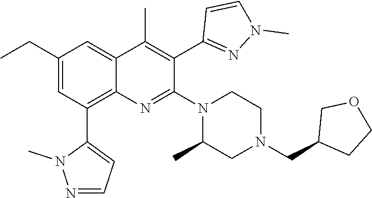 [SMILES: CCc1cc(-c2ccnn2C)c2nc(N3CCN(C[C@@H]4CCOC4)C[C@H]3C)c(-c3ccn(C)n3)c(C)c2c1] | 97 |
| 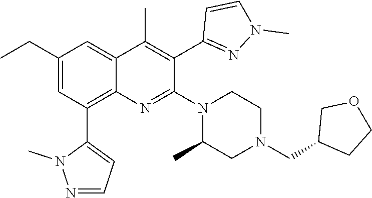 [SMILES: CCc1cc(-c2ccnn2C)c2nc(N3CCN(C[C@H]4CCOC4)C[C@H]3C)c(-c3ccn(C)n3)c(C)c2c1] | 98 |
| 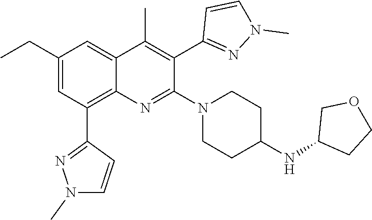 [SMILES: CCc1cc(-c2ccn(C)n2)c2nc(N3CCC(N[C@H]4CCOC4)CC3)c(-c3ccn(C)n3)c(C)c2c1] | 99 |
| 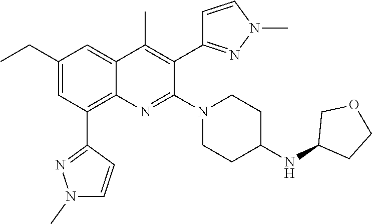 [SMILES: CCc1cc(-c2ccn(C)n2)c2nc(N3CCC(N[C@@H]4CCOC4)CC3)c(-c3ccn(C)n3)c(C)c2c1] | 100 |
| 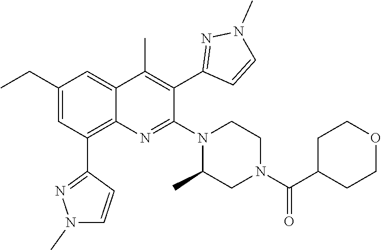 [SMILES: CCc1cc(-c2ccn(C)n2)c2nc(N3CCN(C(=O)C4CCOCC4)C[C@H]3C)c(-c3ccn(C)n3)c(C)c2c1] | 101 |
| 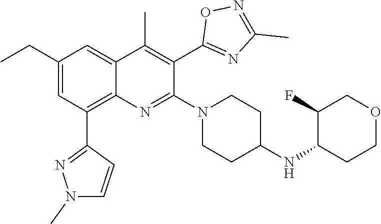 [SMILES: CCc1cc(-c2ccn(C)n2)c2nc(N3CCC(N[C@H]4CCOC[C@@H]4F)CC3)c(-c3nc(C)no3)c(C)c2c1] | 102 |
| 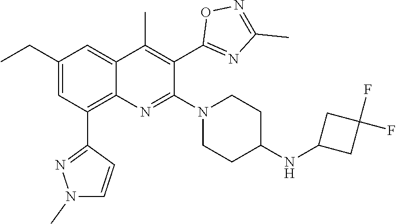 [SMILES: CCc1cc(-c2ccn(C)n2)c2nc(N3CCC(NC4CC(F)(F)C4)CC3)c(-c3nc(C)no3)c(C)c2c1] | 103 |
| 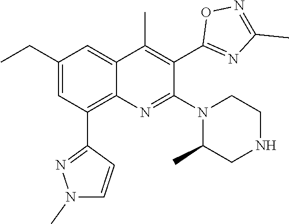 [SMILES: CCc1cc(-c2ccn(C)n2)c2nc(N3CCNC[C@H]3C)c(-c3nc(C)no3)c(C)c2c1] | 104 |
| 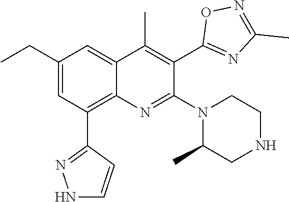 [SMILES: CCc1cc(-c2cc[nH]n2)c2nc(N3CCNC[C@H]3C)c(-c3nc(C)no3)c(C)c2c1] | 105 |
| 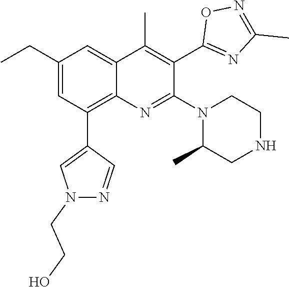 [SMILES: CCc1cc(-c2cnn(CCO)c2)c2nc(N3CCNC[C@H]3C)c(-c3nc(C)no3)c(C)c2c1] | 106 |
| 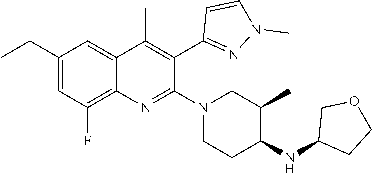 [SMILES: CCc1cc(F)c2nc(N3CC[C@H](N[C@@H]4CCOC4)[C@H](C)C3)c(-c3ccn(C)n3)c(C)c2c1] | 107 |
| 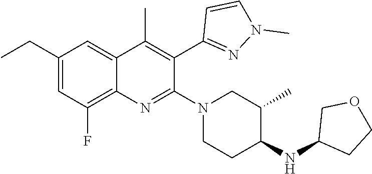 [SMILES: CCc1cc(F)c2nc(N3CC[C@H](N[C@@H]4CCOC4)[C@@H](C)C3)c(-c3ccn(C)n3)c(C)c2c1] | 108 |
| 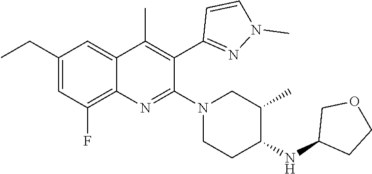 [SMILES: CCc1cc(F)c2nc(N3CC[C@@H](N[C@@H]4CCOC4)[C@@H](C)C3)c(-c3ccn(C)n3)c(C)c2c1] | 109 |
| 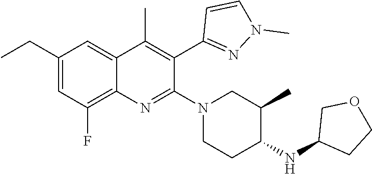 [SMILES: CCc1cc(F)c2nc(N3CC[C@@H](N[C@@H]4CCOC4)[C@H](C)C3)c(-c3ccn(C)n3)c(C)c2c1] | 110 |
| 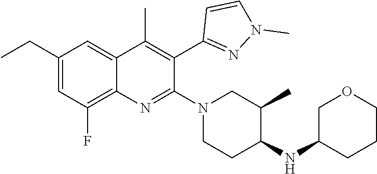 [SMILES: CCc1cc(F)c2nc(N3CC[C@H](N[C@@H]4CCCOC4)[C@H](C)C3)c(-c3ccn(C)n3)c(C)c2c1] | 111 |
| 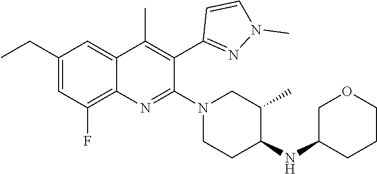 [SMILES: CCc1cc(F)c2nc(N3CC[C@H](N[C@@H]4CCCOC4)[C@@H](C)C3)c(-c3ccn(C)n3)c(C)c2c1] | 112 |
| 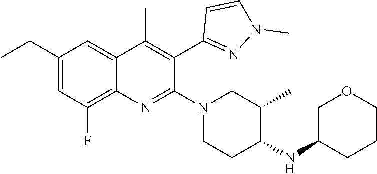 [SMILES: CCc1cc(F)c2nc(N3CC[C@@H](N[C@@H]4CCCOC4)[C@@H](C)C3)c(-c3ccn(C)n3)c(C)c2c1] | 113 |
| 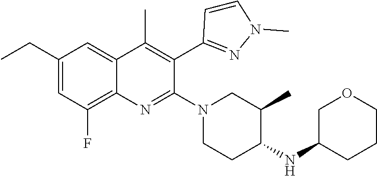 [SMILES: CCc1cc(F)c2nc(N3CC[C@@H](N[C@@H]4CCCOC4)[C@H](C)C3)c(-c3ccn(C)n3)c(C)c2c1] | 114 |
| 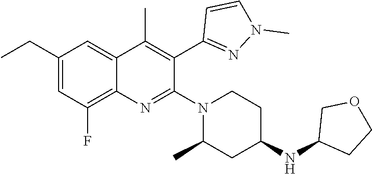 [SMILES: CCc1cc(F)c2nc(N3CC[C@@H](N[C@@H]4CCOC4)C[C@H]3C)c(-c3ccn(C)n3)c(C)c2c1] | 115 |
| 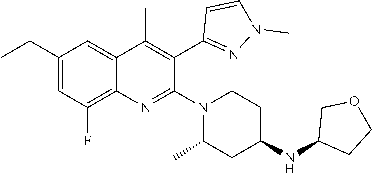 [SMILES: CCc1cc(F)c2nc(N3CC[C@@H](N[C@@H]4CCOC4)C[C@@H]3C)c(-c3ccn(C)n3)c(C)c2c1] | 116 |
| 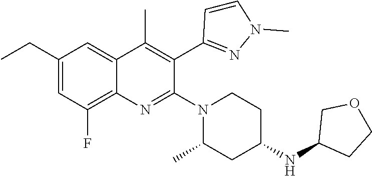 [SMILES: CCc1cc(F)c2nc(N3CC[C@H](N[C@@H]4CCOC4)C[C@@H]3C)c(-c3ccn(C)n3)c(C)c2c1] | 117 |
| 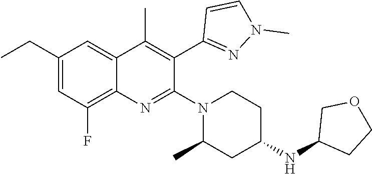 [SMILES: CCc1cc(F)c2nc(N3CC[C@H](N[C@@H]4CCOC4)C[C@H]3C)c(-c3ccn(C)n3)c(C)c2c1] | 118 |
| 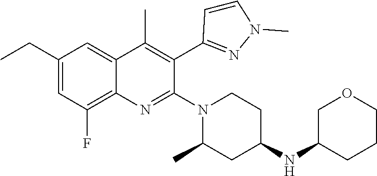 [SMILES: CCc1cc(F)c2nc(N3CC[C@@H](N[C@@H]4CCCOC4)C[C@H]3C)c(-c3ccn(C)n3)c(C)c2c1] | 119 |
| 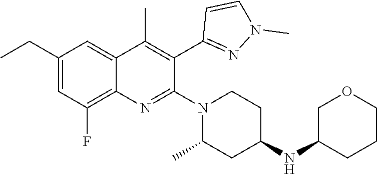 [SMILES: CCc1cc(F)c2nc(N3CC[C@@H](N[C@@H]4CCCOC4)C[C@@H]3C)c(-c3ccn(C)n3)c(C)c2c1] | 120 |
| 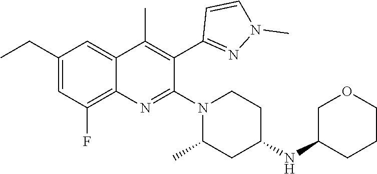 [SMILES: CCc1cc(F)c2nc(N3CC[C@H](N[C@@H]4CCCOC4)C[C@@H]3C)c(-c3ccn(C)n3)c(C)c2c1] | 121 |
| 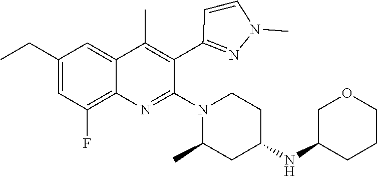 [SMILES: CCc1cc(F)c2nc(N3CC[C@H](N[C@@H]4CCCOC4)C[C@H]3C)c(-c3ccn(C)n3)c(C)c2c1] | 122 |
| 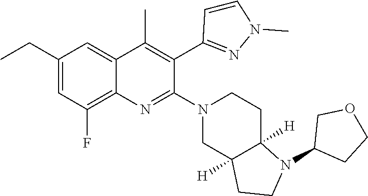 [SMILES: CCc1cc(F)c2nc(N3CC[C@@H]4[C@@H](CCN4[C@@H]4CCOC4)C3)c(-c3ccn(C)n3)c(C)c2c1] | 123 |
| 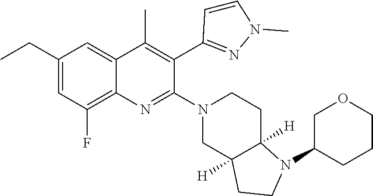 [SMILES: CCc1cc(F)c2nc(N3CC[C@@H]4[C@@H](CCN4[C@@H]4CCCOC4)C3)c(-c3ccn(C)n3)c(C)c2c1] | 124 |
| 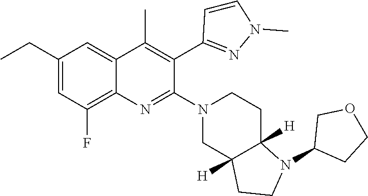 [SMILES: CCc1cc(F)c2nc(N3CC[C@H]4[C@H](CCN4[C@@H]4CCOC4)C3)c(-c3ccn(C)n3)c(C)c2c1] | 125 |
| 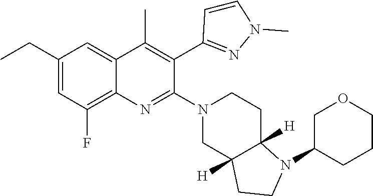 [SMILES: CCc1cc(F)c2nc(N3CC[C@H]4[C@H](CCN4[C@@H]4CCCOC4)C3)c(-c3ccn(C)n3)c(C)c2c1] | 126 |
| 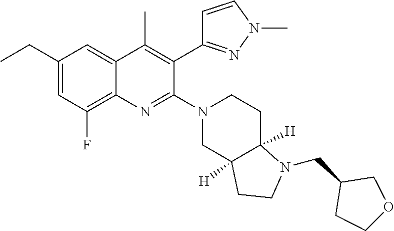 [SMILES: CCc1cc(F)c2nc(N3CC[C@@H]4[C@@H](CCN4C[C@@H]4CCOC4)C3)c(-c3ccn(C)n3)c(C)c2c1] | 127 |
| 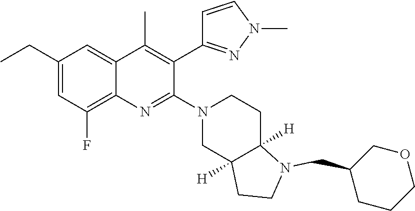 [SMILES: CCc1cc(F)c2nc(N3CC[C@@H]4[C@@H](CCN4C[C@@H]4CCCOC4)C3)c(-c3ccn(C)n3)c(C)c2c1] | 128 |
| 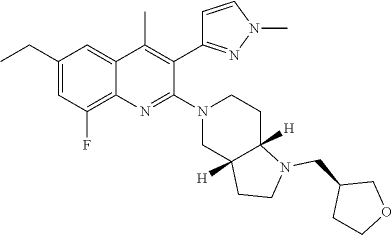 [SMILES: CCc1cc(F)c2nc(N3CC[C@H]4[C@H](CCN4C[C@@H]4CCOC4)C3)c(-c3ccn(C)n3)c(C)c2c1] | 129 |
| 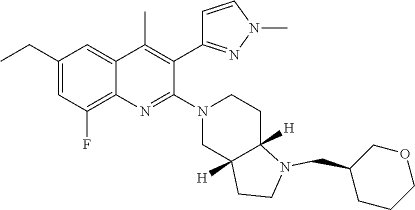 [SMILES: CCc1cc(F)c2nc(N3CC[C@H]4[C@H](CCN4C[C@@H]4CCCOC4)C3)c(-c3ccn(C)n3)c(C)c2c1] | 130 |
| 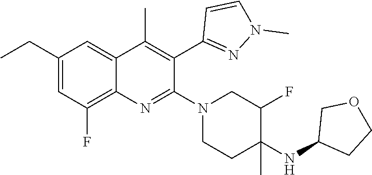 [SMILES: CCc1cc(F)c2nc(N3CCC(C)(N[C@@H]4CCOC4)C(F)C3)c(-c3ccn(C)n3)c(C)c2c1] | 131 |
| 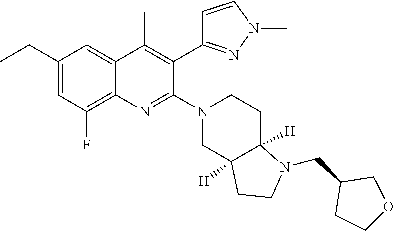 [SMILES: CCc1cc(F)c2nc(N3CC[C@@H]4[C@@H](CCN4C[C@@H]4CCOC4)C3)c(-c3ccn(C)n3)c(C)c2c1] | 132 |
| 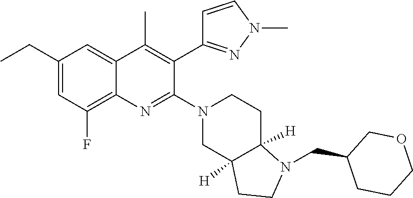 [SMILES: CCc1cc(F)c2nc(N3CC[C@@H]4[C@@H](CCN4C[C@@H]4CCCOC4)C3)c(-c3ccn(C)n3)c(C)c2c1] | 133 |
| 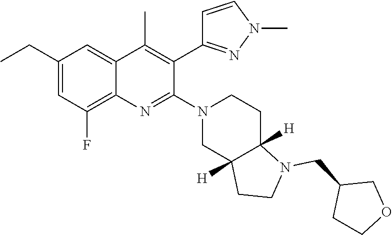 [SMILES: CCc1cc(F)c2nc(N3CC[C@H]4[C@H](CCN4C[C@@H]4CCOC4)C3)c(-c3ccn(C)n3)c(C)c2c1] | 134 |
| 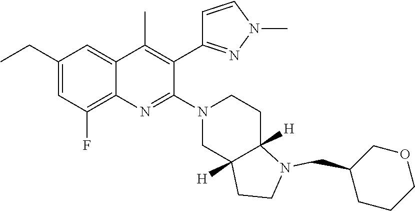 [SMILES: CCc1cc(F)c2nc(N3CC[C@H]4[C@H](CCN4C[C@@H]4CCCOC4)C3)c(-c3ccn(C)n3)c(C)c2c1] | 135 |
| 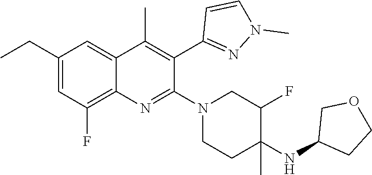 [SMILES: CCc1cc(F)c2nc(N3CCC(C)(N[C@@H]4CCOC4)C(F)C3)c(-c3ccn(C)n3)c(C)c2c1] | 136 |
| 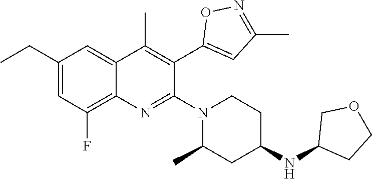 [SMILES: CCc1cc(F)c2nc(N3CC[C@@H](N[C@@H]4CCOC4)C[C@H]3C)c(-c3cc(C)no3)c(C)c2c1] | 137 |
| 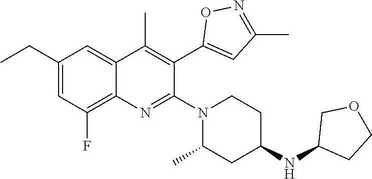 [SMILES: CCc1cc(F)c2nc(N3CC[C@@H](N[C@@H]4CCOC4)C[C@@H]3C)c(-c3cc(C)no3)c(C)c2c1] | 138 |
| 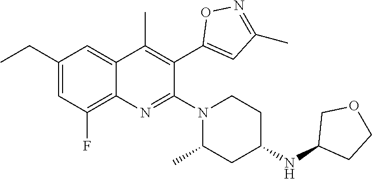 [SMILES: CCc1cc(F)c2nc(N3CC[C@H](N[C@@H]4CCOC4)C[C@@H]3C)c(-c3cc(C)no3)c(C)c2c1] | 139 |
| 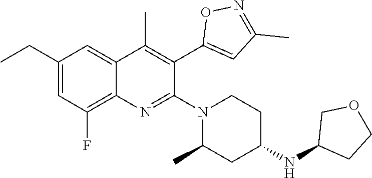 [SMILES: CCc1cc(F)c2nc(N3CC[C@H](N[C@@H]4CCOC4)C[C@H]3C)c(-c3cc(C)no3)c(C)c2c1] | 140 |
| 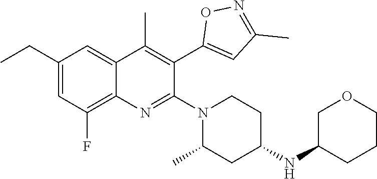 [SMILES: CCc1cc(F)c2nc(N3CC[C@H](N[C@@H]4CCCOC4)C[C@@H]3C)c(-c3cc(C)no3)c(C)c2c1] | 141 |
| 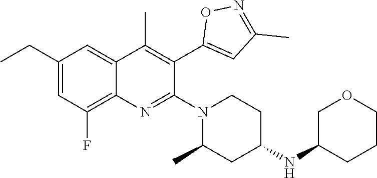 [SMILES: CCc1cc(F)c2nc(N3CC[C@H](N[C@@H]4CCCOC4)C[C@H]3C)c(-c3cc(C)no3)c(C)c2c1] | 142 |
| 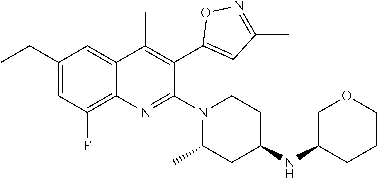 [SMILES: CCc1cc(F)c2nc(N3CC[C@@H](N[C@@H]4CCCOC4)C[C@@H]3C)c(-c3cc(C)no3)c(C)c2c1] | 143 |
| 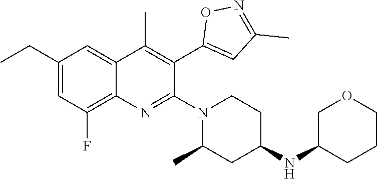 [SMILES: CCc1cc(F)c2nc(N3CC[C@@H](N[C@@H]4CCCOC4)C[C@H]3C)c(-c3cc(C)no3)c(C)c2c1] | 144 |
| 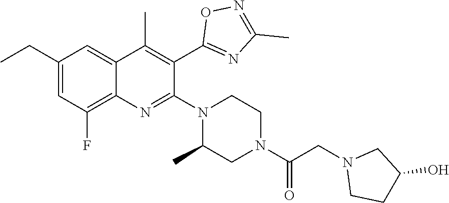 [SMILES: CCc1cc(F)c2nc(N3CCN(C(=O)CN4CC[C@@H](O)C4)C[C@H]3C)c(-c3nc(C)no3)c(C)c2c1] | 145 |
| 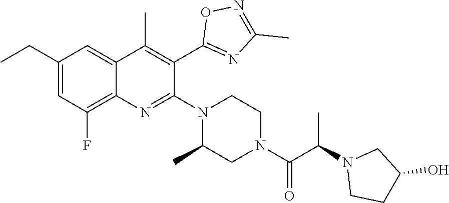 [SMILES: CCc1cc(F)c2nc(N3CCN(C(=O)[C@@H](C)N4CC[C@@H](O)C4)C[C@H]3C)c(-c3nc(C)no3)c(C)c2c1] | 146 |
| 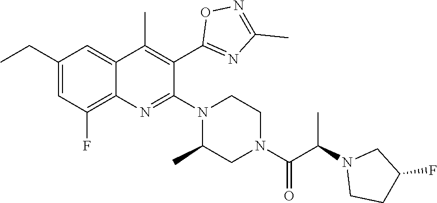 [SMILES: CCc1cc(F)c2nc(N3CCN(C(=O)[C@@H](C)N4CC[C@@H](F)C4)C[C@H]3C)c(-c3nc(C)no3)c(C)c2c1] | 147 |
| 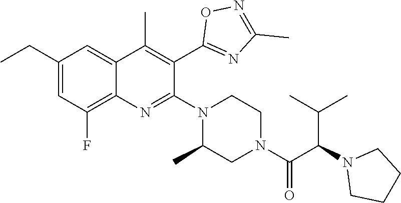 [SMILES: CCc1cc(F)c2nc(N3CCN(C(=O)[C@@H](C(C)C)N4CCCC4)C[C@H]3C)c(-c3nc(C)no3)c(C)c2c1] | 148 |
| 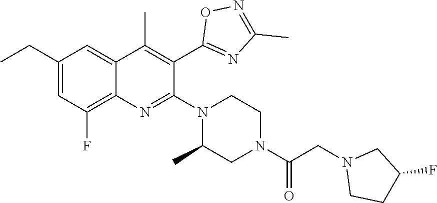 [SMILES: CCc1cc(F)c2nc(N3CCN(C(=O)CN4CC[C@@H](F)C4)C[C@H]3C)c(-c3nc(C)no3)c(C)c2c1] | 149 |
| 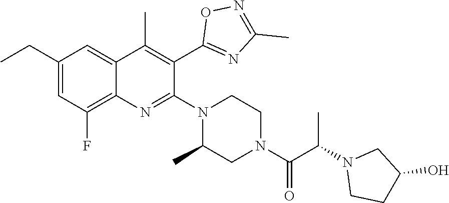 [SMILES: CCc1cc(F)c2nc(N3CCN(C(=O)[C@H](C)N4CC[C@@H](O)C4)C[C@H]3C)c(-c3nc(C)no3)c(C)c2c1] | 150 |
| 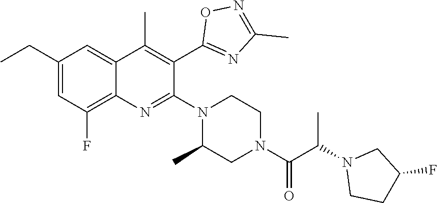 | 151 |
| 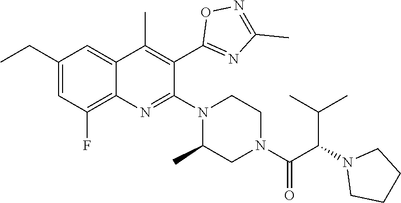 [SMILES: CCc1cc(F)c2nc(N3CCN(C(=O)[C@H](C(C)C)N4CCCC4)C[C@H]3C)c(-c3nc(C)no3)c(C)c2c1] | 152 |
| 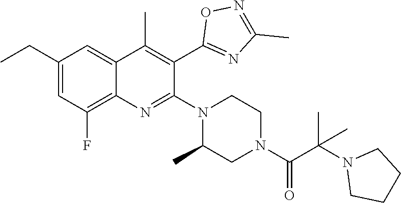 [SMILES: CCc1cc(F)c2nc(N3CCN(C(=O)C(C)(C)N4CCCC4)C[C@H]3C)c(-c3nc(C)no3)c(C)c2c1] | 153 |
| 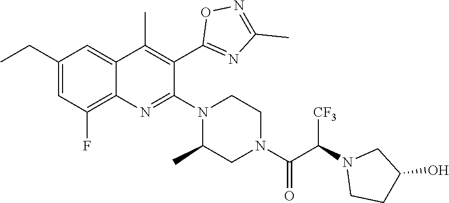 [SMILES: CCc1cc(F)c2nc(N3CCN(C(=O)[C@H](N4CC[C@@H](O)C4)C(F)(F)F)C[C@H]3C)c(-c3nc(C)no3)c(C)c2c1] | 154 |
| 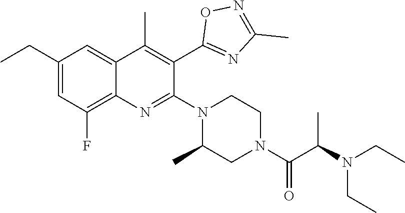 [SMILES: CCc1cc(F)c2nc(N3CCN(C(=O)[C@@H](C)N(CC)CC)C[C@H]3C)c(-c3nc(C)no3)c(C)c2c1] | 155 |
| 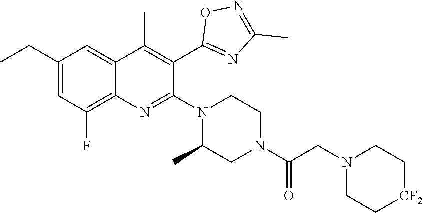 [SMILES: CCc1cc(F)c2nc(N3CCN(C(=O)CN4CCC(F)(F)CC4)C[C@H]3C)c(-c3nc(C)no3)c(C)c2c1] | 156 |
| 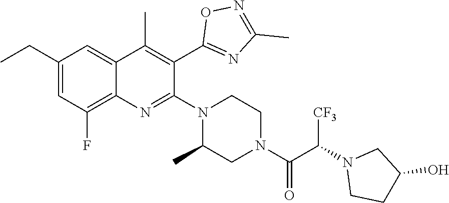 [SMILES: CCc1cc(F)c2nc(N3CCN(C(=O)[C@@H](N4CC[C@@H](O)C4)C(F)(F)F)C[C@H]3C)c(-c3nc(C)no3)c(C)c2c1] | 157 |
| 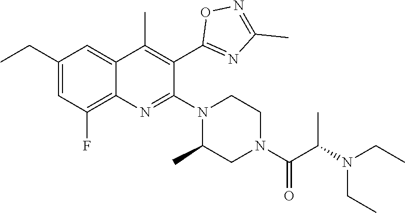 [SMILES: CCc1cc(F)c2nc(N3CCN(C(=O)[C@H](C)N(CC)CC)C[C@H]3C)c(-c3nc(C)no3)c(C)c2c1] | 158 |
| 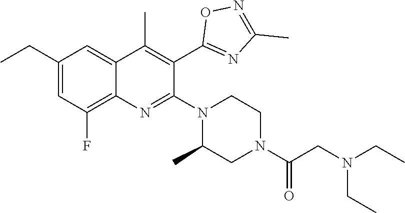 [SMILES: CCc1cc(F)c2nc(N3CCN(C(=O)CN(CC)CC)C[C@H]3C)c(-c3nc(C)no3)c(C)c2c1] | 159 |
| 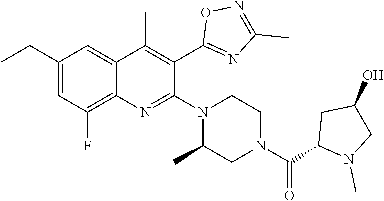 [SMILES: CCc1cc(F)c2nc(N3CCN(C(=O)[C@@H]4C[C@@H](O)CN4C)C[C@H]3C)c(-c3nc(C)no3)c(C)c2c1] | 160 |
| 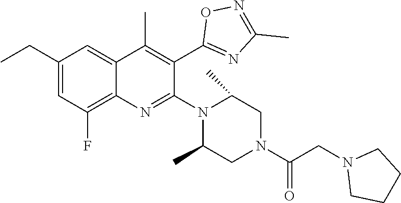 [SMILES: CCc1cc(F)c2nc(N3[C@H](C)CN(C(=O)CN4CCCC4)C[C@H]3C)c(-c3nc(C)no3)c(C)c2c1] | 161 |
| 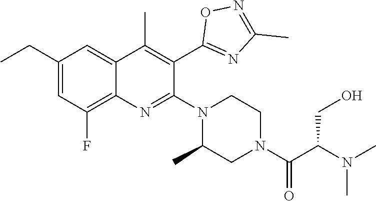 [SMILES: CCc1cc(F)c2nc(N3CCN(C(=O)[C@H](CO)N(C)C)C[C@H]3C)c(-c3nc(C)no3)c(C)c2c1] | 162 |
| 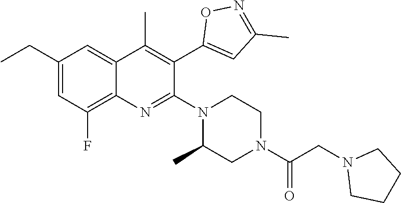 [SMILES: CCc1cc(F)c2nc(N3CCN(C(=O)CN4CCCC4)C[C@H]3C)c(-c3cc(C)no3)c(C)c2c1] | 163 |
| 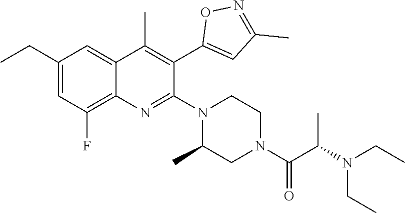 [SMILES: CCc1cc(F)c2nc(N3CCN(C(=O)[C@H](C)N(CC)CC)C[C@H]3C)c(-c3cc(C)no3)c(C)c2c1] | 164 |
| 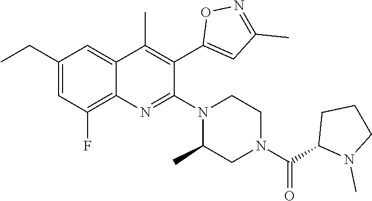 [SMILES: CCc1cc(F)c2nc(N3CCN(C(=O)[C@@H]4CCCN4C)C[C@H]3C)c(-c3cc(C)no3)c(C)c2c1] | 165 |
| 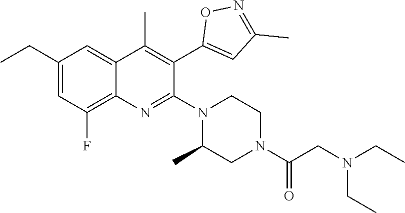 [SMILES: CCc1cc(F)c2nc(N3CCN(C(=O)CN(CC)CC)C[C@H]3C)c(-c3cc(C)no3)c(C)c2c1] | 166 |
| 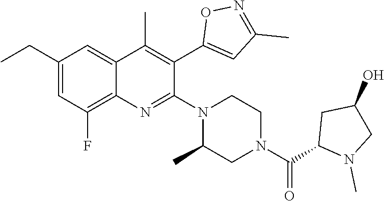 [SMILES: CCc1cc(F)c2nc(N3CCN(C(=O)[C@@H]4C[C@@H](O)CN4C)C[C@H]3C)c(-c3cc(C)no3)c(C)c2c1] | 167 |
| 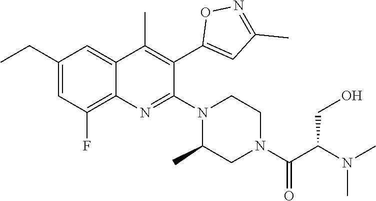 [SMILES: CCc1cc(F)c2nc(N3CCN(C(=O)[C@H](CO)N(C)C)C[C@H]3C)c(-c3cc(C)no3)c(C)c2c1] | 168 |
| 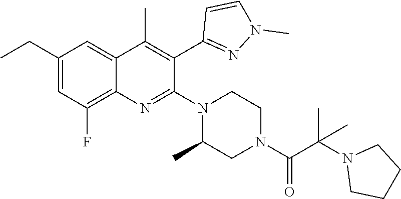 [SMILES: CCc1cc(F)c2nc(N3CCN(C(=O)C(C)(C)N4CCCC4)C[C@H]3C)c(-c3ccn(C)n3)c(C)c2c1] | 169 |
| 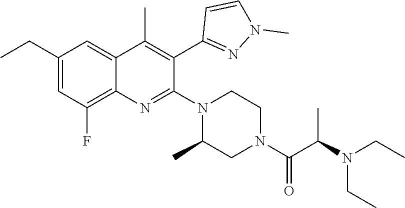 [SMILES: CCc1cc(F)c2nc(N3CCN(C(=O)[C@@H](C)N(CC)CC)C[C@H]3C)c(-c3ccn(C)n3)c(C)c2c1] | 170 |
| 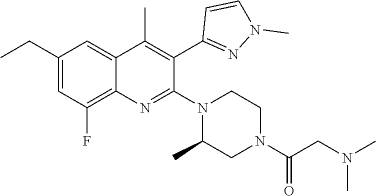 [SMILES: CCc1cc(F)c2nc(N3CCN(C(=O)CN(C)C)C[C@H]3C)c(-c3ccn(C)n3)c(C)c2c1] | 171 |
| 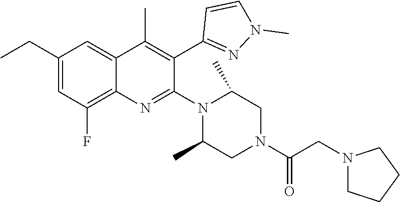 [SMILES: CCc1cc(F)c2nc(N3[C@H](C)CN(C(=O)CN4CCCC4)C[C@H]3C)c(-c3ccn(C)n3)c(C)c2c1] | 172 |
| 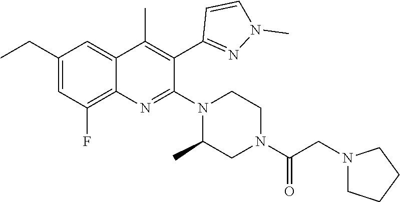 [SMILES: CCc1cc(F)c2nc(N3CCN(C(=O)CN4CCCC4)C[C@H]3C)c(-c3ccn(C)n3)c(C)c2c1] | 173 |
| 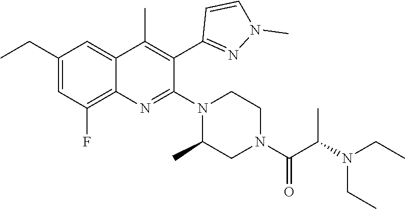 [SMILES: CCc1cc(F)c2nc(N3CCN(C(=O)[C@H](C)N(CC)CC)C[C@H]3C)c(-c3ccn(C)n3)c(C)c2c1] | 174 |
| 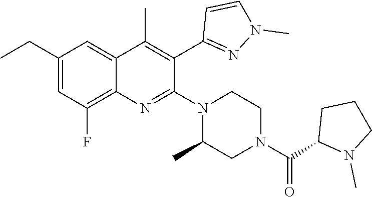 [SMILES: CCc1cc(F)c2nc(N3CCN(C(=O)[C@@H]4CCCN4C)C[C@H]3C)c(-c3ccn(C)n3)c(C)c2c1] | 175 |
| 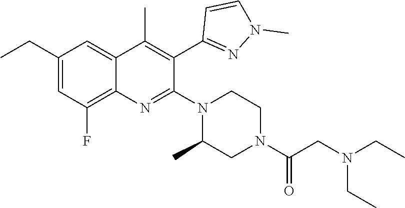 [SMILES: CCc1cc(F)c2nc(N3CCN(C(=O)CN(CC)CC)C[C@H]3C)c(-c3ccn(C)n3)c(C)c2c1] | 176 |
| 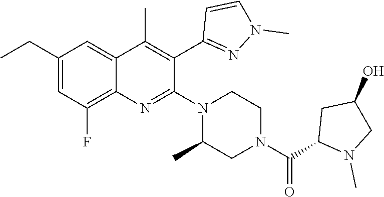 [SMILES: CCc1cc(F)c2nc(N3CCN(C(=O)[C@@H]4C[C@@H](O)CN4C)C[C@H]3C)c(-c3ccn(C)n3)c(C)c2c1] | 177 |
| 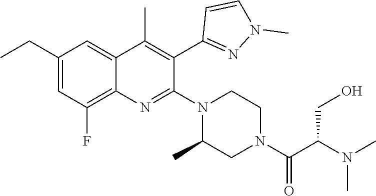 [SMILES: CCc1cc(F)c2nc(N3CCN(C(=O)[C@H](CO)N(C)C)C[C@H]3C)c(-c3ccn(C)n3)c(C)c2c1] | 178 |
| 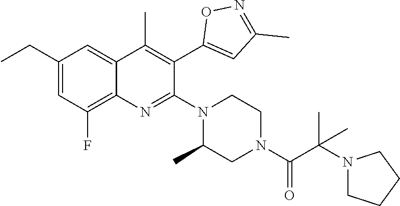 [SMILES: CCc1cc(F)c2nc(N3CCN(C(=O)C(C)(C)N4CCCC4)C[C@H]3C)c(-c3cc(C)no3)c(C)c2c1] | 179 |
| 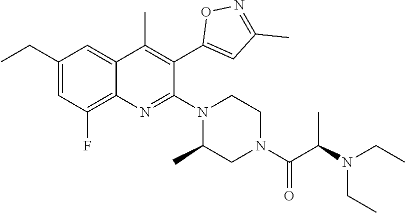 [SMILES: CCc1cc(F)c2nc(N3CCN(C(=O)[C@@H](C)N(CC)CC)C[C@H]3C)c(-c3cc(C)no3)c(C)c2c1] | 180 |
| 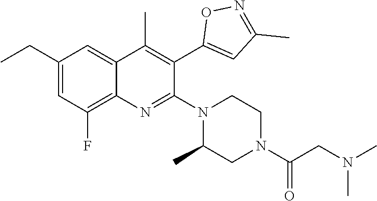 [SMILES: CCc1cc(F)c2nc(N3CCN(C(=O)CN(C)C)C[C@H]3C)c(-c3cc(C)no3)c(C)c2c1] | 181 |
| 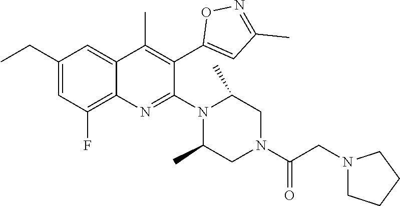 [SMILES: CCc1cc(F)c2nc(N3[C@H](C)CN(C(=O)CN4CCCC4)C[C@H]3C)c(-c3cc(C)no3)c(C)c2c1] | 182 |
| 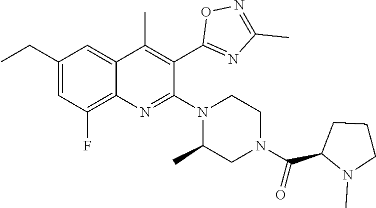 [SMILES: CCc1cc(F)c2nc(N3CCN(C(=O)[C@H]4CCCN4C)C[C@H]3C)c(-c3nc(C)no3)c(C)c2c1] | 183 |
| 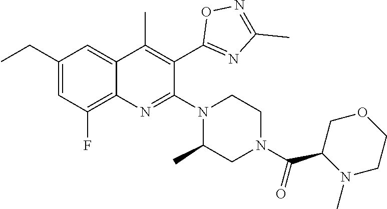 [SMILES: CCc1cc(F)c2nc(N3CCN(C(=O)[C@H]4COCCN4C)C[C@H]3C)c(-c3nc(C)no3)c(C)c2c1] | 184 |
| 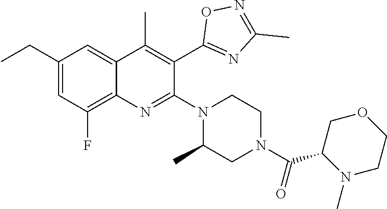 [SMILES: CCc1cc(F)c2nc(N3CCN(C(=O)[C@@H]4COCCN4C)C[C@H]3C)c(-c3nc(C)no3)c(C)c2c1] | 185 |
| 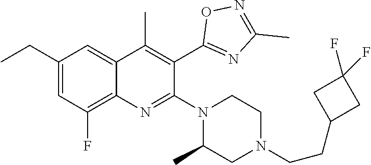 [SMILES: CCc1cc(F)c2nc(N3CCN(CCC4CC(F)(F)C4)C[C@H]3C)c(-c3nc(C)no3)c(C)c2c1] | 186 |
| 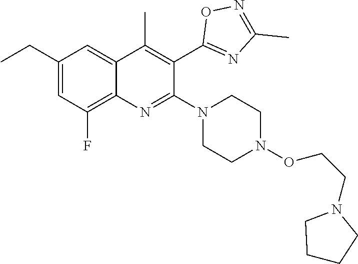 [SMILES: CCc1cc(F)c2nc(N3CCN(OCCN4CCCC4)CC3)c(-c3nc(C)no3)c(C)c2c1] | 187 |
| 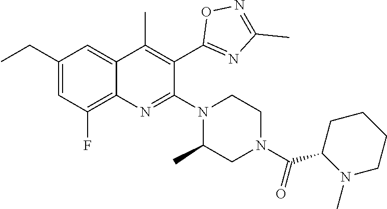 [SMILES: CCc1cc(F)c2nc(N3CCN(C(=O)[C@@H]4CCCCN4C)C[C@H]3C)c(-c3nc(C)no3)c(C)c2c1] | 188 |
| 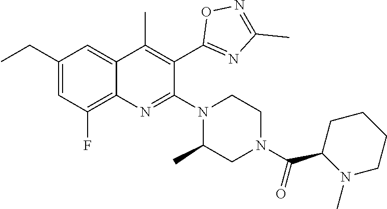 [SMILES: CCc1cc(F)c2nc(N3CCN(C(=O)[C@H]4CCCCN4C)C[C@H]3C)c(-c3nc(C)no3)c(C)c2c1] | 189 |
| 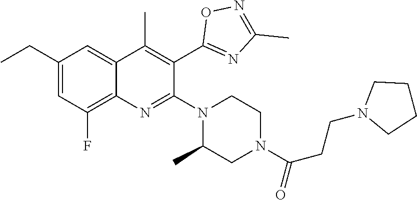 [SMILES: CCc1cc(F)c2nc(N3CCN(C(=O)CCN4CCCC4)C[C@H]3C)c(-c3nc(C)no3)c(C)c2c1] | 190 |
| 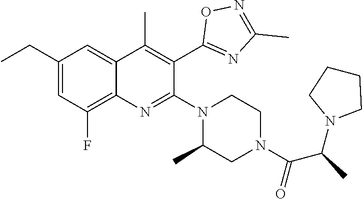 [SMILES: CCc1cc(F)c2nc(N3CCN(C(=O)[C@H](C)N4CCCC4)C[C@H]3C)c(-c3nc(C)no3)c(C)c2c1] | 191 |
| 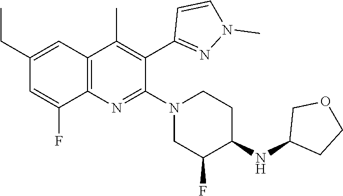 [SMILES: CCc1cc(F)c2nc(N3CC[C@@H](N[C@@H]4CCOC4)[C@@H](F)C3)c(-c3ccn(C)n3)c(C)c2c1] | 192 |
| 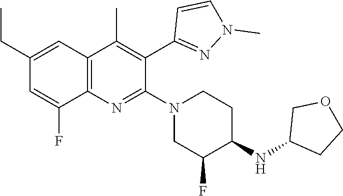 [SMILES: CCc1cc(F)c2nc(N3CC[C@@H](N[C@H]4CCOC4)[C@@H](F)C3)c(-c3ccn(C)n3)c(C)c2c1] | 193 |
| 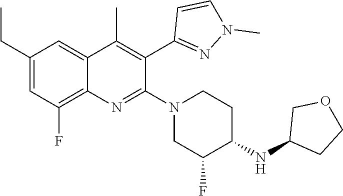 [SMILES: CCc1cc(F)c2nc(N3CC[C@H](N[C@@H]4CCOC4)[C@H](F)C3)c(-c3ccn(C)n3)c(C)c2c1] | 194 |
| 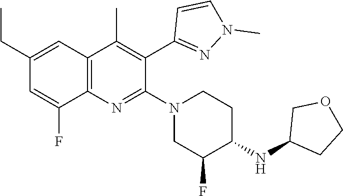 [SMILES: CCc1cc(F)c2nc(N3CC[C@H](N[C@@H]4CCOC4)[C@@H](F)C3)c(-c3ccn(C)n3)c(C)c2c1] | 195 |
| 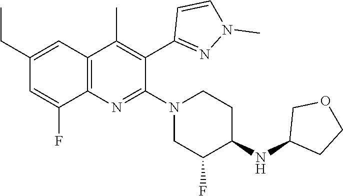 [SMILES: CCc1cc(F)c2nc(N3CC[C@@H](N[C@@H]4CCOC4)[C@H](F)C3)c(-c3ccn(C)n3)c(C)c2c1] | 196 |
| 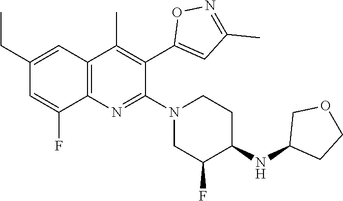 [SMILES: CCc1cc(F)c2nc(N3CC[C@@H](N[C@@H]4CCOC4)[C@@H](F)C3)c(-c3cc(C)no3)c(C)c2c1] | 197 |
| 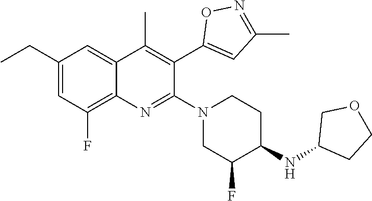 [SMILES: CCc1cc(F)c2nc(N3CC[C@@H](N[C@H]4CCOC4)[C@@H](F)C3)c(-c3cc(C)no3)c(C)c2c1] | 198 |
| 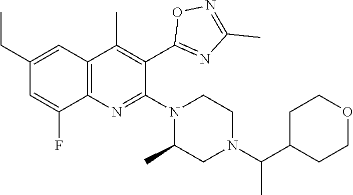 [SMILES: CCc1cc(F)c2nc(N3CCN(C(C)C4CCOCC4)C[C@H]3C)c(-c3nc(C)no3)c(C)c2c1] | 199 |
| 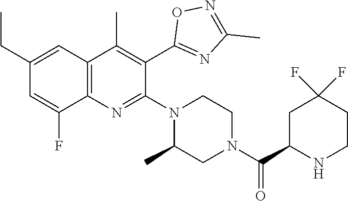 [SMILES: CCc1cc(F)c2nc(N3CCN(C(=O)[C@H]4CC(F)(F)CCN4)C[C@H]3C)c(-c3nc(C)no3)c(C)c2c1] | 200 |
| 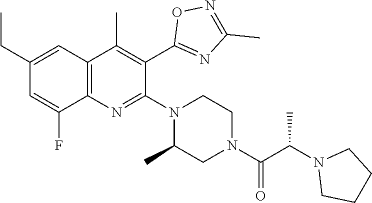 [SMILES: CCc1cc(F)c2nc(N3CCN(C(=O)[C@H](C)N4CCCC4)C[C@H]3C)c(-c3nc(C)no3)c(C)c2c1] | 201 |
| 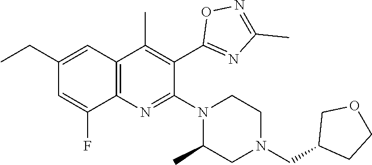 [SMILES: CCc1cc(F)c2nc(N3CCN(C[C@H]4CCOC4)C[C@H]3C)c(-c3nc(C)no3)c(C)c2c1] | 202 |
| 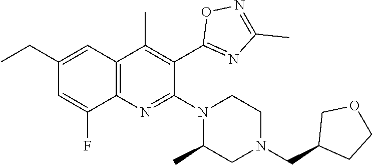 [SMILES: CCc1cc(F)c2nc(N3CCN(C[C@@H]4CCOC4)C[C@H]3C)c(-c3nc(C)no3)c(C)c2c1] | 203 |
| 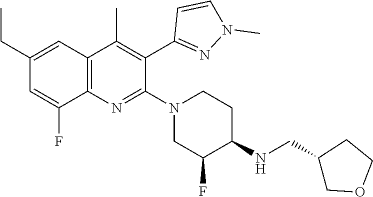 [SMILES: CCc1cc(F)c2nc(N3CC[C@@H](NC[C@@H]4CCOC4)[C@@H](F)C3)c(-c3ccn(C)n3)c(C)c2c1] | 204 |
| 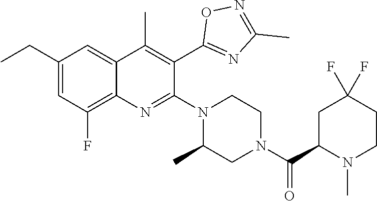 [SMILES: CCc1cc(F)c2nc(N3CCN(C(=O)[C@H]4CC(F)(F)CCN4C)C[C@H]3C)c(-c3nc(C)no3)c(C)c2c1] | 205 |
| 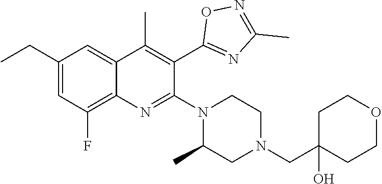 [SMILES: CCc1cc(F)c2nc(N3CCN(CC4(O)CCOCC4)C[C@H]3C)c(-c3nc(C)no3)c(C)c2c1] | 206 |
| 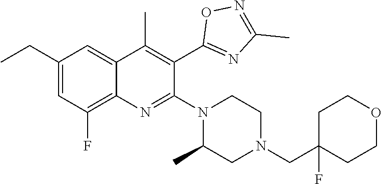 [SMILES: CCc1cc(F)c2nc(N3CCN(CC4(F)CCOCC4)C[C@H]3C)c(-c3nc(C)no3)c(C)c2c1] | 207 |
| 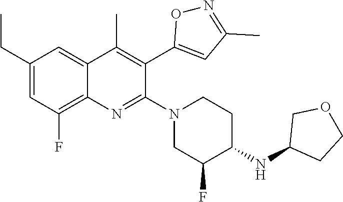 [SMILES: CCc1cc(F)c2nc(N3CC[C@H](N[C@@H]4CCOC4)[C@@H](F)C3)c(-c3cc(C)no3)c(C)c2c1] | 208 |

41. A pharmaceutical composition comprising a compound or pharmaceutically acceptable salt thereof according to claim 2 and a pharmaceutically acceptable carrier.

42. A method for treating a disorder in a subject suffering therefrom, wherein the disorder is one for which antagonism of kappa-opioid receptor (KOR) is therapeutically indicated, comprising administering to the subject a compound or pharmaceutically acceptable salt thereof according to claim 2.

43. A method for treating a disorder in a subject suffering therefrom, comprising administering to the subject a compound or pharmaceutically acceptable salt thereof according to claim 2, wherein the disorder is selected from substance abuse or addiction, psychiatric disorder, obesity and eating disorders, migraine, postnatal depression, neurodegenerative disease or disorder, epilepsy, status epilepticus, seizure, and disruption of sleep associated with pain, a psychiatric disorder, or a drug therapy of a psychiatric disorder.

44. The method according to claim 43, wherein the disorder is substance abuse or addiction.

45. The method according to claim 44, wherein substance abuse or addiction is selected from gambling, drug addiction, drug abuse, alcohol dependence, alcohol abuse, and substance-induced depression or mood disorders.

46. The method according to claim 43, wherein the disorder is a psychiatric disorder.

47. The method according to claim 46, wherein the psychiatric disorder is selected from an anxiety disorder, depressive disorder, mood disorder, schizophrenia spectrum disorders, stress-related disorder, obsessive-compulsive disorder, social phobia, generalized anxiety disorder (GAD), social anxiety disorder, post-traumatic stress disorder (PTSD), personality disorders, and autism spectrum disorders (ASD).

48. The method according to claim 43, wherein the condition is disruption of sleep associated with pain, a psychiatric disorder, or a drug therapy of a psychiatric disorder.

49. The method according to claim 48, wherein the pain is chronic pain or neuropathic pain.

50. The method according to claim 43, wherein the sleep is REM sleep.

51. The method according to claim 43, wherein the disruption of sleep is disturbance of sleep, loss of sleep, or a combination thereof.
